SEQUENCE LISTING (530 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 22 bp, Chlamydomonas reinhardtii
  source 1..22: mol_type = genomic DNA
caaaacgtcgtgagacagtttc

SEQ ID NO: 4 - DNA, 22 bp, Chlamydomonas reinhardtii
  source 1..22: mol_type = genomic DNA
gttttgcagcactctgtcaaac

SEQ ID NO: 5 - skipped

SEQ ID NO: 6 - DNA, 22 bp, synthetic construct
  misc_feature 1..22: note = Synthesized
  source 1..22: mol_type = other DNA
gtatagcatacattatacgaag

SEQ ID NO: 7 - DNA, 22 bp, synthetic construct
  misc_feature 1..22: note = Synthesized
  source 1..22: mol_type = other DNA
catatcgtatgtaatatgcttc

SEQ ID NO: 8 - protein, 354 aa, synthetic construct
  REGION 1..354: note = Synthesized
MNTKYNKEFLLYLAGFVDADGSIYATIAPKQQLKFKHQLQLVFVVAQKTQRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTSRRWFLDKLVDEIGVGYVYDLSTHSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 9 - DNA, 22 bp, synthetic construct
  misc_feature 1..22: note = Synthesized
  source 1..22: mol_type = other DNA
gtatagcatacaatatacgaag

SEQ ID NO: 10 - DNA, 22 bp, synthetic construct
  misc_feature 1..22: note = Synthesized
  source 1..22: mol_type = other DNA
catatcgtatgttatatgcttc

SEQ ID NO: 11 - protein, 354 aa, synthetic construct
  REGION 1..354: note = Synthesized
MNTKYNKEFLLYLAGFVDGDGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGRVTQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 12 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEVLRAGAGSGTGYNKEFLLYLAGFVDGDGSICASIRPCQVAKFKHALELRFTVGQTTRRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 13 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGTITQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 14 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGNISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 15 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATKRRWFLDKLVDEIGVGYVYDLGSVTQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 16 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLAGISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 17 - protein, 354 aa, synthetic construct
  REGION 1..354: note = Synthesized
MNTKYNKEFLLYLAGFVDGDGSIYATIAPKQQLKFKHQLQLVFVVAQKTCRRWFLDKLVDEIGVGYVHDYRQCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTCRRWFLDKLVDEIGVGYVYDLGSVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 18 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 19 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTERRWFLDKLVDEIGVGYVYDLGRIAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 20 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDGLPGSVGGLSPSQASSAASSASSSPGSGISEALRAGAGSGTGYNKEFLLYLAGFVDADGSICASIRPCQVAKFKHALELRFTVGQSTTRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 21 - protein, 354 aa, synthetic construct
  REGION 1..354: note = Synthesized
MNTKYNKEFLLYLAGFVDGDGSIYATIAPKQQLKFKHQLQLVFVVAQKTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGRVAQYNLSEIKPLHNFLTQLRPFLKLKQKQANLVLKIIEQLPSAKESPDKFLEVCTWVDQIAALNDSRTRKTTSETVRAVLDSLSEKKKSSP

SEQ ID NO: 22 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGSISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 23 - protein, 354 aa, synthetic construct
  REGION 1..354: note = Synthesized
MNTKYNKEFLLYLAGFVDGDGSIYATIAPKQQLKFKHQLQLVFVVAQKTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATERRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 24 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQSTKRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 25 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGAISQYNLSEIKPLHNFLTQLQPFLKLKQKQANLVLKIIEQLPSAKGSPDKFLEVCTWVDQIAALNDSKTRKTTSETVRAVLDSLSEKKKSSP

SEQ ID NO: 26 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 27 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGCVYDLGTITQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 28 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGSVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 29 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGRITQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 30 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATKRRWFLDKLVDEIGVGYVYDLGPIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 31 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 32 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGRVSQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 33 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQTTKRRWFLDKLVDEIGVGYVYDLGPVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 34 - DNA, 22 bp, synthetic construct
  misc_feature 1..22: note = Synthesized
  source 1..22: mol_type = other DNA
gtatagcatacagtatacgaag

SEQ ID NO: 35 - DNA, 22 bp, synthetic construct
  misc_feature 1..22: note = Synthesized
  source 1..22: mol_type = other DNA
catatcgtatgtcatatgcttc

SEQ ID NO: 36 - protein, 354 aa, synthetic construct
  REGION 1..354: note = Synthesized
MNTKYNKEFLLYLAGFVDADGSIYATIAPKQQLKFKHQLQLVFVVAQKTRRRWILDKLVDEIGVGYVHDYGKASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 37 - protein, 354 aa, synthetic construct
  REGION 1..354: note = Synthesized
MNTKYNKEFLLYLAGFVDADGSIYATIAPKQQLKFKHQLQLVFVVAQKTRRRWLLDKLVDEIGVGYVHDYRQCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 38 - protein, 354 aa, synthetic construct
  REGION 1..354: note = Synthesized
MNTKYNKEFLLYLAGFVDADGSIYATIAPKQQLKFKHQLQLVFVVAQKTRRRWILDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 39 - protein, 355 aa, synthetic construct
  REGION 1..355: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLDDRRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 40 - protein, 355 aa, synthetic construct
  REGION 1..355: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLDSRRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 41 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGPASYYRLSEIKPLHNFLTQLQPFLKLKQKQANLVLKIIEQLPSAKESPDKFLEVCTWVDQIAALNDSKTRKTTSETVRAVLDSLPGPVGGLSPSQASSAASSASSSPGSGISEALRAGAGSGTGYNKEFLLYLAGFVDSDGSICASIRPCQVAKFKHALELRFTVGQKTCRRWFLDKLADEIGVGHVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 42 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGTCSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 43 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGPASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQTKPLHNFLTQLQPFLKLKQKQANLVLKIIEQLPSAKESPDKFLEVCTWVDQIAALNDSKTRKTTSETVRAVLDSLSEKKKSSP

SEQ ID NO: 44 - DNA, 22 bp, synthetic construct
  misc_feature 1..22: note = Synthesized
  source 1..22: mol_type = other DNA
gtatagcatacattatacgaag

SEQ ID NO: 45 - DNA, 22 bp, synthetic construct
  misc_feature 1..22: note = Synthesized
  source 1..22: mol_type = other DNA
catatcgtatgtaatatgcttc

SEQ ID NO: 46 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYRTASYYRLSEIKPLHNFLTQLQPFLKLKQKQANLVLKIIEQLPSAKESPDKFLEVCTWVDQIAALNDSHTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTKRRWFLDKLVDEIGVGYVYDLGKACQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 47 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTCRRWFLDKLVDEIGVGYVYDLGAASQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 48 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTNRRWFLDKLVDEIGVGYVYDLRSCCQYNLSQTKPLHNFLTQLQPFLKLKQKQANLVLKIIEQLPSAKESPDKFLEVCTWVDQIAALNDSRTRKTTSETVRAVLDSLSEKKKSSP

SEQ ID NO: 49 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLTRCCQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 50 - protein, 354 aa, synthetic construct
  REGION 1..354: note = Synthesized
MNTKYNKEFLLYLAGFVDGDGSIYATIAPKQQLKFKHQLQLVFVVAQITRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTKRRWFLDKLVDEIGVGYVYDLGHASQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 51 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTKRRWFLDKLVDEIGVGYVYDLKHCCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 52 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQGTKRRWFLDKLVDEIGVGYVYDLGKACQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 53 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATKRRWFLDKLVDEIGVGYVYDLRHSCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 54 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLTKCCQYNLSQIKPLHNFLTQLQPFLKLKQKQANLVLKIIEQLPSAKESPDKFLEVCTWVDQIAALNDSTTRKTTSETVRAVLDSLSEKKKSSP

SEQ ID NO: 55 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTCRRWFLDKLVDEIGVGYVYDLGTACQYNLSQIKHLHNFLTQLQPFLKLKQKQANLVLKIIEQLPSAKGSPDKFLEVCTWVDQIAALNDSTTRKTTSETVRAVLDSLSEKKKSSP

SEQ ID NO: 56 - protein, 354 aa, synthetic construct; identical to SEQ ID NO: 8
  REGION 1..354: note = Synthesized

SEQ ID NO: 57 - protein, 354 aa, synthetic construct
  REGION 1..354: note = Synthesized
MNTKYNKEFLLYLAGFVDGDGSIYATIAPKQQLKFKHQLQLVFVVAQLTKRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTKRRWFLDKLVDEIGVGYVYDLKACAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 58 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTGRRWFLDKLVDEIGVGYVYDLGRGCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 59 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLRGGCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 60 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQATSRRWFLDKLVDEIGVGYVYDLGRACQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 61 - protein, 354 aa, synthetic construct
  REGION 1..354: note = Synthesized
MNTKYNKEFLLYLAGFVDADGSIYATIAPKQQLKFKHQLQLVFVVAQLTKRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLRSCCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 62 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATQRRWFLDKLVDEIGVGYVYDLKSGCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 63 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATCRRWFLDKLVDEIGVGYVYDLGTCCQYNLSQIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 64 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTCRRWFLDKLVDEIGVGYVYDLGHACQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 65 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTQRRWFLDKLVDEIGVGYVYDLGNSCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 66 - protein, 354 aa, synthetic construct
  REGION 1..354: note = Synthesized
MNTKYNKEFLLYLAGFVDADGSIYATIAPKQQLKFKHQLQLVFVVAQNTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLERACQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 67 - protein, 354 aa, synthetic construct
  REGION 1..354: note = Synthesized
MNTKYNKEFLLYLAGFVDADGSIYATIAPKQQLKFKHQLQLVFVVAQKTRRRWILDKLVDEIGVGYVHDYGRGSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 68 - DNA, 22 bp, synthetic construct
  misc_feature 1..22: note = Synthesized
  source 1..22: mol_type = other DNA
gtatagcatacgatatacgaag

SEQ ID NO: 69 - DNA, 22 bp, synthetic construct
  misc_feature 1..22: note = Synthesized
  source 1..22: mol_type = other DNA
catatcgtatgctatatgcttc

SEQ ID NO: 70 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 71 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATIRRWFLDKLVDEIGVGYVYDLGRVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 72 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLGHIAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 73 - protein, 354 aa, synthetic construct
  REGION 1..354: note = Synthesized
MNTKYNKEFLLYLAGFVDADGSIYATIAPKQQLKFKHQLQLVFVVAQKTWRRWFLDKLVDEIGVGYVHDYPPASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 74 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGHIAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 75 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLGRVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 76 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTVRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 77 - protein, 354 aa, synthetic construct
  REGION 1..354: note = Synthesized
MNTKYNKEFLLYLAGFVDGDGSIYATIAPKQQLKFKHQLQLVFVVAQKTARRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 78 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTGRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 79 - protein, 354 aa, synthetic construct; identical to SEQ ID NO: 70
  REGION 1..354: note = Synthesized

SEQ ID NO: 80 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTCRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 81 - protein, 354 aa, synthetic construct
  REGION 1..354: note = Synthesized
MNTKYNKEFLLYLAGFVDGDGSIYATIAPKQQLKFKHQLQLVFVVAQKTVRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 82 - protein, 354 aa, synthetic construct
  REGION 1..354: note = Synthesized
MNTKYNKEFLLYLAGFVDGDGSIYATIAPKQQLKFKHQLQLVFVVAQKTVRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTSRRWFLDKLVDEIGVGYVYDLGRISQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 83 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQTTRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 84 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 85 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 86 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQGTRRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 87 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQQTRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 88 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATIRRWFLDKLVDEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 89 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLGRVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 90 - DNA, 22 bp, synthetic construct
  misc_feature 1..22: note = Synthesized
  source 1..22: mol_type = other DNA
gtatagcatacgctatacgaag

SEQ ID NO: 91 - DNA, 22 bp, synthetic construct
  misc_feature 1..22: note = Synthesized
  source 1..22: mol_type = other DNA
catatcgtatgcgatatgcttc

SEQ ID NO: 92 - protein, 354 aa, synthetic construct
  REGION 1..354: note = Synthesized
MNTKYNKEFLLYLAGFVDGDGSIYATIAPKQQLKFKHQLQLVFVVAQHTQRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLGRTAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 93 - protein, 354 aa, synthetic construct
  REGION 1..354: note = Synthesized
MNTKYNKEFLLYLAGFVDADGSIYATIAPKQQLKFKHQLQLVFVVAQQTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLGRVAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 94 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLKRIAQYNLSQIKPLHNFLTQLQPFLKLKQKQANLVLKIIEQLPSAKESPDKFLEVCTWVDQIAALNDSNTRKTTSETVRAVLDSLSEKKKSSP

SEQ ID NO: 95 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTKRRWFLDKLVDEIGVGYVYDLGRVTQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 96 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYRRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGRVAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 97 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGGGYVYDLGRIAQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 98 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLSRVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 99 - protein, 354 aa, synthetic construct
  REGION 1..354: note = Synthesized
MNTKYNKEFLLYLAGFVDADGSIYATIAPKQQLKFKHQLQLVFVVAQHTKRRWFLDKLVDEIGVGYVHDYAPASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLGAISQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 100 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYRRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTIRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 101 - protein, 354 aa, synthetic construct
  REGION 1..354: note = Synthesized
MNTKYNKEFLLYLAGFVDGDGSIYATIAPKQQLKFKHQLQLVFVVAQLTKRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTNRRWFLDKLVDEIGVGYVYDLGRISQYNLSQIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 102 - protein, 354 aa, synthetic construct
  REGION 1..354: note = Synthesized
MNTKYNKEFLLYLAGFVDGDGSIYATIAPKQQLKFKHQLQLVFVVAQLTKRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATKRRWFLDKLADEIGVGYVYDLARISQYNLSQIKPLHNLLTQLQPFLKLKQKQANLVLKIIEQLPSAKESPDKFLEVCTWVDQIAALNDSHTRKTTSETVRAVLDSLSEKKKSSP

SEQ ID NO: 103 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYRHASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGTVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 104 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYRRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGRTAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 105 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTIRRWFLDKLVDEIGVGYVYDLGSVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 106 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTNRRWFLDKLVDEIGVGYVYDLGRVAQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 107 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTVRRWFLDKLVDEIGVGYVYDLGHTAQYNLSQIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 108 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLRRVAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 109 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQLTNRRWFLDKLVDEIGVGYVYDLGRVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 110 - protein, 354 aa, synthetic construct
  REGION 1..354: note = Synthesized
MNTKYNKEFLLYLAGFVDGDGSIYATIAPKQQLKFKHQLQLVFVVAQATRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTIRRWFLDKLVDEIGVGYVYDLGRCAQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 111 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDQIGVGYVYDLGRITQYNLSQIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 112 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTERRWFLDKLVDEIGVGYVYDLGRVAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 113 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTKRRWFLDKLVDEIGVGYVYDLGSISQYNLSQIKPLHNFLTQLQPFLKLKQKQANLVLKIIEQLPSAKESPDKFLEVCTWVDQIAALNDSATRKTTSETVRAVLDSLSEKKKSSP

SEQ ID NO: 114 - protein, 354 aa, synthetic construct
  REGION 1..354: note = Synthesized
MNTKYNKEFLLYLAGFVDGDGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTKRRWFLDKLVDEIGVGYVYDLGGIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 115 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQNTSRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 116 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTIRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 117 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLGRCAQYNLSEIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 118 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATKRRWFLDKLVDEIGVGYVYDLGWIAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 119 - DNA, 22 bp, synthetic construct
  misc_feature 1..22: note = Synthesized
  source 1..22: mol_type = other DNA
gtatagcatacggtatacgaag

SEQ ID NO: 120 - DNA, 22 bp, synthetic construct
  misc_feature 1..22: note = Synthesized
  source 1..22: mol_type = other DNA
catatcgtatgccatatgcttc

SEQ ID NO: 121 - protein, 355 aa, synthetic construct
  REGION 1..355: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTRRRWFLDKLVDEIGVGYVYDLDGRRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 122 - protein, 355 aa, synthetic construct
  REGION 1..355: note = Synthesized
MNTKYNKEFLLYLAGFVDGDGSIYATIAPKQQLKFKHQLQLVFVVAQKTKRRWLLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTRRRWFLDKLVDEIGVGYVYDLDGRRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 123 - protein, 355 aa, synthetic construct; identical to SEQ ID NO: 121
  REGION 1..355: note = Synthesized

SEQ ID NO: 124 - protein, 355 aa, synthetic construct; identical to SEQ ID NO: 121
  REGION 1..355: note = Synthesized

SEQ ID NO: 125 - protein, 355 aa, synthetic construct; identical to SEQ ID NO: 121
  REGION 1..355: note = Synthesized

SEQ ID NO: 126 - protein, 355 aa, synthetic construct; identical to SEQ ID NO: 121
  REGION 1..355: note = Synthesized

SEQ ID NO: 127 - protein, 355 aa, synthetic construct; identical to SEQ ID NO: 121
  REGION 1..355: note = Synthesized

SEQ ID NO: 128 - protein, 355 aa, synthetic construct
  REGION 1..355: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLFLAGFVDADGSICASIRPRQVAKFKHALELRFTVGQKTPRRWFLDKLVDEIGVGYVYDLDGRRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 129 - protein, 355 aa, synthetic construct; identical to SEQ ID NO: 121
  REGION 1..355: note = Synthesized

SEQ ID NO: 130 - protein, 355 aa, synthetic construct; identical to SEQ ID NO: 121
  REGION 1..355: note = Synthesized

SEQ ID NO: 131 - protein, 355 aa, synthetic construct; identical to SEQ ID NO: 121
  REGION 1..355: note = Synthesized

SEQ ID NO: 132 - protein, 355 aa, synthetic construct; identical to SEQ ID NO: 121
  REGION 1..355: note = Synthesized

SEQ ID NO: 133 - protein, 355 aa, synthetic construct; identical to SEQ ID NO: 121
  REGION 1..355: note = Synthesized

SEQ ID NO: 134 - protein, 355 aa, synthetic construct
  REGION 1..355: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGIFEALRGGAGSGTGYNKEFLLFLAGFVVAYGSICASIRPRQVVKFKHPLEVRFTVGQKTPRRWFLVNMVEEIGVGYVYDLDGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 135 - protein, 355 aa, synthetic construct; identical to SEQ ID NO: 121
  REGION 1..355: note = Synthesized

SEQ ID NO: 136 - DNA, 22 bp, synthetic construct
  misc_feature 1..22: note = Synthesized
  source 1..22: mol_type = other DNA
gtatagcatacgttatacgaag

SEQ ID NO: 137 - DNA, 22 bp, synthetic construct
  misc_feature 1..22: note = Synthesized
  source 1..22: mol_type = other DNA
catatcgtatgcaatatgcttc

SEQ ID NO: 138 - protein, 354 aa, synthetic construct
  REGION 1..354: note = Synthesized
MNTKYNKEFLLYLAGFVDGDGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLPRCSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 139 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQLTCRRWFLDKLVDEIGVGYVYDLGKASQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 140 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTQRRWFLDKLVDEIGVGYVYDLGAASQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 141 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTERRWFLDKLVDEIGVGYVYDLGKAAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 142 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLPKCAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 143 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTCRRWFLDKLVDEIGVGYVYDLGKASQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 144 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLTRCAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 145 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLAKCAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 146 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTERRWFLDKLVDEIGVGYVYDLGRAAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 147 - protein, 354 aa, synthetic construct
  REGION 1..354: note = Synthesized
MNTKYNKEFLLYLAGFVDGDGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLGRAAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 148 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLRRAAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 149 - protein, 354 aa, synthetic construct
  REGION 1..354: note = Synthesized
MNTKYNKEFLLYLAGFVDGDGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTERRWFLDKLVDEIGVGYVYDLGKCTQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 150 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTERRWFLDKLVDEIGVGYVYDLGKCTQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 151 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQLTERRWFLDKLVDEIGVGYVYDLGRCAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 152 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGRASQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 153 - protein, 354 aa, synthetic construct
  REGION 1..354: note = Synthesized
MNTKYNKEFLLYLAGFVDGDGSIYATIAPKQQLKFKHQLQLVFVVAQKTVRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTARRWFLDKLVDEIGVGYVYDLGRAAQYNLSQIKPLYNFLTQLQPFLKLKQKQANLVLKIIEQLPSAKESPDKFLEVCTWVDQIAALNDSKTRKTTSETVRAVLDSLSEKKKSSP

SEQ ID NO: 154 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTARRWFLDKLVDEIGVGYVYDLGRAAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 155 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLNRCSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 156 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGKSSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 157 - DNA, 22 bp, synthetic construct
  misc_feature 1..22: note = Synthesized
  source 1..22: mol_type = other DNA
gtatagcatataatatacgaag

SEQ ID NO: 158 - DNA, 22 bp, synthetic construct
  misc_feature 1..22: note = Synthesized
  source 1..22: mol_type = other DNA
catatcgtatattatatgcttc

SEQ ID NO: 159 - protein, 354 aa, synthetic construct
  REGION 1..354: note = Synthesized
MNTKYNKEFLLYLAGFVDGDGSIYATIAPKQQLKFKHQLQLVFVVAQKTTRRWFLDKLVDEIGVGYVHDYGRASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDSDGSICASIRPCQVAKFKHALELRFTVGQSTRRRWFLDKLVDEIGVGYVYDLGTIAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 160 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLGRCAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 161 - protein, 354 aa, synthetic construct
  REGION 1..354: note = Synthesized
MNTKYNKEFLLYLAGFVDADGSIYATIAPKQQLKFKHQLQLVFVVAQHTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGQVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 162 - protein, 354 aa, synthetic construct
  REGION 1..354: note = Synthesized
MNTKYNKEFLLYLAGFVDADGSIYATIAPKQQLKFKHQLQLVFVVAQATIRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGGVAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 163 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYGRASYYRLSEIKPLHNFLTQLQPFLKLKQKQANLVLKIIEQLPSAKESPDKFLEVCTWVDQIAALNDSRTRKTTSETVRAVLDGLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLKAIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 164 - protein, 354 aa, synthetic construct
  REGION 1..354: note = Synthesized
MNTKYNKEFLLYLAGFVDADGSIYATIAPKQQLKFKHQLQLVFVVVQHTRRRWFLDKLVDEIGVGYVHDYGAASYYRLSQIKPLHNFLTQLQPFLKLKQEQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATARRWFLDKLVDEIGVGYVYDLGGVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 165 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYKGTAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 166 - protein, 354 aa, synthetic construct
  REGION 1..354: note = Synthesized
MNTKYNKEFLLYLAGFVDADGSIYATIAPKQQLKFKHQLQLVFVVAQKTGRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATCRRWFLDKLADEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 167 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYSGASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQNTKRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 168 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTTRRWFLDKLVDEIGVGYVYDLGYVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 169 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYHAASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 170 - protein, 354 aa, synthetic construct
  REGION 1..354: note = Synthesized
MNTKYNKEFLLYLAGFVDADGSIYATIAPKQQLKFKHQLQLVFVVAQKTDRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTTRRWFLDKLVDEIGVGYVYDLSGVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 171 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYNQAAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTTRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 172 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGHCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLGGISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 173 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGLCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGRVTQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 174 - protein, 354 aa, synthetic construct
  REGION 1..354: note = Synthesized
MNTKYNKEFLLYLAGFVDGDGSIYATIAPKQQLKFKHQLQLVFVVAQATCRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTKRRWFLDKLVDEIGVGYVYDLGSVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 175 - protein, 354 aa, synthetic construct
  REGION 1..354: note = Synthesized
MNTKYNKEFLLYLAGFVDGDGSIYATIAPKQQLKFKHQLQLVFVVAQKTVRRWFLDKLVDEIGVGYVHDYGACSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAAASASSSPGSGISEALRAGAGSGTGYNKEFLLYLAGFVDSDGSICASIRPCQVAKFKHALELRFTVGQSTKRRWFLDKLVDEIGVGYVYDLGNVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 176 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGSVSQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 177 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGSASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLGRCAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 178 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYHRCSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGKVSQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 179 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGQVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 180 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLADEIGVGYVYDLRSIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 181 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTTRRWFLDKLVDEIGVGYVYDLGSVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 182 - protein, 354 aa, synthetic construct
  REGION 1..354: note = Synthesized
MNTKYNKEFLLYLAGFVDADGSIYATIAPKQQLKFKHQLQLVFVVAQSTQRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRVSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 183 - protein, 354 aa, synthetic construct
  REGION 1..354: note = Synthesized
MNTKYNKEFLLYLAGFVDGDGSIYATIAPKQQLKFKHQLQLVFVVAQQTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVSQSTKRRWFLDKLVDEIGVGYVYDLGYVAQYNLSQIKPLHNFLTQLQPFLKLKQKQANLVLKIIEQLPSAKEFPDKFLEVCTWVDQIAALNDSRTRKTTSETVRAVLDSLSEKKKSSP

SEQ ID NO: 184 - DNA, 22 bp, synthetic construct
  misc_feature 1..22: note = Synthesized
  source 1..22: mol_type = other DNA
gtatagcatatagtatacgaag

SEQ ID NO: 185 - DNA, 22 bp, synthetic construct
  misc_feature 1..22: note = Synthesized
  source 1..22: mol_type = other DNA
catatcgtatatcatatgcttc

SEQ ID NO: 186 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 187 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVSQKTCRRWFLDKLVDEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 188 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLADEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 189 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGGASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 190 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGSASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTRRRWFLDKLADEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 191 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFRHALELRFTVGQKTCRRWFLDKLADEIGVGYVYDLGSRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 192 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGGCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAVSGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 193 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYRACSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 194 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 195 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLADEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 196 - protein, 354 aa, synthetic construct
  REGION 1..354: note = Synthesized
  SITE 241: note = misc_feature - Xaa can be any naturally occurring amino acid
MNTKYNKEFLLYLAGFVDADGSIYATIAPKQQLKFKHQLQLVFVVAQHTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTXRRWFLDKLADEIGVGYVYDLGGRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 197 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGPASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 198 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYHQASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLADEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 199 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLADEIGVGYVYDLGGRSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 200 - DNA, 22 bp, synthetic construct
  misc_feature 1..22: note = Synthesized
  source 1..22: mol_type = other DNA
gtatagcatatattatacgaag

SEQ ID NO: 201 - DNA, 22 bp, synthetic construct
  misc_feature 1..22: note = Synthesized
  source 1..22: mol_type = other DNA
catatcgtatataatatgcttc

SEQ ID NO: 202 - protein, 354 aa, synthetic construct
  REGION 1..354: note = Synthesized
MNTKYNKEFLLYLAGFVDADGSIYATIAPKQQLKFKHQLQLVFVVAQHTNRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLKTCCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 203 - protein, 354 aa, synthetic construct
  REGION 1..354: note = Synthesized
MNTKYNKEFLLYLAGFVDGDGSIYATIAPKQQLKFKHQLQLVFVVAQHTCRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTKRRWFLDKLVDEIGVGYVYDLKACCQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 204 - protein, 354 aa, synthetic construct
  REGION 1..354: note = Synthesized
MNTKYNKEFLLYLAGFVDADGSIYATIAPKQQLKFKHQLQLVFVVAQCTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATCRRWFLDKLVDEIGVGYVYDLKRCAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 205 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTSRRWFLDKLVDEIGVGYVYDLKSASQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 206 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLERCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 207 - protein, 354 aa, synthetic construct
  REGION 1..354: note = Synthesized
MNTKYNKEFLLYLAGFVDADGSIYATIAPKQQLKFKHQLQLVFVVAQATKRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLIKCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 208 - protein, 354 aa, synthetic construct
  REGION 1..354: note = Synthesized
MNTKYNKEFLLYLAGFVDADGSIYATIAPKQQLKFKHQLQLVFVVAQATKRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGKCAQYNLSQIKPLHNFLTQLQPFLKLKQKQANLVLKIIEQLPSAKESPDKFLEVCTWVDQIAALNDSPTRKTTSETVRAVLDSLSEKKKSSP

SEQ ID NO: 209 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLKACAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 210 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTQRRWFLDKLVDEIGVGYVYDLRKCCQYNLSQIKPLHNFLTQLQPFLKLKQKQANLVLKIIEQLPSAKESPDKFLEVCTWVDQIAALNDSNTRKTTSETVRAVLDSLSEKKKSSP

SEQ ID NO: 211 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYGRASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLEGCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 212 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYHAASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLRRCCQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 213 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYINCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTNRRWFLDKLVDEIGVGYVYDLRGSCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 214 - protein, 354 aa, synthetic construct
  REGION 1..354: note = Synthesized
MNTKYNKEFLLYLAGFVDADGSIYATIAPKQQLKFKHQLQLVFVVAQDTKRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLRGGSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 215 - protein, 354 aa, synthetic construct
  REGION 1..354: note = Synthesized
  SITE 19: note = misc_feature - Xaa can be any naturally occurring amino acid
MNTKYNKEFLLYLAGFVDXDGSIYATIAPKQQLKFKHQLQLVFVVAQKTRRRWFLDKLVDEIGVGYVHDYGQSSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTSRRWFLDKLVDEIGVGYVYDLGRCCQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 216 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYGRASYYRLSQIKPLHNFLTQLQPFLKLKQKQANLVLKIIEQLPSAKESPDKFLEVCTWVDQIAALNDSSTRKTTSETVRAVLDSLPGSVGGLSPSQASSAASSASSSPGSGISEALRAGAGSGTGYNKEFLLYLAGFVDGDGSICASIRPCQVAKFKHALELRFTVGQRTCRRWFLDKLADEIGVGYVYDLKTCCQYNLSKIKPLHNFLTQLQPFLKLKQKQANLVLKIIEQLPSAKESPDKFLEVCTWVDQIAALNDSNTRKTTSETVRAVLDSLSEKKKSSP

SEQ ID NO: 217 - protein, 354 aa, synthetic construct
  REGION 1..354: note = Synthesized
MNTKYNKEFLLYLAGFVDADGSIYATIAPKQQLKFKHQLQLVFVVAQTTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTNRRWFLDKLVDEIGVGYVYDLGGCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 218 - protein, 354 aa, synthetic construct
  REGION 1..354: note = Synthesized
MNTKYNKEFLLYLAGFVDADGSIYATIAPKQQLKFKHQLQLVFVVAQKTKRRWFLDKLVDEIGVGYVHDYGRASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLRNCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 219 - protein, 180 aa, synthetic construct
  REGION 1..180: note = Synthesized
MNTKYNKEFLLYLAGFVDADGSIYATIAPKQQLKFKHQLQLVFVVAQKTVRRWFLDKLVDEIGVGYVHDYGAASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGS

SEQ ID NO: 220 - DNA, 22 bp, synthetic construct
  misc_feature 1..22: note = Synthesized
  source 1..22: mol_type = other DNA
gtatagcatatgatatacgaag

SEQ ID NO: 221 - DNA, 22 bp, synthetic construct
  misc_feature 1..22: note = Synthesized
  source 1..22: mol_type = other DNA
catatcgtatactatatgcttc

SEQ ID NO: 222 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYGTASYYRLSQIKPLHNFLTQLRPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 223 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQRTIRRWFLDKLADEIGVGYVYDLGHIAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 224 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGSASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTSRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 225 - protein, 354 aa, synthetic construct
  REGION 1..354: note = Synthesized
MNTKYNKEFLLYLAGFVDSDGSIYATIAPKQQLKFKHQLQLVFVVAQKTERRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 226 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQKTRRRWFLDKLVDEIGVGYVYDLGRIAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 227 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTIRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 228 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGRVAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 229 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGAASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLADEIGVGYVYDLGHISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 230 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGTASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTIRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 231 - protein, 354 aa, synthetic construct
  REGION 1..354: note = Synthesized
MNTKYNKEFLLYLAGFVDGDGSIYATIAPKQQLKFKHQLQLVFVVAQLTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTARRWFLDKLVDEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 232 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTSRRWFLDKLVDEIGVGYVYDLGHVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 233 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRVAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 234 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGHISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 235 - protein, 354 aa, synthetic construct
  REGION 1..354: note = Synthesized
MNTKYNKEFLLYLAGFVDGDGSIYATIAPKQQLKFKHQLQLVFVVAQATRRRWFLDKLVDEIGVGYVHDYGRASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGRVSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 236 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGTASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 237 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGHIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 238 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGRITQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 239 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGSSSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGHVSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 240 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGSASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRVAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 241 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYGRASYYRLSQIKPLHNLLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLGRVAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 242 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTCRRWFLDKLVDEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 243 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGKASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGPGISEALRAGAGSGTGYNKEFLLYLAGFVDGDGSICASIRPCQVAKFKHALELRFTVGQKTIRRWFLDKLADEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 244 - DNA, 22 bp, synthetic construct
  misc_feature 1..22: note = Synthesized
  source 1..22: mol_type = other DNA
gtatagcatatggtatacgaag

SEQ ID NO: 245 - DNA, 22 bp, synthetic construct
  misc_feature 1..22: note = Synthesized
  source 1..22: mol_type = other DNA
catatcgtataccatatgcttc

SEQ ID NO: 246 - protein, 355 aa, synthetic construct
  REGION 1..355: note = Synthesized
MNTKYNKEFLLYLAGFVDGDGSIYATIAPKQQLKFKHQLQLVFVVAQKTRRRWFLDKLVDEIGVGYVHDYGPASYYRLSEIEPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTRRRWFLDKLVDEIGVGYVYDLDGRRSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 247 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGCCYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTRRRWFLDKLVDEIGVGYVYDLGGRSQYNLSRIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 248 - DNA, 22 bp, synthetic construct
  misc_feature 1..22: note = Synthesized
  source 1..22: mol_type = other DNA
gtatagcatttggtatacgaag

SEQ ID NO: 249 - DNA, 22 bp, synthetic construct
  misc_feature 1..22: note = Synthesized
  source 1..22: mol_type = other DNA
catatcgtaaaccatatgcttc

SEQ ID NO: 250 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGQISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 251 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGHVYDLKKISQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 252 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTTRRWFLDKLVDEIGVGYVYDLGTIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 253 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTERRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 254 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGHIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 255 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGAIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 256 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGSVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 257 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGTISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 258 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 259 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGKIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 260 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVEGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGKISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 261 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGKISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 262 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGSVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 263 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTTRRWFLDKLVDEIGVGYVYDLGAISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 264 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGRISQYNLSQIKPLRNFLTQLQPFLKLKQKQANLVLKIIEQLPSAKESPDKFLEVCTWVDQIAALNDSRTRKTTSETVRAVLDSLSEKKKSSP

SEQ ID NO: 265 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGTISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 266 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSATSSASSSPGSGISEALRAGAGSGTGYNKEFLLYLAGFVDADGSICASIRPCQVAKFKHALELRFTVGQSTRRRWFLDKLVDEIGVGYVYDLGAVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 267 - DNA, 22 bp, synthetic construct
  misc_feature 1..22: note = Synthesized
  source 1..22: mol_type = other DNA
gtatagcatgcaatatacgaag

SEQ ID NO: 268 - DNA, 22 bp, synthetic construct
  misc_feature 1..22: note = Synthesized
  source 1..22: mol_type = other DNA
catatcgtacgttatatgcttc

SEQ ID NO: 269 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGPTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGGCAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 270 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYNSTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTCRRWFLDKLVDEIGVGYVYDLGSVSQYTLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 271 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYTNTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCPVAKFKHALELRFTVGQATRRRWFLDKLVDEIGVGYVYDLGACAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 272 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYRQVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGTCSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 273 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYSGVCYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGEVSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 274 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYRGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLRNIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 275 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGATSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTTRRWFLDKLVDEIGVGYVYDLGSVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 276 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYRNVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGGVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 277 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYRGVAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTKRRWFLDKLVDEIGVGYVYDLGGISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 278 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGATSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGGVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 279 - protein, 325 aa, synthetic construct
  REGION 1..325: note = Synthesized
KQQLKFKHQLQLVFVVAQKTRRRWFLDKLVDEIGVGYVHDYSTVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGKITQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 280 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGTSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGHISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 281 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLAHVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 282 - protein, 354 aa, synthetic construct
  REGION 1..354: note = Synthesized
MNTKYNKEFLLYLAGFVDGDGSIYATIAPKQQLKFKHQLQLVFVVAQKTCRRWFLDKLVDEIGVGYVHDYRMVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQTTKRRWFLDKLVDEIGVGYVYDLGRVSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 283 - protein, 354 aa, synthetic construct
  REGION 1..354: note = Synthesized
MNTKYNKEFLLYLAGFVDGDGSIYATIAPKQQLKFKHQLQLVFVVAQKTLRRWFLDKLVDEIGVGYVHDYRMVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGRISQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 284 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYHATSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGCCSQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 285 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYTRVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTKRRWFLDKLVDEIGVGYVYDLGKVAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 286 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRVTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGSISQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 287 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSSTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGYVAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 288 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGPTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 289 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYTNVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTRRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 290 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYRNTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLGKIAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 291 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLHKIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 292 - DNA, 22 bp, synthetic construct
  misc_feature 1..22: note = Synthesized
  source 1..22: mol_type = other DNA
gtatagcatgcattatacgaag

SEQ ID NO: 293 - DNA, 22 bp, synthetic construct
  misc_feature 1..22: note = Synthesized
  source 1..22: mol_type = other DNA
catatcgtacgtaatatgcttc

SEQ ID NO: 294 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYATTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLKACSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 295 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYHGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLRGGCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 296 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYHSTSYYRLSQIKPLHNFLTQLQPFLKLKQKQANLVLKIIEQLPSAKESPDKFLEVCTWVDQIAALNDSHTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTSRRWFLDKLVDEIGVGYVYDLANCCQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 297 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRQTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQATRRRWFLDKLVDEIGVGYVYDLKTGCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 298 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYTRTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLKGSSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 299 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYHQTSYYRLSQIKPLHNFLTQLQPFLKLKQKQANLVLKIIEQLPSAKESPDKFLEVCTWVDQIAALNDSKTRKITSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLKTCAQYNLSEIKPLHNFLTQLQPFLKLKQKQANLVLKIIEQLPSAKESPDKFLEACTWVDQIAALNDSKTRKTTSETVRAVLDSLSEKKKSSP

SEQ ID NO: 300 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYRTTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGSGSQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 301 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYRRVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLKGASQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 302 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGTVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTQRRWFLDKLVDEIGVGYVYDLGRSCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 303 - protein, 354 aa, synthetic construct
  REGION 1..354: note = Synthesized
MNTKYNKEFLLYLAGFVDADGSIYATIAPKQQLKFKHQLQLVFVVAQKTKRRWFLDKLVDEIGVGYVHDYNGTAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATHRRWFLDKLVDEIGVGYVYDLRRCAQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 304 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYSRTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQLTRRRWFLDKLVDEIGVGYVYDLGHSCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 305 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGRTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTKRRWFLDKLVDEIGVGYVYDLTQCCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 306 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYRNCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTKRRWFLDKLVDEIGVGYVYDLRRSCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 307 - protein, 354 aa, synthetic construct
  REGION 1..354: note = Synthesized
MNTKYNKEFLLYLAGFVDGDGSIYATIAPKQQLKFKHQLQLVFVVAQHTRRRWFLDKLVDEIGVGYVHDYNGTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQLTKRRWFLDKLVDEIGVGYVYDLRACCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 308 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYRNTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQITKRRWFLDKLVDEIGVGYVYDLGNSCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 309 - protein, 354 aa, synthetic construct
  REGION 1..354: note = Synthesized
MNTKYNKEFLLYLAGFVDGDGSIYATIAPKQQLKFKHQLQLVFVVAQRTRRRWFLDKLVDEIGVGYVHDYTGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQATKRRWFLDKLVDEIGVGYVYDLKACCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 310 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYHATSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTVRRWFLDKLVDEIGVGYVYDLRKCSQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 311 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRGVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATSRRWFLDKLVDEIGVGYVYDLHGACQYNLSQIKPLHNFLTQLQPFLKLKQKQANLVLKIIEQLPSAKESPDKFLEVCTWVDQIAALNDSHTRKTTSETVRAVLDSLSEKKKSSP

SEQ ID NO: 312 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYAATSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATHRRWFLDKLVDEIGVGYVYDLGTSAQYNLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 313 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYASTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQHTKRRWFLDKLVDEIGVGYVYDLYRCCQYNLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 314 - DNA, 22 bp, synthetic construct
  misc_feature 1..22: note = Synthesized
  source 1..22: mol_type = other DNA
gtatagcatgcgatatacgaag

SEQ ID NO: 315 - DNA, 22 bp, synthetic construct
  misc_feature 1..22: note = Synthesized
  source 1..22: mol_type = other DNA
catatcgtacgctatatgcttc

SEQ ID NO: 316 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYRNVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQKTCRRWFLDKLVDEIGVGYVYDLGRVSQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 317 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQTTRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 318 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYSGTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 319 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRAVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 320 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYAGIAYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 321 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRGVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQQTRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 322 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 323 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQQTRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 324 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYNRVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGRISQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 325 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYRQVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSICASIRPCQVAKFKHALELRFTVGQATRRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 326 - DNA, 22 bp, synthetic construct
  misc_feature 1..22: note = Synthesized
  source 1..22: mol_type = other DNA
gtatagcatgcagtatacgaag

SEQ ID NO: 327 - DNA, 22 bp, synthetic construct
  misc_feature 1..22: note = Synthesized
  source 1..22: mol_type = other DNA
catatcgtacgtcatatgcttc

SEQ ID NO: 328 - protein, 351 aa, synthetic construct
  REGION 1..351: note = Synthesized
MNTKYNKEFLLLAFVDADSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 329 - protein, 351 aa, synthetic construct
  REGION 1..351: note = Synthesized
MNTKYNKEFLLLAFVDADSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 330 - protein, 351 aa, synthetic construct
  REGION 1..351: note = Synthesized
MNTKYNKEFLLLAFVDADSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQHTRRRWFLDKLVDEIGVGYVYDLGRTSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 331 - DNA, 22 bp, synthetic construct
  misc_feature 1..22: note = Synthesized
  source 1..22: mol_type = other DNA
gtatagcattcaatatacgaag

SEQ ID NO: 332 - DNA, 22 bp, synthetic construct
  misc_feature 1..22: note = Synthesized
  source 1..22: mol_type = other DNA
catatcgtaagttatatgcttc

SEQ ID NO: 333 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 334 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYRSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGQIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 335 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYGSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 336 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGPVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGRIAQYNLSEIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 337 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGTVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 338 - protein, 354 aa, synthetic construct
  REGION 1..354: note = Synthesized
MNTKYNKEFLLYLAGFVDADGSIYATIAPKQQLKFKHQLQLVFVVAQKTTRRWFLDKLVDEIGVGYVHDYGRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTCRRWFLDKLVDEIGVGYVYDLGNIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 339 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYTGVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGQISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 340 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDSDGSICASIRPCQVAKFKHALELRFTVGQSTKRRWFLDKLVDEIGVGYVYDLGSIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 341 - DNA, 22 bp, synthetic construct
  misc_feature 1..22: note = Synthesized
  source 1..22: mol_type = other DNA
gtatagcatttaatatacgaag

SEQ ID NO: 342 - DNA, 22 bp, synthetic construct
  misc_feature 1..22: note = Synthesized
  source 1..22: mol_type = other DNA
catatcgtaaattatatgcttc

SEQ ID NO: 343 - protein, 354 aa, synthetic construct
  REGION 1..354: note = Synthesized
MNTKYNKEFLLYLAGFVDGDGSIYATIAPKQQLKFKHQLQLVFVVAQNTRRRWFLDKLVDEIGVGYVHDYGTVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQKTCRRWFLDKLVDEIGVGYVYDLGKISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 344 - protein, 354 aa, synthetic construct
  REGION 1..354: note = Synthesized
MNTKYNKEFLLYLAGFVDSDGSIYATIAPKQQLKFKHQLQLVFVVAQKTVRRWFLDKLVDEIGVGYVHDYRSVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTKRRWFLDKLVDEIGVGYVYDLGRIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 345 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYNNVAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGYVYDLGKISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 346 - protein, 354 aa, synthetic construct
  REGION 1..354: note = Synthesized
MNTKYNKEFLLYLAGFVDGDGSIYATIAPKQQLKFKHQLQLVFVVAQKTKRRWFLDKLVDEIGVGYVHDYGSVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGAISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 347 - protein, 354 aa, synthetic construct
  REGION 1..354: note = Synthesized
MNTKYNKEFLLYLAGFVDADGSIYATIAPKQQLKFKHQLQLVFVVAQKTKRRWFLDKLVDEIGVGYVHDYNRVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGKIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 348 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYSDVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQSTRRRWFLDKLVDEIGVGHVYDLGSIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 349 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYGTVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTTRRWFLDKLVDEIGVGYVYDLGRVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 350 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYRRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATKRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 351 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYAQVAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQSTKRRWFLDKLVDEIGVGYVYDLGRISQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 352 - protein, 354 aa, synthetic construct
  REGION 1..354: note = Synthesized
MNTKYNKEFLLYLAGFVDADGSIYATIAPKQQLKFKHQLQLVFVVAQKTKRRWFLDKLVDEIGVGYVHDYGTVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTERRWFLDKLVDEIGVGYVYDLGQIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 353 - protein, 354 aa, synthetic construct
  REGION 1..354: note = Synthesized
MNTKYNKEFLLYLAGFVDGDGSIYATIAPKQQLKFKHQLQLVFVVAQKTRRRWFLDKLVDEIGVGYVHDYNKVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDSDGSICASIRPCQVAKFKHALELRFTVGQSTKRRWFLDKLVDEIGVGYVYDLGSIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 354 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGAVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQATRRRWFLDKLVDEIGVGYVYDLGTIAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 355 - protein, 354 aa, synthetic construct; identical to SEQ ID NO: 345
  REGION 1..354: note = Synthesized

SEQ ID NO: 356 - protein, 354 aa, synthetic construct; identical to SEQ ID NO: 343
  REGION 1..354: note = Synthesized

SEQ ID NO: 357 - protein, 354 aa, synthetic construct
  REGION 1..354: note = Synthesized
MNTKYNKEFLLYLAGFVDGDGSIYATIAPKQQLKFKHQLQLVFVVAQKTSRRWFLDKLVDEIGVGYVHDYGSVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDADGSICASIRPCQVAKFKHALELRFTVGQTTKRRWFLDKLVDEIGVGYVYDLGRVAQYNLSQIKPLHNFLTQLQPFLKLKQKQANLVLKIIEQLPSAKESPDKFLEVCTWVDQIAALNDSRTRKTTSETVRAVLDSLSEKKKSSP

SEQ ID NO: 358 - DNA, 22 bp, synthetic construct
  misc_feature 1..22: note = Synthesized
  source 1..22: mol_type = other DNA
gtatagcatgtaatatacgaag

SEQ ID NO: 359 - DNA, 22 bp, synthetic construct
  misc_feature 1..22: note = Synthesized
  source 1..22: mol_type = other DNA
catatcgtacattatatgcttc

SEQ ID NO: 360 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSQVTYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 361 - protein, 354 aa, synthetic construct
  REGION 1..354: note = Synthesized
MNTKYNKEFLLYLAGFVDADGSIYATIAPKQQLKFKHQLQLVFVVAQATRRRWFLDKLVDEIGVGYVHDYTNVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 362 - protein, 354 aa, synthetic construct
  REGION 1..354: note = Synthesized
MNTKYNKEFLLYLAGFVDSDGSIYATIAPKQQLKFKHQLQLVFVVAQKTARRWFLDKLVDEIGVGYVHDYASVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 363 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYRSVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 364 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYAKVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 365 - protein, 354 aa, synthetic construct
  REGION 1..354: note = Synthesized
MNTKYNKEFLLYLAGFVDADGSIYATIAPKQQLKFKHQLQLVFVVAQRTRRRWFLDKLVDEIGVGYVHDYNAVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 366 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYSHCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 367 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYAHVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 368 - protein, 354 aa, synthetic construct
  REGION 1..354: note = Synthesized
MNTKYNKEFLLYLAGFVDSDGSIYATIAPKQQLKFKHQLQLVFVVAQRTKRRWFLDKLVDEIGVGYVHDYTGVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 369 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYHSVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 370 - protein, 354 aa, synthetic construct
  REGION 1..354: note = Synthesized
MNTKYNKEFLLYLAGFVDSDGSIYATIAPKQQLKFKHQLQLVFVVAQNTRRRWFLDKLVDEIGVGYVHDYKRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 371 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYRAVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 372 - protein, 354 aa, synthetic construct
  REGION 1..354: note = Synthesized
MNTKYNKEFLLYLAGFVDSDGSIYATIAPKQQLKFKHQLQLVFVVAQKTARRWFLDKLVDEIGVGYVHDYNRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 373 - protein, 354 aa, synthetic construct
  REGION 1..354: note = Synthesized
MNTKYNKEFLLYLAGFVDADGSIYATIAPKQQLKFKHQLQLVFVVAQSTKRRWFLDKLVDEIGVGYVHDYHSVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 374 - protein, 354 aa, synthetic construct
  REGION 1..354: note = Synthesized
MNTKYNKEFLLYLAGFVDADGSIYATIAPKQQLKFKHQLQLVFVVAQSTARRWFLDKLVDEIGVGYVHDYGTVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 375 - protein, 354 aa, synthetic construct; identical to SEQ ID NO: 367
  REGION 1..354: note = Synthesized

SEQ ID NO: 376 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYSTVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 377 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYSDVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 378 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYSGVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 379 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYSSVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 380 - protein, 354 aa, synthetic construct
  REGION 1..354: note = Synthesized
MNTKYNKEFLLYLAGFVDADGSIYATIAPKQQLKFKHQLQLVFVVAQKTCRRWFLDKLVDEIGVGYVHDYRTVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 381 - protein, 354 aa, synthetic construct
  REGION 1..354: note = Synthesized
MNTKYNKEFLLYLAGFVDSDGSIYATIAPKQQLKFKHQLQLVFVVAQNTRRRWFLDKLVDEIGVGYVHDYGRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 382 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYRNVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 383 - protein, 354 aa, synthetic construct
  REGION 1..354: note = Synthesized
MNTKYNKEFLLYLAGFVDSDGSIYATIAPKQQLKFKHQLQLVFVVAQTTRRRWFLDKLVDEIGVGYVHDYGTVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 384 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYRSVAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 385 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYRHVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 386 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYNYISYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 387 - protein, 354 aa, synthetic construct
  REGION 1..354: note = Synthesized
MNTKYNKEFLLYLAGFVDADGSIYATIAPKQQLKFKHQLQLVFVVAQRTRRRWFLDKLVDEIGVGYVHDYATCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 388 - protein, 354 aa, synthetic construct
  REGION 1..354: note = Synthesized
MNTKYNKEFLLYLAGFVDSDGSIYATIAPKQQLKFKHQLQLVFVVAQRTCRRWFLDKLVDEIGVGYVHDYSNVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 389 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYHPTSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 390 - DNA, 22 bp, synthetic construct
  misc_feature 1..22: note = Synthesized
  source 1..22: mol_type = other DNA
gtatagcatgtagtatacgaag

SEQ ID NO: 391 - DNA, 22 bp, synthetic construct
  misc_feature 1..22: note = Synthesized
  source 1..22: mol_type = other DNA
catatcgtacatcatatgcttc

SEQ ID NO: 392 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSNRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 393 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYDGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 394 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 395 - protein, 354 aa, synthetic construct
  REGION 1..354: note = Synthesized
MNTKYNKEFLLYLAGFVDSDGSIYATIAPKQQLKFKHQLQLVFVVAQKTCRRWFLDKLVDEIGVGYVHDYSNRSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 396 - protein, 354 aa, synthetic construct; identical to SEQ ID NO: 394
  REGION 1..354: note = Synthesized

SEQ ID NO: 397 - protein, 354 aa, synthetic construct; identical to SEQ ID NO: 392
  REGION 1..354: note = Synthesized

SEQ ID NO: 398 - protein, 354 aa, synthetic construct; identical to SEQ ID NO: 395
  REGION 1..354: note = Synthesized

SEQ ID NO: 399 - protein, 354 aa, synthetic construct; identical to SEQ ID NO: 393
  REGION 1..354: note = Synthesized

SEQ ID NO: 400 - DNA, 22 bp, synthetic construct
  misc_feature 1..22: note = Synthesized
  source 1..22: mol_type = other DNA
gtatagcatgtattatacgaag

SEQ ID NO: 401 - DNA, 22 bp, synthetic construct
  misc_feature 1..22: note = Synthesized
  source 1..22: mol_type = other DNA
catatcgtacataatatgcttc

SEQ ID NO: 402 - protein, 354 aa, synthetic construct
  REGION 1..354: note = Synthesized
MNTKYNKEFLLYLAGFVDSDGSIYATIAPKQQLKFKHQLQLVFVVAQKTVRRWFLDKLVDEIGVGYVHDYTKCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 403 - protein, 354 aa, synthetic construct
  REGION 1..354: note = Synthesized
MNTKYNKEFLLYLAGFVDSDGSIYATIAPKQQLKFKHQLQLVFVVAQHTSRRWFLDKLVDEIGVGYVHDYGYCSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 404 - protein, 354 aa, synthetic construct
  REGION 1..354: note = Synthesized
MNTKYNKEFLLYLAGFVDSDGSIYATIAPKQQLKFKHQLQLVFVVAQKTCRRWFLDKLVDEIGVGYVHDYGWCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 405 - protein, 354 aa, synthetic construct
  REGION 1..354: note = Synthesized
MNTKYNKEFLLYLAGFVDSDGSIYATIAPKQQLKFKHQLQLVFVVAQHTSRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 406 - protein, 354 aa, synthetic construct
  REGION 1..354: note = Synthesized
MNTKYNKEFLLYLAGFVDSDGSIYATIAPKQQLKFKHQLQLVFVVAQGTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 407 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYKNCCYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 408 - protein, 354 aa, synthetic construct
  REGION 1..354: note = Synthesized
MNTKYNKEFLLYLAGFVDSDGSIYATIAPKQQLKFKHQLQLVFVVAQATQRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 409 - protein, 354 aa, synthetic construct
  REGION 1..354: note = Synthesized
MNTKYNKEFLLYLAGFVDADGSIYATIAPKQQLKFKHQLQLVFVVAQTTRRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 410 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYASCAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 411 - protein, 354 aa, synthetic construct
  REGION 1..354: note = Synthesized
MNTKYNKEFLLYLAGFVDSDGSIYATIAPKQQLKFKHQLQLVFVVAQKTTRRWFLDKLVDEIGVGYVHDYHTASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 412 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYYGSSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 413 - protein, 354 aa, synthetic construct
  REGION 1..354: note = Synthesized
MNTKYNKEFLLYLAGFVDSDGSIYATIAPKQQLKFKHQLQLVFVVAQMTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 414 - protein, 354 aa, synthetic construct
  REGION 1..354: note = Synthesized
MNTKYNKEFLLYLAGFVDADGSIYATIAPKQQLKFKHQLQLVFVVAQSTGRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 415 - protein, 354 aa, synthetic construct
  REGION 1..354: note = Synthesized
MNTKYNKEFLLYLAGFVDSDGSIYATIAPKQQLKFKHQLQLVFVVAQATKRRWFLDKLVDEIGVGYVHDYRSASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 416 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYHTASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 417 - protein, 354 aa, synthetic construct
  REGION 1..354: note = Synthesized
MNTKYNKEFLLYLAGFVDADGSIYATIAPKQQLKFKHQLQLVFVVAQLTKRRWFLDKLVDEIGVGYVHDYRKASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 418 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 419 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYTHCSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 420 - protein, 354 aa, synthetic construct
  REGION 1..354: note = Synthesized
MNTKYNKEFLLYLAGFVDADGSIYATIAPKQQLKFKHQLQLVFVVAQHTRRRWFLDKLVDEIGVGYVHDYGRASYYRLSQIKPLHNFLTQLQPFLKLKQKQANLVLKIIEQLPSAKESPDKFLEVCTWVDQIAALNDSTTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 421 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYRAASYYRLSQIKPLHNFLTQLQPFLKLKQKQANLVLKIIEQLPSAKESPDKFLEVCTWVDQIAALNDSHTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 422 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYLGCAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 423 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYSGSSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 424 - protein, 354 aa, synthetic construct; identical to SEQ ID NO: 419
  REGION 1..354: note = Synthesized

SEQ ID NO: 425 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 426 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYRRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 427 - protein, 354 aa, synthetic construct
  REGION 1..354: note = Synthesized
MNTKYNKEFLLYLAGFVDSDGSIYATIAPKQQLKFKHQLQLVFVVAQATKRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 428 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGNASYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 429 - protein, 354 aa, synthetic construct
  REGION 1..354: note = Synthesized
MNTKYNKEFLLYLAGFVDSDGSIYATIAPKQQLKFKHQLQLVFVVAQTTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 430 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYNRAAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 431 - protein, 354 aa, synthetic construct
  REGION 1..354: note = Synthesized
MNTKYNKEFLLYLAGFVDSDGSIYATIAPKQQLKFKHQLQLVFVVAQLTSRRWFLDKLVDEIGVGYVHDYKRCSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 432 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYNRTSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 433 - protein, 354 aa, synthetic construct
  REGION 1..354: note = Synthesized
MNTKYNKEFLLYLAGFVDSDGSIYATIAPKQQLKFKHQLQLVFVVAQKTLRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 434 - DNA, 22 bp, synthetic construct
  misc_feature 1..22: note = Synthesized
  source 1..22: mol_type = other DNA
gtatagcatgtgatatacgaag

SEQ ID NO: 435 - DNA, 22 bp, synthetic construct
  misc_feature 1..22: note = Synthesized
  source 1..22: mol_type = other DNA
catatcgtacactatatgcttc

SEQ ID NO: 436 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYRTVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 437 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYRTVAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 438 - protein, 354 aa, synthetic construct
  REGION 1..354: note = Synthesized
MNTKYNKEFLLYLAGFVDADGSIYATIAPKQQLKFKHQLQLVFVVAQGTRRRWFLDKLVDEIGVGYVHDYGSVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 439 - protein, 354 aa, synthetic construct
  REGION 1..354: note = Synthesized
MNTKYNKEFLLYLAGFVDADGSIYATIAPKQQLKFKHQLQLVFVVAQRTRRRWFLDKLVDEIGVGYVHDYRGVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 440 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYRHVTYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 441 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGTVSYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 442 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYVSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 443 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYSKVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 444 - protein, 354 aa, synthetic construct
  REGION 1..354: note = Synthesized
MNTKYNKEFLLYLAGFVDSDGSIYATIAPKQQLKFKHQLQLVFVVAQGTRRRWFLDKLVDEIGVGYVHDYSGVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 445 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYAKVAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 446 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYRSVAYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 447 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYTRVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 448 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYRRVAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 449 - protein, 354 aa, synthetic construct
  REGION 1..354: note = Synthesized
MNTKYNKEFLLYLAGFVDSDGSIYATIAPKQQLKFKHQLQLVFVVAQRTVRRWFLDKLVDEIGVGYVHDYNGVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 450 - protein, 354 aa, synthetic construct
  REGION 1..354: note = Synthesized
MNTKYNKEFLLYLAGFVDADGSIYATIAPKQQLKFKHQLQLVFVVAQRTRRRWFLDKLVDEIGVGYVHDYGSVSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 451 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYRRVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 452 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYRHVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 453 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYRKVGYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 454 - protein, 354 aa, synthetic construct
  REGION 1..354: note = Synthesized
MNTKYNKEFLLYLAGFVDADGSIYATIAPKQQLKFKHQLQLVFVVAQGTRRRWFLDKLVDEIGVGYVHDYSRVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 455 - protein, 354 aa, synthetic construct
  REGION 1..354: note = Synthesized
MNTKYNKEFLLYLAGFVDSDGSIYATIAPKQQLKFKHQLQLVFVVAQKTCRRWFLDKLVDEIGVGYVHDYTGVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 456 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYDTVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 457 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYHRVAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 458 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYHTVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 459 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYRYVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 460 - protein, 354 aa, synthetic construct
  REGION 1..354: note = Synthesized
MNTKYNKEFLLYLAGFVDSDGSIYATIAPKQQLKFKHQLQLVFVVAQHTRRRWFLDKLVDEIGVGYVHDYRRVAYYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 461 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYSRTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 462 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYATASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 463 - DNA, 22 bp, synthetic construct
  misc_feature 1..22: note = Synthesized
  source 1..22: mol_type = other DNA
gtatagcatgtgctatacgaag

SEQ ID NO: 464 - DNA, 22 bp, synthetic construct
  misc_feature 1..22: note = Synthesized
  source 1..22: mol_type = other DNA
catatcgtacacgatatgcttc

SEQ ID NO: 465 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYSKVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 466 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYSSTAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 467 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYKMCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 468 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYGPCSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 469 - protein, 354 aa, synthetic construct
  REGION 1..354: note = Synthesized
MNTKYNKEFLLYLAGFVDSDGSIYATIAPKQQLKFKHQLQLVFVVAQHTRRRWFLDKLVDEIGVGYVHDYNGTSYYRLSQIKPLHNFLTQLQPFLKLKQKQANLVLKIIEQLPSAKESPDKFLEVCTWVDQIAALNDSTTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 470 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYACTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 471 - protein, 354 aa, synthetic construct
  REGION 1..354: note = Synthesized
MNTKYNKEFLLYLAGFVDADGSIYATIAPKQQLKFKHQLQLVFVVAQKTKRRWFLDKLVDEIGVGYVHDYTTTAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 472 - protein, 354 aa, synthetic construct
  REGION 1..354: note = Synthesized
MNTKYNKEFLLYLAGFVDSDGSIYATIAPKQQLKFKHQLQLVFVVAQATRRRWFLDKLVDEIGVGYVHDYSHTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 473 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYIPTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 474 - protein, 354 aa, synthetic construct
  REGION 1..354: note = Synthesized
MNTKYNKEFLLYLAGFVDADGSIYATIAPKQQLKFKHQLQLVFVVAQKTSRRWFLDKLVDEIGVGYVHDYEPTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 475 - protein, 354 aa, synthetic construct
  REGION 1..354: note = Synthesized
MNTKYNKEFLLYLAGFVDSDGSIYATIAPKQQLKFKHQLQLVFVVAQRTRRRWFLDKLVDEIGVGYVHDYYTTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 476 - protein, 354 aa, synthetic construct
  REGION 1..354: note = Synthesized
MNTKYNKEFLLYLAGFVDSDGSIYATIAPKQQLKFKHQLQLVFVVAQHTRRRWFLDKLVDEIGVGYVHDYRSASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 477 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYQSNSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 478 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYYNNSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 479 - protein, 354 aa, synthetic construct
  REGION 1..354: note = Synthesized
MNTKYNKEFLLYLAGFVDADGSIYATIAPKQQLKFKHQLQLVFVVAQKTVRRWFLDKLVDEIGVGYVHDYRATTYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 480 - protein, 354 aa, synthetic construct
  REGION 1..354: note = Synthesized
MNTKYNKEFLLYLAGFVDSDGSIYATIAPKQQLKFKHQLQLVFVVAQRTKRRWFLDKLVDEIGVGYVHDYSSTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 481 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYSMVSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 482 - protein, 354 aa, synthetic construct
  REGION 1..354: note = Synthesized
MNTKYNKEFLLYLAGFVDADGSIYATIAPKQQLKFKHQLQLVFVVAQLTKRRWFLDKLVDEIGVGYVHDYTATSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 483 - protein, 354 aa, synthetic construct
  REGION 1..354: note = Synthesized
MNTKYNKEFLLYLAGFVDSDGSIYATIAPKQQLKFKHQLQLVFVVAQNTRRRWFLDKLVDEIGVGYVHDYRSTAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 484 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYYRTSYYRLSQIKPLHNFLTQLQPFLKLKQKQANLVLKIIEQLPSAKESPDKFLEVCTWVDQIAALNDSHTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 485 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGPTSYYRLSQIKPLHNFLTQLQPFLKLKQKQANLVLKIIEQLPSAKESPDKFLEVCTWVDQIAALNDSVTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 486 - protein, 354 aa, synthetic construct
  REGION 1..354: note = Synthesized
MNTKYNKEFLLYLAGFVDSDGSIYATIAPKQQLKFKHQLQLVFVVAQRTVRRWFLDKLVDEIGVGYVHDYNSTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 487 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYSQTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 488 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYFRTSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 489 - protein, 354 aa, synthetic construct
  REGION 1..354: note = Synthesized
MNTKYNKEFLLYLAGFVDADGSIYATIAPKQQLKFKHQLQLVFVVAQRTIRRWFLDKLVDEIGVGYVHDYGAVSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 490 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYRNCSYYRLSQIKPLHNFLTQLQPFLKLKQKQANLVLKIIEQLPSAKESPDKFLEVCTWVDQIAALNDSTTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 491 - protein, 354 aa, synthetic construct
  REGION 1..354: note = Synthesized
MNTKYNKEFLLYLAGFVDSDGSIYATIAPKQQLKFKHQLQLVFVVAQNTGRRWFLDKLVDEIGVGYVHDYVGVTYYRLSQIKPLHNFLTQLQPFLKLKQKQANLVLKIIEQLPSAKESPDKFLEVCTWVDQIAALNDSHTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 492 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYRMLSYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 493 - protein, 354 aa, synthetic construct
  REGION 1..354: note = Synthesized
MNTKYNKEFLLYLAGFVDSDGSIYATIAPKQQLKFKHQLQLVFVVAQHTRRRWFLDKLVDEIGVGYVHDYSRTSYYRLSQIKPLHNFLTQLQPFLKLKQKQANLVLKIIEQLPSAKESPDKFLEVCTWVDQIAALNDSSTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 494 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYTDTAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 495 - protein, 354 aa, synthetic construct
  REGION 1..354: note = Synthesized
MNTKYNKEFLLYLAGFVDSDGSIYATIAPKQQLKFKHQLQLVFVVAQKTSRRWFLDKLVDEIGVGYVHDYSKTSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 496 - DNA, 22 bp, synthetic construct
  misc_feature 1..22: note = Synthesized
  source 1..22: mol_type = other DNA
gtatagcatgtggtatacgaag

SEQ ID NO: 497 - DNA, 22 bp, synthetic construct
  misc_feature 1..22: note = Synthesized
  source 1..22: mol_type = other DNA
catatcgtacaccatatgcttc

SEQ ID NO: 498 - protein, 354 aa, synthetic construct
  REGION 1..354: note = Synthesized
MNTKYNKEFLLYLAGFVDADGSIYATIAPKQQLKFKHQLQLVFVVAQKTRRRWFLDKLVDEIGVGYVHDYSGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSIYACILPMQARKFKHQLSLTFTVYQKTQRRWFLDKLVDEIGVGYVCDKGSVSAYMLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 499 - protein, 354 aa, synthetic construct
  REGION 1..354: note = Synthesized
MNTKYNKEFLLYLAGFVDGDGSIYAVIQPGQKYKFKHNLRLTFRVSQKTQRRWFLDKLVDEVGVGYVSDHGSVSSYLLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSIYACILPMQARKFKHQLSLTFTVYQKTQRRWFLDKLVDEIGVGYVCDKGSVSAYMLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 500 - protein, 354 aa, synthetic construct
  REGION 1..354: note = Synthesized
MNTKYNKEFLLYLAGFVDGDGSIYAGIGPNQACKFKHQLYLRFRVSQKTQRRWFLDKLVDEIGVGYVTDEGSVSIYTLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSIYACILPMQARKFKHQLSLTFTVYQKTQRRWFLDKLVDEIGVGYVCDKGSVSAYMLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 501 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYDGRSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDADGSIYACILPMQARKFKHQLSLTFTVYQKTQRRWFLDKLVDEIGVGYVCDKGSVSAYMLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 502 - DNA, 22 bp, synthetic construct
  misc_feature 1..22: note = Synthesized
  source 1..22: mol_type = other DNA
gtatagcatgtgttatacgaag

SEQ ID NO: 503 - DNA, 22 bp, synthetic construct
  misc_feature 1..22: note = Synthesized
  source 1..22: mol_type = other DNA
catatcgtacacaatatgcttc

SEQ ID NO: 504 - protein, 354 aa, synthetic construct
  REGION 1..354: note = Synthesized
MNTKYNKEFLLYLAGFVDSDGSIYATIAPKQQLKFKHQLQLVFVVAQKTVRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 505 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYGPSSYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 506 - protein, 354 aa, synthetic construct
  REGION 1..354: note = Synthesized
MNTKYNKEFLLYLAGFVDSDGSIYATIAPKQQLKFKHQLQLVFVVAQSTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 507 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 508 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYRACSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 509 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYRQASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 510 - protein, 354 aa, synthetic construct
  REGION 1..354: note = Synthesized
MNTKYNKEFLLYLAGFVDSDGSIYATIAPKQQLKFKHQLQLVFVVAQKTQRRWFLDKLVDEIGVGYVHDYGKAAYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 511 - protein, 354 aa, synthetic construct
  REGION 1..354: note = Synthesized
MNTKYNKEFLLYLAGFVDSDGSIYATIAPKQQLKFKHQLQLVFVVAQVTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 512 - protein, 354 aa, synthetic construct
  REGION 1..354: note = Synthesized
MNTKYNKEFLLYLAGFVDSDGSIYATIAPKQQLKFKHQLQLVFVVAQKTSRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 513 - protein, 354 aa, synthetic construct
  REGION 1..354: note = Synthesized
MNTKYNKEFLLYLAGFVDSDGSIYATIAPKQQLKFKHQLQLVFVVAQLTKRRWFLDKLVDEIGVGYVHDYNRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 514 - protein, 354 aa, synthetic construct
  REGION 1..354: note = Synthesized
MNTKYNKEFLLYLAGFVDSDGSIYATIAPKQQLKFKHQLQLVFVVAQKTARRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 515 - protein, 354 aa, synthetic construct
  REGION 1..354: note = Synthesized
MNTKYNKEFLLYLAGFVDSDGSIYATIAPKQQLKFKHQLQLVFVVAQLTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 516 - protein, 354 aa, synthetic construct
  REGION 1..354: note = Synthesized
MNTKYNKEFLLYLAGFVDSDGSIYATIAPKQQLKFKHQLQLVFVVAQKTERRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 517 - protein, 354 aa, synthetic construct
  REGION 1..354: note = Synthesized
MNTKYNKEFLLYLAGFVDSDGSIYATIAPKQQLKFKHQLQLVFVVAQKTKRRWFLDKLVDEIGVGYVHDYHRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 518 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYATASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 519 - protein, 354 aa, synthetic construct
  REGION 1..354: note = Synthesized
MNTKYNKEFLLYLAGFVDSDGSIYATIAPKQQLKFKHQLQLVFVVAQKTRRRWFLDKLVDEIGVGYVHDYTGTSYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 520 - protein, 354 aa, synthetic construct
  REGION 1..354: note = Synthesized
MNTKYNKEFLLYLAGFVDADGSIYATIAPKQQLKFKHQLQLVFVVAQLTRRRWFLDKLVDEIGVGYVHDYNKASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 521 - protein, 354 aa, synthetic construct; identical to SEQ ID NO: 509
  REGION 1..354: note = Synthesized

SEQ ID NO: 522 - protein, 354 aa, synthetic construct
  REGION 1..354: note = Synthesized
MNTKYNKEFLLYLAGFVDADGSIYATIAPKQQLKFKHQLQLVFVVAQGTRRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 523 - protein, 354 aa, synthetic construct; identical to SEQ ID NO: 425
  REGION 1..354: note = Synthesized

SEQ ID NO: 524 - protein, 354 aa, synthetic construct
  REGION 1..354: note = Synthesized
MNTKYNKEFLLYLAGFVDSDGSIYATIAPKQQLKFKHQLQLVFVVAQKTCRRWFLDKLVDEIGVGYVHDYARASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 525 - protein, 354 aa, synthetic construct
  REGION 1..354: note = Synthesized
MNTKYNKEFLLYLAGFVDSDGSIYATIAPKQQLKFKHQLQLVFVVAQSTVRRWFLDKLVDEIGVGYVHDYGRASYYRLSQIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 526 - protein, 354 aa, synthetic construct
  REGION 1..354: note = Synthesized
MNTKYNKEFLLYLAGFVDADGSIYATIAPKQQLKFKHQLQLVFVVAQSTRRRWFLDKLVDEIGVGYVHDYGVATYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 527 - protein, 354 aa, synthetic construct
  REGION 1..354: note = Synthesized
MNTKYNKEFLLYLAGFVDADGSIYATIAPKQQLKFKHQLQLVFVVAQKTQRRWFLDKLVDEIGVGYVHDYGRASYYRLSEIKPLHNFLTQLQPFLKLKQKQANLVLKIIEQLPSAKESPDKFLEVCTWVDQIAALNDSKTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 528 - protein, 354 aa, synthetic construct
  REGION 1..354: note = Synthesized
MNTKYNKEFLLYLAGFVDSDGSIYATIAPKQQLKFKHQLQLVFVVAQNTRRRWFLDKLVDEIGVGYVHDYGRASYYRLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSICASIRPCQVAKFKHALELRFTVGQKTQRRWFLDKLVDEIGVGYVYDLGSVSQYTLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 529 - protein, 354 aa, synthetic construct; identical to SEQ ID NO: 406
  REGION 1..354: note = Synthesized

SEQ ID NO: 530 - protein, 42 aa, synthetic construct
  REGION 1..42: note = Synthesized
SLPGSVGGLSPSQASSAASSASSSPGSGISEALRAGAGSGTG

(19) United States
(12) Patent Application Publication
(10) Pub. No.: US 2023/0340434 A1
(43) Pub. Date: Oct. 26, 2023

(54) OPTIMIZATION OF ENGINEERED MEGANUCLEASES FOR RECOGNITION SEQUENCES

(71) Applicant: Precision BioSciences, Inc., Durham, NC (US)

(72) Inventors: James Jefferson Smith, Morrisville, NC (US); Hui Li, Apex, NC (US)

(73) Assignee: Precision BioSciences, Inc., Durham, NC (US)

(21) Appl. No.: 17/819,227

(22) Filed: Aug. 11, 2022

Related U.S. Application Data
(63) Continuation of application No. 17/609,244, filed on Nov. 5, 2021, now abandoned.
(60) Provisional application No. 62/936,306, filed on Nov. 15, 2019.
(60) Provisional application No. 62/844,586, filed on May 7, 2019.

(51) Int. Cl. C12N 9/22; C12N 15/10; C12N 15/86

(52) U.S. Cl. CPC C12N 9/22; C12N 15/102; C12N 15/86; C12N 2710/10043

(57) ABSTRACT

The invention provides engineered meganucleases, derived from I-Cre1, which have substitutions at particular positions that increase the activity of the nucleases for recognition sequences containing certain center sequences. The invention also provides methods of cleaving double-stranded DNA using such engineered meganucleases. The invention further provides methods for improving the activity of engineered meganucleases for recognition sequences containing certain center sequences.

### FIELD OF THE INVENTION

The invention relates to the field of molecular biology and recombinant nucleic acid technology. In particular, the invention relates to the optimization of engineered, I-CreI-derived meganucleases for recognition sequences comprising certain center sequences.

### REFERENCE TO A SEQUENCE LISTING SUBMITTED AS A TEXT FILE VIA EFS-WEB

The instant application contains a Sequence Listing which has been submitted in ASCTI format via EFS-Web and is hereby incorporated by reference in its entirety. Said ASCII copy, created on May 7, 2020, is named P109070040WO00-SEQ-EPG, and is 1,457 kilobytes in size.

### BACKGROUND OF THE INVENTION

Genome engineering requires the ability to insert, delete, substitute and otherwise manipulate specific genetic sequences within a genome, and has numerous therapeutic and biotechnological applications. The development of effective means for genome modification remains a major goal in gene therapy, agrotechnology, and synthetic biology (Porteus et al. (2005), Nat. Biotechnol. 23: 967-73; Tzfira et al. (2005), Trends Biotechnol. 23: 567-9; McDaniel et al. (2005), Curr. Opin. Biotechnol. 16: 476-83). One approach to achieving this goal is utilizing site specific, rare cutting nucleases, such as meganucleases (i.e., homing endonucleases).

Meganucleases are commonly grouped into four families: the LAGLIDADG (SEQ ID NO: 2) family, the GIY-YIG family, the His-Cys box family and the HNH family. These families are characterized by structural motifs, which affect catalytic activity and recognition sequence. For instance, members of the LAGLIDADG (SEQ ID NO: 2) family are characterized by having either one or two copies of the conserved LAGLIDADG (SEQ ID NO: 2) motif (see Chevalier et al. (2001), Nucleic Acids Res. 29(18): 3757-3774). The LAGLIDADG (SEQ ID NO: 2) meganucleases with a single copy of the LAGLIDADG (SEQ ID NO: 2) motif form homodimers, whereas members with two copies of the LAGLIDADG (SEQ ID NO: 2) motif are found as monomers.

I-CreI (SEQ ID NO: 1) is a member of the LAGLIDADG (SEQ ID NO: 2) family, which recognizes and cleaves a 22 base pair recognition sequence in the chloroplast chromosome. Genetic selection techniques have been used to modify the wild-type I-CreI recognition site preference (Sussman et al. (2004). J. Mol. Biol. 342: 31-41; Chames et al. (2005), Nucleic Acids Res. 33: e178; Seligman et al. (2002), Nucleic Acids Res. 30: 3870-9, Arnould et al. (2006), J. Mol. Biol. 355: 443-58). Methods of engineering I-CreI to target widely-divergent DNA sites, including sites in mammalian, yeast, plant, bacterial, and viral genomes, have previously been disclosed, for example, in WO 2007/047859.

The DNA sequences recognized by I-CreI are 22 base pairs in length. One example of a naturally-occurring I-CreI recognition site is provided in SEQ ID NO: 3, but the enzyme will bind to a variety of related sequences with varying affinity. The wild-type I-CreI enzyme binds DNA as a homodimer in which each monomer makes direct contacts with a nine base pair “half-site”. The two half-sites of a recognition sequence are separated by a four base pair “center sequence”. These four central bases are not directly contacted by the enzyme. Following cleavage, wild-type I-CreI, and engineered I-CreI-derived meganucleases, produce a staggered double-strand break at the center of the recognition sequence, resulting in the production of a four base pair 3′-overhang (FIG. 1).

The present invention concerns the central four base pairs (i.e., the center sequence) in an meganuclease recognition sequence that become the 3′ overhang following cleavage. In the case of the native I-CreI recognition sequence in the Chlamydomonas reinhardtii 23S rRNA gene, the center sequence is 5′-GTGA-3′. A number of published studies concerning I-CreI or its derivatives evaluated the enzyme, either wild-type or genetically-engineered, using DNA substrates that employed either the native 5′-GTGA-3′ center sequence or the palindromic sequence 5′-GTAC-3′. Arnould et. al. (Arnould et al. (2007), J. Mol. Biol. 371: 49-65) reported that a set of genetically-engineered meganucleases derived from I-CreI cleaved DNA substrates with varying efficiencies depending on whether the substrate sequences were centered around 5′-GTAC-3′, 5′-TTGA-3′, 5′-GAAA-3′, or 5′-ACAC-3′.

Furthermore, WO 2010/009147 (the '147 publication) disclosed that engineered meganucleases will cleave different recognition sequences with varying efficiencies depending on the center sequence. The '147 publication describes general rules for engineered meganuclease targeting and cleaving of recognition sequences based on their center sequences, and the efficiency with which such sequences can be cleaved.

However, the '147 publication does not describe whether I-CreI-derived meganucleases can be modified to improve their activity and/or specificity for cleaving a recognition sequence with specific center sequences. Indeed, it was previously believed that subunits of wild-type I-Cre and I-CreI-derived meganucleases did not directly interact with the center sequence. Accordingly, the present invention advances the art by identifying particular positions and residues which allow for the optimization of I-CreI-derived meganucleases for recognizing and cleaving recognition sequences having specific center sequences.

### SUMMARY OF THE INVENTION

One aspect is an engineered meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, and wherein the first subunit and the second subunit each comprise a substitution at one or more positions corresponding to positions 48, 50, 71, 72, 73, and 74 of SEQ ID NO: 1.

In some embodiments, the center sequence consists of ACAA.

In some embodiments, the first subunit comprises one or more of the following residues: (a) a K or L residue at a position corresponding to position 48 of SEQ ID NO: 1; (b) a C, R, T, K, or S residue at a position corresponding to position 50 of SEQ ID NO: 1; (c) a G or R residue at a position corresponding to position 71 of SEQ ID NO: 1; (d) an R or Q residue at a position corresponding to position 72 of SEQ ID NO: 1; and (e) an A or C residue at a position corresponding to position 73 of SEQ ID NO: 1.

In some embodiments, the second subunit comprises one or more of the following residues: (a) a K, T, S, or A residue at a position corresponding to position 48 of SEQ ID NO: 1; (b) a C, R, E, K, or T residue at a position corresponding to position 50 of SEQ ID NO: 1; (c) a G or A residue at a position corresponding to position 71 of SEQ ID NO: 1; (d) a T, R, S, P, N, G, or A residue at a position corresponding to position 72 of SEQ ID NO: 1; (e) a V or I residue at a position corresponding to position 73 of SEQ ID NO: 1; and (f) an S, T, or A residue at a position corresponding to position 74 of SEQ ID NO: 1.

In some embodiments, the first subunit comprises residues corresponding to residues 48, 50, 71, 72, and 73 of any one of SEQ ID NOs: 11-33. In some embodiments, the second subunit comprises residues corresponding to residues 239, 241, 262, 263, 264, and 265 of any one of SEQ ID NOs: 11-33. In some embodiments, the first subunit comprises one or more of the following residues: (a) an A or G residue at a position corresponding to position 19 of SEQ ID NO: 1; (b) a Q or E residue at a position corresponding to position 80 of SEQ ID NO: 1; (c) a K or R residue at a position corresponding to position 139 of SEQ ID NO: 1; and (d) an S or G residue at a position corresponding to position 154 of SEQ ID NO: 1.

In some embodiments, the second subunit comprises one or more of the following residues: (a) a G, A, or S residue at a position corresponding to position 19 of SEQ ID NO: 1; (b) a Y or C residue at a position corresponding to position 66 of SEQ ID NO: 1; (c) a Q or E residue at a position corresponding to position 80 of SEQ ID NO: 1; (d) a Q or R residue at a position corresponding to position 92 of SEQ ID NO: 1; (e) an E or G residue at a position corresponding to position 117 of SEQ ID NO: 1; and (f) a K or R residue at a position corresponding to position 139 of SEQ ID NO: 1.

In some embodiments, the first subunit comprises residues corresponding to residues 19, 80, 139, and 154 of any one of SEQ ID NOs: 11-33.

In some embodiments, the second subunit comprises residues corresponding to residues 19, 66, 80, 92, 117, and 139 of any one of SEQ ID NOs: 11-33.

Another aspect is a method for cleaving double-stranded DNA at a target site comprising a meganuclease recognition sequence comprising a center sequence consisting of ACAA, the method comprising contacting the double-stranded DNA having the target site with an engineered meganuclease described herein, wherein the engineered meganuclease binds and cleaves the recognition sequence.

In some embodiments, the center sequence consists of ACAG.

In some embodiments, the first subunit comprises one or more of the following residues: (a) an R residue at a position corresponding to position 50 of SEQ ID NO: 1; (b) a G or R residue at a position corresponding to position 71 of SEQ ID NO: 1; (c) an R, K, Q, P, or T residue at a position corresponding to position 72 of SEQ ID NO: 1; and (d) an A or C residue at a position corresponding to position 73 of SEQ ID NO: 1.

In some embodiments, the second subunit comprises one or more of the following residues: (a) a C residue at a position corresponding to position 50 of SEQ ID NO: 1; (b) a G, S, or D residue at a position corresponding to position 71 of SEQ ID NO: 1; (c) an R or G residue at a position corresponding to position 72 of SEQ ID NO: 1; (d) an R residue at a position corresponding to position 73 of SEQ ID NO: 1; and optionally (e) an R residue at a position following a position corresponding to position 73 of SEQ ID NO: 1.

In some embodiments, the first subunit comprises residues corresponding to residues 50, 71, 72, and 73 of any one of SEQ ID NOs: 36-43.

In some embodiments, the first subunit comprises residues corresponding to residues 50, 71, 72, and 73 of any one of SEQ ID NOs: 36-43.

In some embodiments, the first subunit comprises one or more of the following residues: (a) an A or G residue at a position corresponding to position 19 of SEQ ID NO: 1; (b) an F, I, or L residue at a position corresponding to position 54 of SEQ ID NO: 1; (c) a Q or E residue at a position corresponding to position 80 of SEQ ID NO: 1; and (d) an S or P residue at a position corresponding to position 158 of SEQ ID NO: 1.

In some embodiments, the second subunit comprises one or more of the following residues: (a) a G, A, or S residue at a position corresponding to position 19 of SEQ ID NO: 1; (b) a V or A residue at a position corresponding to position 59 of SEQ ID NO: 1; (c) a Y or H residue at a position corresponding to position 66 of SEQ ID NO: 1; (d) a Q residue at a position corresponding to position 80 of SEQ ID NO: 1; (e) an I or T residue at a position corresponding to position 81 of SEQ ID NO: 1; and (f) a K or R residue at a position corresponding to position 139 of SEQ ID NO: 1.

In some embodiments, the first subunit comprises residues corresponding to residues 19, 54, 80, and 158 of any one of SEQ ID NOs: 36-43.

In some embodiments, the second subunit comprises residues corresponding to residues 19, 59, 66, 80, 81, and 139 of any one of SEQ ID NOs: 36-43.

In some embodiments, the second subunit further comprises an R residue inserted between positions corresponding to positions 73 and 74 of SEQ ID NO: 1.

Another aspect is a method for cleaving double-stranded DNA at a target site comprising a meganuclease recognition sequence comprising a center sequence consisting of ACAG, the method comprising contacting the double-stranded DNA having the target site with an engineered meganuclease described herein, wherein the engineered meganuclease binds and cleaves the recognition sequence.

In some embodiments, the center sequence consists of ACAT.

In some embodiments, the first subunit comprises one or more of the following residues: (a) a K, S, I, L, or N residue at a position corresponding to position 48 of SEQ ID NO: 1; (b) a Q, S, R, or K residue at a position corresponding to position 50 of SEQ ID NO: 1; (c) a G or R residue at a position corresponding to position 71 of SEQ ID NO: 1; (d) an R or T residue at a position corresponding to position 72 of SEQ ID NO: 1; and (e) an A or G residue at a position corresponding to position 73 of SEQ ID NO: 1.

In some embodiments, the second subunit comprises one or more of the following residues: (a) an H, T, G, A, S, L, or K residue at a position corresponding to position 48 of SEQ ID NO: 1; (b) an S, K, C, N R, G, or Q residue at a position corresponding to position 50 of SEQ ID NO: 1; (c) an S, G, R, T, K, or E residue at a position corresponding to position 71 of SEQ ID NO: 1; (d) a T, K, A, S, R, H, G, or N residue at a position corresponding to position 72 of SEQ ID NO: 1; (e) an H, A, C, S, G, or R residue at a position corresponding to position 73 of SEQ ID NO: 1; and (f) an S, C, or A residue at a position corresponding to position 74 of SEQ ID NO: 1.

In some embodiments, the first subunit comprises residues corresponding to residues 48, 50, 71, 72, and 73 of any one of SEQ ID NOs: 46-67.

In some embodiments, the second subunit comprises residues corresponding to residues 239, 241, 262, 263, 264, and 265 of any one of SEQ ID NOs: 46-67.

In some embodiments, the first subunit comprises one or more of the following residues: (a) an A, G, or S residue at a position corresponding to position 19 of SEQ ID NO: 1; (b) an F or I residue at a position corresponding to position 54 of SEQ ID NO: 1; (c) a Q or E residue at a position corresponding to position 80 of SEQ ID NO: 1; and (d) a K, H, or R residue at a position corresponding to position 139 of SEQ ID NO: 1.

In some embodiments, the second subunit comprises one or more of the following residues: (a) an A, G, or S residue at a position corresponding to position 19 of SEQ ID NO: 1; (b) a Q or E residue at a position corresponding to position 80 of SEQ ID NO: 1; (c) an I or T residue at a position corresponding to position 81 of SEQ ID NO: 1; (d) a P or H residue at a position corresponding to position 83 of SEQ ID NO: 1; (e) an E or G residue at a position corresponding to position 117 of SEQ ID NO: 1; and (f) a K, R, T, or H residue at a position corresponding to position 139 of SEQ ID NO: 1.

In some embodiments, the first subunit comprises residues corresponding to residues 19, 54, 80, and 139 of any one of SEQ ID NOs: 46-67.

In some embodiments, the second subunit comprises residues corresponding to residues 19, 80, 81, 83, 117, and 139 of any one of SEQ ID NOs: 46-67.

Another aspect is a method for cleaving double-stranded DNA at a target site comprising a meganuclease recognition sequence comprising a center sequence consisting of ACAT, the method comprising contacting the double-stranded DNA having the target site with an engineered meganuclease described herein, wherein the engineered meganuclease binds and cleaves the recognition sequence.

In some embodiments, the center sequence consists of ACGA.

In some embodiments, the first subunit comprises one or more of the following residues: (a) a K residue at a position corresponding to position 48 of SEQ ID NO: 1; (b) a V, R, T, W, or A residue at a position corresponding to position 50 of SEQ ID NO: 1; (c) a G or P residue at a position corresponding to position 71 of SEQ ID NO: 1; (d) an R or P residue at a position corresponding to position 72 of SEQ ID NO: 1; and (c) an A residue at a position corresponding to position 73 of SEQ ID NO: 1.

In some embodiments, the second subunit comprises one or more of the following residues: (a) a K, H, T, A, G, or Q residue at a position corresponding to position 48 of SEQ ID NO: 1; (b) an R, S, C, I, V, or G residue at a position corresponding to position 50 of SEQ ID NO: 1; (c) a G residue at a position corresponding to position 71 of SEQ ID NO: 1; (d) an R or H residue at a position corresponding to position 72 of SEQ ID NO: 1; (e) an I or V residue at a position corresponding to position 73 of SEQ ID NO: 1; and (f) an S or A residue at a position corresponding to position 74 of SEQ ID NO: 1.

In some embodiments, the first subunit comprises residues corresponding to residues 48, 50, 71, 72, and 73 of any one of SEQ ID NOs: 70-89.

In some embodiments, the second subunit comprises residues corresponding to residues 239, 241, 262, 263, 264, and 265 of any one of SEQ ID NOs: 70-89.

In some embodiments, the first subunit comprises one or more of the following residues: (a) an A, G, or S residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments, the second subunit comprises one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the first subunit comprises residues corresponding to residues 19, 80, and 139 of any one of SEQ ID NOs: 70-89.

In some embodiments, the second subunit comprises residues corresponding to residues 19, 80, and 139 of any one of SEQ ID NOs: 70-89.

Another aspect is a method for cleaving double-stranded DNA at a target site comprising a meganuclease recognition sequence comprising a center sequence consisting of ACGA, the method comprising contacting the double-stranded DNA having the target site with an engineered meganuclease described herein, wherein the engineered meganuclease binds and cleaves the recognition sequence.

In some embodiments, the center sequence consists of ACGC.

In some embodiments, the first subunit comprises one or more of the following residues: (a) a K, H, Q, L, A, or S residue at a position corresponding to position 48 of SEQ ID NO: 1; (b) a Q, R, K, S, T, or C residue at a position corresponding to position 50 of SEQ ID NO: 1; (c) a G, R, or A residue at a position corresponding to position 71 of SEQ ID NO: 1; (d) an R, P, or H residue at a position corresponding to position 72 of SEQ ID NO: 1; and (e) an A residue at a position corresponding to position 73 of SEQ ID NO: 1.

In some embodiments, the second subunit comprises one or more of the following residues: (a) an H, K, L, A, S, or N residue at a position corresponding to position 48 of SEQ ID NO: 1; (b) an S, E, K, I, N, or V residue at a position corresponding to position 50 of SEQ ID NO: 1; (c) an S, G, K, A, or R residue at a position corresponding to position 71 of SEQ ID NO: 1; (d) a T, R, A, S, H, or G residue at a position corresponding to position 72 of SEQ ID NO: 1; (e) an H, T, V, I, or C residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, and 73 of any one of SEQ ID NOs: 92-118.

In some embodiments, the second subunit comprises residues corresponding to residues 239, 241, 262, 263, 264, and 265 of any one of SEQ ID NOs: 92-118.

In some embodiments, the first subunit comprises one or more of the following residues: (a) an A, G, or S residue at a position corresponding to position 19 of SEQ ID NO: 1; and (b) a Q or E residue at a position corresponding to position 80 of SEQ ID NO: 1.

In some embodiments, the second subunit comprises one or more of the following residues: (a) an A or G residue at a position corresponding to position 19 of SEQ ID NO: 1; (b) a Q or E residue at a position corresponding to position 80 of SEQ ID NO: 1; (c) an F or L residue at a position corresponding to position 87 of SEQ ID NO: 1; and (d) a K, R, N, H, or A residue at a position corresponding to position 139 of SEQ ID NO: 1.

In some embodiments, the first subunit comprises residues corresponding to residues 19 and 80 of any one of SEQ ID NOs: 92-118.

In some embodiments, the second subunit comprises residues corresponding to residues 19, 80, 87, and 139 of any one of SEQ ID NOs: 92-118.

Another aspect is a method for cleaving double-stranded DNA at a target site comprising a meganuclease recognition sequence recognition sequence comprises a center sequence consisting of ACGC, the method comprising contacting the double-stranded DNA having the target site with an engineered meganuclease described herein, wherein the engineered meganuclease binds and cleaves the recognition sequence.

In some embodiments, the center sequence consists of ACGG.

In some embodiments, the first subunit comprises one or more of the following residues: (a) an R or K residue at a position corresponding to position 50 of SEQ ID NO: 1; (b) an R residue at a position corresponding to position 72 of SEQ ID NO: 1; and (c) an A residue at a position corresponding to position 73 of SEQ ID NO: 1.

In some embodiments, the second subunit comprises one or more of the following residues: (a) a K residue at a position corresponding to position 48 of SEQ ID NO: 1; (b) an R or P residue at a position corresponding to position 50 of SEQ ID NO: 1; (c) a D residue at a position corresponding to position 71 of SEQ ID NO: 1; (d) a G residue at a position corresponding to position 72 of SEQ ID NO: 1; and (e) an R or G residue at a position corresponding to position 73 of SEQ ID NO: 1.

In some embodiments, the first subunit comprises residues corresponding to residues 50, 72, and 73 of any one of SEQ ID NOs: 121-135.

In some embodiments, the second subunit comprises residues corresponding to residues 239, 241, 262, 263, and 264 of any one of SEQ ID NOs: 121-135.

In some embodiments, the first subunit comprises one or more of the following residues: (a) an F or L residue at a position corresponding to position 54 of SEQ ID NO: 1; and (b) a Q residue at a position corresponding to position 80 of SEQ ID NO: 1.

In some embodiments, the second subunit comprises one or more of the following residues: (a) an A residue at a position corresponding to position 19 of SEQ ID NO: 1; and (b) a Q residue at a position corresponding to position 80 of SEQ ID NO: 1.

In some embodiments, the first subunit comprises residues corresponding to residues 54 and 80 of any one of SEQ ID NOs: 121-135.

In some embodiments, the second subunit comprises residues corresponding to residues 19 and 80 of any one of SEQ ID NOs: 121-135.

In some embodiments, the second subunit further comprises an R residue inserted between positions corresponding to positions 73 and 74 of SEQ ID NO: 1.

Another aspect is a method for cleaving double-stranded DNA at a target site comprising a meganuclease recognition sequence recognition sequence comprising a center sequence consisting of ACGG, the method comprising contacting the double-stranded DNA having the target site with an engineered meganuclease described herein, wherein the engineered meganuclease binds and cleaves the recognition sequence.

In some embodiments, the center sequence consists of ACGT.

In some embodiments, the first subunit comprises one or more of the following residues: (a) a K, L, S, or H residue at a position corresponding to position 48 of SEQ ID NO: 1; (b) a Q, R, C, S, or V residue at a position corresponding to position 50 of SEQ ID NO: 1; (c) a G residue at a position corresponding to position 71 of SEQ ID NO: 1; (d) an R residue at a position corresponding to position 72 of SEQ ID NO: 1; and (e) an A residue at a position corresponding to position 73 of SEQ ID NO: 1.

In some embodiments, the second subunit comprises one or more of the following residues: (a) an H, K, L, or S residue at a position corresponding to position 48 of SEQ ID NO: 1; (b) an S, C, Q, E, or A residue at a position corresponding to position 50 of SEQ ID NO: 1; (c) an S, P, G, T, A, R, or N residue at a position corresponding to position 71 of SEQ ID NO: 1; (d) a T, R, K, or A residue at a position corresponding to position 72 of SEQ ID NO: 1; (e) an H, C, A, or S residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, and 73 of any one of SEQ ID NOs: 138-156.

In some embodiments, the second subunit comprises residues corresponding to residues 239, 241, 262, 263, 264, and 265 of any one of SEQ ID NOs: 138-156.

In some embodiments, the first subunit comprises one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the second subunit comprises one or more of the following residues: (a) an A or G residue at a position corresponding to position 19 of SEQ ID NO: 1; (b) a Q or E residue at a position corresponding to position 80 of SEQ ID NO: 1; (c) an H or Y residue at a position corresponding to position 85 of SEQ ID NO: 1; and (d) a K or R residue at a position corresponding to position 139 of SEQ ID NO: 1.

In some embodiments, the first subunit comprises residues corresponding to residues 19, 80, and 139 of any one of SEQ ID NOs: 138-156.

In some embodiments, the second subunit comprises residues corresponding to residues 19, 80, 85, and 139 of any one of SEQ ID NOs: 138-156.

Another aspect is a method for cleaving double-stranded DNA at a target site comprising a meganuclease recognition sequence recognition sequence comprising a center sequence consisting of ACGT, the method comprising contacting the double-stranded DNA having the target site with an engineered meganuclease described herein, wherein the engineered meganuclease binds and cleaves the recognition sequence.

In some embodiments, the center sequence consists of ATAA.

In some embodiments, the first subunit comprises one or more of the following residues: (a) a K, A, H, S, L, or Q residue at a position corresponding to position 48 of SEQ ID NO: 1; (b) a Q, T, R, I, G, K, D, C, or V residue at a position corresponding to position 50 of SEQ ID NO: 1; (c) a G, K, S, H, or N residue at a position corresponding to position 71 of SEQ ID NO: 1; (d) an R, A, G, Q, H, L, or S residue at a position corresponding to position 72 of SEQ ID NO: 1; (e) an A, T, or C residue at a position corresponding to position 73 of SEQ ID NO: 1; and (f) an S or A residue at a position corresponding to position 74 of SEQ ID NO: 1.

In some embodiments, the second subunit comprises one or more of the following residues: (a) an S, T, A, K, or N residue at a position corresponding to position 48 of SEQ ID NO: 1; (b) an R, K, E, A, C, or T residue at a position corresponding to position 50 of SEQ ID NO: 1; (c) an S, G, K, or R residue at a position corresponding to position 71 of SEQ ID NO: 1; (d) a T, R, Q, G, A, Y, S, N, or K residue at a position corresponding to position 72 of SEQ ID NO: 1; (e) an I, C, or V residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, 73, and 74 of any one of SEQ ID NOs: 159-183.

In some embodiments, the second subunit comprises residues corresponding to residues 239, 241, 262, 263, 264, and 265 of any one of SEQ ID NOs: 159-183.

In some embodiments, the first subunit comprises one or more of the following residues: (a) an A, G, or S residue at a position corresponding to position 19 of SEQ ID NO: 1; (b) a Q or E residue at a position corresponding to position 80 of SEQ ID NO: 1; (c) a K or E residue at a position corresponding to position 100 of SEQ ID NO: 1; (d) a K or R residue at a position corresponding to position 139 of SEQ ID NO: 1; (e) an S or G residue at a position corresponding to position 154 of SEQ ID NO: 1; and (f) an S or A residue at a position corresponding to position 172 of SEQ ID NO: 1.

In some embodiments, the second subunit comprises one or more of the following residues: (a) a G, S, or A residue at a position corresponding to position 19 of SEQ ID NO: 1; (b) a V or A residue at a position corresponding to position 59 of SEQ ID NO: 1; (c) an L residue at a position corresponding to position 78 of SEQ ID NO: 1; (d) an S residue at a position corresponding to position 79 of SEQ ID NO: 1; (e) a Q or E residue at a position corresponding to position 80 of SEQ ID NO: 1; (f) an S or F residue at a position corresponding to position 118 of SEQ ID NO: 1; and (g) a K or R residue at a position corresponding to position 139 of SEQ ID NO: 1.

In some embodiments, the first subunit comprises residues corresponding to residues 19, 80, 100, 139, 154, and 172 of any one of SEQ ID NOs: 159-183.

In some embodiments, the second subunit comprises residues corresponding to residues 19, 59, 78, 79, 80, 118, and 139 of any one of SEQ ID NOs: 159-183.

Another aspect is a method for cleaving double-stranded DNA at a target site comprising a meganuclease recognition sequence recognition sequence comprising a center sequence consisting of ATAA, the method comprising contacting the double-stranded DNA having the target site with an engineered meganuclease described herein, wherein the engineered meganuclease binds and cleaves the recognition sequence.

In some embodiments, the center sequence consists of ATAG.

In some embodiments, the first subunit comprises one or more of the following residues: (a) a K or H residue at a position corresponding to position 48 of SEQ ID NO: 1; (b) an R residue at a position corresponding to position 50 of SEQ ID NO: 1; (c) a G, R, or H residue at a position corresponding to position 71 of SEQ ID NO: 1; (d) an R, G, S, A, P, or Q residue at a position corresponding to position 72 of SEQ ID NO: 1; and (e) an A or C residue at a position corresponding to position 73 of SEQ ID NO: 1.

In some embodiments, the second subunit comprises one or more of the following residues: (a) a C or R residue at a position corresponding to position 50 of SEQ ID NO: 1; (b) a G or S residue at a position corresponding to position 72 of SEQ ID NO: 1; and (c) an R residue at a position corresponding to position 73 of SEQ ID NO: 1.

In some embodiments, the first subunit comprises residues corresponding to residues 48, 50, 71, 72, and 73 of any one of SEQ ID NOs: 186-199.

In some embodiments, the second subunit comprises residues corresponding to residues 241, 263, and 264 of any one of SEQ ID NOs: 186-199.

In some embodiments, the first subunit comprises one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the second subunit comprises one or more of the following residues: (a) a G or A residue at a position corresponding to position 19 of SEQ ID NO: 1; (b) a K or R residue at a position corresponding to position 36 of SEQ ID NO: 1; (c) a V or A residue at a position corresponding to position 59 of SEQ ID NO: 1; (d) a Q residue at a position corresponding to position 80 of SEQ ID NO: 1; and (e) a K or R residue at a position corresponding to position 139 of SEQ ID NO: 1.

In some embodiments, the first subunit comprises residues corresponding to residues 19, 80, and 139 of any one of SEQ ID NOs: 186-199.

In some embodiments, the second subunit comprises residues corresponding to residues 19, 36, 59, 80, and 139 of any one of SEQ ID NOs: 186-199.

Another aspect is a method for cleaving double-stranded DNA at a target site comprising a meganuclease recognition sequence recognition sequence comprising a center sequence consisting of ATAG, the method comprising contacting the double-stranded DNA having the target site with an engineered meganuclease described herein, wherein the engineered meganuclease binds and cleaves the recognition sequence.

In some embodiments, the center sequence consists of ATAT.

In some embodiments, the first subunit comprises one or more of the following residues: (a) a K, H, C, A, S, D, or T residue at a position corresponding to position 48 of SEQ ID NO: 1; (b) a Q, N, C, R, K, S, T, or V residue at a position corresponding to position 50 of SEQ ID NO: 1; (c) a G, H, or I residue at a position corresponding to position 71 of SEQ ID NO: 1; (d) an R, A, N, or Q residue at a position corresponding to position 72 of SEQ ID NO: 1; and (e) an A, C, or S residue at a position corresponding to position 73 of SEQ ID NO: 1.

In some embodiments, the second subunit comprises one or more of the following residues: (a) an H, K, A, S, R, or T residue at a position corresponding to position 48 of SEQ ID NO: 1; (b) an S, C, K, R, Q, or N residue at a position corresponding to position 50 of SEQ ID NO: 1; (c) an S, K, E, I, G, or R residue at a position corresponding to position 71 of SEQ ID NO: 1; (d) a T, A, R, S, K, G, or N residue at a position corresponding to position 72 of SEQ ID NO: 1; (e) an H, C, A, S, or G residue at a position corresponding to position 73 of SEQ ID NO: 1; and (f) an S, C, or A residue at a position corresponding to position 74 of SEQ ID NO: 1.

In some embodiments, the first subunit comprises residues corresponding to residues 48, 50, 71, 72, and 73 of any one of SEQ ID NOs: 202-219.

In some embodiments, the second subunit comprises residues corresponding to residues 239, 241, 262, 263, 264, and 265 of any one of SEQ ID NOs: 202-219.

In some embodiments, the first subunit comprises one or more of the following residues: (a) an A or G residue at a position corresponding to position 19 of SEQ ID NO: 1; (b) a Q or E residue at a position corresponding to position 80 of SEQ ID NO: 1; and (c) a K, R, or S residue at a position corresponding to position 139 of SEQ ID NO: 1.

In some embodiments, the second subunit comprises one or more of the following residues: (a) a G or A residue at a position corresponding to position 19 of SEQ ID NO: 1; (b) a V or A residue at a position corresponding to position 59 of SEQ ID NO: 1; (c) a Q, E, or K residue at a position corresponding to position 80 of SEQ ID NO: 1; and (d) a K, R, P, or N residue at a position corresponding to position 139 of SEQ ID NO: 1.

In some embodiments, the first subunit comprises residues corresponding to residues 19, 80, and 139 of any one of SEQ ID NOs: 202-219.

In some embodiments, the second subunit comprises residues corresponding to residues 19, 59, 80, and 139 of any one of SEQ ID NOs: 202-219.

Another aspect is a method for cleaving double-stranded DNA at a target site comprising a meganuclease recognition sequence recognition sequence comprising a center sequence consisting of ATAT, the method comprising contacting the double-stranded DNA having the target site with an engineered meganuclease described herein, wherein the engineered meganuclease binds and cleaves the recognition sequence.

In some embodiments, the center sequence consists of ATGA.

In some embodiments, the first subunit comprises one or more of the following residues: (a) a K, A, H, or L residue at a position corresponding to position 48 of SEQ ID NO: 1; (b) an R, T, E, S, C, or V residue at a position corresponding to position 50 of SEQ ID NO: 1; (c) an R, T, S, A, or K residue at a position corresponding to position 72 of SEQ ID NO: 1; and (d) an A or S residue at a position corresponding to position 73 of SEQ ID NO: 1.

In some embodiments, the second subunit comprises one or more of the following residues: (a) an H, K, R, A, or S residue at a position corresponding to position 48 of SEQ ID NO: 1; (b) an S, I, R, C, A, or Q residue at a position corresponding to position 50 of SEQ ID NO: 1; (c) an R or H residue at a position corresponding to position 72 of SEQ ID NO: 1; (d) an I or V residue at a position corresponding to position 73 of SEQ ID NO: 1; and (e) an S.

A, or T residue at a position corresponding to position 74 of SEQ ID NO: 1.

In some embodiments, the first subunit comprises residues corresponding to residues 48, 50, 72, and 73 of any one of SEQ ID NOs: 222-243.

In some embodiments, the second subunit comprises residues corresponding to residues 239, 241, 263, 264, and 265 of any one of SEQ ID NOs: 222-243.

In some embodiments, the first subunit comprises one or more of the following residues: (a) an A, G, or S residue at a position corresponding to position 19 of SEQ ID NO: 1; (b) a Q or E residue at a position corresponding to position 80 of SEQ ID NO: 1; (c) an F or L residue at a position corresponding to position 87 of SEQ ID NO: 1; (d) a Q or R residue at a position corresponding to position 92 of SEQ ID NO: 1; and (e) a K or R residue at a position corresponding to position 139 of SEQ ID NO: 1.

In some embodiments, the second subunit comprises one or more of the following residues: (a) a G, A, or S residue at a position corresponding to position 19 of SEQ ID NO: 1; (b) a V or A residue at a position corresponding to position 59 of SEQ ID NO: 1; (c) a Q or E residue at a position corresponding to position 80 of SEQ ID NO: 1; and (d) a K or R residue at a position corresponding to position 139 of SEQ ID NO: 1.

In some embodiments, the first subunit comprises residues corresponding to residues 19, 80, 87, 92, and 139 of any one of SEQ ID NOs: 222-243.

In some embodiments, the second subunit comprises residues corresponding to residues 19, 59, 80, and 139 of any one of SEQ ID NOs: 222-243.

Another aspect is a method for cleaving double-stranded DNA at a target site comprising a meganuclease recognition sequence recognition sequence comprising a center sequence consisting of ATGA, the method comprising contacting the double-stranded DNA having the target site with an engineered meganuclease described herein, wherein the engineered meganuclease binds and cleaves the recognition sequence.

In some embodiments, the center sequence consists of ATGG.

In some embodiments, the first subunit comprises one or more of the following residues: (a) an R residue at a position corresponding to position 50 of SEQ ID NO: 1; (b) a G or S residue at a position corresponding to position 71 of SEQ ID NO: 1; (c) a P or G residue at a position corresponding to position 72 of SEQ ID NO: 1; and (d) an A or C residue at a position corresponding to position 73 of SEQ ID NO: 1; (e) an S or C residue at a position corresponding to position 74 of SEQ ID NO: 1.

In some embodiments, the second subunit comprises one or more of the following residues: (a) a R residue at a position corresponding to position 50 of SEQ ID NO: 1; (b) a D or G residue at a position corresponding to position 71 of SEQ ID NO: 1; (c) a G residue at a position corresponding to position 72 of SEQ ID NO: 1; and (d) an R residue at a position corresponding to position 73 of SEQ ID NO: 1.

In some embodiments, the first subunit comprises residues corresponding to residues 50, 71, 72, 73, and 74 of any one of SEQ ID NOs: 246-247.

In some embodiments, the second subunit comprises residues corresponding to residues 239, 241, 262, 263, and 264 of any one of SEQ ID NOs: 246-247.

In some embodiments, the first subunit comprises one or more of the following residues: (a) an A or G residue at a position corresponding to position 19 of SEQ ID NO: 1; (b) an E or Q residue at a position corresponding to position 80 of SEQ ID NO: 1; (c) an E or K residue at a position corresponding to position 82 of SEQ ID NO: 1; and (d) an R or K residue at a position corresponding to position 139 of SEQ ID NO: 1.

In some embodiments, the second subunit comprises one or more of the following residues: (a) an A or G residue at a position corresponding to position 19 of SEQ ID NO: 1; (b) a N residue at a position corresponding to position 77 of SEQ ID NO: 1; and (c) a Q or R residue at a position corresponding to position 80 of SEQ ID NO: 1.

In some embodiments, the first subunit comprises residues corresponding to residues 19, 80, 82, and 139 of any one of SEQ ID NOs: 246-247.

In some embodiments, the second subunit comprises residues corresponding to residues 19, 77, and 80 of any one of SEQ ID NOs: 246-247.

In some embodiments, the second subunit further comprises an R residue inserted between positions corresponding to positions 73 and 74 of SEQ ID NO: 1.

Another aspect is a method for cleaving double-stranded DNA at a target site comprising a meganuclease recognition sequence recognition sequence comprising a center sequence consisting of ATGG, the method comprising contacting the double-stranded DNA having the target site with an engineered meganuclease described herein, wherein the engineered meganuclease binds and cleaves the recognition sequence.

In some embodiments, the center sequence consists of TTGG.

In some embodiments, the first subunit comprises one or more of the following residues: (a) an R residue at a position corresponding to position 50 of SEQ ID NO: 1; (b) an S residue at a position corresponding to position 71 of SEQ ID NO: 1; (c) a G residue at a position corresponding to position 72 of SEQ ID NO: 1; and (d) an R residue at a position corresponding to position 73 of SEQ ID NO: 1.

In some embodiments, the second subunit comprises one or more of the following residues: (a) a K or S residue at a position corresponding to position 48 of SEQ ID NO: 1; (b) a C, T, E, K, or R residue at a position corresponding to position 50 of SEQ ID NO: 1; (c) a G or K residue at a position corresponding to position 71 of SEQ ID NO: 1; (d) a T, Q, K, R, H, A, or S residue at a position corresponding to position 72 of SEQ ID NO: 1; (e) an I or V residue at a position corresponding to position 73 of SEQ ID NO: 1; and (f) an S or A residue at a position corresponding to position 74 of SEQ ID NO: 1.

In some embodiments, the first subunit comprises residues corresponding to residues 50, 71, 72, and 73 of any one of SEQ ID NOs: 250-266.

In some embodiments, the second subunit comprises residues corresponding to residues 239, 241, 262, 263, 264, and 265 of any one of SEQ ID NOs: 250-266.

In some embodiments, the first subunit comprises one or more of the following residues: (a) an A or G residue at a position corresponding to position 19 of SEQ ID NO: 1; and (b) a Q residue at a position corresponding to position 80 of SEQ ID NO: 1.

In some embodiments, the second subunit comprises one or more of the following residues: (a) a G or A residue at a position corresponding to position 19 of SEQ ID NO: 1; (b) a Y or H residue at a position corresponding to position 66 of SEQ ID NO: 1; (c) a Q residue at a position corresponding to position 80 of SEQ ID NO: 1; (d) an H or R residue at a position corresponding to position 85 of SEQ ID NO: 1; and (e) a K or R residue at a position corresponding to position 139 of SEQ ID NO: 1.

In some embodiments, the first subunit comprises residues corresponding to residues 19 and 80 of any one of SEQ ID NOs: 250-266.

In some embodiments, the second subunit comprises residues corresponding to residues 19, 66, 80, 85, and 139 of any one of SEQ ID NOs: 250-266.

Another aspect is a method for cleaving double-stranded DNA at a target site comprising a meganuclease recognition sequence recognition sequence comprising a center sequence consisting of TTGG, the method comprising contacting the double-stranded DNA having the target site with an engineered meganuclease described herein, wherein the engineered meganuclease binds and cleaves the recognition sequence.

In some embodiments, the center sequence consists of GCAA.

In some embodiments, the first subunit comprises one or more of the following residues: (a) a K or H residue at a position corresponding to position 48 of SEQ ID NO: 1; (b) an R, C, K, T, or L residue at a position corresponding to position 50 of SEQ ID NO: 1; (c) a G, N, T, R, S, or H residue at a position corresponding to position 71 of SEQ ID NO: 1; (d) an R, P, S, N, Q, G, A, T, M, or V residue at a position corresponding to position 72 of SEQ ID NO: 1; (e) a T or V residue at a position corresponding to position 73 of SEQ ID NO: 1; and (f) an S, C, or A residue at a position corresponding to position 74 of SEQ ID NO: 1.

In some embodiments, the second subunit comprises one or more of the following residues: (a) an S, A, K, or T residue at a position corresponding to position 48 of SEQ ID NO: 1; (b) an R, C, T, K, or E residue at a position corresponding to position 50 of SEQ ID NO: 1; (c) a G, R, A, or H residue at a position corresponding to position 71 of SEQ ID NO: 1; (d) a T, G, S, A, E, N, K, H, R, C, or Y residue at a position corresponding to position 72 of SEQ ID NO: 1; (e) a C, V, or I residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, 73, and 74 of any one of SEQ ID NOs: 269-291.

In some embodiments, the second subunit comprises residues corresponding to residues 239, 241, 262, 263, 264, and 265 of any one of SEQ ID NOs: 269-291.

In some embodiments, the first subunit comprises one or more of the following residues: (a) an A, G, or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the second subunit comprises one or more of the following residues: (a) a G or A residue at a position corresponding to position 19 of SEQ ID NO: 1; (b) a Q or P residue at a position corresponding to position 31 of SEQ ID NO: 1; (c) a Q or E residue at a position corresponding to position 80 of SEQ ID NO: 1; and (d) a K or R residue at a position corresponding to position 139 of SEQ ID NO: 1.

In some embodiments, the first subunit comprises residues corresponding to residues 19, 80, and 139 of any one of SEQ ID NOs: 269-291.

In some embodiments, the second subunit comprises residues corresponding to residues 19, 31, 80, and 139 of any one of SEQ ID NOs: 269-291.

Another aspect is a method for cleaving double-stranded DNA at a target site comprising a meganuclease recognition sequence recognition sequence comprising a center sequence consisting of GCAA, the method comprising contacting the double-stranded DNA having the target site with an engineered meganuclease described herein, wherein the engineered meganuclease binds and cleaves the recognition sequence.

In some embodiments, the center sequence consists of GCAT.

In some embodiments, the first subunit comprises one or more of the following residues: (a) a K, A, H, or R residue at a position corresponding to position 48 of SEQ ID NO: 1; (b) a Q, V, R, K, or S residue at a position corresponding to position 50 of SEQ ID NO: 1; (c) a G, A, H, R, T, N, or S residue at a position corresponding to position 71 of SEQ ID NO: 1; (d) an R, T, G, S, Q, N, or A residue at a position corresponding to position 72 of SEQ ID NO: 1; (c) an A, T, V, or C residue at a position corresponding to position 73 of SEQ ID NO: 1; and (f) an S or A residue at a position corresponding to position 74 of SEQ ID NO: 1.

In some embodiments, the second subunit comprises one or more of the following residues: (a) an H, A, K, T, L, or I residue at a position corresponding to position 48 of SEQ ID NO: 1; (b) an S, R, K, Q, H, or V residue at a position corresponding to position 50 of SEQ ID NO: 1; (c) an S, K, R, A, G, T, H, or Y residue at a position corresponding to position 71 of SEQ ID NO: 1; (d) a T, A, G, N, S, R, H, Q, or K residue at a position corresponding to position 72 of SEQ ID NO: 1; (c) an H, C, G, S, or A residue at a position corresponding to position 73 of SEQ ID NO: 1; and (f) an S, C, or A residue at a position corresponding to position 74 of SEQ ID NO: 1.

In some embodiments, the first subunit comprises residues corresponding to residues 48, 50, 71, 72, 73, and 74 of any one of SEQ ID NOs: 294-313.

In some embodiments, the second subunit comprises residues corresponding to residues 239, 241, 262, 263, 264, and 265 of any one of SEQ ID NOs: 294-313.

In some embodiments, the first subunit comprises one or more of the following residues: (a) an A or G residue at a position corresponding to position 19 of SEQ ID NO: 1; (b) a Q or E residue at a position corresponding to position 80 of SEQ ID NO: 1; (c) a K, H, or R residue at a position corresponding to position 139 of SEQ ID NO: 1; and (d) a T or I residue at a position corresponding to position 143 of SEQ ID NO: 1.

In some embodiments, the second subunit comprises one or more of the following residues: (a) a G, S, or A residue at a position corresponding to position 19 of SEQ ID NO: 1; (b) a Q or E residue at a position corresponding to position 80 of SEQ ID NO: 1; (c) a V or A residue at a position corresponding to position 125 of SEQ ID NO: 1; and (d) a K, R, or H residue at a position corresponding to position 139 of SEQ ID NO: 1.

In some embodiments, the first subunit comprises residues corresponding to residues 19, 80, 139, and 143 of any one of SEQ ID NOs: 294-313.

In some embodiments, the second subunit comprises residues corresponding to residues 19, 80, 125, and 139 of any one of SEQ ID NOs: 294-313.

Another aspect is a method for cleaving double-stranded DNA at a target site comprising a meganuclease recognition sequence recognition sequence comprising a center sequence consisting of GCAT, the method comprising contacting the double-stranded DNA having the target site with an engineered meganuclease described herein, wherein the engineered meganuclease binds and cleaves the recognition sequence.

In some embodiments, the center sequence consists of GCGA.

In some embodiments, the first subunit comprises one or more of the following residues: (a) a K or R residue at a position corresponding to position 50 of SEQ ID NO: 1; (b) a G, R, S, A, or N residue at a position corresponding to position 71 of SEQ ID NO: 1; (c) an R, N, G, A, or Q residue at a position corresponding to position 72 of SEQ ID NO: 1; (d) a V, T, or I residue at a position corresponding to position 73 of SEQ ID NO: 1; and (e) an S or A residue at a position corresponding to position 74 of SEQ ID NO: 1.

In some embodiments, the second subunit comprises one or more of the following residues: (a) a K, T, S, A, or Q residue at a position corresponding to position 48 of SEQ ID NO: 1; (b) a C or R residue at a position corresponding to position 50 of SEQ ID NO: 1; (c) an R residue at a position corresponding to position 72 of SEQ ID NO: 1; (d) a V or I residue at a position corresponding to position 73 of SEQ ID NO: 1; and (e) an S or A residue at a position corresponding to position 74 of SEQ ID NO: 1.

In some embodiments, the first subunit comprises residues corresponding to residues 50, 71, 72, 73, and 74 of any one of SEQ ID NOs: 316-325.

In some embodiments, the second subunit comprises residues corresponding to residues 239, 241, 263, 264, and 265 of any one of SEQ ID NOs: 316-325.

In some embodiments, the first subunit comprises one or more of the following residues: (a) an A, G or S residue at a position corresponding to position 19 of SEQ ID NO: 1; and (b) a Q or E residue at a position corresponding to position 80 of SEQ ID NO: 1.

In some embodiments, the second subunit comprises one or more of the following residues: (a) a G, S, or A residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments, the first subunit comprises residues corresponding to residues 19 and 80 of any one of SEQ ID NOs: 316-325.

In some embodiments, the second subunit comprises residues corresponding to residues 19, 80, and 139 of any one of SEQ ID NOs: 316-325.

Another aspect is a method for cleaving double-stranded DNA at a target site comprising a meganuclease recognition sequence recognition sequence comprising a center sequence consisting of GCGA, the method comprising contacting the double-stranded DNA having the target site with an engineered meganuclease described herein, wherein the engineered meganuclease binds and cleaves the recognition sequence.

In some embodiments, the center sequence consists of GCAG.

In some embodiments, the first subunit comprises one or more of the following residues: (a) a R residue at a position corresponding to position 50 of SEQ ID NO: 1; (b) a S residue at a position corresponding to position 71 of SEQ ID NO: 1; (c) an G residue at a position corresponding to position 72 of SEQ ID NO: 1; and (d) a R residue at a position corresponding to position 73 of SEQ ID NO: 1; In some embodiments, the second subunit comprises one or more of the following residues: (a) a K or H residue at a position corresponding to position 48 of SEQ ID NO: 1; (b) a Q or R residue at a position corresponding to position 50 of SEQ ID NO: 1; (c) a S or R residue at a position corresponding to position 72 of SEQ ID NO: 1; (d) a V or T residue at a position corresponding to position 73 of SEQ ID NO: 1; and

In some embodiments, the first subunit comprises residues corresponding to residues 50, 71, 72, 73, and 74 of any one of SEQ ID NOs: 328-330.

In some embodiments, the second subunit comprises residues corresponding to residues 239, 241, 263, 264, and 265 of any one of SEQ ID NOs: 328-330.

In some embodiments, the second subunit comprises an E residue at a position corresponding to position 80 of SEQ ID NO: 1.

In some embodiments, the second subunit comprises residues corresponding to residues 80 of any one of SEQ ID NOs: 328-330.

Another aspect is a method for cleaving double-stranded DNA at a target site comprising a meganuclease recognition sequence recognition sequence comprising a center sequence consisting of GCAG, the method comprising contacting the double-stranded DNA having the target site with an engineered meganuclease described herein, wherein the engineered meganuclease binds and cleaves the recognition sequence.

In some embodiments, the center sequence consists of TCAA.

In some embodiments, the first subunit comprises one or more of the following residues: (a) a K or S residue at a position corresponding to position 48 of SEQ ID NO: 1; (b) an R, T, or C residue at a position corresponding to position 50 of SEQ ID NO: 1; (c) a G, R, or T residue at a position corresponding to position 71 of SEQ ID NO: 1; and (d) an R, S, P, T, or G residue at a position corresponding to position 72 of SEQ ID NO: 1.

In some embodiments, the second subunit comprises one or more of the following residues: (a) an S or K residue at a position corresponding to position 48 of SEQ ID NO: 1; (b) a K, R, C, or E residue at a position corresponding to position 50 of SEQ ID NO: 1; (c) an R, Q, N, or S residue at a position corresponding to position 72 of SEQ ID NO: 1; (d) an I residue at a position corresponding to position 73 of SEQ ID NO: 1; and (e) an S or A residue at a position corresponding to position 74 of SEQ ID NO: 1.

In some embodiments, the first subunit comprises residues corresponding to residues 48, 50, 71, and 72 of any one of SEQ ID NOs: 333-340.

In some embodiments, the second subunit comprises residues corresponding to residues 239, 241, 263, 264, and 265 of any one of SEQ ID NOs: 333-340.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the second subunit comprises one or more of the following residues: (a) a G or S residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments, the first subunit comprises residues corresponding to residues 19, 80, and 139 of any one of SEQ ID NOs: 333-340.

In some embodiments, the second subunit comprises residues corresponding to residues 19, 80, and 139 of any one of SEQ ID NOs: 333-340.

Another aspect is a method for cleaving double-stranded DNA at a target site comprising a meganuclease recognition sequence recognition sequence comprising a center sequence consisting of TCAA, the method comprising contacting the double-stranded DNA having the target site with an engineered meganuclease described herein, wherein the engineered meganuclease binds and cleaves the recognition sequence.

In some embodiments, the center sequence consists of TTAA.

(a) a K, N, S, or R residue at a position corresponding to position 48 of SEQ ID NO: 1; (b) an R, V, K, or S residue at a position corresponding to position 50 of SEQ ID NO: 1; (c) a G, R, N, S, or A residue at a position corresponding to position 71 of SEQ ID NO: 1; (d) an R, T, S, N, D, Q, K, or A residue at a position corresponding to position 72 of SEQ ID NO: 1; and (e) an S or A residue at a position corresponding to position 74 of SEQ ID NO: 1.

In some embodiments, the second subunit comprises one or more of the following residues: (a) a K, S, A, or T residue at a position corresponding to position 48 of SEQ ID NO: 1; (b) a C, K, R, T, or E residue at a position corresponding to position 50 of SEQ ID NO: 1; (c) a T, K, R, A, S, or Q residue at a position corresponding to position 72 of SEQ ID NO: 1; (d) an I or V residue at a position corresponding to position 73 of SEQ ID NO: 1; and (e) an S or A residue at a position corresponding to position 74 of SEQ ID NO: 1.

In some embodiments, the first subunit comprises residues corresponding to residues 48, 50, 71, 72, and 74 of any one of SEQ ID NOs: 343-357.

In some embodiments, the second subunit comprises residues corresponding to residues 239, 241, 263, 264, and 265 of any one of SEQ ID NOs: 343-357.

In some embodiments, the first subunit comprises one or more of the following residues: (a) an A, G, or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the second subunit comprises one or more of the following residues: (a) a G, A, or S residue at a position corresponding to position 19 of SEQ ID NO: 1; (b) a Y or H residue at a position corresponding to position 66 of SEQ ID NO: 1; (c) a Q residue at a position corresponding to position 80 of SEQ ID NO: 1; and (d) an R residue at a position corresponding to position 139 of SEQ ID NO: 1.

In some embodiments, the first subunit comprises residues corresponding to residues 19, 80, and 139 of any one of SEQ ID NOs: 343-357.

In some embodiments, the second subunit comprises residues corresponding to residues 19, 66, 80, and 139 of any one of SEQ ID NOs: 343-357.

Another aspect is a method for cleaving double-stranded DNA at a target site comprising a meganuclease recognition sequence recognition sequence comprising a center sequence consisting of TTAA, the method comprising contacting the double-stranded DNA having the target site with an engineered meganuclease described herein, wherein the engineered meganuclease binds and cleaves the recognition sequence.

Another aspect is a method for increasing the cleavage activity of an engineered meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, the method comprising modifying each of the first subunit and the second subunit at one or more positions corresponding to positions 48, 50, 71, 72, 73, and 74 of SEQ ID NO: 1, wherein the modified nuclease has increased cleavage activity when compared to a control engineered meganuclease.

In some embodiments of the method, the center sequence consists of ACAA.

In some embodiments of the method, the modifying step comprises modifying the first subunit to comprise one or more of the following residues: (a) a K or L residue at a position corresponding to position 48 of SEQ ID NO: 1; (b) a C, R, T, K, or S residue at a position corresponding to position 50 of SEQ ID NO: 1; (c) a G or R residue at a position corresponding to position 71 of SEQ ID NO: 1; (d) an R or Q residue at a position corresponding to position 72 of SEQ ID NO: 1; and (e) an A or C residue at a position corresponding to position 73 of SEQ ID NO: 1.

In some embodiments of the method, the modifying step comprises modifying the second subunit to comprise one or more of the following residues: (a) a K, T, S, or A residue at a position corresponding to position 48 of SEQ ID NO: 1; (b) a C, R, E, K, or T residue at a position corresponding to position 50 of SEQ ID NO: 1; (c) a G or A residue at a position corresponding to position 71 of SEQ ID NO: 1; (d) a T, R, S, P, N, G, or A residue at a position corresponding to position 72 of SEQ ID NO: 1; (e) a V or I residue at a position corresponding to position 73 of SEQ ID NO: 1; and (f) an S, T, or A residue at a position corresponding to position 74 of SEQ ID NO: 1.

In some embodiments of the method, the first subunit is modified to comprise residues corresponding to residues 48, 50, 71, 72, and 73 of any one of SEQ ID NOs: 8-30.

In some embodiments of the method, the second subunit is modified to comprise residues corresponding to residues 239, 241, 262, 263, 264, and 265 of any one of SEQ ID NOs: 8-30.

In some embodiments of the method, the method further comprises modifying the first subunit to comprise one or more of the following residues: (a) an A or G residue at a position corresponding to position 19 of SEQ ID NO: 1; (b) a Q or E residue at a position corresponding to position 80 of SEQ ID NO: 1; (c) a K or R residue at a position corresponding to position 139 of SEQ ID NO: 1; and (d) an S or G residue at a position corresponding to position 154 of SEQ ID NO: 1.

In some embodiments of the method, the method further comprises modifying the second subunit to comprise one or more of the following residues: (a) a G, A, or S residue at a position corresponding to position 19 of SEQ ID NO: 1; (b) a Y or C residue at a position corresponding to position 66 of SEQ ID NO: 1; (c) a Q or E residue at a position corresponding to position 80 of SEQ ID NO: 1; (d) a Q or R residue at a position corresponding to position 92 of SEQ ID NO: 1; (e) an E or G residue at a position corresponding to position 117 of SEQ ID NO: 1; and (f) a K or R residue at a position corresponding to position 139 of SEQ ID NO: 1.

In some embodiments of the method, the first subunit is modified to comprise residues corresponding to residues 19, 80, 139, and 154 of any one of SEQ ID NOs: 8-30.

In some embodiments of the method, the second subunit is modified to comprise residues corresponding to residues 19, 66, 80, 92, 117, and 139 of any one of SEQ ID NOs: 8-30.

In some embodiments of the method, the center sequence consists of ACAG.

In some embodiments of the method, the modifying step comprises modifying the first subunit to comprise one or more of the following residues: (a) an R residue at a position corresponding to position 50 of SEQ ID NO: 1; (b) a G or R residue at a position corresponding to position 71 of SEQ ID NO: 1; (c) an R, K, Q, P, or T residue at a position corresponding to position 72 of SEQ ID NO: 1; (d) an A or C residue at a position corresponding to position 73 of SEQ ID NO: 1; and optionally (e) an R residue at a position following a position corresponding to position 73 of SEQ ID NO: 1.

In some embodiments of the method, the modifying step comprises modifying the second subunit to comprise one or more of the following residues: (a) a C residue at a position corresponding to position 50 of SEQ ID NO: 1; (b) a G, S, or D residue at a position corresponding to position 71 of SEQ ID NO: 1; (c) an R or G residue at a position corresponding to position 72 of SEQ ID NO: 1; and (d) an R residue at a position corresponding to position 73 of SEQ ID NO: 1.

In some embodiments of the method, the first subunit is modified to comprise residues corresponding to residues 50, 71, 72, and 73 of any one of SEQ ID NOs: 33-40.

In some embodiments of the method, the second subunit is modified to comprise residues corresponding to residues 241, 262, 263, and 264 of any one of SEQ ID NOs: 33-40.

In some embodiments of the method, the method further comprises modifying the first subunit to comprise one or more of the following residues: (a) an A or G residue at a position corresponding to position 19 of SEQ ID NO: 1; (b) a F, I, or L residue at a position corresponding to position 54 of SEQ ID NO: 1; (c) a Q or E residue at a position corresponding to position 80 of SEQ ID NO: 1; and (d) a S or P residue at a position corresponding to position 158 of SEQ ID NO: 1.

In some embodiments of the method, the method further comprises modifying the second subunit to comprise one or more of the following residues: (a) a G, A, or S residue at a position corresponding to position 19 of SEQ ID NO: 1; (b) a V or A residue at a position corresponding to position 59 of SEQ ID NO: 1; (c) a Y or H residue at a position corresponding to position 66 of SEQ ID NO: 1; (d) a Q residue at a position corresponding to position 80 of SEQ ID NO: 1; (e) an I or T residue at a position corresponding to position 81 of SEQ ID NO: 1; and (f) a K or R residue at a position corresponding to position 139 of SEQ ID NO: 1.

In some embodiments of the method, the first subunit is modified to comprise residues corresponding to residues 19, 54, 80, and 158 of any one of SEQ ID NOs: 33-40.

In some embodiments of the method, the second subunit is modified to comprise residues corresponding to residues 19, 59, 66, 80, 81, and 139 of any one of SEQ ID NOs: 33-40.

In some embodiments of the method, the second subunit is further modified by inserting an R residue between positions corresponding to positions 73 and 74 of SEQ ID NO: 1.

In some embodiments of the method, the center sequence consists of ACAT.

In some embodiments of the method, the modifying step comprises modifying the first subunit to comprise one or more of the following residues: (a) a K, S, I, L, or N residue at a position corresponding to position 48 of SEQ ID NO: 1; (b) a Q, S, R, or K residue at a position corresponding to position 50 of SEQ ID NO: 1; (c) a G or R residue at a position corresponding to position 71 of SEQ ID NO: 1; (d) an R or T residue at a position corresponding to position 72 of SEQ ID NO: 1; and (e) an A or G residue at a position corresponding to position 73 of SEQ ID NO: 1.

In some embodiments of the method, the modifying step comprises modifying the second subunit to comprise one or more of the following residues: (a) an H, T, G, A, S, L, or K residue at a position corresponding to position 48 of SEQ ID NO: 1; (b) an S, K, C, N R, G, or Q residue at a position corresponding to position 50 of SEQ ID NO: 1; (c) an S, G, R, T, K, or E residue at a position corresponding to position 71 of SEQ ID NO: 1; (d) a T, K, A, S, R, H, G, or N residue at a position corresponding to position 72 of SEQ ID NO: 1; (e) an H, A, C, S, G, or R residue at a position corresponding to position 73 of SEQ ID NO: 1; and (f) an S, C, or A residue at a position corresponding to position 74 of SEQ ID NO: 1. In some embodiments of the method, the first subunit is modified to comprise residues corresponding to residues 48, 50, 71, 72, and 73 of any one of SEQ ID NOs: 43-64.

In some embodiments of the method, the second subunit is modified to comprise residues corresponding to residues 239, 241, 262, 263, 264, and 265 of any one of SEQ ID NOs: 43-64.

In some embodiments of the method, the method further comprises modifying the first subunit to comprise one or more of the following residues: (a) an A, G, or S residue at a position corresponding to position 19 of SEQ ID NO: 1; (b) an F or I residue at a position corresponding to position 54 of SEQ ID NO: 1; (c) a Q or E residue at a position corresponding to position 80 of SEQ ID NO: 1; and (d) a K, H, or R residue at a position corresponding to position 139 of SEQ ID NO: 1.

In some embodiments of the method, the method further comprises modifying the second subunit to comprise one or more of the following residues: (a) an A, G, or S residue at a position corresponding to position 19 of SEQ ID NO: 1; (b) a Q or E residue at a position corresponding to position 80 of SEQ ID NO: 1; (c) an I or T residue at a position corresponding to position 81 of SEQ ID NO: 1; (d) a P or H residue at a position corresponding to position 83 of SEQ ID NO: 1; (e) an E or G residue at a position corresponding to position 117 of SEQ ID NO: 1; and (f) a K, R, T, or H residue at a position corresponding to position 139 of SEQ ID NO: 1.

In some embodiments of the method, the first subunit is modified to comprise residues corresponding to residues 19, 54, 80, and 139 of any one of SEQ ID NOs: 43-64.

In some embodiments of the method, the second subunit is modified to comprise residues corresponding to residues 19, 80, 81, 83, 117, and 139 of any one of SEQ ID NOs: 43-64.

In some embodiments of the method, the center sequence consists of ACGA.

In some embodiments of the method, the modifying step comprises modifying the first subunit to comprise one or more of the following residues: (a) a K residue at a position corresponding to position 48 of SEQ ID NO: 1; (b) a V, R, T, W, or A residue at a position corresponding to position 50 of SEQ ID NO: 1; (c) a G or P residue at a position corresponding to position 71 of SEQ ID NO: 1; (d) an R or P residue at a position corresponding to position 72 of SEQ ID NO: 1; and (e) an A residue at a position corresponding to position 73 of SEQ ID NO: 1.

In some embodiments of the method, the modifying step comprises modifying the second subunit to comprise one or more of the following residues: (a) a K, H, T, A, G, or Q residue at a position corresponding to position 48 of SEQ ID NO: 1; (b) an R, S, C, I, V, or G residue at a position corresponding to position 50 of SEQ ID NO: 1; (c) a G residue at a position corresponding to position 71 of SEQ ID NO: 1; (d) an R or H residue at a position corresponding to position 72 of SEQ ID NO: 1; (e) an I or V residue at a position corresponding to position 73 of SEQ ID NO: 1; and (f) an S or A residue at a position corresponding to position 74 of SEQ ID NO: 1.

In some embodiments of the method, the first subunit is modified to comprise residues corresponding to residues 48, 50, 71, 72, and 73 of any one of SEQ ID NOs: 67-89.

In some embodiments of the method, the second subunit is modified to comprise residues corresponding to residues 239, 241, 262, 263, 264, and 265 of any one of SEQ ID NOs: 67-89.

In some embodiments of the method, the method further comprises modifying the first subunit to comprise one or more of the following residues: (a) an A, G, or S residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments of the method, the method further comprises modifying the second subunit to comprise one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the first subunit is modified to comprise residues corresponding to residues 19, 80, and 139 of any one of SEQ ID NOs: 67-89.

In some embodiments of the method, the second subunit is modified to comprise residues corresponding to residues 19, 80, and 139 of any one of SEQ ID NOs: 67-89.

In some embodiments of the method, the center sequence consists of ACGC.

In some embodiments of the method, the modifying step comprises modifying the first subunit to comprise one or more of the following residues: (a) a K, H, Q, L, A, or S residue at a position corresponding to position 48 of SEQ ID NO: 1; (b) a Q, R, K, S, T, or C residue at a position corresponding to position 50 of SEQ ID NO: 1; (c) a G, R, or A residue at a position corresponding to position 71 of SEQ ID NO: 1; (d) an R, P, or H residue at a position corresponding to position 72 of SEQ ID NO: 1; and (c) an A residue at a position corresponding to position 73 of SEQ ID NO: 1.

In some embodiments of the method, the modifying step comprises modifying the second subunit to comprise one or more of the following residues: (a) an H, K, L, A, S, or N residue at a position corresponding to position 48 of SEQ ID NO: 1; (b) an S, E, K, I, N, or V residue at a position corresponding to position 50 of SEQ ID NO: 1; (c) an S, G, K, A, or R residue at a position corresponding to position 71 of SEQ ID NO: 1; (d) a T, R, A, S, H, or G residue at a position corresponding to position 72 of SEQ ID NO: 1; (e) an H, T, V, I, or C residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, and 73 of any one of SEQ ID NOs: 92-118.

In some embodiments of the method, the second subunit is modified to comprise residues corresponding to residues 239, 241, 262, 263, 264, and 265 of any one of SEQ ID NOs: 92-118.

In some embodiments of the method, the method further comprises modifying the first subunit to comprise one or more of the following residues: (a) an A, G, or S residue at a position corresponding to position 19 of SEQ ID NO: 1; and (b) a Q or E residue at a position corresponding to position 80 of SEQ ID NO: 1.

In some embodiments of the method, the method further comprises modifying the second subunit to comprise one or more of the following residues: (a) an A or G residue at a position corresponding to position 19 of SEQ ID NO: 1; (b) a Q or E residue at a position corresponding to position 80 of SEQ ID NO: 1; (c) an F or L residue at a position corresponding to position 87 of SEQ ID NO: 1; and (d) a K, R, N, H, or A residue at a position corresponding to position 139 of SEQ ID NO: 1.

In some embodiments of the method, the first subunit is modified to comprise residues corresponding to residues 19 and 80 of any one of SEQ ID NOs: 92-118.

In some embodiments of the method, the second subunit is modified to comprise residues corresponding to residues 19, 80, 87, and 139 of any one of SEQ ID NOs: 92-118.

In some embodiments of the method, the center sequence consists of ACGG.

In some embodiments of the method, the modifying step comprises modifying the first subunit to comprise one or more of the following residues: (a) an R or K residue at a position corresponding to position 50 of SEQ ID NO: 1; (b) an R residue at a position corresponding to position 72 of SEQ ID NO: 1; and (c) an A residue at a position corresponding to position 73 of SEQ ID NO: 1.

In some embodiments of the method, the modifying step comprises modifying the second subunit to comprise one or more of the following residues: (a) a K residue at a position corresponding to position 48 of SEQ ID NO: 1; (b) an R or P residue at a position corresponding to position 50 of SEQ ID NO: 1; (c) a D residue at a position corresponding to position 71 of SEQ ID NO: 1; (d) a G residue at a position corresponding to position 72 of SEQ ID NO: 1; and (e) an R or G residue at a position corresponding to position 73 of SEQ ID NO: 1.

In some embodiments of the method, the first subunit is modified to comprise residues corresponding to residues 50, 72, and 73 of any one of SEQ ID NOs: 121-135.

In some embodiments of the method, the second subunit is modified to comprise residues corresponding to residues 239, 241, 262, 263, and 264 of any one of SEQ ID NOs: 121-135.

In some embodiments of the method, the method further comprises modifying the first subunit to comprise one or more of the following residues: (a) an F or L residue at a position corresponding to position 54 of SEQ ID NO: 1; and (b) a Q residue at a position corresponding to position 80 of SEQ ID NO: 1.

In some embodiments of the method, the method further comprises modifying the second subunit to comprise one or more of the following residues: (a) an A residue at a position corresponding to position 19 of SEQ ID NO: 1; and (b) a Q residue at a position corresponding to position 80 of SEQ ID NO: 1.

In some embodiments of the method, the first subunit is modified to comprise residues corresponding to residues 54 and 80 of any one of SEQ ID NOs: 121-135.

In some embodiments of the method, the second subunit is modified to comprise residues corresponding to residues 19 and 80 of any one of SEQ ID NOs: 121-135.

In some embodiments of the method, the second subunit is further modified by inserting an R residue between positions corresponding to positions 73 and 74 of SEQ ID NO: 1.

In some embodiments of the method, the center sequence consists of ACGT.

In some embodiments of the method, the modifying step comprises modifying the first subunit to comprise one or more of the following residues: (a) a K, L, S, or H residue at a position corresponding to position 48 of SEQ ID NO: 1; (b) a Q, R, C, S, or V residue at a position corresponding to position 50 of SEQ ID NO: 1; (c) a G residue at a position corresponding to position 71 of SEQ ID NO: 1; (d) an R residue at a position corresponding to position 72 of SEQ ID NO: 1; and (e) an A residue at a position corresponding to position 73 of SEQ ID NO: 1.

In some embodiments of the method, the modifying step comprises modifying the second subunit to comprise one or more of the following residues: (a) an H, K, L, or S residue at a position corresponding to position 48 of SEQ ID NO: 1; (b) an S, C, Q, E, or A residue at a position corresponding to position 50 of SEQ ID NO: 1; (c) an S, P, G, T, A, R, or N residue at a position corresponding to position 71 of SEQ ID NO: 1; (d) a T, R, K, or A residue at a position corresponding to position 72 of SEQ ID NO: 1; (e) an H, C, A, or S residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, and 73 of any one of SEQ ID NOs: 138-156.

In some embodiments of the method, the second subunit is modified to comprise residues corresponding to residues 239, 241, 262, 263, 264, and 265 of any one of SEQ ID NOs: 138-156.

In some embodiments of the method, the method further comprises modifying the first subunit to comprise one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) an A or G residue at a position corresponding to position 19 of SEQ ID NO: 1; (b) a Q or E residue at a position corresponding to position 80 of SEQ ID NO: 1; (c) an H or Y residue at a position corresponding to position 85 of SEQ ID NO: 1; and (d) a K or R residue at a position corresponding to position 139 of SEQ ID NO: 1.

In some embodiments of the method, the first subunit is modified to comprise residues corresponding to residues 19, 80, and 139 of any one of SEQ ID NOs: 138-156.

In some embodiments of the method, the second subunit is modified to comprise residues corresponding to residues 19, 80, 85, and 139 of any one of SEQ ID NOs: 138-156.

In some embodiments of the method, the center sequence consists of ATAA.

In some embodiments of the method, the modifying step comprises modifying the first subunit to comprise one or more of the following residues: (a) a K, A, H, S, L, or Q residue at a position corresponding to position 48 of SEQ ID NO: 1; (b) a Q, T, R, I, G, K, D, C, or V residue at a position corresponding to position 50 of SEQ ID NO: 1; (c) a G, K, S, H, or N residue at a position corresponding to position 71 of SEQ ID NO: 1; (d) an R, A, G, Q, H, L, or S residue at a position corresponding to position 72 of SEQ ID NO: 1; (e) an A, T, or C residue at a position corresponding to position 73 of SEQ ID NO: 1; and (f) an S or A residue at a position corresponding to position 74 of SEQ ID NO: 1.

In some embodiments of the method, the modifying step comprises modifying the second subunit to comprise one or more of the following residues: (a) an S, T, A, K, or N residue at a position corresponding to position 48 of SEQ ID NO: 1; (b) an R, K, E, A, C, or T residue at a position corresponding to position 50 of SEQ ID NO: 1; (c) an S, G, K, or R residue at a position corresponding to position 71 of SEQ ID NO: 1; (d) a T, R, Q, G, A, Y, S, N, or K residue at a position corresponding to position 72 of SEQ ID NO: 1; (e) an 1, C, or V residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 159-183.

In some embodiments of the method, the second subunit is modified to comprise residues corresponding to residues 239, 241, 262, 263, 264, and 265 of any one of SEQ ID NOs: 159-183.

In some embodiments of the method, the method further comprises modifying the first subunit to comprise one or more of the following residues: (a) an A, G, or S residue at a position corresponding to position 19 of SEQ ID NO: 1; (b) a Q or E residue at a position corresponding to position 80 of SEQ ID NO: 1; (c) a K or E residue at a position corresponding to position 100 of SEQ ID NO: 1; (d) a K or R residue at a position corresponding to position 139 of SEQ ID NO: 1; (e) an S or G residue at a position corresponding to position 154 of SEQ ID NO: 1; and (f) an S or A residue at a position corresponding to position 172 of SEQ ID NO: 1.

In some embodiments of the method, the method further comprises modifying the second subunit to comprise one or more of the following residues: (a) a G, S, or A residue at a position corresponding to position 19 of SEQ ID NO: 1; (b) a V or A residue at a position corresponding to position 59 of SEQ ID NO: 1; (c) an L residue at a position corresponding to position 78 of SEQ ID NO: 1; (d) an S residue at a position corresponding to position 79 of SEQ ID NO: 1; (e) a Q or E residue at a position corresponding to position 80 of SEQ ID NO: 1; (f) an S or F residue at a position corresponding to position 118 of SEQ ID NO: 1; and (g) a K or R residue at a position corresponding to position 139 of SEQ ID NO: 1.

In some embodiments of the method, the first subunit is modified to comprise residues corresponding to residues 19, 80, 100, 139, 154, and 172 of any one of SEQ ID NOs: 159-183.

In some embodiments of the method, the second subunit is modified to comprise residues corresponding to residues 19, 59, 78, 79, 80, 118, and 139 of any one of SEQ ID NOs: 159-183.

In some embodiments of the method, the center sequence consists of ATAG.

In some embodiments of the method, the modifying step comprises modifying the first subunit to comprise one or more of the following residues: (a) a K or H residue at a position corresponding to position 48 of SEQ ID NO: 1; (b) an R residue at a position corresponding to position 50 of SEQ ID NO: 1; (c) a G, R, or H residue at a position corresponding to position 71 of SEQ ID NO: 1; (d) an R, G, S, A, P, or Q residue at a position corresponding to position 72 of SEQ ID NO: 1; and (e) an A or C residue at a position corresponding to position 73 of SEQ ID NO: 1.

In some embodiments of the method, the modifying step comprises modifying the second subunit to comprise one or more of the following residues: (a) a C or R residue at a position corresponding to position 50 of SEQ ID NO: 1; (b) a G or S residue at a position corresponding to position 72 of SEQ ID NO: 1; and (c) an R residue at a position corresponding to position 73 of SEQ ID NO: 1.

In some embodiments of the method, the first subunit is modified to comprise residues corresponding to residues 48, 50, 71, 72, and 73 of any one of SEQ ID NOs: 186-199.

In some embodiments of the method, the second subunit is modified to comprise residues corresponding to residues 241, 263, and 264 of any one of SEQ ID NOs: 186-199.

In some embodiments of the method, the method further comprises modifying the first subunit to comprise one or more of the following residues: (a) an A or G residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) a G or A residue at a position corresponding to position 19 of SEQ ID NO: 1; (b) a K or R residue at a position corresponding to position 36 of SEQ ID NO: 1; (c) a V or A residue at a position corresponding to position 59 of SEQ ID NO: 1; (d) a Q residue at a position corresponding to position 80 of SEQ ID NO: 1; and (e) a K or R residue at a position corresponding to position 139 of SEQ ID NO: 1.

In some embodiments of the method, the first subunit is modified to comprise residues corresponding to residues 19, 80, and 139 of any one of SEQ ID NOs: 186-199. In some embodiments of the method, the second subunit is modified to comprise residues corresponding to residues 19, 36, 59, 80, and 139 of any one of SEQ ID NOs: 186-199.

In some embodiments of the method, the center sequence consists of ATAT.

In some embodiments of the method, the modifying step comprises modifying the first subunit to comprise one or more of the following residues: (a) a K, H, C, A, S, D, or T residue at a position corresponding to position 48 of SEQ ID NO: 1; (b) a Q, N, C, R, K, S, T, or V residue at a position corresponding to position 50 of SEQ ID NO: 1; (c) a G, H, or I residue at a position corresponding to position 71 of SEQ ID NO: 1; (d) an R, A, N, or Q residue at a position corresponding to position 72 of SEQ ID NO: 1; and (e) an A, C, or S residue at a position corresponding to position 73 of SEQ ID NO: 1.

In some embodiments of the method, the modifying step comprises modifying the second subunit to comprise one or more of the following residues: (a) an H, K, A, S, R, or T residue at a position corresponding to position 48 of SEQ ID NO: 1; (b) an S, C, K, R, Q, or N residue at a position corresponding to position 50 of SEQ ID NO: 1; (c) an S, K, E, I, G, or R residue at a position corresponding to position 71 of SEQ ID NO: 1; (d) a T, A, R, S, K, G, or N residue at a position corresponding to position 72 of SEQ ID NO: 1; (e) an H, C, A, S, or G residue at a position corresponding to position 73 of SEQ ID NO: 1: and (f) an S, C, or A residue at a position corresponding to position 74 of SEQ ID NO: 1.

In some embodiments of the method, the first subunit is modified to comprise residues corresponding to residues 48, 50, 71, 72, and 73 of any one of SEQ ID NOs: 202-219.

In some embodiments of the method, the second subunit is modified to comprise residues corresponding to residues 239, 241, 262, 263, 264, and 265 of any one of SEQ ID NOs: 202-219.

In some embodiments of the method, the method further comprises modifying the first subunit to comprise one or more of the following residues: (a) an A or G residue at a position corresponding to position 19 of SEQ ID NO: 1; (b) a Q or E residue at a position corresponding to position 80 of SEQ ID NO: 1; and (c) a K, R, or S residue at a position corresponding to position 139 of SEQ ID NO: 1.

In some embodiments of the method, the method further comprises modifying the second subunit to comprise one or more of the following residues: (a) a G or A residue at a position corresponding to position 19 of SEQ ID NO: 1; (b) a V or A residue at a position corresponding to position 59 of SEQ ID NO: 1; (c) a Q, E, or K residue at a position corresponding to position 80 of SEQ ID NO: 1; and (d) a K, R, P, or N residue at a position corresponding to position 139 of SEQ ID NO: 1.

In some embodiments of the method, the first subunit is modified to comprise residues corresponding to residues 19, 80, and 139 of any one of SEQ ID NOs: 202-219.

In some embodiments of the method, the second subunit is modified to comprise residues corresponding to residues 19, 59, 80, and 139 of any one of SEQ ID NOs: 202-219.

In some embodiments of the method, the center sequence consists of ATGA.

In some embodiments of the method, the modifying step comprises modifying the first subunit to comprise one or more of the following residues: (a) a K, A, H, or L residue at a position corresponding to position 48 of SEQ ID NO: 1; (b) an R, T, E, S, C, or V residue at a position corresponding to position 50 of SEQ ID NO: 1; (c) an R, T, S, A, or K residue at a position corresponding to position 72 of SEQ ID NO: 1; and (d) an A or S residue at a position corresponding to position 73 of SEQ ID NO: 1.

In some embodiments of the method, the modifying step comprises modifying the second subunit to comprise one or more of the following residues: (a) an H, K, R, A, or S residue at a position corresponding to position 48 of SEQ ID NO: 1; (b) an S, I, R, C, A, or Q residue at a position corresponding to position 50 of SEQ ID NO: 1; (c) an R or H residue at a position corresponding to position 72 of SEQ ID NO: 1; (d) an I or V residue at a position corresponding to position 73 of SEQ ID NO: 1; and (e) an S, A, or T residue at a position corresponding to position 74 of SEQ ID NO: 1.

In some embodiments of the method, the first subunit is modified to comprise residues corresponding to residues 48, 50, 72, and 73 of any one of SEQ ID NOs: 222-243.

In some embodiments of the method, the second subunit is modified to comprise residues corresponding to residues 239, 241, 263, 264, and 265 of any one of SEQ ID NOs: 222-243.

In some embodiments of the method, the method further comprises modifying the first subunit to comprise one or more of the following residues: (a) an A, G, or S residue at a position corresponding to position 19 of SEQ ID NO: 1; (b) a Q or E residue at a position corresponding to position 80 of SEQ ID NO: 1; (c) an F or L residue at a position corresponding to position 87 of SEQ ID NO: 1; (d) a Q or R residue at a position corresponding to position 92 of SEQ ID NO: 1; and (e) a K or R residue at a position corresponding to position 139 of SEQ ID NO: 1.

In some embodiments of the method, the method further comprises modifying the second subunit to comprise one or more of the following residues: (a) a G, A, or S residue at a position corresponding to position 19 of SEQ ID NO: 1; (b) a V or A residue at a position corresponding to position 59 of SEQ ID NO: 1; (c) a Q or E residue at a position corresponding to position 80 of SEQ ID NO: 1; and (d) a K or R residue at a position corresponding to position 139 of SEQ ID NO: 1.

In some embodiments of the method, the first subunit is modified to comprise residues corresponding to residues 19, 80, 87, 92, and 139 of any one of SEQ ID NOs: 222-243.

In some embodiments of the method, the second subunit is modified to comprise residues corresponding to residues 19, 59, 80, and 139 of any one of SEQ ID NOs: 222-243.

In some embodiments of the method, the center sequence consists of ATGG.

In some embodiments of the method, the modifying step comprises modifying the first subunit to comprise one or more of the following residues: (a) a R residue at a position corresponding to position 50 of SEQ ID NO: 1; (b) a G or S residue at a position corresponding to position 71 of SEQ ID NO: 1; (c) a P or G residue at a position corresponding to position 72 of SEQ ID NO: 1; (d) an A or C residue at a position corresponding to position 73 of SEQ ID NO: 1; and (e) a S or C residue at a position corresponding to position 74 of SEQ ID NO: 1.

In some embodiments of the method, the modifying step comprises modifying the second subunit to comprise one or more of the following residues: (a) an R residue at a position corresponding to position 50 of SEQ ID NO: 1; (b) a D or G residue at a position corresponding to position 71 of SEQ ID NO: 1; (c) a G residue at a position corresponding to position 72 of SEQ ID NO: 1; and (d) a R residue at a position corresponding to position 73 of SEQ ID NO: 1.

In some embodiments of the method, the first subunit is modified to comprise residues corresponding to residues 50, 71, 72, and 73 of any one of SEQ ID NOs: 246-247.

In some embodiments of the method, the second subunit is modified to comprise residues corresponding to residues 241, 262, 263, and 264 of any one of SEQ ID NOs: 246-247.

In some embodiments of the method, the method further comprises modifying the first subunit to comprise one or more of the following residues: (a) an A or G residue at a position corresponding to position 19 of SEQ ID NO: 1; (b) an E or Q residue at a position corresponding to position 80 of SEQ ID NO: 1; (c) an E or K residue at a position corresponding to position 82 of SEQ ID NO: 1; and (d) a R or K residue at a position corresponding to position 139 of SEQ ID NO: 1.

In some embodiments of the method, the method further comprises modifying the second subunit to comprise one or more of the following residues: (a) an A or G residue at a position corresponding to position 19 of SEQ ID NO: 1; (b) a N residue at a position corresponding to position 77 of SEQ ID NO: 1; and (c) a Q or R residue at a position corresponding to position 80 of SEQ ID NO: 1.

In some embodiments of the method, the first subunit is modified to comprise residues corresponding to residues 19, 80, 82, and 139 of any one of SEQ ID NOs: 246-247.

In some embodiments of the method, the second subunit is modified to comprise residues corresponding to residues 19, 77, 80 of any one of SEQ ID NOs: 246-247.

In some embodiments of the method, the second subunit is further modified by inserting an R residue between positions corresponding to positions 73 and 74 of SEQ ID NO: 1.

In some embodiments of the method, the center sequence consists of TTGG.

In some embodiments of the method, the modifying step comprises modifying the first subunit to comprise one or more of the following residues: (a) an R residue at a position corresponding to position 50 of SEQ ID NO: 1; (b) an S residue at a position corresponding to position 71 of SEQ ID NO: 1; (c) a G residue at a position corresponding to position 72 of SEQ ID NO: 1; and (d) an R residue at a position corresponding to position 73 of SEQ ID NO: 1.

In some embodiments of the method, the modifying step comprises modifying the second subunit to comprise one or more of the following residues: (a) a K or S residue at a position corresponding to position 48 of SEQ ID NO: 1; (b) a C, T, E, K, or R residue at a position corresponding to position 50 of SEQ ID NO: 1; (c) a G or K residue at a position corresponding to position 71 of SEQ ID NO: 1; (d) a T, Q, K, R, H, A, or S residue at a position corresponding to position 72 of SEQ ID NO: 1; (e) an 1 or V residue at a position corresponding to position 73 of SEQ ID NO: 1; and (f) an S or A residue at a position corresponding to position 74 of SEQ ID NO: 1.

In some embodiments of the method, the first subunit is modified to comprise residues corresponding to residues 50, 71, 72, and 73 of any one of SEQ ID NOs: 250-266. In some embodiments of the method, the second subunit is modified to comprise residues corresponding to residues 239, 241, 262, 263, 264, and 265 of any one of SEQ ID NOs: 250-266.

In some embodiments of the method, the method further comprises modifying the first subunit to comprise one or more of the following residues: (a) an A or G residue at a position corresponding to position 19 of SEQ ID NO: 1; and (b) a Q residue at a position corresponding to position 80 of SEQ ID NO: 1.

In some embodiments of the method, the method further comprises modifying the second subunit to comprise one or more of the following residues: (a) a G or A residue at a position corresponding to position 19 of SEQ ID NO: 1; (b) a Y or H residue at a position corresponding to position 66 of SEQ ID NO: 1; (c) a Q residue at a position corresponding to position 80 of SEQ ID NO: 1; (d) an H or R residue at a position corresponding to position 85 of SEQ ID NO: 1; and (e) a K or R residue at a position corresponding to position 139 of SEQ ID NO: 1.

In some embodiments of the method, the first subunit is modified to comprise residues corresponding to residues 19 and 80 of any one of SEQ ID NOs: 250-266.

In some embodiments of the method, the second subunit is modified to comprise residues corresponding to residues 19, 66, 80, 85, and 139 of any one of SEQ ID NOs: 250-266.

In some embodiments of the method, the center sequence consists of GCAA.

In some embodiments of the method, the modifying step comprises modifying the first subunit to comprise one or more of the following residues: (a) a K or H residue at a position corresponding to position 48 of SEQ ID NO: 1; (b) an R, C, K, T, or L residue at a position corresponding to position 50 of SEQ ID NO: 1; (c) a G, N, T, R, S, or H residue at a position corresponding to position 71 of SEQ ID NO: 1; (d) an R, P, S, N, Q, G, A, T, M, or V residue at a position corresponding to position 72 of SEQ ID NO: 1; (e) a T or V residue at a position corresponding to position 73 of SEQ ID NO: 1; and (f) an S, C, or A residue at a position corresponding to position 74 of SEQ ID NO: 1.

In some embodiments of the method, the modifying step comprises modifying the second subunit to comprise one or more of the following residues: (a) an S, A, K, or T residue at a position corresponding to position 48 of SEQ ID NO: 1; (b) an R, C, T, K, or E residue at a position corresponding to position 50 of SEQ ID NO: 1; (c) a G, R, A, or H residue at a position corresponding to position 71 of SEQ ID NO: 1; (d) a T, G, S, A, E, N, K, H, R, C, or Y residue at a position corresponding to position 72 of SEQ ID NO: 1; (e) a C, V, or I residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 269-291.

In some embodiments of the method, the second subunit is modified to comprise residues corresponding to residues 239, 241, 262, 263, 264, and 265 of any one of SEQ ID NOs: 269-291.

In some embodiments of the method, the method further comprises modifying the first subunit to comprise one or more of the following residues: (a) an A, G, or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) a G or A residue at a position corresponding to position 19 of SEQ ID NO: 1; (b) a Q or P residue at a position corresponding to position 31 of SEQ ID NO: 1; (c) a Q or E residue at a position corresponding to position 80 of SEQ ID NO: 1; and (d) a K or R residue at a position corresponding to position 139 of SEQ ID NO: 1.

In some embodiments of the method, the first subunit is modified to comprise residues corresponding to residues 19, 80, and 139 of any one of SEQ ID NOs: 269-291.

In some embodiments of the method, the second subunit is modified to comprise residues corresponding to residues 19, 31, 80, and 139 of any one of SEQ ID NOs: 269-291.

In some embodiments of the method, the center sequence consists of GCAT.

In some embodiments of the method, the modifying step comprises modifying the first subunit to comprise one or more of the following residues: (a) a K, A, H, or R residue at a position corresponding to position 48 of SEQ ID NO: 1; (b) a Q, V, R, K, or S residue at a position corresponding to position 50 of SEQ ID NO: 1; (c) a G, A, H, R, T, N, or S residue at a position corresponding to position 71 of SEQ ID NO: 1; (d) an R, T, G, S, Q, N, or A residue at a position corresponding to position 72 of SEQ ID NO: 1; (e) an A, T, V, or C residue at a position corresponding to position 73 of SEQ ID NO: 1; and (f) an S or A residue at a position corresponding to position 74 of SEQ ID NO: 1.

In some embodiments of the method, the modifying step comprises modifying the second subunit to comprise one or more of the following residues: (a) an H, A, K, T, L, or I residue at a position corresponding to position 48 of SEQ ID NO: 1; (b) an S, R, K, Q, H, or V residue at a position corresponding to position 50 of SEQ ID NO: 1; (c) an S, K, R, A, G, T, H, or Y residue at a position corresponding to position 71 of SEQ ID NO: 1; (d) a T, A, G, N, S, R, H, Q, or K residue at a position corresponding to position 72 of SEQ ID NO: 1; (e) an H, C, G, S, or A residue at a position corresponding to position 73 of SEQ ID NO: 1; and (f) an S, C, or A residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 294-313.

In some embodiments of the method, the second subunit is modified to comprise residues corresponding to residues 239, 241, 262, 263, 264, and 265 of any one of SEQ ID NOs: 294-313.

In some embodiments of the method, the method further comprises modifying the first subunit to comprise one or more of the following residues: (a) an A or G residue at a position corresponding to position 19 of SEQ ID NO: 1; (b) a Q or E residue at a position corresponding to position 80 of SEQ ID NO: 1; (c) a K, H, or R residue at a position corresponding to position 139 of SEQ ID NO: 1; and (d) a T or I residue at a position corresponding to position 143 of SEQ ID NO: 1.

In some embodiments of the method, the method further comprises modifying the second subunit to comprise one or more of the following residues: (a) a G, S, or A residue at a position corresponding to position 19 of SEQ ID NO: 1; (b) a Q or E residue at a position corresponding to position 80 of SEQ ID NO: 1; (c) a V or A residue at a position corresponding to position 125 of SEQ ID NO: 1; and (d) a K, R, or H residue at a position corresponding to position 139 of SEQ ID NO: 1.

In some embodiments of the method, the first subunit is modified to comprise residues corresponding to residues 19, 80, 139, and 143 of any one of SEQ ID NOs: 294-313.

In some embodiments of the method, the second subunit is modified to comprise residues corresponding to residues 19, 80, 125, and 139 of any one of SEQ ID NOs: 294-313.

In some embodiments of the method, the center sequence consists of GCGA.

In some embodiments of the method, the modifying step comprises modifying the first subunit to comprise one or more of the following residues: (a) a K or R residue at a position corresponding to position 50 of SEQ ID NO: 1; (b) a G, R, S, A, or N residue at a position corresponding to position 71 of SEQ ID NO: 1; (c) an R, N, G, A, or Q residue at a position corresponding to position 72 of SEQ ID NO: 1; (d) a V, T, or I residue at a position corresponding to position 73 of SEQ ID NO: 1; and (e) an S or A residue at a position corresponding to position 74 of SEQ ID NO: 1.

In some embodiments of the method, the modifying step comprises modifying the second subunit to comprise one or more of the following residues: (a) a K, T, S, A, or Q residue at a position corresponding to position 48 of SEQ ID NO: 1; (b) a C or R residue at a position corresponding to position 50 of SEQ ID NO: 1; (c) an R residue at a position corresponding to position 72 of SEQ ID NO: 1; (d) a V or I residue at a position corresponding to position 73 of SEQ ID NO: 1; and (c) an S or A residue at a position corresponding to position 74 of SEQ ID NO: 1.

In some embodiments of the method, the first subunit is modified to comprise residues corresponding to residues 50, 71, 72, 73, and 74 of any one of SEQ ID NOs: 316-325.

In some embodiments of the method, the second subunit is modified to comprise residues corresponding to residues 239, 241, 263, 264, and 265 of any one of SEQ ID NOs: 316-325.

In some embodiments of the method, the method further comprises modifying the first subunit to comprise one or more of the following residues: (a) an A, G or S residue at a position corresponding to position 19 of SEQ ID NO: 1; and (b) a Q or E residue at a position corresponding to position 80 of SEQ ID NO: 1.

In some embodiments of the method, the method further comprises modifying the second subunit to comprise one or more of the following residues: (a) a G, S, or A residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments of the method, the first subunit is modified to comprise residues corresponding to residues 19 and 80 of any one of SEQ ID NOs: 316-325.

In some embodiments of the method, the second subunit is modified to comprise residues corresponding to residues 19, 80, and 139 of any one of SEQ ID NOs: 316-325.

In some embodiments of the method, the center sequence consists of GCAG.

In some embodiments of the method, the modifying step comprises modifying the first subunit to comprise one or more of the following residues: (a) a R residue at a position corresponding to position 50 of SEQ ID NO: 1; (b) a S residue at a position corresponding to position 71 of SEQ ID NO: 1; (c) an G residue at a position corresponding to position 72 of SEQ ID NO: 1; (d) a R residue at a position corresponding to position 73 of SEQ ID NO: 1; and

In some embodiments of the method, the modifying step comprises modifying the second subunit to comprise one or more of the following residues: (a) a K or H residue at a position corresponding to position 48 of SEQ ID NO: 1; (b) a Q or R residue at a position corresponding to position 50 of SEQ ID NO: 1; and (c) an S or R residue at a position corresponding to position 72 of SEQ ID NO: 1; In some embodiments of the method, the first subunit is modified to comprise residues corresponding to residues 50, 71, 72, 73, and 74 of any one of SEQ ID NOs: 328-330.

In some embodiments of the method, the second subunit is modified to comprise residues corresponding to residues 239, 241, 263, 264, and 265 of any one of SEQ ID NOs: 328-330.

In some embodiments of the method, the method further comprises modifying the second subunit to comprise a Q or E residue at a position corresponding to position 80 of SEQ ID NO: 1.

In some embodiments of the method, the second subunit is modified to comprise residues corresponding to residues 80 of any one of SEQ ID NOs: 328-330.

In some embodiments of the method, the center sequence consists of TCAA.

In some embodiments of the method, the modifying step comprises modifying the first subunit to comprise one or more of the following residues: (a) a K or S residue at a position corresponding to position 48 of SEQ ID NO: 1; (b) an R, T, or C residue at a position corresponding to position 50 of SEQ ID NO: 1; (c) a G, R, or T residue at a position corresponding to position 71 of SEQ ID NO: 1; and (d) an R, S, P, T, or G residue at a position corresponding to position 72 of SEQ ID NO: 1.

In some embodiments of the method, the modifying step comprises modifying the second subunit to comprise one or more of the following residues: (a) an S or K residue at a position corresponding to position 48 of SEQ ID NO: 1; (b) a K, R, C, or E residue at a position corresponding to position 50 of SEQ ID NO: 1; (c) an R, Q, N, or S residue at a position corresponding to position 72 of SEQ ID NO: 1; (d) an I residue at a position corresponding to position 73 of SEQ ID NO: 1; and (e) an S or A residue at a position corresponding to position 74 of SEQ ID NO: 1.

In some embodiments of the method, the first subunit is modified to comprise residues corresponding to residues 48, 50, 71, and 72 of any one of SEQ ID NOs: 333-340.

In some embodiments of the method, the second subunit is modified to comprise residues corresponding to residues 239, 241, 263, 264, and 265 of any one of SEQ ID NOs: 333-340.

In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) a G or S residue at a position corresponding to position 19 of SEQ ID NO: 1; (b) a Q or E residue at a position corresponding to position 80 of SEQ ID NO: 1; and (c) an R residue at a position corresponding to position 139 of SEQ ID NO: 1.

In some embodiments of the method, the first subunit is modified to comprise residues corresponding to residues 19, 80, and 139 of any one of SEQ ID NOs: 333-340.

In some embodiments of the method, the second subunit is modified to comprise residues corresponding to residues 19, 80, and 139 of any one of SEQ ID NOs: 333-340.

In some embodiments of the method, the center sequence consists of TTAA.

In some embodiments of the method, the modifying step comprises modifying the first subunit to comprise one or more of the following residues: (a) a K, N, S, or R residue at a position corresponding to position 48 of SEQ ID NO: 1; (b) an R, V, K, or S residue at a position corresponding to position 50 of SEQ ID NO: 1; (c) a G, R, N, S, or A residue at a position corresponding to position 71 of SEQ ID NO: 1; (d) an R, T, S, N, D, Q, K, or A residue at a position corresponding to position 72 of SEQ ID NO: 1; and (e) an S or A residue at a position corresponding to position 74 of SEQ ID NO: 1.

In some embodiments of the method, the modifying step comprises modifying the second subunit to comprise one or more of the following residues: (a) a K, S, A, or T residue at a position corresponding to position 48 of SEQ ID NO: 1; (b) a C, K, R, T, or E residue at a position corresponding to position 50 of SEQ ID NO: 1; (c) a T, K, R, A, S, or Q residue at a position corresponding to position 72 of SEQ ID NO: 1; (d) an I or V residue at a position corresponding to position 73 of SEQ ID NO: 1; and (e) an S or A residue at a position corresponding to position 74 of SEQ ID NO: 1.

In some embodiments of the method, the first subunit is modified to comprise residues corresponding to residues 48, 50, 71, 72, and 74 of any one of SEQ ID NOs: 343-357.

In some embodiments of the method, the second subunit is modified to comprise residues corresponding to residues 239, 241, 263, 264, and 265 of any one of SEQ ID NOs: 343-357.

In some embodiments of the method, the method further comprises modifying the first subunit to comprise one or more of the following residues: (a) an A, G, or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) a G, A, or S residue at a position corresponding to position 19 of SEQ ID NO: 1; (b) a Y or H residue at a position corresponding to position 66 of SEQ ID NO: 1; (c) a Q residue at a position corresponding to position 80 of SEQ ID NO: 1; and (d) an R residue at a position corresponding to position 139 of SEQ ID NO: 1.

In some embodiments of the method, the first subunit is modified to comprise residues corresponding to residues 19, 80, and 139 of any one of SEQ ID NOs: 343-357.

In some embodiments of the method, the second subunit is modified to comprise residues corresponding to residues 19, 66, 80, and 139 of any one of SEQ ID NOs: 343-357.

Another aspect is an engineered meganuclease that binds and cleaves a recognition sequence comprising a center sequence consisting of GTAA, GTAG, GTAT, GTGA, GTGC, GTGG, or GTGT, wherein said engineered meganuclease comprises a first subunit and a second subunit, wherein said first subunit comprises an amino acid sequence derived from SEQ ID NO: 1, and wherein said first subunit comprises a substitution at one or more positions corresponding to positions 48, 50, 71, 72, 73, and 74 of SEQ ID NO: 1.

In some embodiments, the center sequence consists of GTAA.

In some embodiments, the first subunit comprises one or more of the following residues: (a) a K, S, A, R, N, or T residue at a position corresponding to position 48 of SEQ ID NO: 1; (b) a T, R, A, K, or C residue at a position corresponding to position 50 of SEQ ID NO: 1; (c) a G, R, S, T, A, N, H, or K residue at a position corresponding to position 71 of SEQ ID NO: 1; (d) an R, S, C, N, K, A, H, G, T, D, Y, P or Q residue at a position corresponding to position 72 of SEQ ID NO: 1; (e) a V, C, I, or T residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, 73, and 74 of any one of SEQ ID NOs: 360-389.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the first subunit comprises residues corresponding to residues 19, 80, and 139 of any one of SEQ ID NOs: 360-389.

Another aspect is a method for cleaving double-stranded DNA at a target site comprising a meganuclease recognition sequence recognition sequence comprising a center sequence consisting of GTAA, the method comprising contacting the double-stranded DNA having the target site with an engineered meganuclease described herein, wherein the engineered meganuclease binds and cleaves the recognition sequence.

In some embodiments, the center sequence consists of GTAG.

In some embodiments, the first subunit comprises one or more of the following residues: (a) an R or C residue at a position corresponding to position 50 of SEQ ID NO: 1; (b) an S or D residue at a position corresponding to position 71 of SEQ ID NO: 1; (c) a G or N residue at a position corresponding to position 72 of SEQ ID NO: 1; and (d) an R residue at a position corresponding to position 73 of SEQ ID NO: 1.

In some embodiments, the first subunit comprises residues corresponding to residues 50, 71, 72, and 73 of any one of SEQ ID NOs: 392-399.

In some embodiments, the first subunit comprises one or more of the following residues: (a) an A or S residue at a position corresponding to position 19 of SEQ ID NO: 1; (b) a Q residue at a position corresponding to position 80 of SEQ ID NO: 1; and (c) a K or R residue at a position corresponding to position 139 of SEQ ID NO: 1.

In some embodiments, the first subunit comprises residues corresponding to residues 19, 80, and 139 of any one of SEQ ID NOs: 392-399.

Another aspect is a method for cleaving double-stranded DNA at a target site comprising a meganuclease recognition sequence recognition sequence comprising a center sequence consisting of GTAG, the method comprising contacting the double-stranded DNA having the target site with an engineered meganuclease described herein, wherein the engineered meganuclease binds and cleaves the recognition sequence.

In some embodiments, the center sequence consists of GTAT.

In some embodiments, the first subunit comprises one or more of the following residues: (a) a K, G, T, A, M, H, S, L, or R residue at a position corresponding to position 48 of SEQ ID NO: 1; (b) a Q, V, R, S, T, G, K, C, or L residue at a position corresponding to position 50 of SEQ ID NO: 1; (c) a G, T, A, K, H, R, Y, L, S, or N residue at a position corresponding to position 71 of SEQ ID NO: 1; (d) an R, K, S, Y, N, T, G, W, H, or A residue at a position corresponding to position 72 of SEQ ID NO: 1; (e) an A, C, S, or T residue at a position corresponding to position 73 of SEQ ID NO: 1; and (f) an S, A, or C residue at a position corresponding to position 74 of SEQ ID NO: 1.

In some embodiments, the first subunit comprises residues corresponding to residues 48, 50, 71, 72, 73, and 74 of any one of SEQ ID NOs: 402-433.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K, R, T, or H residue at a position corresponding to position 139 of SEQ ID NO: 1.

In some embodiments, the first subunit comprises residues corresponding to residues 19, 80, and 139 of any one of SEQ ID NOs: 402-433.

Another aspect is a method for cleaving double-stranded DNA at a target site comprising a meganuclease recognition sequence recognition sequence comprising a center sequence consisting of GTAT, the method comprising contacting the double-stranded DNA having the target site with an engineered meganuclease described herein, wherein the engineered meganuclease binds and cleaves the recognition sequence.

In some embodiments, the center sequence consists of GTGA.

In some embodiments, the first subunit comprises one or more of the following residues: (a) a K, A, G, R, S, or H residue at a position corresponding to position 48 of SEQ ID NO: 1; (b) an R, V, C, or S residue at a position corresponding to position 50 of SEQ ID NO: 1; (c) a G, R, V, S, A, T, N, D, or H residue at a position corresponding to position 71 of SEQ ID NO: 1; (d) an R, T, S, G, H, K, or Y residue at a position corresponding to position 72 of SEQ ID NO: 1; (e) an A, V, or T residue at a position corresponding to position 73 of SEQ ID NO: 1; and (f) an S, T, A, or G residue at a position corresponding to position 74 of SEQ ID NO: 1.

In some embodiments, the first subunit comprises residues corresponding to residues 48, 50, 71, 72, 73, and 74 of any one of SEQ ID NOs: 436-462.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the first subunit comprises residues corresponding to residues 19, 80, and 139 of any one of SEQ ID NOs: 436-462.

Another aspect is a method for cleaving double-stranded DNA at a target site comprising a meganuclease recognition sequence recognition sequence comprising a center sequence consisting of GTGA, the method comprising contacting the double-stranded DNA having the target site with an engineered meganuclease described herein, wherein the engineered meganuclease binds and cleaves the recognition sequence.

In some embodiments, the center sequence consists of GTGC.

In some embodiments, the first subunit comprises one or more of the following residues: (a) a K, L, H, A, R, N, or S residue at a position corresponding to position 48 of SEQ ID NO: 1; (b) an R, S, V, K, I, or G residue at a position corresponding to position 50 of SEQ ID NO: 1; (c) a G, S, N, I, R, A, E, Q, Y, T, K, F, or V residue at a position corresponding to position 71 of SEQ ID NO: 1; (d) an R, K, G, H, P, S, C, N, T, A, M, D, or Q residue at a position corresponding to position 72 of SEQ ID NO: 1; (e) an A, V, T, N, C, or L residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, 73, and 74 of any one of SEQ ID NOs: 465-495.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K, T, S, R, H, or V residue at a position corresponding to position 139 of SEQ ID NO: 1. In some embodiments, the first subunit comprises residues corresponding to residues 19, 80, and 139 of any one of SEQ ID NOs: 465-495.

Another aspect is a method for cleaving double-stranded DNA at a target site comprising a meganuclease recognition sequence recognition sequence comprising a center sequence consisting of GTGC, the method comprising contacting the double-stranded DNA having the target site with an engineered meganuclease described herein, wherein the engineered meganuclease binds and cleaves the recognition sequence.

In some embodiments, the center sequence consists of GTGG.

In some embodiments, the first subunit comprises one or more of the following residues: (a) an R residue at a position corresponding to position 50 of SEQ ID NO: 1; (b) an S residue at a position corresponding to position 71 of SEQ ID NO: 1; (c) a G residue at a position corresponding to position 72 of SEQ ID NO: 1; and (d) an R residue at a position corresponding to position 73 of SEQ ID NO: 1.

In some embodiments, the first subunit comprises residues corresponding to residues 50, 71, 72, and 73 of SEQ ID NO: 498-501.

In some embodiments, the first subunit comprises one or more of the following residues: (a) an A residue at a position corresponding to position 19 of SEQ ID NO: 1; (b) an I residue at a position corresponding to position 62 of SEQ ID NO: 1; and (c) a Q residue at a position corresponding to position 80 of SEQ ID NO: 1.

In some embodiments, the first subunit comprises residues corresponding to residues 19, 62, and 80 of SEQ ID NO: 498-501.

Another aspect is a method for cleaving double-stranded DNA at a target site comprising a meganuclease recognition sequence recognition sequence comprising a center sequence consisting of GTGG, the method comprising contacting the double-stranded DNA having the target site with an engineered meganuclease described herein, wherein the engineered meganuclease binds and cleaves the recognition sequence.

In some embodiments, the center sequence consists of GTGT.

In some embodiments, the first subunit comprises one or more of the following residues: (a) a K, S, L, V, G, R, or N residue at a position corresponding to position 48 of SEQ ID NO: 1; (b) a Q, V, R, S, K, A, E, or C residue at a position corresponding to position 50 of SEQ ID NO: 1; (c) a G, R, N, H, A, or T residue at a position corresponding to position 71 of SEQ ID NO: 1; (d) an R, P, A, Q, K, T, G, or V residue at a position corresponding to position 72 of SEQ ID NO: 1; (e) an A, S, C, or T residue at a position corresponding to position 73 of SEQ ID NO: 1; and (f) an S, A, or T residue at a position corresponding to position 74 of SEQ ID NO: 1.

In some embodiments, the first subunit comprises residues corresponding to residues 48, 50, 71, 72, 73, and 74 of any one of SEQ ID NOs: 504-529.

In some embodiments, the first subunit comprises one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments, the first subunit comprises residues corresponding to residues 19, 80, and 139 of any one of SEQ ID NOs: 504-529 Another aspect is a method for cleaving double-stranded DNA at a target site comprising a meganuclease recognition sequence recognition sequence comprising a center sequence consisting of GTGT, the method comprising contacting the double-stranded DNA having the target site with an engineered meganuclease described herein, wherein the engineered meganuclease binds and cleaves the recognition sequence.

Another aspect is a method for increasing the cleavage activity of an engineered meganuclease that binds and cleaves a recognition sequence comprising a center sequence consisting of GTAA, GTAG, GTAT, GTGA, GTGC, GTGG, or GTGT, wherein said engineered meganuclease comprises a first subunit and a second subunit, wherein said first subunit comprises an amino acid sequence derived from SEQ ID NO: 1, said method comprising modifying said first subunit at one or more positions corresponding to positions 48, 50, 71, 72, 73, and 74 of SEQ ID NO: 1, wherein said modified nuclease has increased cleavage activity when compared to a control engineered meganuclease.

In some embodiments of the method, the center sequence consists of GTAA.

In some embodiments of the method, the modifying step comprises modifying the first subunit to comprise one or more of the following residues: (a) a K, S, A, R, N, or T residue at a position corresponding to position 48 of SEQ ID NO: 1; (b) a T, R, A, K, or C residue at a position corresponding to position 50 of SEQ ID NO: 1; (c) a G, R, S, T, A, N, H, or K residue at a position corresponding to position 71 of SEQ ID NO: 1; (d) an R, S, C, N, K, A, H, G, T, D, Y, P, or Q residue at a position corresponding to position 72 of SEQ ID NO: 1; (e) a V, C, I, or T residue at a position corresponding to position 73 of SEQ ID NO: 1; and (f) an S, A, or T residue at a position corresponding to position 74 of SEQ ID NO: 1. In some embodiments of the method, the first subunit is modified to comprise residues corresponding to residues 48, 50, 71, 72, 73, and 74 of any one of SEQ ID NOs: 360-389.

In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the method further comprises modifying the second subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the center sequence consists of GTAG.

In some embodiments of the method, the modifying step comprises modifying the first subunit to comprise one or more of the following residues: (a) an R or C residue at a position corresponding to position 50 of SEQ ID NO: 1; (b) an S or D residue at a position corresponding to position 71 of SEQ ID NO: 1; (c) a G or N residue at a position corresponding to position 72 of SEQ ID NO: 1; and (d) an R residue at a position corresponding to position 73 of SEQ ID NO: 1.

In some embodiments of the method, the first subunit is modified to comprise residues corresponding to residues 50, 71, 72, and 73 of any one of SEQ ID NOs: 392-399.

In some embodiments of the method, the method further comprises modifying the first subunit to comprise one or more of the following residues: (a) an A or S residue at a position corresponding to position 19 of SEQ ID NO: 1; (b) a Q residue at a position corresponding to position 80 of SEQ ID NO: 1; and (c) a K or R residue at a position corresponding to position 139 of SEQ ID NO: 1.

In some embodiments of the method, the first subunit is modified to comprise residues corresponding to residues 19, 80, and 139 of any one of SEQ ID NOs: 392-399.

In some embodiments of the method, the first subunit is modified to comprise residues corresponding to residues 19, 80, and 139 of any one of SEQ ID NOs: 360-389.

In some embodiments of the method, the center sequence consists of GTAT.

In some embodiments of the method, the modifying step comprises modifying the first subunit to comprise one or more of the following residues: (a) a K, G, T, A, M, H, S, L, or R residue at a position corresponding to position 48 of SEQ ID NO: 1; (b) a Q, V, R, S, T, G, K, C, or L residue at a position corresponding to position 50 of SEQ ID NO: 1; (c) a G, T, A, K, H, R, Y, L, S, or N residue at a position corresponding to position 71 of SEQ ID NO: 1; (d) an R, K, S, Y, N, T, G, W, H, A residue at a position corresponding to position 72 of SEQ ID NO: 1; (e) an A, C, S, or T residue at a position corresponding to position 73 of SEQ ID NO: 1; and (f) an S, A, or C residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 402-433.

In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K, R, T, or H residue at a position corresponding to position 139 of SEQ ID NO: 1.

In some embodiments of the method, the first subunit is modified to comprise residues corresponding to residues 19, 80, and 139 of any one of SEQ ID NOs: 402-433.

In some embodiments of the method, the center sequence consists of GTGA.

In some embodiments of the method, the modifying step comprises modifying the first subunit to comprise one or more of the following residues: (a) a K, A, G, R, S, or H residue at a position corresponding to position 48 of SEQ ID NO: 1; (b) an R, V, C, or S residue at a position corresponding to position 50 of SEQ ID NO: 1; (c) a G, R, V, S, A, T, N, D, or H residue at a position corresponding to position 71 of SEQ ID NO: 1; (d) an R, T, S, G, H, K, or Y residue at a position corresponding to position 72 of SEQ ID NO: 1; (e) an A. V, or T residue at a position corresponding to position 73 of SEQ ID NO: 1; and (f) an S, T, A, or G residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 436-462.

In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the first subunit is modified to comprise residues corresponding to residues 19, 80, and 139 of any one of SEQ ID NOs: 436-462.

In some embodiments of the method, the center sequence consists of GTGC.

In some embodiments of the method, the modifying step comprises modifying the first subunit to comprise one or more of the following residues: (a) a K, L, H, A, R, N, or S residue at a position corresponding to position 48 of SEQ ID NO: 1; (b) an R, S, V, K, I, or G residue at a position corresponding to position 50 of SEQ ID NO: 1; (c) a G, S, N, I, R, A, E, Q, Y, T, K, F, or V residue at a position corresponding to position 71 of SEQ ID NO: 1; (d) an R, K, G, H, P, S, C, N, T, A, M, D, or Q residue at a position corresponding to position 72 of SEQ ID NO: 1; (e) an A, V, T, N, C, or L residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 465-495.

In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K, T, S, R, H, or V residue at a position corresponding to position 139 of SEQ ID NO: 1.

In some embodiments of the method, the first subunit is modified to comprise residues corresponding to residues 19, 80, and 139 of any one of SEQ ID NOs: 465-495.

In some embodiments of the method, the center sequence consists of GTGG.

In some embodiments of the method, the modifying step comprises modifying the first subunit to comprise one or more of the following residues: (a) an R residue at a position corresponding to position 50 of SEQ ID NO: 1; (b) an S residue at a position corresponding to position 71 of SEQ ID NO: 1; (c) a G residue at a position corresponding to position 72 of SEQ ID NO: 1; and (d) an R residue at a position corresponding to position 73 of SEQ ID NO: 1.

In some embodiments of the method, the first subunit is modified to comprise residues corresponding to residues 50, 71, 72, and 73 of SEQ ID NO: 498-501.

In some embodiments of the method, the method further comprises modifying the first subunit to comprise one or more of the following residues: (a) an A residue at a position corresponding to position 19 of SEQ ID NO: 1; (b) an I residue at a position corresponding to position 62 of SEQ ID NO: 1; and (c) a Q residue at a position corresponding to position 80 of SEQ ID NO: 1.

In some embodiments of the method, the first subunit is modified to comprise residues corresponding to residues 19, 62, and 80 of SEQ ID NO: 498-501.

In some embodiments of the method, the center sequence consists of GTGT.

In some embodiments of the method, the modifying step comprises modifying the first subunit to comprise one or more of the following residues: (a) a K, S, L, V, G, R, or N residue at a position corresponding to position 48 of SEQ ID NO: 1; (b) a Q, V, R, S, K, A, E, or C residue at a position corresponding to position 50 of SEQ ID NO: 1; (c) a G, R, N, H, A, or T residue at a position corresponding to position 71 of SEQ ID NO: 1; (d) an R, P, A, Q, K, T, G, or V residue at a position corresponding to position 72 of SEQ ID NO: 1; (e) an A, S, C, or T residue at a position corresponding to position 73 of SEQ ID NO: 1; and (f) an S, A, or T residue at a position corresponding to position 74 of SEQ ID NO: 1.

In some embodiments of the method, the first subunit is modified to comprise residues corresponding to residues 48, 50, 71, 72, 73, and 74 of any one of SEQ ID NOs: 504-529 In some embodiments of the method, the method further comprises modifying the first subunit to comprise one or more of the following residues: (a) an A or S residue at a position corresponding to position 19 of SEQ ID NO: 1; (b) a Q or E residue at a position corresponding to position 80 of SEQ ID NO: 1; and (c) a K or R residue at a position corresponding to position 139 of SEQ ID NO: 1.

In some embodiments of the method, the first subunit is modified to comprise residues corresponding to residues 19, 80, and 139 of any one of SEQ ID NOs: 504-529.

Another aspect is an I-Cre derived engineered meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, and wherein the first subunit and the second subunit each comprise a substitution at one or more positions corresponding to positions 48, 50, 71, 72, 73, and 74 of SEQ ID NO: 1.

Another aspect is an improved engineered I-CreI-derived meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, the improvement comprising any amino acid substitution described herein that improves cleavage activity of the engineered I-CrcI-derived meganuclease for a recognition sequence comprising an ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA center sequence.

In some embodiments, the first subunit comprises one or more of the following residues: (a) an A, C, D, G, H, I, K, L, N, Q, R, S, or T residue at a position corresponding to position 48 of SEQ ID NO: 1; (b) an A, C, D, E, G, I, K, L, N, Q, R, S, T, V, or W residue at a position corresponding to position 50 of SEQ ID NO: 1; (c) an A, C, G, H, I, K, N, P, R, S. or T residue at a position corresponding to position 71 of SEQ ID NO: 1; (d) an A, D, G, H, K, L, M, N, P, Q, R, S, T, or V residue at a position corresponding to position 72 of SEQ ID NO: 1; (e) an A, C, G, I, S, T, or V residue at a position corresponding to position 73 of SEQ ID NO: 1; and (f) an A, C, T, or S residue at a position corresponding to position 74 of SEQ ID NO: 1.

In some embodiments, the second subunit comprises one or more of the following residues (a) an A, C, G, H, I, K, L, N, Q, R, S, or T residue at a position corresponding to position 48 of SEQ ID NO: 1; (b) an A, C, E, G, H, I, K, N, P, Q, R, S, T, or V residue at a position corresponding to position 50 of SEQ ID NO: 1; (c) an A, D, E, G, H, I, K, N, P, Q, R, S, T, or Y residue at a position corresponding to position 71 of SEQ ID NO: 1; (d) an A, C, E, G, H, I, K, M, N, P, Q, R, S, T, V, or Y residue at a position corresponding to position 72 of SEQ ID NO: 1; (e) an A, C, G, H, I, R, S, T, or V residue at a position corresponding to position 73 of SEQ ID NO: 1; and (f) an A, C, S, or T residue at a position corresponding to position 74 of SEQ ID NO: 1.

In some embodiments, the center sequence consists of ACAA, ACAG, ACAT, ACGC, ACGG, or ACGT, wherein the first subunit comprises one or more of the following residues (a) an A, C, G, H, I, K, L, N, Q, or S residue at a position corresponding to position 48 of SEQ ID NO: 1; (b) an A, C, K, Q, R, S, T, V, or W residue at a position corresponding to position 50 of SEQ ID NO: 1; (c) an A, G, P, or R residue at a position corresponding to position 71 of SEQ ID NO: 1; (d) an H, K, P, Q, R, or T residue at a position corresponding to position 72 of SEQ ID NO: 1; (e) an A, C, G, or V residue at a position corresponding to position 73 of SEQ ID NO: 1; and (f) a S residue at a position corresponding to position 74 of SEQ ID NO: 1.

In some embodiments, the center sequence consists of ATAA, ATAG, ATAT, ATGA, ATGG, wherein the first subunit comprises one or more of the following residues: (a) an A, C, D, G, H, K, L, N, Q, S, or T residue at a position corresponding to position 48 of SEQ ID NO: 1; (b) a C, D, E, G, I, K, N, R, S, T, or V residue at a position corresponding to position 50 of SEQ ID NO: 1; (c) a G, H, I, K, N, R, or S residue at a position corresponding to position 71 of SEQ ID NO: 1; (d) an A, G, H, K, L, N, P, Q, R, S, or T residue at a position corresponding to position 72 of SEQ ID NO: 1; (e) an A, C, S, or T residue at a position corresponding to position 73 of SEQ ID NO: 1; and (f) an A, C, or S residue at a position corresponding to position 74 of SEQ ID NO: 1.

In some embodiments, the center sequence consists of GCAA, GCAT, GCGA, or GCAG, wherein the first subunit comprises one or more of the following residues: (a) an A, H, K, or R residue at a position corresponding to position 48 of SEQ ID NO: 1; (b) a C, K, L, Q, R, S, T, or V residue at a position corresponding to position 50 of SEQ ID NO: 1; (c) an A, G, H, N, R, S, or T residue at a position corresponding to position 71 of SEQ ID NO: 1; (d) an A, G, H, M, N, P, Q, R, S, T, or V residue at a position corresponding to position 72 of SEQ ID NO: 1; (e) an A, C, I, T, or V residue at a position corresponding to position 73 of SEQ ID NO: 1; and (f) an A or S residue at a position corresponding to position 74 of SEQ ID NO: 1.

In some embodiments, the center sequence consists of TTGG or TTAA, wherein the first subunit comprises one or more of the following residues: (a) a K, N, R, or S residue at a position corresponding to position 48 of SEQ ID NO: 1; (b) a C, E, K, R, S, T, or V residue at a position corresponding to position 50 of SEQ ID NO: 1; (c) an A, G, K, N, R, or S residue at a position corresponding to position 71 of SEQ ID NO: 1; (d) an A, D, H, K, N, Q, R, S, or T residue at a position corresponding to position 72 of SEQ ID NO: 1; (e) an I or V residue at a position corresponding to position 73 of SEQ ID NO: 1; and (f) an A, S or T residue at a position corresponding to position 74 of SEQ ID NO: 1.

In some embodiments, the center sequence consists of TCAA, wherein the first subunit comprises one or more of the following residues: (a) an A, G, H, K, N, Q, R, or S residue at a position corresponding to position 48 of SEQ ID NO: 1; (b) a C, R, S, or T residue at a position corresponding to position 50 of SEQ ID NO: 1; (c) a G, R, S, or T residue at a position corresponding to position 71 of SEQ ID NO: 1; (d) a G, H, P, R, S, or T residue at a position corresponding to position 72 of SEQ ID NO: 1; (e) an I or V residue at a position corresponding to position 73 of SEQ ID NO: 1; and (f) an A or S residue at a position corresponding to position 74 of SEQ ID NO: 1.

In some embodiments, the center sequence consists of ACAA, ACAG, ACAT, ACGC, ACGG, or ACGT, wherein the second subunit comprises one or more of the following residues (a) an A, C, G, H, K, L, N, Q, R, S, or T residue at a position corresponding to position 48 of SEQ ID NO: 1; (b) an A, C, G, H, K, L, N, Q, R, S, or T residue at a position corresponding to position 50 of SEQ ID NO: 1; (c) an A, D, E, G, H, K, N, P, R, S, or T residue at a position corresponding to position 71 of SEQ ID NO: 1; (d) an A, G, H, K, M, N, P, P, Q, R, S, or T residue at a position corresponding to position 72 of SEQ ID NO: 1; (e) an A, C, G, H, I, R, S, T, or V residue at a position corresponding to position 73 of SEQ ID NO: 1; (f) optionally an R residue at a position directly following position corresponding to position 73 of SEQ ID NO: 1 (73B); and (g) an A, C, S, or T residue at a position corresponding to position 74 of SEQ ID NO: 1.

In some embodiments, the center sequence consists of ATAA, ATAG, ATAT, ATGA, or ATGG, wherein the second subunit comprises one or more of the following residues: (a) an A, C, G, H, K, N, Q, R, S, or T residue at a position corresponding to position 48 of SEQ ID NO: 1; (b) an A, C, E, I, K, N, Q, R, S, or T residue at a position corresponding to position 50 of SEQ ID NO: 1; (c) an A, C, E, I, K, N, Q, R, S, or T residue at a position corresponding to position 71 of SEQ ID NO: 1; (d) an A, G, H, K, N, Q, R, S, T, V, or Y residue at a position corresponding to position 72 of SEQ ID NO: 1; (e) an A, C, G, H, I, R, S, or V residue at a position corresponding to position 73 of SEQ ID NO: 1; (f) optionally an R residue at a position directly following position corresponding to position 73 of SEQ ID NO: 1 (73B); and (g) an A, C, S, or T residue at a position corresponding to position 74 of SEQ ID NO: 1.

In some embodiments, the center sequence consists of GCAA, GCAT, GCGA, or GCAG, wherein the second subunit comprises one or more of the following residues: (a) an A, C, G, H, I, K, L, N, Q, R, S, or T residue at a position corresponding to position 48 of SEQ ID NO: 1; (h) a C, E, H, K, Q, R, S, T, or V residue at a position corresponding to position 50 of SEQ ID NO: 1; (c) an A, G, H, K, R, S, T, or Y residue at a position corresponding to position 71 of SEQ ID NO: 1; (d) an A, C, E, G, H, K, N, Q, R, S, T, or Y residue at a position corresponding to position 72 of SEQ ID NO: 1; (e) an A, C, G, H, I, R, S, or V residue at a position corresponding to position 73 of SEQ ID NO: 1; and (f) an A, S, or T residue at a position corresponding to position 74 of SEQ ID NO: 1.

In some embodiments, the center sequence consists of TTGG or TTAA, wherein the second subunit comprises one or more of the following residues: (a) an A, K, S, or T residue at a position corresponding to position 48 of SEQ ID NO: 1; (b) a C, E, K, R, or T residue at a position corresponding to position 50 of SEQ ID NO: 1; (c) an A, D, G, K, Q, R, S, or T residue at a position corresponding to position 71 of SEQ ID NO: 1; (d) a G, I, R, S, T, or V residue at a position corresponding to position 72 of SEQ ID NO: 1; (e) an I, R, or V residue at a position corresponding to position 73 of SEQ ID NO: 1; and (f) an A, S, or T residue at a position corresponding to position 74 of SEQ ID NO: 1.

In some embodiments, the center sequence consists of TCAA, wherein the second subunit comprises one or more of the following residues: (a) a K or S residue at a position corresponding to position 48 of SEQ ID NO: 1; (b) a C, K, R, or T residue at a position corresponding to position 50 of SEQ ID NO: 1; (c) a G, R, or T residue at a position corresponding to position 71 of SEQ ID NO: 1; (d) a G, P, R, S, or T residue at a position corresponding to position 72 of SEQ ID NO: 1; (e) a I or V residue at a position corresponding to position 73 of SEQ ID NO: 1; and (f) an A, S, or T residue at a position corresponding to position 74 of SEQ ID NO: 1.

In some embodiments: (a) the center sequence is ACAA and the first subunit comprises residues corresponding to residues 48, 50, 71, 72, 73, and 74 of any one of SEQ ID NOs: 11-33, (b) the center sequence is ACAG and the first subunit comprises residues corresponding to residues 48, 50, 71, 72, 73, and 74 of any one of SEQ ID NOs: 36-43, (c) the center sequence is ACAT and the first subunit comprises residues corresponding to residues 48, 50, 71, 72, 73, and 74 of any one of SEQ ID NOs: 46-67, (d) the center sequence is ACGA and the first subunit comprises residues corresponding to residues 48, 50, 71, 72, 73, and 74 of any one of SEQ ID NOs: 70-89, (e) the center sequence is ACGC and the first subunit comprises residues corresponding to residues 48, 50, 71, 72, 73, and 74 of any one of SEQ ID NOs: 92-118, (f) the center sequence is ACGG and the first subunit comprises residues corresponding to residues 48, 50, 71, 72, 73, and 74 of any one of SEQ ID NOs: 121-135, (g) the center sequence is ACGT and the first subunit comprises residues corresponding to residues 48, 50, 71, 72, 73, and 74 of any one of SEQ ID NOs: 138-156, (h) the center sequence is ATAA and the first subunit comprises residues corresponding to residues 48, 50, 71, 72, 73, and 74 of any one of SEQ ID NOs: 159-183, (i) the center sequence is ATAG and the first subunit comprises residues corresponding to residues 48, 50, 71, 72, 73, and 74 of any one of SEQ ID NOs: 186-199, (j) the center sequence is ATAT and the first subunit comprises residues corresponding to residues 48, 50, 71, 72, 73, and 74 of any one of SEQ ID NOs: 202-219, (k) the center sequence is ATGA and the first subunit comprises residues corresponding to residues 48, 50, 71, 72, 73, and 74 of any one of SEQ ID NOs: 222-243, (l) the center sequence is ATGG and the first subunit comprises residues corresponding to residues 48, 50, 71, 72, 73, and 74 of any one of SEQ ID NOs: 246-247, (m) the center sequence is TTGG and the first subunit comprises residues corresponding to residues 48, 50, 71, 72, 73, and 74 of any one of SEQ ID NOs: 250-266, (n) the center sequence is GCAA and the first subunit comprises residues corresponding to residues 48, 50, 71, 72, 73, and 74 of any one of SEQ ID NOs: 269-291, (o) the center sequence is GCAT and the first subunit comprises residues corresponding to residues 48, 50, 71, 72, 73, and 74 of any one of SEQ ID NOs: 294-313, (p) the center sequence is GCGA and the first subunit comprises residues corresponding to residues 48, 50, 71, 72, 73, and 74 of any one of SEQ ID NOs: 316-325, (q) the center sequence is GCAG and the first subunit comprises residues corresponding to residues 48, 50, 71, 72, 73, and 74 of any one of SEQ ID NOs: 328-330, (r) the center sequence is TCAA and the first subunit comprises residues corresponding to residues 48, 50, 71, 72, 73, and 74 of any one of SEQ ID NOs: 333-340, or (s) the center sequence is TTAA and the first subunit comprises residues corresponding to residues 48, 50, 71, 72, 73, and 74 of any one of SEQ ID NOs: 343-357.

In some embodiments: (a) the center sequence is ACAA and the second subunit comprises residues corresponding to residues 48, 50, 71, 72, 73, and 74 of any one of SEQ ID NOs: 11-33, (b) the center sequence is ACAG and the second subunit comprises residues corresponding to residues 48, 50, 71, 72, 73, and 74 of any one of SEQ ID NOs: 36-43, (c) the center sequence is ACAT and the second subunit comprises residues corresponding to residues 48, 50, 71, 72, 73, and 74 of any one of SEQ ID NOs: 46-67, (d) the center sequence is ACGA and the second subunit comprises residues corresponding to residues 48, 50, 71, 72, 73, and 74 of any one of SEQ ID NOs: 70-89, (e) the center sequence is ACGC and the second subunit comprises residues corresponding to residues 48, 50, 71, 72, 73, and 74 of any one of SEQ ID NOs: 92-118, (f) the center sequence is ACGG and the second subunit comprises residues corresponding to residues 48, 50, 71, 72, 73, and 74 of any one of SEQ ID NOs: 121-135, (g) the center sequence is ACGT and the second subunit comprises residues corresponding to residues 48, 50, 71, 72, 73, and 74 of any one of SEQ ID NOs: 138-156, (h) the center sequence is ATAA and the second subunit comprises residues corresponding to residues 48, 50, 71, 72, 73, and 74 of any one of SEQ ID NOs: 159-183, (i) the center sequence is ATAG and the second subunit comprises residues corresponding to residues 48, 50, 71, 72, 73, and 74 of any one of SEQ ID NOs: 186-199, (j) the center sequence is ATAT and the second subunit comprises residues corresponding to residues 48, 50, 71, 72, 73, and 74 of any one of SEQ ID NOs: 202-219, (k) the center sequence is ATGA and the second subunit comprises residues corresponding to residues 48, 50, 71, 72, 73, and 74 of any one of SEQ ID NOs: 222-243, (l) the center sequence is ATGG and the second subunit comprises residues corresponding to residues 48, 50, 71, 72, 73, and 74 of any one of SEQ ID NOs: 246-247, (m) the center sequence is TTGG and the second subunit comprises residues corresponding to residues 48, 50, 71, 72, 73, and 74 of any one of SEQ ID NOs: 250-266, (n) the center sequence is GCAA and the second subunit comprises residues corresponding to residues 48, 50, 71, 72, 73, and 74 of any one of SEQ ID NOs: 269-291, (o) the center sequence is GCAT and the second subunit comprises residues corresponding to residues 48, 50, 71, 72, 73, and 74 of any one of SEQ ID NOs: 294-313, (p) the center sequence is GCGA and the second subunit comprises residues corresponding to residues 48, 50, 71, 72, 73, and 74 of any one of SEQ ID NOs: 316-325, (q) the center sequence is GCAG and the second subunit comprises residues corresponding to residues 48, 50, 71, 72, 73, and 74 of any one of SEQ ID NOs: 328-330, (r) the center sequence is TCAA and the second subunit comprises residues corresponding to residues 48, 50, 71, 72, 73, and 74 of any one of SEQ ID NOs: 333-340, or (s) the center sequence is TTAA and the second subunit comprises residues corresponding to residues 48, 50, 71, 72, 73, and 74 of any one of SEQ ID NOs: 343-357.

Another aspect is a method for cleaving double-stranded DNA at a target site comprising a meganuclease recognition sequence, wherein the recognition sequence comprises a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the method comprises contacting the double-stranded DNA having the target site with any engineered meganuclease described herein, wherein the engineered meganuclease binds and cleaves the recognition sequence.

Another aspect is an improved method for cleaving double-stranded DNA at a target site comprising a meganuclease recognition sequence by contacting said double-stranded DNA having said target site with an engineered I-CreI-derived meganuclease, wherein the engineered meganuclease comprises a first subunit and a second subunit, wherein the first subunit and the second subunit each comprise an amino acid sequence derived from SEQ ID NO: 1, wherein said recognition sequence comprises a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, the improvement comprising: use of an engineered I-CreI-derived meganuclease described herein, wherein said engineered I-CreI-derived meganuclease binds and cleaves said recognition sequence.

Another aspect is a method for increasing the cleavage activity of an I-CreI engineered meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, the method comprising modifying each of the first subunit and the second subunit at one or more positions corresponding to positions 48, 50, 71, 72, 73, and 74 of SEQ ID NO: 1, wherein the modified nuclease has increased cleavage activity when compared to a control engineered meganuclease.

Another aspect is an improved method for increasing the cleavage activity of an engineered I-CreI-derived meganuclease that binds and cleaves a recognition sequence comprising a center sequence consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein the engineered meganuclease comprises a first subunit and a second subunit, wherein the first subunit and the second subunit each comprise an amino acid sequence derived from SEQ ID NO: 1, the improvement comprising use of an engineered I-CreI-derived meganuclease described herein, wherein said engineered I-CreI-derived meganuclease binds and cleaves said recognition sequence.

In some embodiments of the method, the modifying step comprises modifying the first subunit to comprise one or more of the following residues: (a) an A, C, D, G, H, I, K, L, N, Q, R, S, or T residue at a position corresponding to position 48 of SEQ ID NO: 1; (b) an A, C, D, E, G, T, K, L, N, Q, R, S, T, V, or W residue at a position corresponding to position 50 of SEQ ID NO: 1; (c) an A, C, G, H, I, K, N, P, R, S, or T residue at a position corresponding to position 71 of SEQ ID NO: 1; (d) an A, D, G, H, K, L, M, N, P, Q, R, S, T. or V residue at a position corresponding to position 72 of SEQ ID NO: 1; (e) an A, C, G, I, S, T, or V residue at a position corresponding to position 73 of SEQ ID NO: 1; and (f) an A, C, T, or S residue at a position corresponding to position 74 of SEQ ID NO: 1.

In some embodiments of the method, the modifying step comprises modifying the second subunit to comprise one or more of the following residues: (a) an A, C, G, H, I, K, L, N, Q, R, S, or T residue at a position corresponding to position 48 of SEQ ID NO: 1; (b) an A, C, E, G, H, I, K, N, P, Q, R, S, T, or V residue at a position corresponding to position 50 of SEQ ID NO: 1; (c) an A, D, E, G, H, I, K, N, P, Q, R, S, T, or Y residue at a position corresponding to position 71 of SEQ ID NO: 1; (d) an A, C, E, G, H, I, K, M, N, P, Q, R, S, T, V, or Y residue at a position corresponding to position 72 of SEQ ID NO: 1; (e) an A, C, G, H, I, R, S, T, or V residue at a position corresponding to position 73 of SEQ ID NO: 1; and (f) an A, C, S, or T residue at a position corresponding to position 74 of SEQ ID NO: 1.

In some embodiments of the method, the center sequence consists of ACAA, ACAG, ACAT, ACGC, ACGG, or ACGT, and wherein the modifying step comprises modifying the first subunit to comprise one or more of the following residues: (a) an A, C, G, H, I, K, L, N, Q, or S residue at a position corresponding to position 48 of SEQ ID NO: 1; (b) an A, C, K, Q, R, S, T, V, or W residue at a position corresponding to position 50 of SEQ ID NO: 1; (c) an A, G, P, or R residue at a position corresponding to position 71 of SEQ ID NO: 1; (d) an H, K, P, Q, R, or T residue at a position corresponding to position 72 of SEQ ID NO: 1; (e) an A, C, G, or V residue at a position corresponding to position 73 of SEQ ID NO: 1; and (f) a S residue at a position corresponding to position 74 of SEQ ID NO: 1.

In some embodiments of the method, the center sequence consists of ATAA, ATAG, ATAT, ATGA, or ATGG, and wherein the modifying step comprises modifying the first subunit to comprise one or more of the following residues: (a) an A, C, D, G, H, K, L, N, Q, S, or T residue at a position corresponding to position 48 of SEQ ID NO: 1; (b) a C, D, E, G, I, K, N, R, S, T, or V residue at a position corresponding to position 50 of SEQ ID NO: 1; (c) a G, H, I, K, N, R, or S residue at a position corresponding to position 71 of SEQ ID NO: 1; (d) an A, G, H, K, L, N, P, Q, R, S, or T residue at a position corresponding to position 72 of SEQ ID NO: 1; (e) an A, C, S, or T residue at a position corresponding to position 73 of SEQ ID NO: 1; and (f) an A, C, or S residue at a position corresponding to position 74 of SEQ ID NO: 1.

In some embodiments of the method, the center sequence consists of GCAA, GCAT, GCGA, or GCAG, and wherein the modifying step comprises modifying the first subunit to comprise one or more of the following residues: (a) an A, H, K, or R residue at a position corresponding to position 48 of SEQ ID NO: 1; (b) a C, K, L, Q, R, S, T, or V residue at a position corresponding to position 50 of SEQ ID NO: 1; (c) an A, G, H, N, R, S, or T residue at a position corresponding to position 71 of SEQ ID NO: 1; (d) an A, G, H, M, N, P, Q, R, S, T, or V residue at a position corresponding to position 72 of SEQ ID NO: 1; (c) an A, C, I, T, or V residue at a position corresponding to position 73 of SEQ ID NO: 1; and (f) an A or S residue at a position corresponding to position 74 of SEQ ID NO: 1.

In some embodiments of the method, the center sequence consists of TTGG or TTAA, and wherein the modifying step comprises modifying the first subunit to comprise one or more of the following residues: (a) a K, N, R, or S residue at a position corresponding to position 48 of SEQ ID NO: 1; (b) a C, E, K, R, S, T, or V residue at a position corresponding to position 50 of SEQ ID NO: 1; (c) an A, G, K, N, R, or S residue at a position corresponding to position 71 of SEQ ID NO: 1; (d) an A, D, H, K, N, Q, R, S, or T residue at a position corresponding to position 72 of SEQ ID NO: 1; (e) an I or V residue at a position corresponding to position 73 of SEQ ID NO: 1; and (f) an A, S or T residue at a position corresponding to position 74 of SEQ ID NO: 1.

In some embodiments of the method, the center sequence consists of TCAA, and wherein the modifying step comprises modifying the first subunit to comprise one or more of the following residues: (a) an A, G, H, K, N, Q, R, or S residue at a position corresponding to position 48 of SEQ ID NO: 1; (b) a C, R, S, or T residue at a position corresponding to position 50 of SEQ ID NO: 1; (c) a G, R, S, or T residue at a position corresponding to position 71 of SEQ ID NO: 1; (d) a G, H, P, R, S, or T residue at a position corresponding to position 72 of SEQ ID NO: 1; (e) an I or V residue at a position corresponding to position 73 of SEQ ID NO: 1; and (f) an A or S residue at a position corresponding to position 74 of SEQ ID NO: 1.

In some embodiments of the method, the center sequence consists of ACAA, ACAG, ACAT, ACGC, ACGG, or ACGT, and wherein the modifying step comprises modifying the second subunit to comprise one or more of the following residues: (a) an A, C, G, H, K, L, N, Q, R, S, or T residue at a position corresponding to position 48 of SEQ ID NO: 1; (b) an A, C, G, H, K, L, N, Q, R, S, or T residue at a position corresponding to position 50 of SEQ ID NO: 1; (c) an A, D, E, G, H, K, N, P, R, S, or T residue at a position corresponding to position 71 of SEQ ID NO: 1; (d) an A, G, H, K, M, N, P, P, Q, R, S, or T residue at a position corresponding to position 72 of SEQ ID NO: 1; (e) an A, C, G, H, I, R, S, T, or V residue at a position corresponding to position 73 of SEQ ID NO: 1; (f) optionally an R residue at a position directly following position corresponding to position 73 of SEQ ID NO: 1 (73B); and (g) an A, C, S, or T residue at a position corresponding to position 74 of SEQ ID NO: 1.

In some embodiments of the method, the center sequence consists of ATAA, ATAG, ATAT, ATGA, or ATGG, and wherein the modifying step comprises modifying the second subunit to comprise one or more of the following residues: (a) an A, C, G, H, K, N, Q, R, S, or T residue at a position corresponding to position 48 of SEQ ID NO: 1; (b) an A, C, E, I, K, N, Q, R, S, or T residue at a position corresponding to position 50 of SEQ ID NO: 1; (c) an A, C, E, I, K, N, Q, R, S, or T residue at a position corresponding to position 71 of SEQ ID NO: 1; (d) an A, G, H, K, N, Q, R, S, T, V, or Y residue at a position corresponding to position 72 of SEQ ID NO: 1; (e) an A, C, G, H, I, R, S, or V residue at a position corresponding to position 73 of SEQ ID NO: 1; (f) optionally an R residue at a position directly following position corresponding to position 73 of SEQ ID NO: 1 (73B); and (g) an A, C, S, or T residue at a position corresponding to position 74 of SEQ ID NO: 1.

In some embodiments of the method, the center sequence consists of GCAA, GCAT, GCGA, or GCAG, and wherein the modifying step comprises modifying the second subunit to comprise one or more of the following residues: (a) an A, C, G, H, I, K, L, N, Q, R, S, or T residue at a position corresponding to position 48 of SEQ ID NO: 1; (b) a C, E, H, K, Q, R, S, T, or V residue at a position corresponding to position 50 of SEQ ID NO: 1; (c) an A, G, H, K, R, S, T, or Y residue at a position corresponding to position 71 of SEQ ID NO: 1; (d) an A, C, E, G, H, K, N, Q, R, S, T, or Y residue at a position corresponding to position 72 of SEQ ID NO: 1; (e) an A, C, G, H, I, R, S, or V residue at a position corresponding to position 73 of SEQ ID NO: 1; and (f) an A, S, or T residue at a position corresponding to position 74 of SEQ ID NO: 1.

In some embodiments of the method, the center sequence consists of TTGG or TTAA, and wherein the modifying step comprises modifying the second subunit to comprise one or more of the following residues: (a) an A, K, S, or T residue at a position corresponding to position 48 of SEQ ID NO: 1; (b) a C, E, K, R, or T residue at a position corresponding to position 50 of SEQ ID NO: 1; (c) an A, D, G, K, Q, R, S, or T residue at a position corresponding to position 71 of SEQ ID NO: 1; (d) a G, I, R, S, T, or V residue at a position corresponding to position 72 of SEQ ID NO: 1; (e) an I, R, or V residue at a position corresponding to position 73 of SEQ ID NO: 1; and (f) an A, S, or T residue at a position corresponding to position 74 of SEQ ID NO: 1.

In some embodiments of the method, the center sequence consists of TCAA, and wherein the modifying step comprises modifying the second subunit to comprise one or more of the following residues: (a) a K or S residue at a position corresponding to position 48 of SEQ ID NO: 1; (b) a C, K, R, or T residue at a position corresponding to position 50 of SEQ ID NO: 1; (c) a G, R, or T residue at a position corresponding to position 71 of SEQ ID NO: 1; (d) a G, P, R, S, or T residue at a position corresponding to position 72 of SEQ ID NO: 1; (e) a I or V residue at a position corresponding to position 73 of SEQ ID NO: 1; and (f) an A, S, or T residue at a position corresponding to position 74 of SEQ ID NO: 1.

In some embodiments of the method: (a) the center sequence is ACAA and the first subunit comprises residues corresponding to residues 48, 50, 71, 72, 73, and 74 of any one of SEQ ID NOs: 11-33, (b) the center sequence is ACAG and the first subunit comprises residues corresponding to residues 48, 50, 71, 72, 73, and 74 of any one of SEQ ID NOs: 36-43, (c) the center sequence is ACAT and the first subunit comprises residues corresponding to residues 48, 50, 71, 72, 73, and 74 of any one of SEQ ID NOs: 46-67, (d) the center sequence is ACGA and the first subunit comprises residues corresponding to residues 48, 50, 71, 72, 73, and 74 of any one of SEQ ID NOs: 70-89, (e) the center sequence is ACGC and the first subunit comprises residues corresponding to residues 48, 50, 71, 72, 73, and 74 of any one of SEQ ID NOs: 92-118, (f) the center sequence is ACGG and the first subunit comprises residues corresponding to residues 48, 50, 71, 72, 73, and 74 of any one of SEQ ID NOs: 121-135, (g) the center sequence is ACGT and the first subunit comprises residues corresponding to residues 48, 50, 71, 72, 73, and 74 of any one of SEQ ID NOs: 138-156, (h) the center sequence is ATAA and the first subunit comprises residues corresponding to residues 48, 50, 71, 72, 73, and 74 of any one of SEQ ID NOs: 159-183, (i) the center sequence is ATAG and the first subunit comprises residues corresponding to residues 48, 50, 71, 72, 73, and 74 of any one of SEQ ID NOs: 186-199, (j) the center sequence is ATAT and the first subunit comprises residues corresponding to residues 48, 50, 71, 72, 73, and 74 of any one of SEQ ID NOs: 202-219, (k) the center sequence is ATGA and the first subunit comprises residues corresponding to residues 48, 50, 71, 72, 73, and 74 of any one of SEQ ID NOs: 222-243, (l) the center sequence is ATGG and the first subunit comprises residues corresponding to residues 48, 50, 71, 72, 73, and 74 of any one of SEQ ID NOs: 246-247, (m) the center sequence is TTGG and the first subunit comprises residues corresponding to residues 48, 50, 71, 72, 73, and 74 of any one of SEQ ID NOs: 250-266, (n) the center sequence is GCAA and the first subunit comprises residues corresponding to residues 48, 50, 71, 72, 73, and 74 of any one of SEQ ID NOs: 269-291, (o) the center sequence is GCAT and the first subunit comprises residues corresponding to residues 48, 50, 71, 72, 73, and 74 of any one of SEQ ID NOs: 294-313. (p) the center sequence is GCGA and the first subunit comprises residues corresponding to residues 48, 50, 71, 72, 73, and 74 of any one of SEQ ID NOs: 316-325, (q) the center sequence is GCAG and the first subunit comprises residues corresponding to residues 48, 50, 71, 72, 73, and 74 of any one of SEQ ID NOs: 328-330, (r) the center sequence is TCAA and the first subunit comprises residues corresponding to residues 48, 50, 71, 72, 73, and 74 of any one of SEQ ID NOs: 333-340, or (s) the center sequence is TTAA and the first subunit comprises residues corresponding to residues 48, 50, 71, 72, 73, and 74 of any one of SEQ ID NOs: 343-357.

In some embodiments of the method: (a) the center sequence is ACAA and the second subunit comprises residues corresponding to residues 48, 50, 71, 72, 73, and 74 of any one of SEQ ID NOs: 11-33, (b) the center sequence is ACAG and the second subunit comprises residues corresponding to residues 48, 50, 71, 72, 73, and 74 of any one of SEQ ID NOs: 36-43, (c) the center sequence is ACAT and the second subunit comprises residues corresponding to residues 48, 50, 71, 72, 73, and 74 of any one of SEQ ID NOs: 46-67, (d) the center sequence is ACGA and the second subunit comprises residues corresponding to residues 48, 50, 71, 72, 73, and 74 of any one of SEQ ID NOs: 70-89, (e) the center sequence is ACGC and the second subunit comprises residues corresponding to residues 48, 50, 71, 72, 73, and 74 of any one of SEQ ID NOs: 92-118, (f) the center sequence is ACGG and the second subunit comprises residues corresponding to residues 48, 50, 71, 72, 73, and 74 of any one of SEQ ID NOs: 121-135, (g) the center sequence is ACGT and the second subunit comprises residues corresponding to residues 48, 50, 71, 72, 73, and 74 of any one of SEQ ID NOs: 138-156, (h) the center sequence is ATAA and the second subunit comprises residues corresponding to residues 48, 50, 71, 72, 73, and 74 of any one of SEQ ID NOs: 159-183, (i) the center sequence is ATAG and the second subunit comprises residues corresponding to residues 48, 50, 71, 72, 73, and 74 of any one of SEQ ID NOs: 186-199, (j) the center sequence is ATAT and the second subunit comprises residues corresponding to residues 48, 50, 71, 72, 73, and 74 of any one of SEQ ID NOs: 202-219, (k) the center sequence is ATGA and the second subunit comprises residues corresponding to residues 48, 50, 71, 72, 73, and 74 of any one of SEQ ID NOs: 222-243, (l) the center sequence is ATGG and the second subunit comprises residues corresponding to residues 48, 50, 71, 72, 73, and 74 of any one of SEQ ID NOs: 246-247, (m) the center sequence is TTGG and the second subunit comprises residues corresponding to residues 48, 50, 71, 72, 73, and 74 of any one of SEQ ID NOs: 250-266, (n) the center sequence is GCAA and the second subunit comprises residues corresponding to residues 48, 50, 71, 72, 73, and 74 of any one of SEQ ID NOs: 269-291, (o) the center sequence is GCAT and the second subunit comprises residues corresponding to residues 48, 50, 71, 72, 73, and 74 of any one of SEQ ID NOs: 294-313, (p) the center sequence is GCGA and the second subunit comprises residues corresponding to residues 48, 50, 71, 72, 73, and 74 of any one of SEQ ID NOs: 316-325, (q) the center sequence is GCAG and the second subunit comprises residues corresponding to residues 48, 50, 71, 72, 73, and 74 of any one of SEQ ID NOs: 328-330, (r) the center sequence is TCAA and the second subunit comprises residues corresponding to residues 48, 50, 71, 72, 73, and 74 of any one of SEQ ID NOs: 333-340, or (s) the center sequence is TTAA and the second subunit comprises residues corresponding to residues 48, 50, 71, 72, 73, and 74 of any one of SEQ ID NOs: 343-357.

Another aspect is an I-CreI derived engineered meganuclease having specificity for a recognition sequence comprising a center sequence consisting of GTAA, GTAG, GTAT, GTGA, GTGC, GTGG, or GTGT, wherein the engineered meganuclease comprises a first subunit and a second subunit, wherein the first subunit comprises an amino acid sequence derived from SEQ ID NO: 1, and wherein the first subunit comprises a substitution at one or more positions corresponding to positions 48, 50, 71, 72, 73, and 74 of SEQ ID NO: 1.

Another aspect is an improved engineered I-CreI-derived meganuclease that binds and cleaves a recognition sequence comprising a center sequence consisting of GTAA, GTAG, GTAT, GTGA, GTGC, GTGG, or GTGT, wherein the engineered meganuclease comprises a first subunit and a second subunit, wherein the first subunit and the second subunit each comprise an amino acid sequence derived from SEQ ID NO: 1, the improvement comprising any amino acid substitution described herein that improves cleavage activity of the GTAA, GTAG, GTAT, GTGA, GTGC, GTGG, or GTGT center sequence.

In some embodiments, the first subunit comprises one or more of the following residues: (a) an A, C, G, H, K, L, M, N, Q, R, S, T, or V residue at a position corresponding to position 48 of SEQ ID NO: 1; (b) an A, C, E, G, I, K, L, Q, R, S, T, or V residue at a position corresponding to position 50 of SEQ ID NO: 1; (c) an A, D, E, F, G, H, I, K, L, N, Q, R, S, T, V, or Y residue at a position corresponding to position 71 of SEQ ID NO: 1; (d) an A, C, D, G, H, K, M, N, P, Q, R, S, T, V, W, or Y residue at a position corresponding to position 72 of SEQ ID NO: 1; (e) an A, C, I, L, N, R, S, T, or V residue at a position corresponding to position 73 of SEQ ID NO: 1; and (f) an A, C, G, S, or T residue at a position corresponding to position 74 of SEQ ID NO: 1.

In some embodiments, the second subunit comprises one or more of the following residues: (a) a K residue at a position corresponding to position 48 of SEQ ID NO: 1; (b) a Q residue at a position corresponding to position 50 of SEQ ID NO: 1; (c) a G residue at a position corresponding to position 71 of SEQ ID NO: 1; (d) a S residue at a position corresponding to position 72 of SEQ ID NO: 1; (e) a V residue at a position corresponding to position 73 of SEQ ID NO: 1; and (f) a S residue at a position corresponding to position 74 of SEQ ID NO: 1.

In some embodiments: (a) the center sequence is GTAA and the first subunit comprises residues corresponding to residues 48, 50, 71, 72, 73, and 74 of any one of SEQ ID NOs: 360-389, (b) the center sequence is GTAG and the first subunit comprises residues corresponding to residues 48, 50, 71, 72, 73, and 74 of any one of SEQ ID NOs: 392-399, (c) the center sequence is GTAT and the first subunit comprises residues corresponding to residues 48, 50, 71, 72, 73, and 74 of any one of SEQ ID NOs: 402-433, (d) the center sequence is GTGA and the first subunit comprises residues corresponding to residues 48, 50, 71, 72, 73, and 74 of any one of SEQ ID NOs: 436-462, (e) the center sequence is GTGC and the first subunit comprises residues corresponding to residues 48, 50, 71, 72, 73, and 74 of any one of SEQ ID NOs: 465-495, (f) the center sequence is GTGG and the first subunit comprises residues corresponding to residues 48, 50, 71, 72, 73, and 74 of any one of SEQ ID NOs: 498-501, or (g) the center sequence is GTGT and the first subunit comprises residues corresponding to residues 48, 50, 71, 72, 73, and 74 of any one of SEQ ID NOs: 504-529.

Another aspect is a method for cleaving double-stranded DNA at a target site comprising a meganuclease recognition sequence, wherein the recognition sequence comprises a center sequence consisting of GTAA, GTAG, GTAT, GTGA, GTGC, GTGG, or GTGT, wherein the method comprises contacting the double-stranded DNA having the target site with any engineered meganuclease described herein, wherein the engineered meganuclease binds and cleaves the recognition sequence.

Another aspect is an improved method for cleaving double-stranded DNA at a target site comprising a meganuclease recognition sequence, by contacting said double-stranded DNA having said target site with an engineered I-CreI-derived meganuclease, wherein the engineered meganuclease comprises a first subunit and a second subunit, wherein the first subunit and the second subunit each comprise an amino acid sequence derived from SEQ ID NO: 1, wherein said recognition sequence comprises a center sequence consisting of GTAA, GTAG, GTAT, GTGA, GTGC, GTGG, or GTGT, the improvement comprising: use of an engineered I-CreI-derived meganuclease described herein, wherein said engineered I-CreI-derived meganuclease binds and cleaves said recognition sequence.

Another aspect is a method for increasing the cleavage activity of an I-CreI derived engineered meganuclease that binds and cleaves a recognition sequence comprising a center sequence consisting of GTAA, GTAG, GTAT, GTGA, GTGC, GTGG, or GTGT, wherein the engineered meganuclease comprises a first subunit and a second subunit, wherein the first subunit comprises an amino acid sequence derived from SEQ ID NO: 1, the method comprising modifying the first subunit at one or more positions corresponding to positions 48, 50, 71, 72, 73, and 74 of SEQ ID NO: 1, wherein the modified nuclease has increased cleavage activity when compared to a control engineered meganuclease.

Another aspect is an improved method for increasing the cleavage activity of an engineered meganuclease that binds and cleaves a recognition sequence comprising a center sequence consisting of GTAA, GTAG, GTAT, GTGA, GTGC, GTGG, or GTGT, wherein the engineered meganuclease comprises a first subunit and a second subunit, wherein the first subunit and the second subunit each comprise an amino acid sequence derived from SEQ ID NO: 1, the improvement comprising use of an engineered I-CreI-derived meganuclease described herein, wherein said engineered I-CreI-derived meganuclease binds and cleaves said recognition sequence.

In some embodiments of the method, the modifying step comprises modifying the first subunit to comprise one or more of the following residues: (a) an A, C, G, H, K, L, M, N, Q, R, S, T, or V residue at a position corresponding to position 48 of SEQ ID NO: 1; (b) an A, C, E, G, I, K, L, Q, R, S, T, or V residue at a position corresponding to position 50 of SEQ ID NO: 1; (c) an A, D, E, F, G, H, I, K, L, N, Q, R, S, T, V, or Y residue at a position corresponding to position 71 of SEQ ID NO: 1; (d) an A, C, D, G, H, K, M, N, P, Q, R, S, T, V, W, or Y residue at a position corresponding to position 72 of SEQ ID NO: 1; (e) an A, C, I, L, N, R, S, T, or V residue at a position corresponding to position 73 of SEQ ID NO: 1; and (f) an A, C, G, S, or T residue at a position corresponding to position 74 of SEQ ID NO: 1.

In some embodiments of the method, the second subunit comprises one or more of the following residues: (a) a K residue at a position corresponding to position 48 of SEQ ID NO: 1; (b) a Q residue at a position corresponding to position 50 of SEQ ID NO: 1; (c) a G residue at a position corresponding to position 71 of SEQ ID NO: 1; (d) a S residue at a position corresponding to position 72 of SEQ ID NO: 1; (e) a V residue at a position corresponding to position 73 of SEQ ID NO: 1; and (f) a S residue at a position corresponding to position 74 of SEQ ID NO: 1.

In some embodiments of the method: (a) the center sequence is GTAA and the first subunit comprises residues corresponding to residues 48, 50, 71, 72, 73, and 74 of any one of SEQ ID NOs: 360-389, (b) the center sequence is GTAG and the first subunit comprises residues corresponding to residues 48, 50, 71, 72, 73, and 74 of any one of SEQ ID NOs: 392-399, (c) the center sequence is GTAT and the first subunit comprises residues corresponding to residues 48, 50, 71, 72, 73, and 74 of any one of SEQ ID NOs: 402-433, (d) the center sequence is GTGA and the first subunit comprises residues corresponding to residues 48, 50, 71, 72, 73, and 74 of any one of SEQ ID NOs: 436-462, (e) the center sequence is GTGC and the first subunit comprises residues corresponding to residues 48, 50, 71, 72, 73, and 74 of any one of SEQ ID NOs: 465-495, (f) the center sequence is GTGG and the first subunit comprises residues corresponding to residues 48, 50, 71, 72, 73, and 74 of any one of SEQ ID NOs: 498-501, or (g) the center sequence is GTGT and the first subunit comprises residues corresponding to residues 48, 50, 71, 72, 73, and 74 of any one of SEQ ID NOs: 504-529.

Another aspect is an engineered I-CreI-derived meganuclease that binds and cleaves a recognition sequence comprising a center sequence selected from the group consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, or TTAA, wherein said engineered meganuclease comprises a first subunit and a second subunit, wherein at least one of said first or second subunit comprises at least 75%, at least 80%, at least 85%, at least 88%, at least 90%, at least 92%, at least 94%, at least 96%, at least 97%, at least 98%, or at least 99% sequence identity to SEQ ID NO: 1 with the exception of an amino acid substitution at one or more positions corresponding to positions 48, 50, 71, 72, 73, and 74 of SEQ ID NO: 1.

In some embodiments, at least one of said first or second subunit comprises at least 85% sequence identity to SEQ ID NO: 1 with the exception of an amino acid substitution at one or more positions corresponding to positions 48, 50, 71, 72, 73, and 74 of SEQ ID NO: 1. Another aspect is a polynucleotide comprising a nucleic acid sequence encoding any engineered meganuclease described herein. In some embodiments, the polynucleotide an mRNA.

Another aspect is a recombinant DNA construct comprising a polynucleotide comprising a nucleic acid sequence encoding any engineered meganuclease described herein. In some embodiments, the recombinant DNA construct encodes a recombinant virus comprising the polynucleotide. In some embodiments, the recombinant virus is a recombinant adenovirus, a recombinant lentivirus, a recombinant retrovirus, or a recombinant adeno-associated virus (AAV). In some embodiments, the recombinant virus is a recombinant AAV.

Another aspect is a recombinant virus comprising a polynucleotide comprising a nucleic acid sequence encoding any engineered meganuclease described herein. In some embodiments, the recombinant virus is a recombinant adenovirus, a recombinant lentivirus, a recombinant retrovirus, or a recombinant AAV. In some embodiments, the recombinant virus is a recombinant AAV.

Another aspect is a method for producing a genetically-modified eukaryotic cell having a disrupted target sequence in a chromosome of the genetically-modified eukaryotic cell, the method comprising: introducing into a eukaryotic cell a polynucleotide comprising a nucleic acid sequence encoding any engineered meganuclease described herein, wherein the engineered meganuclease is expressed in the eukaryotic cell; wherein the engineered meganuclease produces a cleavage site in the chromosome at a recognition sequence, and wherein the target sequence is disrupted by non-homologous end-joining at the cleavage site.

In some embodiments of the method, the nucleic acid is introduced into the eukaryotic cell by an mRNA or a recombinant virus. In some embodiments of the method, the eukaryotic cell is a mammalian cell. In some embodiments of the method, the eukaryotic cell is a human cell. In some embodiments of the method, the eukaryotic cell is a plant cell.

Another aspect is a method for producing a genetically-modified eukaryotic having a disrupted target sequence in a chromosome of the genetically-modified eukaryotic cell, the method comprising: introducing into a eukaryotic cell any engineered meganuclease described herein; wherein the engineered meganuclease produces a cleavage site in the chromosome at a recognition sequence, and wherein the target sequence is disrupted by non-homologous end-joining at the cleavage site.

In some embodiments of the method, the eukaryotic cell is a mammalian cell. In some embodiments of the method, the eukaryotic cell is a human cell. In some embodiments of the method the eukaryotic cell is a plant cell.

Another aspect is a method for producing a genetically-modified eukaryotic cell comprising an exogenous sequence of interest inserted into a chromosome of the genetically-modified eukaryotic cell, the method comprising introducing into a eukaryotic cell one or more polynucleotides comprising: (a) a first nucleic acid sequence encoding any engineered meganuclease described herein, wherein the engineered meganuclease is expressed in the eukaryotic cell; and (b) a second nucleic acid sequence comprising the sequence of interest; wherein the engineered meganuclease produces a cleavage site in the chromosome at a recognition sequence; and wherein the sequence of interest is inserted into the chromosome at the cleavage site.

In some embodiments of the method, the second nucleic acid sequence further comprises sequences homologous to sequences flanking the cleavage site and the sequence of interest is inserted at the cleavage site by homologous recombination. In some embodiments of the method, the first nucleic acid sequence is introduced into the eukaryotic cell by an mRNA or a recombinant virus. In some embodiments of the method, the second nucleic acid is introduced into the eukaryotic cell by a recombinant virus. In some embodiments of the method, the eukaryotic cell is a mammalian cell. In some embodiments of the method, the eukaryotic cell is a human cell. In some embodiments of the method, the eukaryotic cell is a plant cell.

Another aspect is a method for producing a genetically-modified eukaryotic cell comprising an exogenous sequence of interest inserted into a chromosome of the genetically modified eukaryotic cell, the method comprising: (a) introducing any engineered meganuclease described herein into a eukaryotic cell; and (b) introducing a polynucleotide comprising a nucleic acid sequence comprising the sequence of interest into the eukaryotic cell; wherein the engineered meganuclease produces a cleavage site in the chromosome at a recognition sequence; and wherein the sequence of interest is inserted into the chromosome at the cleavage site.

In some embodiments of the method, the polynucleotide further comprises sequences homologous to sequences flanking the cleavage site and the sequence of interest is inserted at the cleavage site by homologous recombination. In some embodiments of the method, the polynucleotide is introduced into the eukaryotic cell by a recombinant virus. In some embodiments of the method, the eukaryotic cell is a mammalian cell. In some embodiments of the method, the eukaryotic cell is a human cell. In some embodiments of the method, the eukaryotic cell is a plant cell.

Another aspect is a genetically-modified eukaryotic cell prepared by any method of preparing a genetically-modified cell described herein.

Another aspect is a pharmaceutical composition comprising a pharmaceutically-acceptable carrier and any engineered meganuclease described herein, or a polynucleotide comprising a nucleic acid sequence encoding any engineered meganuclease described herein.

In some embodiments, the polynucleotide is an mRNA. In some embodiments, the mRNA is encapsulated in a lipid nanoparticle. In some embodiments, the pharmaceutical composition comprises a recombinant DNA construct comprising the polynucleotide. In some embodiments, the pharmaceutical composition comprises a recombinant virus comprising the polynucleotide. In some embodiments, the recombinant virus is a recombinant AAV.

These and other aspects and embodiments of the invention will be apparent to one of ordinary skill in the art from the following detailed description of the invention, figures and appended claims.

### BRIEF DESCRIPTION OF THE FIGURES

FIG. 1. Schematic illustration of a 22 base pair wild type I-CreI recognition sequence. The bases of each DNA half-site are numbered −1 through −9. The four base pairs one each strand that comprise the center sequence are numbered +1 to +4.

FIG. 2. Engineered meganucleases described herein comprise two subunits. The first subunit comprises a first hypervariable (HVR1) region which binds to a first recognition half-site of the recognition sequence. Similarly, the second subunit comprises a second hypervariable (HVR2) region which binds to a second recognition half-site of the recognition sequence. In embodiments where the recombinant meganuclease is a single-chain meganuclease, the first subunit comprising the HVR1 region can be positioned as either the N-terminal or C-terminal subunit. Likewise, the second subunit comprising the HVR2 region can be positioned as either the N-terminal or C-terminal subunit.

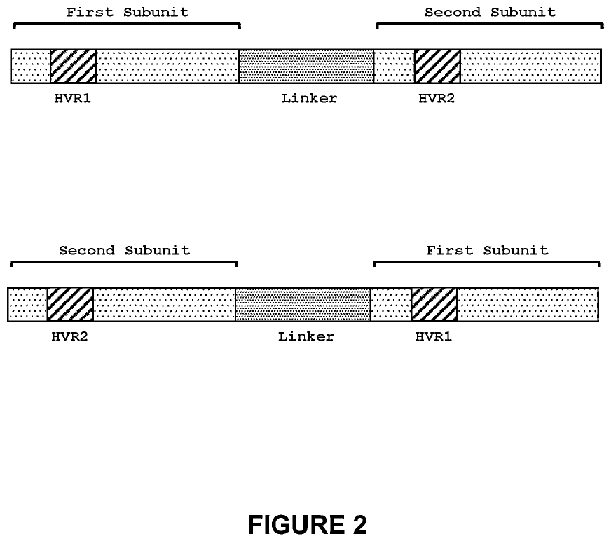

FIG. 3. Schematic of reporter assay in CHO cells for evaluating recombinant meganucleases targeting test recognition sequences having different four base pair center sequences. For the recombinant meganucleases described herein, a CHO cell line was produced in which a reporter cassette was integrated stably into the genome of the cell. The reporter cassette comprised, in 5′ to 3′ order: an SV40 Early Promoter; the 5′2/3 of the GFP gene; the recognition sequence for an engineered meganuclease described herein (e.g., LOX 3-4; SEQ ID NO: 6); the recognition sequence for the CHO-23/24 meganuclease (WO/2012/167192); and the 3′ 2/3 of the GFP gene. Cells stably transfected with this cassette do not express GFP in the absence of a DNA break-inducing agent. Meganucleases are introduced by transduction of an mRNA encoding each meganuclease. When a DNA break is induced at either of the meganuclease recognition sequences, the duplicated regions of the GFP gene recombine with one another to produce a functional GFP gene. The percentage of GFP-expressing cells can then be determined by flow cytometry as an indirect measure of the frequency of genome cleavage by the meganucleases.

FIG. 4. Crystal structure of a modified I-CreI derived meganuclease (light color) overlaid with a wild type I-CreI meganuclease (dark color). The variant meganuclease has modified residues Q50R, G71S, S72G, and V73R, which increases the variant meganuclease cleavage activity of a recognition sequence comprising the four base pair center sequence GCAG. The nucleotide G from the variant I-CreI meganuclease and the nucleotide A from the wild type I-CreI meganuclease are shown. Further presented is the overlaid alignment of positions 47, 48, 49, 50, 71, 72, and 73, which are arranged around the nucleotides of the center four base pair center sequence. Lastly, the small spheres depict the overlaid metal co-factors which are thought to be coordinated, at least in part, by the residues 48, 50, 71, 72, 73, and 74.

### BRIEF DESCRIPTION OF THE SEQUENCES

SEQ ID NO: 1 sets forth the amino acid sequence of wild-type I-CreI.

SEQ ID NO: 2 sets forth the amino acid sequence of the LAGLIDADG motif.

SEQ ID NO: 3 sets forth the nucleic acid sequence of the wild-type I-CrcI recognition sequence (sense).

SEQ ID NO: 4 sets forth the nucleic acid sequence of the wild-type I-CreI recognition sequence (antisense).

SEQ ID NO: 5 sets forth the nucleic acid sequence of the center sequence of the wild-type I-CreI recognition sequence.

SEQ ID NO: 6 sets forth the nucleic acid sequence of the LOX 3-4 recognition sequence (sense).

SEQ ID NO: 7 sets forth the nucleic acid sequence of the LOX 3-4 recognition sequence (antisense).

SEQ ID NO: 8 sets forth the amino acid sequence of the LOX 3-4x.109 meganuclease.

SEQ ID NO: 9 sets forth the nucleic acid of the LOX 3-4 recognition sequence (sense) with an ACAA center sequence.

SEQ ID NO: 10 sets forth the nucleic acid of the LOX 3-4 recognition sequence (antisense) with an ACAA center sequence.

SEQ ID NO: 11 sets forth the amino acid sequence of the LOX 3-4 m.680 meganuclease.

SEQ ID NO: 12 sets forth the amino acid sequence of the LOX 3-4 m.683 meganuclease.

SEQ ID NO: 13 sets forth the amino acid sequence of the LOX 3-4 m.684 meganuclease.

SEQ ID NO: 14 sets forth the amino acid sequence of the LOX 3-4 m.691 meganuclease.

SEQ ID NO: 15 sets forth the amino acid sequence of the LOX 3-4 m.693 meganuclease.

SEQ ID NO: 16 sets forth the amino acid sequence of the LOX 3-4 m.701 meganuclease.

SEQ ID NO: 17 sets forth the amino acid sequence of the LOX 3-4 m.708 meganuclease.

SEQ ID NO: 18 sets forth the amino acid sequence of the LOX 3-4 m.714 meganuclease.

SEQ ID NO: 19 sets forth the amino acid sequence of the LOX 3-4 m.731 meganuclease.

SEQ ID NO: 20 sets forth the amino acid sequence of the LOX 3-4 m.739 meganuclease.

SEQ ID NO: 21 sets forth the amino acid sequence of the LOX 3-4 m.741 SEQ ID NO: 22 sets forth the amino acid sequence of the LOX 3-4 m.742 meganuclease.

SEQ ID NO: 23 sets forth the amino acid sequence of the LOX 3-4 m.743 meganuclease.

SEQ ID NO: 24 sets forth the amino acid sequence of the LOX 3-4 m.744 meganuclease.

SEQ ID NO: 25 sets forth the amino acid sequence of the LOX 3-4 m.747 meganuclease.

SEQ ID NO: 26 sets forth the amino acid sequence of the LOX 3-4 m.750 meganuclease.

SEQ ID NO: 27 sets forth the amino acid sequence of the LOX 3-4 m.756 meganuclease.

SEQ ID NO: 28 sets forth the amino acid sequence of the LOX 3-4 m.757 meganuclease.

SEQ ID NO: 29 sets forth the amino acid sequence of the LOX 3-4 m.759 meganuclease.

SEQ ID NO: 30 sets forth the amino acid sequence of the LOX 3-4 m.762 meganuclease.

SEQ ID NO: 31 sets forth the amino acid sequence of the LOX 3-4 m.765 meganuclease.

SEQ ID NO: 32 sets forth the amino acid sequence of the LOX 3-4 m.770 meganuclease.

SEQ ID NO: 33 sets forth the amino acid sequence of the LOX 3-4 m.771 meganuclease.

SEQ ID NO: 34 sets forth the nucleic acid of the LOX 3-4 recognition sequence (sense) with an ACAG center sequence.

SEQ ID NO: 35 sets forth the nucleic acid of the LOX 3-4 recognition sequence (antisense) with an ACAG center sequence.

SEQ ID NO: 36 sets forth the amino acid sequence of the LOX3-4 m.775 meganuclease.

SEQ ID NO: 37 sets forth the amino acid sequence of the LOX3-4 m.776 SEQ ID NO: 38 sets forth the amino acid sequence of the LOX3-4 m.785 meganuclease.

SEQ ID NO: 39 sets forth the amino acid sequence of the LOX3-4 m.788 meganuclease.

SEQ ID NO: 40 sets forth the amino acid sequence of the LOX3-4 m.815 meganuclease.

SEQ ID NO: 41 sets forth the amino acid sequence of the LOX3-4 m.831 meganuclease.

SEQ ID NO: 42 sets forth the amino acid sequence of the LOX3-4 m.856 meganuclease.

SEQ ID NO: 43 sets forth the amino acid sequence of the LOX3-4 m.863 meganuclease.

SEQ ID NO: 44 sets forth the nucleic acid of the LOX 3-4 recognition sequence (sense) with an ACAT center sequence.

SEQ ID NO: 45 sets forth the nucleic acid of the LOX 3-4 recognition sequence (antisense) with an ACAT center sequence.

SEQ ID NO: 46 sets forth the amino acid sequence of the LOX3-4 m.869 meganuclease.

SEQ ID NO: 47 sets forth the amino acid sequence of the LOX3-4 m.873 meganuclease.

SEQ ID NO: 48 sets forth the amino acid sequence of the LOX3-4 m.877 meganuclease.

SEQ ID NO: 49 sets forth the amino acid sequence of the LOX3-4 m.883 meganuclease.

SEQ ID NO: 50 sets forth the amino acid sequence of the LOX3-4 m.885 meganuclease.

SEQ ID NO: 51 sets forth the amino acid sequence of the LOX3-4 m.886 meganuclease.

SEQ ID NO: 52 sets forth the amino acid sequence of the LOX3-4 m.893 meganuclease.

SEQ ID NO: 53 sets forth the amino acid sequence of the LOX3-4 m.901 SEQ ID NO: 54 sets forth the amino acid sequence of the LOX3-4 m.910 meganuclease.

SEQ ID NO: 55 sets forth the amino acid sequence of the LOX3-4 m.917 meganuclease.

SEQ ID NO: 56 sets forth the amino acid sequence of the LOX3-4 m.919 meganuclease.

SEQ ID NO: 57 sets forth the amino acid sequence of the LOX3-4 m.922 meganuclease.

SEQ ID NO: 58 sets forth the amino acid sequence of the LOX3-4 m.925 meganuclease.

SEQ ID NO: 59 sets forth the amino acid sequence of the LOX3-4 m.929 meganuclease.

SEQ ID NO: 60 sets forth the amino acid sequence of the LOX3-4 m.930 meganuclease.

SEQ ID NO: 61 sets forth the amino acid sequence of the LOX3-4 m.933 meganuclease.

SEQ ID NO: 62 sets forth the amino acid sequence of the LOX3-4 m.937 meganuclease.

SEQ ID NO: 63 sets forth the amino acid sequence of the LOX3-4 m.941 meganuclease.

SEQ ID NO: 64 sets forth the amino acid sequence of the LOX3-4 m.942 meganuclease.

SEQ ID NO: 65 sets forth the amino acid sequence of the LOX3-4 m.945 meganuclease.

SEQ ID NO: 66 sets forth the amino acid sequence of the LOX3-4 m.949 meganuclease.

SEQ ID NO: 67 sets forth the amino acid sequence of the LOX3-4 m.950 meganuclease.

SEQ ID NO: 68 sets forth the nucleic acid of the LOX 3-4 recognition sequence (sense) with an ACGA center sequence.

SEQ ID NO: 69 sets forth the nucleic acid of the LOX 3-4 recognition sequence (antisense) with an ACGA center sequence.

SEQ ID NO: 70 sets forth the amino acid sequence of the LOX 3-4 m.956 meganuclease.

SEQ ID NO: 71 sets forth the amino acid sequence of the LOX 3-4 m.961 meganuclease.

SEQ ID NO: 72 sets forth the amino acid sequence of the LOX 3-4 m.962 meganuclease.

SEQ ID NO: 73 sets forth the amino acid sequence of the LOX 3-4 m.963 meganuclease.

SEQ ID NO: 74 sets forth the amino acid sequence of the LOX 3-4 m.969 meganuclease.

SEQ ID NO: 75 sets forth the amino acid sequence of the LOX 3-4 m.971 meganuclease.

SEQ ID NO: 76 sets forth the amino acid sequence of the LOX 3-4 m.977 meganuclease.

SEQ ID NO: 77 sets forth the amino acid sequence of the LOX 3-4 m.982 meganuclease.

SEQ ID NO: 78 sets forth the amino acid sequence of the LOX 3-4 m.986 meganuclease.

SEQ ID NO: 79 sets forth the amino acid sequence of the LOX 3-4 m.993 meganuclease.

SEQ ID NO: 80 sets forth the amino acid sequence of the LOX 3-4 m.994 meganuclease.

SEQ ID NO: 81 sets forth the amino acid sequence of the LOX 3-4 m.1001 meganuclease.

SEQ ID NO: 82 sets forth the amino acid sequence of the LOX 3-4 m.1013 meganuclease.

SEQ ID NO: 83 sets forth the amino acid sequence of the LOX 3-4 m.1017 meganuclease.

SEQ ID NO: 84 sets forth the amino acid sequence of the LOX 3-4 m.1018 meganuclease.

SEQ ID NO: 85 sets forth the amino acid sequence of the LOX 3-4 m.1021 SEQ ID NO: 86 sets forth the amino acid sequence of the LOX 3-4 m.1029 meganuclease.

SEQ ID NO: 87 sets forth the amino acid sequence of the LOX 3-4 m.1036 meganuclease.

SEQ ID NO: 88 sets forth the amino acid sequence of the LOX 3-4 m.1041 meganuclease.

SEQ ID NO: 89 sets forth the amino acid sequence of the LOX 3-4 m.1044 meganuclease.

SEQ ID NO: 90 sets forth the nucleic acid of the LOX 3-4 recognition sequence (sense) with an ACGC center sequence.

SEQ ID NO: 91 sets forth the nucleic acid of the LOX 3-4 recognition sequence (antisense) with an ACGC center sequence.

SEQ ID NO: 92 sets forth the amino acid sequence of the LOX 3-4 m.1049 meganuclease.

SEQ ID NO: 93 sets forth the amino acid sequence of the LOX 3-4 m.1050 meganuclease.

SEQ ID NO: 94 sets forth the amino acid sequence of the LOX 3-4 m.1052 meganuclease.

SEQ ID NO: 95 sets forth the amino acid sequence of the LOX 3-4 m.1068 meganuclease.

SEQ ID NO: 96 sets forth the amino acid sequence of the LOX 3-4 m.1069 meganuclease.

SEQ ID NO: 97 sets forth the amino acid sequence of the LOX 3-4 m.1074 meganuclease.

SEQ ID NO: 98 sets forth the amino acid sequence of the LOX 3-4 m.1085 meganuclease.

SEQ ID NO: 99 sets forth the amino acid sequence of the LOX 3-4 m.1093 meganuclease.

SEQ ID NO: 100 sets forth the amino acid sequence of the LOX 3-4 m.1095 meganuclease.

SEQ ID NO: 101 sets forth the amino acid sequence of the LOX 3-4 m.1098 meganuclease. SEQ ID NO: 102 sets forth the amino acid sequence of the LOX 3-4 m.1100 meganuclease.

SEQ ID NO: 103 sets forth the amino acid sequence of the LOX 3-4 m.1101 meganuclease.

SEQ ID NO: 104 sets forth the amino acid sequence of the LOX 3-4 m.1107 meganuclease.

SEQ ID NO: 105 sets forth the amino acid sequence of the LOX 3-4 m.1109 meganuclease.

SEQ ID NO: 106 sets forth the amino acid sequence of the LOX 3-4 m.1111 meganuclease.

SEQ ID NO: 107 sets forth the amino acid sequence of the LOX 3-4 m.1113 meganuclease.

SEQ ID NO: 108 sets forth the amino acid sequence of the LOX 3-4 m.1116 meganuclease.

SEQ ID NO: 109 sets forth the amino acid sequence of the LOX 3-4 m.1117 meganuclease.

SEQ ID NO: 110 sets forth the amino acid sequence of the LOX 3-4 m.1118 meganuclease.

SEQ ID NO: 111 sets forth the amino acid sequence of the LOX 3-4 m.1123 meganuclease.

SEQ ID NO: 112 sets forth the amino acid sequence of the LOX 3-4 m.1125 meganuclease.

SEQ ID NO: 113 sets forth the amino acid sequence of the LOX 3-4 m.1126 meganuclease.

SEQ ID NO: 114 sets forth the amino acid sequence of the LOX 3-4 m.1127 meganuclease.

SEQ ID NO: 115 sets forth the amino acid sequence of the LOX 3-4 m.1129 meganuclease.

SEQ ID NO: 116 sets forth the amino acid sequence of the LOX 3-4 m.1131 meganuclease.

SEQ ID NO: 117 sets forth the amino acid sequence of the LOX 3-4 m.1133 SEQ ID NO: 118 sets forth the amino acid sequence of the LOX 3-4 m.1137 meganuclease.

SEQ ID NO: 119 sets forth the nucleic acid of the LOX 3-4 recognition sequence (sense) with an ACGG center sequence.

SEQ ID NO: 120 sets forth the nucleic acid of the LOX 3-4 recognition sequence (antisense) with an ACGG center sequence.

SEQ ID NO: 121 sets forth the amino acid sequence of the LOX 3-4 m.1876 meganuclease.

SEQ ID NO: 122 sets forth the amino acid sequence of the LOX 3-4 m.1894 meganuclease.

SEQ ID NO: 123 sets forth the amino acid sequence of the LOX 3-4 m.1898 meganuclease.

SEQ ID NO: 124 sets forth the amino acid sequence of the LOX 3-4 m.1904 meganuclease.

SEQ ID NO: 125 sets forth the amino acid sequence of the LOX 3-4 m.1910 meganuclease.

SEQ ID NO: 126 sets forth the amino acid sequence of the LOX 3-4 m.1914 meganuclease.

SEQ ID NO: 127 sets forth the amino acid sequence of the LOX 3-4 m.1930 meganuclease.

SEQ ID NO: 128 sets forth the amino acid sequence of the LOX 3-4 m.1938 meganuclease.

SEQ ID NO: 129 sets forth the amino acid sequence of the LOX 3-4 m.1941 meganuclease.

SEQ ID NO: 130 sets forth the amino acid sequence of the LOX 3-4 m.1944 meganuclease.

SEQ ID NO: 131 sets forth the amino acid sequence of the LOX 3-4 m.1946 meganuclease.

SEQ ID NO: 132 sets forth the amino acid sequence of the LOX 3-4 m.1947 meganuclease.

SEQ ID NO: 133 sets forth the amino acid sequence of the LOX 3-4 m.1950 SEQ ID NO: 134 sets forth the amino acid sequence of the LOX 3-4 m.1952 meganuclease.

SEQ ID NO: 135 sets forth the amino acid sequence of the LOX 3-4 m.1960 meganuclease.

SEQ ID NO: 136 sets forth the nucleic acid of the LOX 3-4 recognition sequence (sense) with an ACGT center sequence.

SEQ ID NO: 137 sets forth the nucleic acid of the LOX 3-4 recognition sequence (antisense) with an ACGT center sequence.

SEQ ID NO: 138 sets forth the amino acid sequence of the LOX 3-4 m.1145 meganuclease.

SEQ ID NO: 139 sets forth the amino acid sequence of the LOX 3-4 m.1149 meganuclease.

SEQ ID NO: 140 sets forth the amino acid sequence of the LOX 3-4 m.1152 meganuclease.

SEQ ID NO: 141 sets forth the amino acid sequence of the LOX 3-4 m.1153 meganuclease.

SEQ ID NO: 142 sets forth the amino acid sequence of the LOX 3-4 m.1157 meganuclease.

SEQ ID NO: 143 sets forth the amino acid sequence of the LOX 3-4 m.1158 meganuclease.

SEQ ID NO: 144 sets forth the amino acid sequence of the LOX 3-4 m.1176 meganuclease.

SEQ ID NO: 145 sets forth the amino acid sequence of the LOX 3-4 m.1191 meganuclease.

SEQ ID NO: 146 sets forth the amino acid sequence of the LOX 3-4 m.1198 meganuclease.

SEQ ID NO: 147 sets forth the amino acid sequence of the LOX 3-4 m.1201 meganuclease.

SEQ ID NO: 148 sets forth the amino acid sequence of the LOX 3-4 m.1205 meganuclease.

SEQ ID NO: 149 sets forth the amino acid sequence of the LOX 3-4 m.1206 SEQ ID NO: 150 sets forth the amino acid sequence of the LOX 3-4 m.1208 meganuclease.

SEQ ID NO: 151 sets forth the amino acid sequence of the LOX 3-4 m.1212 meganuclease.

SEQ ID NO: 152 sets forth the amino acid sequence of the LOX 3-4 m.1218 meganuclease.

SEQ ID NO: 153 sets forth the amino acid sequence of the LOX 3-4 m.1224 meganuclease.

SEQ ID NO: 154 sets forth the amino acid sequence of the LOX 3-4 m.1225 meganuclease.

SEQ ID NO: 155 sets forth the amino acid sequence of the LOX 3-4 m.1226 meganuclease.

SEQ ID NO: 156 sets forth the amino acid sequence of the LOX 3-4 m.1227 meganuclease.

SEQ ID NO: 157 sets forth the nucleic acid of the LOX 3-4 recognition sequence (sense) with an ATAA center sequence.

SEQ ID NO: 158 sets forth the nucleic acid of the LOX 3-4 recognition sequence (antisense) with an ATAA center sequence.

SEQ ID NO: 159 sets forth the amino acid sequence of the LOX 3-4 m.1232 meganuclease.

SEQ ID NO: 160 sets forth the amino acid sequence of the LOX 3-4 m.1235 meganuclease.

SEQ ID NO: 161 sets forth the amino acid sequence of the LOX 3-4 m.1236 meganuclease.

SEQ ID NO: 162 sets forth the amino acid sequence of the LOX 3-4 m.1237 meganuclease.

SEQ ID NO: 163 sets forth the amino acid sequence of the LOX 3-4 m.1240 meganuclease.

SEQ ID NO: 164 sets forth the amino acid sequence of the LOX 3-4 m.1250 meganuclease.

SEQ ID NO: 165 sets forth the amino acid sequence of the LOX 3-4 m.1253 SEQ ID NO: 166 sets forth the amino acid sequence of the LOX 3-4 m.1255 meganuclease.

SEQ ID NO: 167 sets forth the amino acid sequence of the LOX 3-4 m.1256 meganuclease.

SEQ ID NO: 168 sets forth the amino acid sequence of the LOX 3-4 m.1260 meganuclease.

SEQ ID NO: 169 sets forth the amino acid sequence of the LOX 3-4 m.1261 meganuclease.

SEQ ID NO: 170 sets forth the amino acid sequence of the LOX 3-4 m.1262 meganuclease.

SEQ ID NO: 171 sets forth the amino acid sequence of the LOX 3-4 m.1268 meganuclease.

SEQ ID NO: 172 sets forth the amino acid sequence of the LOX 3-4 m.1269 meganuclease.

SEQ ID NO: 173 sets forth the amino acid sequence of the LOX 3-4 m.1278 meganuclease.

SEQ ID NO: 174 sets forth the amino acid sequence of the LOX 3-4 m.1284 meganuclease.

SEQ ID NO: 175 sets forth the amino acid sequence of the LOX 3-4 m.1293 meganuclease.

SEQ ID NO: 176 sets forth the amino acid sequence of the LOX 3-4 m.1300 meganuclease.

SEQ ID NO: 177 sets forth the amino acid sequence of the LOX 3-4 m.1301 meganuclease.

SEQ ID NO: 178 sets forth the amino acid sequence of the LOX 3-4 m.1308 meganuclease.

SEQ ID NO: 179 sets forth the amino acid sequence of the LOX 3-4 m.1309 meganuclease.

SEQ ID NO: 180 sets forth the amino acid sequence of the LOX 3-4 m.1311 meganuclease.

SEQ ID NO: 181 sets forth the amino acid sequence of the LOX 3-4 m.1317 SEQ ID NO: 182 sets forth the amino acid sequence of the LOX 3-4 m.1319 meganuclease.

SEQ ID NO: 183 sets forth the amino acid sequence of the LOX 3-4 m.1322 meganuclease.

SEQ ID NO: 184 sets forth the nucleic acid of the LOX 3-4 recognition sequence (sense) with an ATAG center sequence.

SEQ ID NO: 185 sets forth the nucleic acid of the LOX 3-4 recognition sequence (antisense) with an ATAG center sequence.

SEQ ID NO: 186 sets forth the amino acid sequence of the LOX 3-4 m.1329 meganuclease.

SEQ ID NO: 187 sets forth the amino acid sequence of the LOX 3-4 m.1338 meganuclease.

SEQ ID NO: 188 sets forth the amino acid sequence of the LOX 3-4 m.1343 meganuclease.

SEQ ID NO: 189 sets forth the amino acid sequence of the LOX 3-4 m.1345 meganuclease.

SEQ ID NO: 190 sets forth the amino acid sequence of the LOX 3-4 m.1347 meganuclease.

SEQ ID NO: 191 sets forth the amino acid sequence of the LOX 3-4 m.1353 meganuclease.

SEQ ID NO: 192 sets forth the amino acid sequence of the LOX 3-4 m.1361 meganuclease.

SEQ ID NO: 193 sets forth the amino acid sequence of the LOX 3-4 m.1369 meganuclease.

SEQ ID NO: 194 sets forth the amino acid sequence of the LOX 3-4 m.1391 meganuclease.

SEQ ID NO: 195 sets forth the amino acid sequence of the LOX 3-4 m.1392 meganuclease.

SEQ ID NO: 196 sets forth the amino acid sequence of the LOX 3-4 m.1394 meganuclease.

SEQ ID NO: 197 sets forth the amino acid sequence of the LOX 3-4 m.1396 SEQ ID NO: 198 sets forth the amino acid sequence of the LOX 3-4 m.1405 meganuclease.

SEQ ID NO: 199 sets forth the amino acid sequence of the LOX 3-4 m.1415 meganuclease.

SEQ ID NO: 200 sets forth the nucleic acid of the LOX 3-4 recognition sequence (sense) with an ATAT center sequence.

SEQ ID NO: 201 sets forth the nucleic acid of the LOX 3-4 recognition sequence (antisense) with an ATAT center sequence.

SEQ ID NO: 202 sets forth the amino acid sequence of the LOX 3-4 m.2244 meganuclease.

SEQ ID NO: 203 sets forth the amino acid sequence of the LOX 3-4 m.2248 meganuclease.

SEQ ID NO: 204 sets forth the amino acid sequence of the LOX 3-4 m.2254 meganuclease.

SEQ ID NO: 205 sets forth the amino acid sequence of the LOX 3-4 m.2263 meganuclease.

SEQ ID NO: 206 sets forth the amino acid sequence of the LOX 3-4 m.2273 meganuclease.

SEQ ID NO: 207 sets forth the amino acid sequence of the LOX 3-4 m.2274 meganuclease.

SEQ ID NO: 208 sets forth the amino acid sequence of the LOX 3-4 m.2313 meganuclease.

SEQ ID NO: 209 sets forth the amino acid sequence of the LOX 3-4 m.2316 meganuclease.

SEQ ID NO: 210 sets forth the amino acid sequence of the LOX 3-4 m.2327 meganuclease.

SEQ ID NO: 211 sets forth the amino acid sequence of the LOX 3-4 m.2318 meganuclease.

SEQ ID NO: 212 sets forth the amino acid sequence of the LOX 3-4 m.2319 meganuclease.

SEQ ID NO: 213 sets forth the amino acid sequence of the LOX 3-4 m.2320 SEQ ID NO: 214 sets forth the amino acid sequence of the LOX 3-4 m.2322 meganuclease.

SEQ ID NO: 215 sets forth the amino acid sequence of the LOX 3-4 m.2324 meganuclease.

SEQ ID NO: 216 sets forth the amino acid sequence of the LOX 3-4 m.2326 meganuclease.

SEQ ID NO: 217 sets forth the amino acid sequence of the LOX 3-4 m.2329 meganuclease.

SEQ ID NO: 218 sets forth the amino acid sequence of the LOX 3-4 m.2330 meganuclease.

SEQ ID NO: 219 sets forth the amino acid sequence of the LOX 3-4 m.2258 meganuclease.

SEQ ID NO: 220 sets forth the nucleic acid of the LOX 3-4 recognition sequence (sense) with an ATGA center sequence.

SEQ ID NO: 221 sets forth the nucleic acid of the LOX 3-4 recognition sequence (antisense) with an ATGA center sequence.

SEQ ID NO: 222 sets forth the amino acid sequence of the LOX3-4 m.1417 meganuclease.

SEQ ID NO: 223 sets forth the amino acid sequence of the LOX3-4 m.1421 meganuclease.

SEQ ID NO: 224 sets forth the amino acid sequence of the LOX3-4 m.1432 meganuclease.

SEQ ID NO: 225 sets forth the amino acid sequence of the LOX3-4 m.1436 meganuclease.

SEQ ID NO: 226 sets forth the amino acid sequence of the LOX3-4 m.1437 meganuclease.

SEQ ID NO: 227 sets forth the amino acid sequence of the LOX3-4 m.1441 meganuclease.

SEQ ID NO: 228 sets forth the amino acid sequence of the LOX3-4 m.1450 meganuclease.

SEQ ID NO: 229 sets forth the amino acid sequence of the LOX3-4 m.1451 SEQ ID NO: 230 sets forth the amino acid sequence of the LOX3-4 m.1453 meganuclease.

SEQ ID NO: 231 sets forth the amino acid sequence of the LOX3-4 m.1468 meganuclease.

SEQ ID NO: 232 sets forth the amino acid sequence of the LOX3-4 m.1469 meganuclease.

SEQ ID NO: 233 sets forth the amino acid sequence of the LOX3-4 m.1477 meganuclease.

SEQ ID NO: 234 sets forth the amino acid sequence of the LOX3-4 m.1478 meganuclease.

SEQ ID NO: 235 sets forth the amino acid sequence of the LOX3-4 m.1485 meganuclease.

SEQ ID NO: 236 sets forth the amino acid sequence of the LOX3-4 m.1486 meganuclease.

SEQ ID NO: 237 sets forth the amino acid sequence of the LOX3-4 m.1488 meganuclease.

SEQ ID NO: 238 sets forth the amino acid sequence of the LOX3-4 m.1491 meganuclease.

SEQ ID NO: 239 sets forth the amino acid sequence of the LOX3-4 m.1500 meganuclease.

SEQ ID NO: 240 sets forth the amino acid sequence of the LOX3-4 m.1501 meganuclease.

SEQ ID NO: 241 sets forth the amino acid sequence of the LOX3-4 m.1502 meganuclease.

SEQ ID NO: 242 sets forth the amino acid sequence of the LOX3-4 m.1505 meganuclease.

SEQ ID NO: 243 sets forth the amino acid sequence of the LOX3-4 m.1506 meganuclease.

SEQ ID NO: 244 sets forth the nucleic acid sequence of the ATGG LOX 3-4 recognition sequence (sense).

SEQ ID NO: 245 sets forth the nucleic acid sequence of the ATGG LOX 3-4 recognition sequence (antisense).

SEQ ID NO: 246 sets forth the amino acid sequence of the LOX 3-4 m.1508 meganuclease.

SEQ ID NO: 247 sets forth the amino acid sequence of the LOX 3-4 m.1515 meganuclease.

SEQ ID NO: 248 sets forth the nucleic acid of the LOX 3-4 recognition sequence (sense) with a TTGG center sequence.

SEQ ID NO: 249 sets forth the nucleic acid of the LOX 3-4 recognition sequence (antisense) with a TTGG center sequence.

SEQ ID NO: 250 sets forth the amino acid sequence of the LOX 3-4 m.1970 meganuclease.

SEQ ID NO: 251 sets forth the amino acid sequence of the LOX 3-4 m.1973 meganuclease.

SEQ ID NO: 252 sets forth the amino acid sequence of the LOX 3-4 m.1974 meganuclease.

SEQ ID NO: 253 sets forth the amino acid sequence of the LOX 3-4 m.1975 meganuclease.

SEQ ID NO: 254 sets forth the amino acid sequence of the LOX 3-4 m.1979 meganuclease.

SEQ ID NO: 255 sets forth the amino acid sequence of the LOX 3-4 m.1980 meganuclease.

SEQ ID NO: 256 sets forth the amino acid sequence of the LOX 3-4 m.1981 meganuclease.

SEQ ID NO: 257 sets forth the amino acid sequence of the LOX 3-4 m.1982 meganuclease.

SEQ ID NO: 258 sets forth the amino acid sequence of the LOX 3-4 m.1986 meganuclease.

SEQ ID NO: 259 sets forth the amino acid sequence of the LOX 3-4 m.1995 meganuclease.

SEQ ID NO: 260 sets forth the amino acid sequence of the LOX 3-4 m.1997 meganuclease.

SEQ ID NO: 261 sets forth the amino acid sequence of the LOX 3-4 m.2045 SEQ ID NO: 262 sets forth the amino acid sequence of the LOX 3-4 m.2050 meganuclease.

SEQ ID NO: 263 sets forth the amino acid sequence of the LOX 3-4 m.2051 meganuclease.

SEQ ID NO: 264 sets forth the amino acid sequence of the LOX 3-4 m.2052 meganuclease.

SEQ ID NO: 265 sets forth the amino acid sequence of the LOX 3-4 m.2053 meganuclease.

SEQ ID NO: 266 sets forth the amino acid sequence of the LOX 3-4 m.2059 meganuclease.

SEQ ID NO: 267 sets forth the nucleic acid of the LOX 3-4 recognition sequence (sense) with a GCAA center sequence.

SEQ ID NO: 268 sets forth the nucleic acid of the LOX 3-4 recognition sequence (antisense) with a GCAA center sequence.

SEQ ID NO: 269 sets forth the amino acid sequence of the LOX 3-4 m.1784 meganuclease.

SEQ ID NO: 270 sets forth the amino acid sequence of the LOX 3-4 m.1785 meganuclease.

SEQ ID NO: 271 sets forth the amino acid sequence of the LOX 3-4 m.1787 meganuclease.

SEQ ID NO: 272 sets forth the amino acid sequence of the LOX 3-4 m.1789 meganuclease.

SEQ ID NO: 273 sets forth the amino acid sequence of the LOX 3-4 m.1798 meganuclease.

SEQ ID NO: 274 sets forth the amino acid sequence of the LOX 3-4 m.1805 meganuclease.

SEQ ID NO: 275 sets forth the amino acid sequence of the LOX 3-4 m.1809 meganuclease.

SEQ ID NO: 276 sets forth the amino acid sequence of the LOX 3-4 m.1812 meganuclease.

SEQ ID NO: 277 sets forth the amino acid sequence of the LOX 3-4 m.1814 SEQ ID NO: 278 sets forth the amino acid sequence of the LOX 3-4 m.1820 meganuclease.

SEQ ID NO: 279 sets forth the amino acid sequence of the LOX 3-4 m.1827 meganuclease.

SEQ ID NO: 280 sets forth the amino acid sequence of the LOX 3-4 m.1836 meganuclease.

SEQ ID NO: 281 sets forth the amino acid sequence of the LOX 3-4 m.1837 meganuclease.

SEQ ID NO: 282 sets forth the amino acid sequence of the LOX 3-4 m.1838 meganuclease.

SEQ ID NO: 283 sets forth the amino acid sequence of the LOX 3-4 m.1846 meganuclease.

SEQ ID NO: 284 sets forth the amino acid sequence of the LOX 3-4 m.1853 meganuclease.

SEQ ID NO: 285 sets forth the amino acid sequence of the LOX 3-4 m.1854 meganuclease.

SEQ ID NO: 286 sets forth the amino acid sequence of the LOX 3-4 m.1858 meganuclease.

SEQ ID NO: 287 sets forth the amino acid sequence of the LOX 3-4 m.1862 meganuclease.

SEQ ID NO: 288 sets forth the amino acid sequence of the LOX 3-4 m.1868 meganuclease.

SEQ ID NO: 289 sets forth the amino acid sequence of the LOX 3-4 m.1870 meganuclease.

SEQ ID NO: 290 sets forth the amino acid sequence of the LOX 3-4 m.1873 meganuclease.

SEQ ID NO: 291 sets forth the amino acid sequence of the LOX 3-4 m.1875 meganuclease.

SEQ ID NO: 292 sets forth the nucleic acid of the LOX 3-4 recognition sequence (sense) with a GCAT center sequence.

SEQ ID NO: 293 sets forth the nucleic acid of the LOX 3-4 recognition sequence (antisense) with a GCAT center sequence.

SEQ ID NO: 294 sets forth the amino acid sequence of the LOX 3-4 m.1600 meganuclease.

SEQ ID NO: 295 sets forth the amino acid sequence of the LOX 3-4 m.1601 meganuclease.

SEQ ID NO: 296 sets forth the amino acid sequence of the LOX 3-4 m.1605 meganuclease.

SEQ ID NO: 297 sets forth the amino acid sequence of the LOX 3-4 m.1606 meganuclease.

SEQ ID NO: 298 sets forth the amino acid sequence of the LOX 3-4 m.1623 meganuclease.

SEQ ID NO: 299 sets forth the amino acid sequence of the LOX 3-4 m.1660 meganuclease.

SEQ ID NO: 300 sets forth the amino acid sequence of the LOX 3-4 m.1661 meganuclease.

SEQ ID NO: 301 sets forth the amino acid sequence of the LOX 3-4 m.1665 meganuclease.

SEQ ID NO: 302 sets forth the amino acid sequence of the LOX 3-4 m.1667 meganuclease.

SEQ ID NO: 303 sets forth the amino acid sequence of the LOX 3-4 m.1669 meganuclease.

SEQ ID NO: 304 sets forth the amino acid sequence of the LOX 3-4 m.1672 meganuclease.

SEQ ID NO: 305 sets forth the amino acid sequence of the LOX 3-4 m.1674 meganuclease.

SEQ ID NO: 306 sets forth the amino acid sequence of the LOX 3-4 m.1676 meganuclease.

SEQ ID NO: 307 sets forth the amino acid sequence of the LOX 3-4 m.1677 meganuclease.

SEQ ID NO: 308 sets forth the amino acid sequence of the LOX 3-4 m.1679 meganuclease.

SEQ ID NO: 309 sets forth the amino acid sequence of the LOX 3-4 m.1684 SEQ ID NO: 310 sets forth the amino acid sequence of the LOX 3-4 m.1685 meganuclease.

SEQ ID NO: 311 sets forth the amino acid sequence of the LOX 3-4 m.1687 meganuclease.

SEQ ID NO: 312 sets forth the amino acid sequence of the LOX 3-4 m.1689 meganuclease.

SEQ ID NO: 313 sets forth the amino acid sequence of the LOX 3-4 m.1691 meganuclease.

SEQ ID NO: 314 sets forth the nucleic acid of the LOX 3-4 recognition sequence (sense) with a GCGA center sequence.

SEQ ID NO: 315 sets forth the nucleic acid of the LOX 3-4 recognition sequence (antisense) with a GCGA center sequence.

SEQ ID NO: 316 sets forth the amino acid sequence of the LOX 3-4 m.1694 meganuclease.

SEQ ID NO: 317 sets forth the amino acid sequence of the LOX 3-4 m.1745 meganuclease.

SEQ ID NO: 318 sets forth the amino acid sequence of the LOX 3-4 m.1752 meganuclease.

SEQ ID NO: 319 sets forth the amino acid sequence of the LOX 3-4 m.1753 meganuclease.

SEQ ID NO: 320 sets forth the amino acid sequence of the LOX 3-4 m.1765 meganuclease.

SEQ ID NO: 321 sets forth the amino acid sequence of the LOX 3-4 m.1770 meganuclease.

SEQ ID NO: 322 sets forth the amino acid sequence of the LOX 3-4 m.1774 meganuclease.

SEQ ID NO: 323 sets forth the amino acid sequence of the LOX 3-4 m.1780 meganuclease.

SEQ ID NO: 324 sets forth the amino acid sequence of the LOX 3-4 m.1781 meganuclease.

SEQ ID NO: 325 sets forth the amino acid sequence of the LOX 3-4 m.1782 SEQ ID NO: 326 sets forth the nucleic acid sequence of the GCAG LOX 3-4 recognition sequence (sense).

SEQ ID NO: 327 sets forth the nucleic acid sequence of the GCAG LOX 3-4 recognition sequence (antisense).

SEQ ID NO: 328 sets forth the amino acid sequence of the LOX 3-4 m.494 meganuclease.

SEQ ID NO: 329 sets forth the amino acid sequence of the LOX 3-4 m.509 meganuclease.

SEQ ID NO: 330 sets forth the amino acid sequence of the LOX 3-4 m.524 meganuclease.

SEQ ID NO: 331 sets forth the nucleic acid of the LOX 3-4 recognition sequence (sense) with a TCAA center sequence.

SEQ ID NO: 332 sets forth the nucleic acid of the LOX 3-4 recognition sequence (antisense) with a TCAA center sequence.

SEQ ID NO: 333 sets forth the amino acid sequence of the LOX 3-4 m.2157 meganuclease.

SEQ ID NO: 334 sets forth the amino acid sequence of the LOX 3-4 m.2165 meganuclease.

SEQ ID NO: 335 sets forth the amino acid sequence of the LOX 3-4 m.2189 meganuclease.

SEQ ID NO: 336 sets forth the amino acid sequence of the LOX 3-4 m.2207 meganuclease.

SEQ ID NO: 337 sets forth the amino acid sequence of the LOX 3-4 m.2225 meganuclease.

SEQ ID NO: 338 sets forth the amino acid sequence of the LOX 3-4 m.2229 meganuclease.

SEQ ID NO: 339 sets forth the amino acid sequence of the LOX 3-4 m.2235 meganuclease.

SEQ ID NO: 340 sets forth the amino acid sequence of the LOX 3-4 m.2238 meganuclease.

SEQ ID NO: 341 sets forth the nucleic acid of the LOX 3-4 recognition sequence (sense) with a TTAA center sequence.

SEQ ID NO: 342 sets forth the nucleic acid of the LOX 3-4 recognition sequence (antisense) with a TTAA center sequence.

SEQ ID NO: 343 sets forth the amino acid sequence of the LOX 3-4 m.2071 meganuclease.

SEQ ID NO: 344 sets forth the amino acid sequence of the LOX 3-4 m.2077 meganuclease.

SEQ ID NO: 345 sets forth the amino acid sequence of the LOX 3-4 m.2082 meganuclease.

SEQ ID NO: 346 sets forth the amino acid sequence of the LOX 3-4 m.2086 meganuclease.

SEQ ID NO: 347 sets forth the amino acid sequence of the LOX 3-4 m.2087 meganuclease.

SEQ ID NO: 348 sets forth the amino acid sequence of the LOX 3-4 m.2102 meganuclease.

SEQ ID NO: 349 sets forth the amino acid sequence of the LOX 3-4 m.2111 meganuclease.

SEQ ID NO: 350 sets forth the amino acid sequence of the LOX 3-4 m.2116 meganuclease.

SEQ ID NO: 351 sets forth the amino acid sequence of the LOX 3-4 m.2125 meganuclease.

SEQ ID NO: 352 sets forth the amino acid sequence of the LOX 3-4 m.2132 meganuclease.

SEQ ID NO: 353 sets forth the amino acid sequence of the LOX 3-4 m.2138 meganuclease.

SEQ ID NO: 354 sets forth the amino acid sequence of the LOX 3-4 m.2141 meganuclease.

SEQ ID NO: 355 sets forth the amino acid sequence of the LOX 3-4 m.2142 meganuclease.

SEQ ID NO: 356 sets forth the amino acid sequence of the LOX 3-4 m.2145 meganuclease.

SEQ ID NO: 357 sets forth the amino acid sequence of the LOX 3-4 m.2151 SEQ ID NO: 358 sets forth the nucleic acid of the LOX 3-4 recognition sequence (sense) with a GTAA center sequence.

SEQ ID NO: 359 sets forth the nucleic acid of the LOX 3-4 recognition sequence (antisense) with a GTAA center sequence.

SEQ ID NO: 360 sets forth the amino acid sequence of the LOX 3-4 m.1 meganuclease.

SEQ ID NO: 361 sets forth the amino acid sequence of the LOX 3-4 m.2 meganuclease.

SEQ ID NO: 362 sets forth the amino acid sequence of the LOX 3-4 m.3 meganuclease.

SEQ ID NO: 363 sets forth the amino acid sequence of the LOX 3-4 m.4 meganuclease.

SEQ ID NO: 364 sets forth the amino acid sequence of the LOX 3-4 m.5 meganuclease.

SEQ ID NO: 365 sets forth the amino acid sequence of the LOX 3-4 m.6 meganuclease.

SEQ ID NO: 366 sets forth the amino acid sequence of the LOX 3-4 m.7 meganuclease.

SEQ ID NO: 367 sets forth the amino acid sequence of the LOX 3-4 m.8 meganuclease.

SEQ ID NO: 368 sets forth the amino acid sequence of the LOX 3-4 m.9 meganuclease.

SEQ ID NO: 369 sets forth the amino acid sequence of the LOX 3-4 m.10 meganuclease.

SEQ ID NO: 370 sets forth the amino acid sequence of the LOX 3-4 m.11 meganuclease.

SEQ ID NO: 371 sets forth the amino acid sequence of the LOX 3-4 m.12 meganuclease.

SEQ ID NO: 372 sets forth the amino acid sequence of the LOX 3-4 m.13 meganuclease.

SEQ ID NO: 373 sets forth the amino acid sequence of the LOX 3-4 m.14 SEQ ID NO: 374 sets forth the amino acid sequence of the LOX 3-4 m.15 meganuclease.

SEQ ID NO: 375 sets forth the amino acid sequence of the LOX 3-4 m.16 meganuclease.

SEQ ID NO: 376 sets forth the amino acid sequence of the LOX 3-4 m.17 meganuclease.

SEQ ID NO: 377 sets forth the amino acid sequence of the LOX 3-4 m.18 meganuclease.

SEQ ID NO: 378 sets forth the amino acid sequence of the LOX 3-4 m.19 meganuclease.

SEQ ID NO: 379 sets forth the amino acid sequence of the LOX 3-4 m.20 meganuclease.

SEQ ID NO: 380 sets forth the amino acid sequence of the LOX 3-4 m.21 meganuclease.

SEQ ID NO: 381 sets forth the amino acid sequence of the LOX 3-4 m.22 meganuclease.

SEQ ID NO: 382 sets forth the amino acid sequence of the LOX 3-4 m.23 meganuclease.

SEQ ID NO: 383 sets forth the amino acid sequence of the LOX 3-4 m.24 meganuclease.

SEQ ID NO: 384 sets forth the amino acid sequence of the LOX 3-4 m.25 meganuclease.

SEQ ID NO: 385 sets forth the amino acid sequence of the LOX 3-4 m.26 meganuclease.

SEQ ID NO: 386 sets forth the amino acid sequence of the LOX 3-4 m.27 meganuclease.

SEQ ID NO: 387 sets forth the amino acid sequence of the LOX 3-4 m.28 meganuclease.

SEQ ID NO: 388 sets forth the amino acid sequence of the LOX 3-4 m.29 meganuclease.

SEQ ID NO: 389 sets forth the amino acid sequence of the LOX 3-4 m.30 SEQ ID NO: 390 sets forth the nucleic acid of the LOX 3-4 recognition sequence (sense) with a GTAG center sequence.

SEQ ID NO: 391 sets forth the nucleic acid of the LOX 3-4 recognition sequence (antisense) with a GTAG center sequence.

SEQ ID NO: 392 sets forth the amino acid sequence of the LOX 3-4 m.95 meganuclease.

SEQ ID NO: 393 sets forth the amino acid sequence of the LOX 3-4 m.96 meganuclease.

SEQ ID NO: 394 sets forth the amino acid sequence of the LOX 3-4 m.97 meganuclease.

SEQ ID NO: 395 sets forth the amino acid sequence of the LOX 3-4 m.102 meganuclease.

SEQ ID NO: 396 sets forth the amino acid sequence of the LOX 3-4 m.108 meganuclease.

SEQ ID NO: 397 sets forth the amino acid sequence of the LOX 3-4 m.111 meganuclease.

SEQ ID NO: 398 sets forth the amino acid sequence of the LOX 3-4 m.114 meganuclease.

SEQ ID NO: 399 sets forth the amino acid sequence of the LOX 3-4 m.123 meganuclease.

SEQ ID NO: 400 sets forth the nucleic acid of the LOX 3-4 recognition sequence (sense) with a GTAT center sequence.

SEQ ID NO: 401 sets forth the nucleic acid of the LOX 3-4 recognition sequence (antisense) with a GTAT center sequence.

SEQ ID NO: 402 sets forth the amino acid sequence of the LOX 3-4 m.124 meganuclease.

SEQ ID NO: 403 sets forth the amino acid sequence of the LOX 3-4 m.125 meganuclease.

SEQ ID NO: 404 sets forth the amino acid sequence of the LOX 3-4 m.126 meganuclease.

SEQ ID NO: 405 sets forth the amino acid sequence of the LOX 3-4 m.127 SEQ ID NO: 406 sets forth the amino acid sequence of the LOX 3-4 m.128 meganuclease.

SEQ ID NO: 407 sets forth the amino acid sequence of the LOX 3-4 m.129 meganuclease.

SEQ ID NO: 408 sets forth the amino acid sequence of the LOX 3-4 m.130 meganuclease.

SEQ ID NO: 409 sets forth the amino acid sequence of the LOX 3-4 m.131 meganuclease.

SEQ ID NO: 410 sets forth the amino acid sequence of the LOX 3-4 m.132 meganuclease.

SEQ ID NO: 411 sets forth the amino acid sequence of the LOX 3-4 m.133 meganuclease.

SEQ ID NO: 412 sets forth the amino acid sequence of the LOX 3-4 m.134 meganuclease.

SEQ ID NO: 413 sets forth the amino acid sequence of the LOX 3-4 m.135 meganuclease.

SEQ ID NO: 414 sets forth the amino acid sequence of the LOX 3-4 m.136 meganuclease.

SEQ ID NO: 415 sets forth the amino acid sequence of the LOX 3-4 m.137 meganuclease.

SEQ ID NO: 416 sets forth the amino acid sequence of the LOX 3-4 m.138 meganuclease.

SEQ ID NO: 417 sets forth the amino acid sequence of the LOX 3-4 m.139 meganuclease.

SEQ ID NO: 418 sets forth the amino acid sequence of the LOX 3-4 m.140 meganuclease.

SEQ ID NO: 419 sets forth the amino acid sequence of the LOX 3-4 m.141 meganuclease.

SEQ ID NO: 420 sets forth the amino acid sequence of the LOX 3-4 m.142 meganuclease.

SEQ ID NO: 421 sets forth the amino acid sequence of the LOX 3-4 m.143 SEQ ID NO: 422 sets forth the amino acid sequence of the LOX 3-4 m.144 meganuclease.

SEQ ID NO: 423 sets forth the amino acid sequence of the LOX 3-4 m.145 meganuclease.

SEQ ID NO: 424 sets forth the amino acid sequence of the LOX 3-4 m.146 meganuclease.

SEQ ID NO: 425 sets forth the amino acid sequence of the LOX 3-4 m.147 meganuclease.

SEQ ID NO: 426 sets forth the amino acid sequence of the LOX 3-4 m.148 meganuclease.

SEQ ID NO: 427 sets forth the amino acid sequence of the LOX 3-4 m.149 meganuclease.

SEQ ID NO: 428 sets forth the amino acid sequence of the LOX 3-4 m.150 meganuclease.

SEQ ID NO: 429 sets forth the amino acid sequence of the LOX 3-4 m.151 meganuclease.

SEQ ID NO: 430 sets forth the amino acid sequence of the LOX 3-4 m.152 meganuclease.

SEQ ID NO: 431 sets forth the amino acid sequence of the LOX 3-4 m.153 meganuclease.

SEQ ID NO: 432 sets forth the amino acid sequence of the LOX 3-4 m.154 meganuclease.

SEQ ID NO: 433 sets forth the amino acid sequence of the LOX 3-4 m.155 meganuclease.

SEQ ID NO: 434 sets forth the nucleic acid of the LOX 3-4 recognition sequence (sense) with a GTGA center sequence.

SEQ ID NO: 435 sets forth the nucleic acid of the LOX 3-4 recognition sequence (antisense) with a GTGA center sequence.

SEQ ID NO: 436 sets forth the amino acid sequence of the LOX 3-4 m.31 meganuclease.

SEQ ID NO: 437 sets forth the amino acid sequence of the LOX 3-4 m.32 SEQ ID NO: 438 sets forth the amino acid sequence of the LOX 3-4 m.33 meganuclease.

SEQ ID NO: 439 sets forth the amino acid sequence of the LOX 3-4 m.35 meganuclease.

SEQ ID NO: 440 sets forth the amino acid sequence of the LOX 3-4 m.36 meganuclease.

SEQ ID NO: 441 sets forth the amino acid sequence of the LOX 3-4 m.37 meganuclease.

SEQ ID NO: 442 sets forth the amino acid sequence of the LOX 3-4 m.38 meganuclease.

SEQ ID NO: 443 sets forth the amino acid sequence of the LOX 3-4 m.39 meganuclease.

SEQ ID NO: 444 sets forth the amino acid sequence of the LOX 3-4 m.40 meganuclease.

SEQ ID NO: 445 sets forth the amino acid sequence of the LOX 3-4 m.41 meganuclease.

SEQ ID NO: 446 sets forth the amino acid sequence of the LOX 3-4 m.42 meganuclease.

SEQ ID NO: 447 sets forth the amino acid sequence of the LOX 3-4 m.43 meganuclease.

SEQ ID NO: 448 sets forth the amino acid sequence of the LOX 3-4 m.44 meganuclease.

SEQ ID NO: 449 sets forth the amino acid sequence of the LOX 3-4 m.46 meganuclease.

SEQ ID NO: 450 sets forth the amino acid sequence of the LOX 3-4 m.47 meganuclease.

SEQ ID NO: 451 sets forth the amino acid sequence of the LOX 3-4 m.48 meganuclease.

SEQ ID NO: 452 sets forth the amino acid sequence of the LOX 3-4 m.49 meganuclease.

SEQ ID NO: 453 sets forth the amino acid sequence of the LOX 3-4 m.50 SEQ ID NO: 454 sets forth the amino acid sequence of the LOX 3-4 m.51 meganuclease.

SEQ ID NO: 455 sets forth the amino acid sequence of the LOX 3-4 m.52 meganuclease.

SEQ ID NO: 456 sets forth the amino acid sequence of the LOX 3-4 m.53 meganuclease.

SEQ ID NO: 457 sets forth the amino acid sequence of the LOX 3-4 m.54 meganuclease.

SEQ ID NO: 458 sets forth the amino acid sequence of the LOX 3-4 m.56 meganuclease.

SEQ ID NO: 459 sets forth the amino acid sequence of the LOX 3-4 m.57 meganuclease.

SEQ ID NO: 460 sets forth the amino acid sequence of the LOX 3-4 m.58 meganuclease.

SEQ ID NO: 461 sets forth the amino acid sequence of the LOX 3-4 m.59 meganuclease.

SEQ ID NO: 462 sets forth the amino acid sequence of the LOX 3-4 m.61 meganuclease.

SEQ ID NO: 463 sets forth the nucleic acid of the LOX 3-4 recognition sequence (sense) with a GTGC center sequence.

SEQ ID NO: 464 sets forth the nucleic acid of the LOX 3-4 recognition sequence (antisense) with a GTGC center sequence.

SEQ ID NO: 465 sets forth the amino acid sequence of the LOX 3-4 m.156 meganuclease.

SEQ ID NO: 466 sets forth the amino acid sequence of the LOX 3-4 m.157 meganuclease.

SEQ ID NO: 467 sets forth the amino acid sequence of the LOX 3-4 m.158 meganuclease.

SEQ ID NO: 468 sets forth the amino acid sequence of the LOX 3-4 m.159 meganuclease.

SEQ ID NO: 469 sets forth the amino acid sequence of the LOX 3-4 m.160 SEQ ID NO: 470 sets forth the amino acid sequence of the LOX 3-4 m.161 meganuclease.

SEQ ID NO: 471 sets forth the amino acid sequence of the LOX 3-4 m.162 meganuclease.

SEQ ID NO: 472 sets forth the amino acid sequence of the LOX 3-4 m.163 meganuclease.

SEQ ID NO: 473 sets forth the amino acid sequence of the LOX 3-4 m.164 meganuclease.

SEQ ID NO: 474 sets forth the amino acid sequence of the LOX 3-4 m.165 meganuclease.

SEQ ID NO: 475 sets forth the amino acid sequence of the LOX 3-4 m.166 meganuclease.

SEQ ID NO: 476 sets forth the amino acid sequence of the LOX 3-4 m.167 meganuclease.

SEQ ID NO: 477 sets forth the amino acid sequence of the LOX 3-4 m.168 meganuclease.

SEQ ID NO: 478 sets forth the amino acid sequence of the LOX 3-4 m.169 meganuclease.

SEQ ID NO: 479 sets forth the amino acid sequence of the LOX 3-4 m.170 meganuclease.

SEQ ID NO: 480 sets forth the amino acid sequence of the LOX 3-4 m.171 meganuclease.

SEQ ID NO: 481 sets forth the amino acid sequence of the LOX 3-4 m.172 meganuclease.

SEQ ID NO: 482 sets forth the amino acid sequence of the LOX 3-4 m.173 meganuclease.

SEQ ID NO: 483 sets forth the amino acid sequence of the LOX 3-4 m.174 meganuclease.

SEQ ID NO: 484 sets forth the amino acid sequence of the LOX 3-4 m.175 meganuclease.

SEQ ID NO: 485 sets forth the amino acid sequence of the LOX 3-4 m.176 SEQ ID NO: 486 sets forth the amino acid sequence of the LOX 3-4 m.177 meganuclease.

SEQ ID NO: 487 sets forth the amino acid sequence of the LOX 3-4 m.178 meganuclease.

SEQ ID NO: 488 sets forth the amino acid sequence of the LOX 3-4 m.179 meganuclease.

SEQ ID NO: 489 sets forth the amino acid sequence of the LOX 3-4 m.180 meganuclease.

SEQ ID NO: 490 sets forth the amino acid sequence of the LOX 3-4 m.181 meganuclease.

SEQ ID NO: 491 sets forth the amino acid sequence of the LOX 3-4 m.182 meganuclease.

SEQ ID NO: 492 sets forth the amino acid sequence of the LOX 3-4 m.183 meganuclease.

SEQ ID NO: 493 sets forth the amino acid sequence of the LOX 3-4 m.184 meganuclease.

SEQ ID NO: 494 sets forth the amino acid sequence of the LOX 3-4 m.185 meganuclease.

SEQ ID NO: 495 sets forth the amino acid sequence of the LOX 3-4 m.186 meganuclease.

SEQ ID NO: 496 sets forth the nucleic acid of the LOX 3-4 recognition sequence (sense) with a GTGG center sequence.

SEQ ID NO: 497 sets forth the nucleic acid of the LOX 3-4 recognition sequence (antisense) with a GTGG center sequence.

SEQ ID NO: 498 sets forth the amino acid sequence of the LOX 3-4 m.187 meganuclease.

SEQ ID NO: 499 sets forth the amino acid sequence of the LOX 3-4 m.192 meganuclease.

SEQ ID NO: 500 sets forth the amino acid sequence of the LOX 3-4 m.201 meganuclease.

SEQ ID NO: 501 sets forth the amino acid sequence of the LOX 3-4 m.203 SEQ ID NO: 502 sets forth the nucleic acid of the LOX 3-4 recognition sequence (sense) with a GTGT center sequence.

SEQ ID NO: 503 sets forth the nucleic acid of the LOX 3-4 recognition sequence (antisense) with a GTGT center sequence.

SEQ ID NO: 504 sets forth the amino acid sequence of the LOX 3-4 m.63 meganuclease.

SEQ ID NO: 505 sets forth the amino acid sequence of the LOX 3-4 m.64 meganuclease.

SEQ ID NO: 506 sets forth the amino acid sequence of the LOX 3-4 m.65 meganuclease.

SEQ ID NO: 507 sets forth the amino acid sequence of the LOX 3-4 m.66 meganuclease.

SEQ ID NO: 508 sets forth the amino acid sequence of the LOX 3-4 m.67 meganuclease.

SEQ ID NO: 509 sets forth the amino acid sequence of the LOX 3-4 m.68 meganuclease.

SEQ ID NO: 510 sets forth the amino acid sequence of the LOX 3-4 m.69 meganuclease.

SEQ ID NO: 511 sets forth the amino acid sequence of the LOX 3-4 m.70 meganuclease.

SEQ ID NO: 512 sets forth the amino acid of the meganuclease with a LOX 3-4 m.71 center sequence.

SEQ ID NO: 513 sets forth the amino acid of the meganuclease with a LOX 3-4 m.73 center sequence.

SEQ ID NO: 514 sets forth the amino acid sequence of the LOX 3-4 m.74 meganuclease.

SEQ ID NO: 515 sets forth the amino acid sequence of the LOX 3-4 m.75 meganuclease.

SEQ ID NO: 516 sets forth the amino acid sequence of the LOX 3-4 m.77 meganuclease.

SEQ ID NO: 517 sets forth the amino acid sequence of the LOX 3-4 m.78 SEQ ID NO: 518 sets forth the amino acid sequence of the LOX 3-4 m.80 meganuclease.

SEQ ID NO: 519 sets forth the amino acid sequence of the LOX 3-4 m.83 meganuclease.

SEQ ID NO: 520 sets forth the amino acid sequence of the LOX 3-4 m.84 meganuclease.

SEQ ID NO: 521 sets forth the amino acid sequence of the LOX 3-4 m.85 meganuclease.

SEQ ID NO: 522 sets forth the amino acid sequence of the LOX 3-4 m.86 meganuclease.

SEQ ID NO: 523 sets forth the amino acid sequence of the LOX 3-4 m.87 meganuclease.

SEQ ID NO: 524 sets forth the amino acid sequence of the LOX 3-4 m.88 meganuclease.

SEQ ID NO: 525 sets forth the amino acid sequence of the LOX 3-4 m.89 meganuclease.

SEQ ID NO: 526 sets forth the amino acid sequence of the LOX 3-4 m.90 meganuclease.

SEQ ID NO: 527 sets forth the amino acid sequence of the LOX 3-4 m.91 meganuclease.

SEQ ID NO: 528 sets forth the amino acid sequence of the LOX 3-4 m.92 meganuclease.

SEQ ID NO: 529 sets forth the amino acid sequence of the LOX 3-4 m.93 meganuclease.

SEQ ID NO: 530 sets forth the amino acid sequence of a polypeptide linker.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example. “a” cell can mean a single cell or a multiplicity of cells.

As used herein, unless specifically indicated otherwise, the word “or” is used in the inclusive sense of “and/or” and not the exclusive sense of “either/or.”

As used herein, the terms “nuclease” and “endonuclease” are used interchangeably to refer to naturally-occurring or engineered enzymes, which cleave a phosphodiester bond within a polynucleotide chain.

As used herein, the terms “cleave” or “cleavage” refer to the hydrolysis of phosphodiester bonds within the backbone of a recognition sequence within a target sequence that results in a double-stranded break within the target sequence, referred to herein as a “cleavage site”. In some embodiments described herein, modification or substitution at one or more positions corresponding to positions 48, 50, 71, 72, 73, 73B and 74 of I-CreI (i.e., SEQ ID NO: 1) increase the cleavage activity of a engineered meganuclease.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI (SEQ ID NO: 1), and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g., WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein such that cells can be transfected and maintained at 37° C. without observing deleterious effects on cell viability or significant reductions in meganuclease cleavage activity when measured using the methods described herein.

As used herein, the term “single-chain meganuclease” refers to a polypeptide comprising a pair of nuclease subunits joined by a linker. A single-chain meganuclease has the organization: N-terminal subunit—Linker—C-terminal subunit. The two meganuclease subunits will generally be non-identical in amino acid sequence and will bind non-identical DNA sequences. Thus, single-chain meganucleases typically cleave pseudo-palindromic or non-palindromic recognition sequences. Engineered I-CrcI-derived meganucleases that are single-chain meganucleases, and methods for producing them, are disclosed in WO 2009/059195, which is incorporated by reference herein. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two nuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to SEQ ID NO: 530, which sets forth residues 154-195 of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529. Although positions 48, 50, 71, 72, 73, and 74 are located within the hypervariable region, it is thought that these positions affect cleavage of a center sequence and not necessarily the binding of the meganuclease to a specific recognition sequence site. Thus, when designing two meganucleases targeting two different recognitions sequences having the same center sequence, it may not be required to modify positions 48, 50, 71, 72, 73, and 74 between the two meganucleases. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity. In some embodiments, variable residues within a hypervariable region correspond to one or more of positions 24, 26, 28, 30, 32, 33, 38, 40, 42, 44, 46, 68, 70, 75, and 77 of any one of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529. In other embodiments, variable residues within a hypervariable region correspond to one or more of positions 215, 217, 219, 221, 223, 224, 229, 231, 233, 235, 237, 259, 261, 266, and 268 of any one of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529.

As used herein, the terms “recombinant” or “engineered,” with respect to a protein, means having an altered amino acid sequence as a result of the application of genetic engineering techniques to nucleic acids that encode the protein and cells or organisms that express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation, and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant or engineered.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein, the term with respect to recombinant proteins, the term “modification” means any insertion, deletion, or substitution of an amino acid residue in the recombinant sequence relative to a reference sequence (e.g., a wild-type or a native sequence).

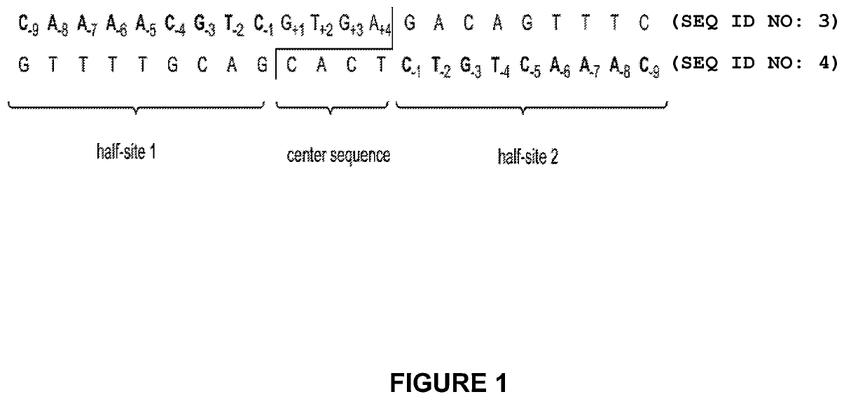

As used herein, the term “recognition sequence” refers to a DNA sequence that is bound and cleaved by wild-type I-CreI or an engineered I-CreI-derived meganuclease of the disclosure. The disclosed recognition sequences cleaved by I-CreI and the disclosed engineered meganucleases are typically 22 nucleotides in length. These recognition sequences comprise a pair of inverted, 9 base pair “half-sites” (each numbered from −1 to −9) which are separated by a four base pair center sequence (numbered +1, +2, +3, and +4) (FIG. 1). In the case of a single-chain meganuclease, the N-terminal domain of the protein recognizes, interacts with and/or contacts one half-site and the C-terminal domain of the protein recognizes, interacts with and/or contacts the other half-site. Cleavage by a meganuclease produces four base pair 3′ “overhangs”. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 base pair recognition sequence. Thus, an I-CreI meganuclease recognition sequence may be defined according to formula I:

X₋₉X₋₈X₋₇X₋₆X₋₅X₋₄X₋₃X₋₂X₋₁N₊₁N₊₂N₊₃N₊₄X₋₁X₋₂X₋₃-X₋₄X₋₅X₋₆ X₋₇X₋₈X₋₉.

wherein X and N are each independently nucleotides selected from an adenine nucleotide, a cytosine nucleotide, a guanine nucleotide, and a thymine nucleotide; wherein N₊₁N₊₂N₊₃N₊₄ is the four base pair center sequence.

As used herein, the term “center sequence” refers to the four base pairs separating half-sites in the meganuclease recognition sequence. These bases are numbered +1 through +4 (FIG. 1 and Formula 1). The center sequence comprises the four bases that become the 3′ single-strand overhangs following meganuclease cleavage. “Center sequence” can refer to the sequence of the sense strand or the antisense (opposite) strand. Meganucleases are symmetric and recognize bases equally on both the sense and antisense strand of the center sequence. For example, the sequence A₊₁A₊₂A₊₃A₊₄ on the sense strand is recognized by, interacted with and/or contacted by a meganuclease as T₊₁T₊₂T₊₃T₊₄ on the antisense strand and, thus, A₊₁A₊₂A₊₃A₊₄ and T₊₁T₊₂T₊₃T₊₄ are functionally equivalent (e.g., both can be cleaved by a given meganuclease). Thus, the sequence C₊₁T₊₂G₊₃C₊₄, is equivalent to its opposite strand sequence, G₊₁C₊₂A₊₃G₊₄ due to the fact that the meganuclease binds its recognition sequence as a symmetric homodimer. In most cases, a first subunit of the meganuclease recognizes, interacts with and/or contacts the first two base pairs of the sense strand of a given center sequence and the second two base pairs on the antisense. For example, taking A₊₁A₊₂A₊₃A₊₄ as the center sequence, a first subunit would recognize, interact with and/or contact the two base pairs A₊₁A₊₂, and a second subunit would recognize, interact with and/or contact the anti-sense strand two base pairs A₊₃A₊₄ on the anti-sense strand, which is T₊₄T₊₃.

As used herein, the term “recognition half-site,” “recognition sequence half-site,” or simply “half-site” means a nucleic acid sequence in a double-stranded DNA molecule which is a monomer a homodimeric or heterodimeric meganuclease binds to (e.g., recognizes), or by one subunit of a single-chain meganuclease.

As used herein, the term “center sequence half-site,” or simply “center half-site” refers to either the 5′ two base pairs or the 3′ two base pairs of a four base pair center sequence of a recognition sequence as described herein. For example, for the center sequence ACAG, the 5′ two base pairs (i.e., the 5′ center half site) of the center sequence is “AC” and the 3′ two base pairs (i.e., the 3′ center half site) is “AG” (reverse complement being “CT”).

As used herein, the terms a meganuclease “derived from I-CreI” or an “I-CreI-derived meganuclease” refers to a recombinant variant of a naturally-occurring I-CrcI homing endonuclease (SEQ ID NO: 1) that has been modified by one or more amino acid insertions, deletions, and/or substitutions that affect one or more of DNA-binding specificity, DNA cleavage activity, and/or DNA-binding affinity and/or dimerization properties. Some genetically-engineered meganucleases are known in the art (see, e.g., Porteus et al. (2005), Nat. Biotechnol. 23: 967-73; Sussman et al. (2004), J. Mol. Biol. 342: 31-41; Epinat et al. (2003), Nucleic Acids Res. 31: 2952-62) and general methods for rationally-designing such variants have been disclosed, for example, in WO 2007/047859. I-CreI derived meganucleases encompass engineered proteins wherein I-CreI was directly modified, engineered proteins wherein an I-CreI derived meganuclease was further modified, and/or proteins that have been synthetically produced based on an I-CreI derived sequence. As used herein, the term “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. In some embodiments, an “I-CreI-derived meganuclease” specifically includes any engineered meganuclease within the scope of the published claims of any of International Publication Nos. WO2007/047859, WO2009059195, WO2010/009147, WO2012/167192, WO2015/138739, WO2016/179112, WO2017/044649, WO2017/062439, WO2017/062451, WO2017/112859, WO2017/192741, WO2018/071849, WO2018/195449, WO2019/005957, WO2019/089913, WO2019/200122, and WO2019/200247, and International Publication Nos. PCT/US2019/068186 and PCT/US2020/013198, each of which is incorporated by reference in its entirety herein. In some embodiments, an “I-CreI-derived meganuclease” specifically includes any engineered meganuclease within the scope of the issued claims of any of U.S. Pat. Nos. 8,021,867, 8,119,361, 8,119,381, 8,124,369, 8,129,134, 8,133,697, 8,143,015, 8,143,016, 8,148,098, 8,163,514, 8,304,222, 8,377,674, 8,445,251, 9,340,777, 9,434,931, 10,041,053, 9,683,257, 10,287,626, 10,273,524, 9,683,257, 10,287,626, 10,273,524, 9,822,381, 10,603,363, 9,889,160, 9,889,161, 9,993,501, 9,993,502, 9,950,010, 9,950,011, 9,969,975, 10,093,899, and 10,093,900, each of which is incorporated by reference herein. In some embodiments, an engineered I-CreI-derived meganuclease comprises a polypeptide having at least 85% sequence identity to residues 2-153 of the I-CreI meganuclease of SEQ ID NO: 1, as in the issued claims of each of U.S. Pat. Nos. 8,021,867, 8,119,361, 8,119,381, 8,124,369, 8,129,134, 8,133,697, 8,143,015, 8,143,016, 8,148,098, 8,163,514, 8,304,222, 8,377,674. In some embodiments, an engineered I-CreI-derived meganuclease comprises a polypeptide having at least 86%, 87%, 88%, 89%, 90%, 91%, 92%, 93%, 94%, 95%, 96%, 97%, 98% or 99% sequence identity to residues 2-153 of the I-CreI meganuclease of SEQ ID NO: 1.

As used herein, the terms “DNA-binding affinity” or “binding affinity” means the tendency of a nuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change relative to a reference nuclease.

As used herein, the term “specificity” means the ability of a nuclease to hind (e.g., recognize) and cleave double-stranded DNA molecules only at a particular sequence of base pairs referred to as the recognition sequence, or only at a particular set of recognition sequences. The set of recognition sequences will share certain conserved positions or sequence motifs but may be degenerate at one or more positions. A highly-specific nuclease is capable of cleaving only one or a very few recognition sequences. Specificity can be determined by any method known in the art.

As used herein, the term “activity” refers to the rate at which a meganuclease of the invention cleaves a particular recognition sequence. Such activity is a measurable enzymatic reaction, involving the hydrolysis of phosphodiester bonds of double-stranded DNA. The activity of a meganuclease acting on a particular DNA substrate is affected by the affinity or avidity of the meganuclease for that particular DNA substrate which is, in turn, affected by both sequence-specific and non-sequence-specific interactions with the DNA.

As used herein, the term “altered specificity,” when referencing to a meganuclease, means that a nuclease binds to and cleaves a recognition sequence, which is not bound to and cleaved by a reference nuclease (e.g., a wild-type) under physiological conditions, or that the rate of cleavage of a recognition sequence is increased or decreased by a biologically significant amount (e.g., at least 2×, or 2×-10×) relative to a reference nuclease.

As used herein, the terms “percent identity,” “sequence identity,” “percentage similarity,” “sequence similarity” and the like, with respect to both amino acid sequences and nucleic acid sequences, refer to a measure of the degree of similarity of two sequences based upon an alignment of the sequences that maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7(1-2):203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=l; gap opening penalty=−5; gap extension penalty=−2; match reward=1; and mismatch penalty=−3.

As used herein, the term “corresponding to” with respect to modifications of two proteins or amino acid sequences is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment and despite the fact that X and Y may be different numbers.

As used herein, the term “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, the term “vector” or “recombinant DNA vector” may be a construct that includes a replication system and sequences that are capable of transcription and translation of a polypeptide-encoding sequence in a given host cell. If a vector is used, then the choice of vector is dependent upon the method that will be used to transform host cells as is well known to those skilled in the art. Vectors can include, without limitation, plasmid vectors and recombinant AAV vectors, or any other vector known in the art suitable for delivering a gene to a target cell. The skilled artisan is well aware of the genetic elements that must be present on the vector in order to successfully transform, select and propagate host cells comprising any of the isolated nucleotides or nucleic acid sequences of the invention. In some embodiments, a “vector” also refers to recombinant viral vector (e.g., a recombinant virus). Recombinant viral vectors (e.g., recombinant viruses) can include, without limitation, retroviral vectors (e.g., retroviruses), lentiviral vectors (e.g., lentiviruses), adenoviral vectors (e.g., adenoviruses), and adeno-associated viral vectors (e.g., adeno-associated viruses (AAVs).

As used herein, the recitation of a numerical range for a variable is intended to convey that the present disclosure may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

The present invention is based, in part, on the identification of positions and residues within I-CreI that can be modified to improve the cleavage activity for recognition sequences containing certain 4 base pair center sequences. There are four DNA bases (A, C, G. and T) and consequently 256 possible DNA sequences that are four base pairs in length. As described in WO2010/009147, these possible sequences are cleaved by engineered, I-CreI-derived meganucleases with differing efficiencies. Previously, it was thought that wild type I-CreI does not appreciably contact or otherwise interact with the four base pair center sequence and thus, it has not been previously contemplated that modification of residues within I-CreI could improve the cleavage efficiency and or specificity of a meganuclease for a recognition sequence having a given center sequence.

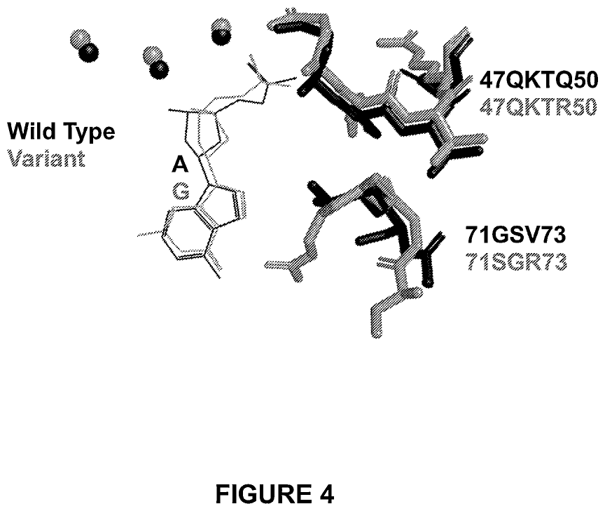

However, as described herein, it has been discovered that modifying particular residues in an I-CreI-derived meganuclease can improve the cleavage efficiency for recognition sequences having certain four base pair center sequences. Positions discovered to affect the ability of an I-CreI-derived meganuclease to cut a center sequence include those corresponding to positions 48, 50, 71, 72, 73, 73B, and 74 of I-CreI. Without being bound by any theory, it is thought that these sequences assist in the positioning of the DNA double helix, water molecules, and/or necessary metal co-factors within the meganuclease binding pocket (see crystal structure shown in FIG. 4). It is to be understood that the modification of residues within the hypervariable regions of the meganuclease does not appreciably affect cleavage of the center sequences described herein because these hypervariable region residues primarily interact with the DNA backbone allowing the meganuclease to bind to a specific 22-base pair recognition sequence. Accordingly, binding does not necessarily confer cleavage activity of the meganuclease. For example, given a recognition sequence having TCAA as a center sequence, a meganuclease having unmodified residues at positions 48, 50, 71, 72, 73, 73B, and 74 corresponding to I-CreI described herein will bind to its recognition sequence but not cut the TCAA center sequence. Modification of one or more of residues 48, 50, 71, 72, 73, 73B, and 74 in that meganuclease as described herein will then confer or improve cleavage activity of that center sequence (e.g., TCAA).

As demonstrated herein, the modification of these particular residues has greatly increased the cleaving efficiency of recognition sequences having specific center sequences that previously were difficult to cleave. For example, the center sequences TTGA (reverse complement TCAA) and CCGT (reverse complement ACGG) were previously described as having a low efficiency of cutting by an engineered meganuclease (see, Arnould, et al. (2007). J. Mol. Biol. 371: 49-65 and WO 2010/009147). However, by making substitutions according to the invention, novel engineered meganucleases exhibited a 38-fold increase in cleavage of a recognition sequence comprising a TCAA (i.e., TTGA) center sequence, and a 21-fold increase in cleaving a recognition sequence comprising an ACGG (i.e., CCGT) center sequence (see Examples 23 and 7, respectively). Accordingly, the invention provides engineered meganucleases, derived from I-CreI, which have substitutions at particular positions, which increase the activity of the nucleases for recognition sequences containing certain four base pair center sequences. The invention also provides methods of cleaving double-stranded DNA using such engineered meganucleases. The invention further provides methods for improving the activity of engineered meganucleases for recognition sequences containing certain four base pair center sequences.

### 2.2 Engineered Meganucleases Optimized for Specific Center Sequences

It is known in the art that it is possible to use a site-specific nuclease to make a DNA break in the genome of a living cell, and that such a DNA break can result in permanent modification of the genome via homologous recombination with a transgenic DNA sequence. The use of nucleases to induce a double-strand break in a target locus is known to stimulate homologous recombination, particularly of transgenic DNA sequences flanked by sequences that are homologous to the genomic target. In this manner, exogenous nucleic acid sequences can be inserted into a target locus.

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the gnomic target. In this manner, exogenous nucleic acid sequences can be inserted into a target locus. Such exogenous nucleic acids can encode any sequence or polypeptide of interest.

As disclosed herein, the nucleases used to practice the invention are meganucleases. In some embodiments, the nucleases used to practice the invention are single-chain meganucleases. A single-chain meganuclease comprises an N-terminal subunit and a C-terminal subunit joined by a linker peptide. Each of the two domains recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. In some embodiments, engineered meganucleases of the invention have been engineered to bind and cleave recognition sequences with specific center sequences.

Engineered meganucleases of the invention comprise a first subunit, comprising a first hypervariable (HVR1) region, and a second subunit, comprising a second hypervariable (HVR2) region. Further, the first subunit binds to a first recognition half-site in the recognition sequence, and the second subunit binds to a second recognition half-site in the recognition sequence. In embodiments where the engineered meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit. As disclosed herein, certain modifications to the meganuclease (e.g., at positions 48, 50, 71, 72, 73, 73B, and 74) confer increased cleavage of recognition sequences having certain four base pair center sequences. Exemplary engineered meganucleases of the invention, which demonstrate improve cleavage of recognition sequences comprising certain center sequences are provided in SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529.

In specific embodiments, an engineered meganuclease of the invention is a homodimer or heterodimer, wherein each of the two subunits of the dimer is derived from SEQ ID NO: 1 (i.e., I-CreI). Engineered meganucleases disclosed herein can comprise modifications (e.g., substitutions) in a single subunit, or modifications in both subunits, which confer increased activity (e.g., increased cleavage activity) of the engineered meganuclease for a recognition sequence comprising a specific center sequence.

In some examples, a first or second subunit of an I-CreI-derived meganuclease may have at least 70%, at least 75%, at least 80%, at least 81%, at least 82%, at least 83%, at least 84%, at least 85%, at least 86%, at least 87%, at least 88%, at least 89% at least 90%, at least 91%, at least 92%, at least 93%, at least 94%, at least 96%, at least 97%, at least 98%, at least 99%, or at least 99.5% sequence identity to a wild-type I-CreI (SEQ ID NO: 1). In some embodiments, a first and/or second subunit of any of the disclosed engineered meganucleases may have at least 75%, at least 80%, at least 85%, at least 88%, at least 90%, at least 92%, at least 94%, at least 96%, at least 97%, at least 98%, or at least 99% sequence identity to SEQ ID NO: 1, with the exception of an amino acid substitution at one or more positions corresponding to positions 48, 50, 71, 72, 73, and 74 of SEQ ID NO: 1.

In some embodiments, a first and/or second subunit of any of the disclosed engineered meganucleases may have at least 75%, at least 80%, at least 85%, at least 88%, at least 90%, at least 92%, at least 94%, at least 96%, at least 97%, at least 98%, or at least 99% sequence identity to SEQ ID NO: 1, with the exception of an amino acid substitution at one or more positions corresponding to positions 48, 50, 71, 72, 73, 73B, and 74 of SEQ ID NO: 1. In particular embodiments, at least one of the first or second subunit comprises at least 85% sequence identity to SEQ ID NO: 1 with the exception of an amino acid substitution at one or more positions corresponding to positions 48, 50, 71, 72, 73, 73B, and 74 of SEQ ID NO: 1. In some embodiments, the substitution at one or more positions of the first and/or second subunit of the disclosed engineered meganucleases corresponding to positions 48, 50, 71, 72, 73, 73B, and 74 of SEQ ID NO: 1 is a conservative substitution, such as exchanging one amino acid with another having similar properties. In some embodiments, one or more of the charged amino acids at these positions (e.g., K48) is substituted with a similarly charged amino acid. In some embodiments, one or more of the polar amino acids at these positions (e.g., Q50, S72 and S74) is substituted with a similarly polar amino acid. In some embodiments, one or more of the charged hydrophobic acids at these positions (e.g., G41 and V73) is substituted with a similarly hydrophobic amino acid.

In some embodiments, the substitutions at one or more positions of the first and/or second subunit of the disclosed engineered meganucleases comprises substitutions at two, three or more than three amino acid positions corresponding to positions 48, 50, 71, 72, 73, 73B, and 74 of SEQ ID NO: 1. In some embodiments, two substitutions are made at positions corresponding to positions 48 and 50 of SEQ ID NO: 1. Without being bound to a particular theory, amino acid positions 48 and 40 of SEQ ID NO: 1 are believed to form a coordination series with water and a magnesium ion. In some embodiments, three or four substitutions are made at positions corresponding to positions 71, 72, 73, and 74 of SEQ ID NO: 1. Without being bound to a particular theory, amino acid positions 71-74 of SEQ ID NO: 1 (which are exposed at the surface of the protein as a loop) are believed to act in concert.

In particular examples, the engineered meganuclease is a single-chain meganuclease, wherein the first subunit and the second subunit are covalently joined by a polypeptide linker. In some embodiments, the polypeptide linker is according to SEQ ID NO: 530.

In specific embodiments, the first subunit, the second subunit, or both subunits can comprise a substitution at one or more positions corresponding to positions 48, 50, 71, 72, 73, 73B, and 74 of wild-type I-CreI (SEQ ID NO: 1). Despite previous reports that I-CreI-derived meganucleases do not interact with the four base pair center sequence, it has been demonstrated herein that modifications at one or more of these positions can increase the activity (e.g., cleavage activity) of the nuclease for a recognition sequence comprising a specific center sequence. It is further disclosed herein that substitutions can be made at additional positions in the first and/or second subunit, which further optimize the engineered meganuclease for a recognition sequence having a specific center sequence.

When generating an I-CreI-derived meganuclease that is optimized for a recognition sequence having a specific center sequence, one or more residues corresponding to positions 48, 50, 71, 72, 73, 73B, and 74 of I-CreI (SEQ ID NO: 1) are modified. Tables 1-90 below describe positions and residues which have been exemplified herein. As shown, residues and positions for a “first subunit” refer to modifications of the subunit of the engineered meganuclease, which binds, interacts with or recognizes (e.g., binds, makes contact, or is generally positioned around and coordinates water and metal cofactors) the half-site of the recognition sequence that is 5′ upstream of positions+1 and +2 of a center sequence. Similarly, residues and positions for a “second subunit” refer to modifications of the subunit of the engineered meganuclease, which interacts with (e.g., binds, makes contact, or is generally positioned around and coordinates water and metal cofactors) the half-site of the recognition sequence that is 3′ downstream of positions+3 and +4 of a center sequence.

In each table below, the term “I-CreI Position” refers to the position of the residue as found in the wild-type I-CreI monomer. The term “EN Position” refers to the actual numerical position of a residue, which corresponds to the wild-type I-CreI residue, in an exemplified engineered meganuclease. For example, in an exemplified engineered meganuclease, nuclease position 239 is within the second subunit and can correspond to position 48 of wild-type I-CreI. In some examples, an amino acid is inserted into the engineered nuclease sequence and the numbering of the nuclease positions changes accordingly. In such cases, the same residues correspond to the wild-type I-CreI residues, even though their numbering in the engineered meganuclease has changed. For example, in some cases an R residue is inserted after position 73 of an engineered meganuclease, referred herein to as 73B or 264B. This causes the residue at position 74 to be at new position 75. In such cases, position 75 still corresponds to the position 74 of wild-type I-CreI.

In some embodiments, the disclosed engineered I-CreI-derived meganucleases bind and cleave a recognition sequence comprising a center sequence selected from the group consisting of ACXX, TlXX, GCXX, and TCXX; a recognition sequence selected from XXTT, XXCT, XXAT, XXTC, XXGC, XXGG, and XXGT; or a recognition sequence selected from XXTT, XXCT, XXAT, XXTC, XXGC, XXGG, and XXGT, wherein X represents a nucleotide selected from A, G, C, or T.

In some embodiments, the disclosed engineered I-CreI-derived meganucleases bind and cleave a recognition sequence comprising a center sequence selected from the group consisting of ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, and TTAA. In particular embodiments, the disclosed engineered meganucleases bind and cleave a recognition sequence selected from ACAA, ACAG, ACAT, ACGC, ACGG, and ACGT. In particular embodiments, the disclosed engineered meganucleases bind and cleave a recognition sequence selected from ATAA, ATAG, ATAT, ATGA, and ATGG. In particular embodiments, the disclosed engineered meganucleases bind and cleave a recognition sequence selected from GCAA, GCAT, GCGA, and GCAG. In particular embodiments, the disclosed engineered meganucleases bind and cleave the recognition sequence TTGG or TTAA.

In particular embodiments, the disclosed engineered meganucleases bind and cleave a recognition sequence selected from ACAA, TTGG, and GTAT.

Tables are provided below for each center sequence. Some tables provide the identified or exemplified residues at one or more positions in a subunit that correspond to positions 48, 50, 71, 72, 73, 73B, and 74 of I-CreI (e.g., Tables 1 and 3 for ACAA). Some tables provide residues at one or more additionally identified or exemplified positions that can be introduced into a subunit when targeting a specific center sequence (e.g., Tables 2 and 4 for ACAA).

TABLE 1
Exemplified Residues for ACAA Center Sequence (First Subunit)

| | | | | |
|---|---|---|---|---|
| I-CreI Position | 48 | 50 | 71 | 72 |
| EN Position | 48 | 50 | 71 | 72 |
| Residue(s) | K, L | C, R, T, K, S | G, R | R, Q |

TABLE 2
Exemplified Residues for ACAA Center Sequence (First Subunit)

| | | | |
|---|---|---|---|
| I-CreI Position | 19 | 80 | 139 |
| EN Position | 19 | 80 | 139 |
| Residue(s) | A, G | Q, E | K, R |

TABLE 3
Additionally Exemplified Residues for
ACAA Center Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | K, T, | C, R, E, | G, A | T, R, S, P, | V, I | S, T, A |
| | S,A | K, T | | N, G, A | | |

TABLE 4
Additionally Exemplified Residues for
ACAA Center Sequence (Second Subunit)

| | | | | | | |
|---|---|---|---|---|---|---|
| I-CreI Position | 19 | 66 | 80 | 92 | 117 | 139 |
| EN Position | 210 | 257 | 271 | 283 | 308 | 330 |
| Residue(s) | G, A, S | Y, C | Q, E | Q, R | E, G | K, R |

TABLE 5
Exemplified Residues for ACAG Center Sequence (First Subunit)

| | | | | |
|---|---|---|---|---|
| I-CreI Position | 50 | 71 | 72 | 73 |
| EN Position | 50 | 71 | 72 | 73 |
| Residue(s) | R | G, R | R, K, Q, P, T | A, C |

TABLE 6
Additionally Exemplified Residues for
ACAG Center Sequence (First Subunit)

| | | | | |
|---|---|---|---|---|
| I-CreI Position | 19 | 54 | 80 | |
| EN Position | 19 | 54 | 80 | 158 |
| Residue(s) | A, G | F, I, L | Q, E | S, P |

TABLE 7
Exemplified Residues for ACAG Center
Sequence (Second Subunit)

| | | | | | |
|---|---|---|---|---|---|
| I-CreI Position | 50 | 71 | 72 | 73 | 73B |
| EN Position | 241 | 262 | 263 | 264 | |
| +1 AA* | 241 | 262 | 263 | 264 | 264B* |
| Residue(s) | C | S, D, G | R, G | R | R or no R |

*Refers to engineered meganucleases having an insertion following a position which corresponds to position 73 of I-CreI.

TABLE 8
Additionally Exemplified Residues for
ACAG Center Sequence (Second Subunit)

| | | | | | | |
|---|---|---|---|---|---|---|
| I-CreI Position | 19 | 59 | 66 | 80 | 81 | 139 |
| EN Position | 210 | 250 | 257 | 271 | 272 | 330 |
| +1 AA* | 210 | 250 | 257 | 272* | 273* | 331* |
| Residue(s) | G, A, S | V, A | Y, H | Q | I, T | K, R |

*Refers to engineered meganucleases having an insertion following a position which corresponds to position 73 of I-CreI.

TABLE 9
Exemplified Residues for ACAT Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, S, I, L, N | Q, S, R, K | G, R | R, T | A, G |

TABLE 10
Additionally Exemplified Residues for
ACAT Center Sequence (First Subunit)

| | | | | |
|---|---|---|---|---|
| I-CreI Position | 19 | 54 | 80 | 139 |
| EN Position | 19 | 54 | 80 | 139 |
| Residue(s) | A, G, S | F, I | Q, E | K, H, R |

TABLE 11
Exemplified Residues for ACAT Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | H, T, | S, K, | S, G, | T, K, | H, A, | S, C, |
| | G, A, | C, N, | R, T, | A, S, | C, S, | A |
| | S, L, | R, G, | K, E | R, H, | G, R | |
| | K | Q | | G, N | | |

TABLE 12
Additionally Exemplified Residues for
ACAT Center Sequence (Second Subunit)

| | | | | | | |
|---|---|---|---|---|---|---|
| I-CreI Position | 19 | 80 | 81 | 83 | 117 | 139 |
| EN Position | 210 | 271 | 272 | 274 | 308 | 330 |
| Residue(s) | A, G, S | Q, E | I, T | P, H | E, G | K, R, |
| | | | | | | T, H |

TABLE 13
Exemplified Residues for ACGA Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K | V, R, T, W, A | G, P | R, P | A |

TABLE 14
Additionally Exemplified Residues for
ACGA Center Sequence (First Subunit)

| | | | |
|---|---|---|---|
| I-CreI Position | 19 | 80 | 139 |
| EN Position | 19 | 80 | 139 |
| Residue(s) | A, G, S | Q, E | K, R |

TABLE 15
Exemplified Residues for ACGA Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | K, H, | R, S, | G | R, H | I, V | S, A |
| | T, A, | C, I, | | | | |
| | G, Q | V, G | | | | |

TABLE 16
Additionally Exemplified Residues for
ACGA Center Sequence (Second Subunit)

| | | | |
|---|---|---|---|
| I-CreI Position | 19 | 80 | 139 |
| EN Position | 210 | 271 | 330 |
| Residue(s) | A, G | Q, E | K, R |

TABLE 17
Exemplified Residues for ACGC Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, H, Q, | Q, R, K, | G, R, A | R, P, H | A |
| | L, A, S | S, T, C | | | |

TABLE 18
Additionally Exemplified Residues for
ACGC Center Sequence (First Subunit)

| | | |
|---|---|---|
| I-CreI Position | 19 | 80 |
| EN Position | 19 | 80 |
| Residue(s) | A, G, S | Q, E |

TABLE 19
Exemplified Residues for ACGC Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | H, K, | S, E, | S, G, | T, R, | H, T, | S, A, |
| | L, A, | K, I, | K, A, | A, S, | V, I, | T |
| | S, N | N, V | R | H, G | C | |

TABLE 20
Additionally Exemplified Residues for
ACGC Center Sequence (Second Subunit)

| | | | | |
|---|---|---|---|---|
| I-CreI Position | 19 | 80 | 87 | 139 |
| EN Position | 210 | 271 | 278 | 330 |
| Residue(s) | A, G | Q, E | F, L | K, R, N, H, A |

TABLE 21
Exemplified Residues for ACGG Center Sequence (First Subunit)

| | | | |
|---|---|---|---|
| I-CreI Position | 50 | 72 | 73 |
| EN Position | 50 | 72 | 73 |
| Residue(s) | R, K | R | A |

TABLE 22
Additionally Exemplified Residues for
ACGG Center Sequence (First Subunit)

| | | |
|---|---|---|
| I-CreI Position | 54 | 80 |
| EN Position | 54 | 80 |
| Residue(s) | F, L | Q |

TABLE 23
Exemplified Residues for ACGG Center
Sequence (Second Subunit)

| | | | | | | |
|---|---|---|---|---|---|---|
| I-CreI Position | 48 | 50 | 71 | 72 | 73 | |
| EN Position | 239 | 241 | 262 | 263 | 264 | |
| +1 AA* | 239 | 241 | 262 | 263 | 264 | 264B* |
| Residue(s) | K | R, P | D | G | R, G | R |

*Refers to engineered meganucleases having an insertion following a position which corresponds to position 73 of I-CreI.

TABLE 24
Additionally Exemplified Residues for
ACGG Center Sequence (Second Subunit)

| | | |
|---|---|---|
| I-CreI Position | 19 | 80 |
| EN Position | 210 | 271 |
| +1 AA* | 210 | 272* |
| Residue(s) | A | Q |

*Refers to engineered meganucleases having an insertion following a position which corresponds to position 73 of I-CreI.

TABLE 25
Exemplified Residues for ACGT Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, L, S, H | Q, R, C, S, V | G | R | A |

TABLE 26
Additionally Exemplified Residues for
ACGT Center Sequence (First Subunit)

| | | | |
|---|---|---|---|
| I-CreI Position | 19 | 80 | 139 |
| EN Position | 19 | 80 | 139 |
| Residue(s) | A, G | Q, E | K, R |

TABLE 27
Exemplified Residues for ACGT Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | H, K, | S, C, | S, P, G, | T, R, | H, C, | S, A, |
| | L, S | Q, E, | T, A, R, | K, A | A, S | T |
| | | A | N | | | |

TABLE 28
Additionally Exemplified Residues for
ACGT Center Sequence (Second Subunit)

| | | | | |
|---|---|---|---|---|
| I-CreI Position | 19 | 80 | 85 | 139 |
| EN Position | 210 | 271 | 276 | 330 |
| Residue(s) | A, G | Q, E | H, Y | K, R |

TABLE 29
Exemplified Residues for ATAA Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, A, | Q, T, R, | G, K, | R, A, G, | A, T, | S, A |
| | H, S, | I, G, K, | S, H | Q, H, L, | C | |
| | L, Q | D, C, V | N | S | | |

TABLE 30
Additionally Exemplified Residues for
ATAA Center Sequence (First Subunit)

| | | | | |
|---|---|---|---|---|
| I-CreI Position | 19 | 80 | 100 | 139 |
| EN Position | 19 | 80 | 100 | 139 |
| Residue(s) | A, G, S | K, A, H, | Q, T, R, I, G, K, | G, K, S, H, N |
| | | S, L, Q | D, C, V | |

TABLE 31
Exemplified Residues for ATAA Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | S, T, A, | R, K, | S, G, | T, R, Q, | I, C, | S, A, |
| | K, N | E, A, | K, R | G, A, Y, | V | T |
| | | C, T | | S, N, K | | |

TABLE 32
Additionally Exemplified Residues for
ATAA Center Sequence (Second Subunit)

| | | | | | |
|---|---|---|---|---|---|
| I-CreI Position | 19 | 59 | 80 | 118 | 139 |
| EN Position | 210 | 250 | 271 | 309 | 330 |
| Residue(s) | G, S, A | V, A | Q, E | S, F | K, R |

TABLE 33
Exemplified Residues for ATAG Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, H | R | G, R, H | R, G, S, A, P, Q | A, C |

TABLE 34
Additionally Exemplified Residues for
ATAG Center Sequence (First Subunit)

| | | | |
|---|---|---|---|
| I-CreI Position | 19 | 80 | 139 |
| EN Position | 19 | 80 | 139 |
| Residue(s) | A, G | Q, E | K, R |

TABLE 35
Exemplified Residues for ATAG Center
Sequence (Second Subunit)

| | | | |
|---|---|---|---|
| I-CreI Position | 50 | 72 | 73 |
| EN Position | 241 | 263 | 264 |
| Residue(s) | C, R | G, S | R |

TABLE 36
Additionally Exemplified Residues for
ATAG Center Sequence (Second Subunit)

| | | | | | |
|---|---|---|---|---|---|
| I-CreI Position | 19 | 36 | 59 | 80 | 139 |
| EN Position | 210 | 227 | 250 | 271 | 330 |
| Residue(s) | G, A | K, R | V, A | Q | K, R |

TABLE 37
Exemplified Residues for ATAT Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, H, C, | Q, N, C, R, | G, H, I | R, A, | A, C, S |
| | A, S, D, T | K, S, T, V | | N, Q | |

TABLE 38
Additionally Exemplified Residues for
ATAT Center Sequence (First Subunit)

| | | | |
|---|---|---|---|
| I-CreI Position | 19 | 80 | 139 |
| EN Position | 19 | 80 | 139 |
| Residue(s) | A, G | Q, E | K, R, S |

TABLE 39
Exemplified Residues for ATAT Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | H, K, | S, C, | S, K, | T, A, R, | H, C, | S, C, |
| | A, S, | K, R, | E, I, | S, K, G, | A, S, | A |
| | R, T | Q, N | G, R | N | G | |

TABLE 40
Additionally Exemplified Residues for
ATAT Center Sequence (Second Subunit)

| | | | | |
|---|---|---|---|---|
| I-CreI Position | 19 | 59 | 80 | 139 |
| EN Position | 210 | 250 | 271 | 330 |
| Residue(s) | G, A, | V, A | Q, E, K | K, R, P, N |

TABLE 41
Exemplified Residues for ATGA Center Sequence (First Subunit)

| | | | | |
|---|---|---|---|---|
| I-CreI Position | 48 | 50 | 72 | 73 |
| EN Position | 48 | 50 | 72 | 73 |
| Residue(s) | K, A, H, L | R, T, E, | R, T, S, | A, S |
| | | S, C, V | A, K | |

TABLE 42
Additionally Exemplified Residues for
ATGA Center Sequence (First Subunit)

| | | | | | |
|---|---|---|---|---|---|
| I-CreI Position | 19 | 80 | 87 | 92 | 139 |
| EN Position | 19 | 80 | 87 | 92 | 139 |
| Residue(s) | A, G, S | Q, E | F, L | Q, R | K, R |

TABLE 43
Exemplified Residues for ATGA Center
Sequence (Second Subunit)

| | | | | | |
|---|---|---|---|---|---|
| I-CreI Position | 48 | 50 | 72 | 73 | 74 |
| EN Position | 239 | 241 | 263 | 264 | 265 |
| Residue(s) | H, K, R, | S, I, R, | R, H | I, V | S, A, T |
| | A, S | C, A, Q | | | |

TABLE 44
Additionally Exemplified Residues for
ATGA Center Sequence (Second Subunit)

| | | | | |
|---|---|---|---|---|
| I-CreI Position | 19 | 59 | 80 | 139 |
| EN Position | 210 | 250 | 271 | 330 |
| Residue(s) | G, A, S | V, A | Q, E | K, R |

TABLE 45
Exemplified Residues for ATGG Center Sequence

| | | | | | |
|---|---|---|---|---|---|
| I-CreI Position | 50 | 71 | 72 | 73 | 74 |
| EN Position | 48 | 50 | 72 | 73 | 74 |
| Residue(s) | R | G, S | P, G | A, C | S, C |

TABLE 46
Additionally Exemplified Residues for
ATGG Center Sequence (First Subunit)

| | | | | |
|---|---|---|---|---|
| I-CreI Position | 19 | 80 | 82 | 139 |
| EN Position | 19 | 80 | 87 | 92 |
| Residue(s) | G, A | E, Q | E, K | R, K |

TABLE 47
Exemplified Residues for ATGG Center
Sequence (Second Subunit)

| | | | | | | |
|---|---|---|---|---|---|---|
| I-CreI Position | 48 | 50 | 71 | 72 | 73 | |
| EN Position | 239 | 241 | 262 | 263 | 264 | |
| +1 AA* | 239 | 241 | 262 | 263 | 264 | 264B |
| Residue(s) | K | R | D, G | G | R | R or no |
| | | | | | | R |

*Refers to engineered meganucleases having an insertion following a position which corresponds to position 73 of I-CreI.

TABLE 48
Additionally Exemplified Residues for
ATGG Center Sequence (Second Subunit)

| | | | |
|---|---|---|---|
| I-CreI Position | 19 | 77 | 80 |
| EN Position | 210 | 268 | 271 |
| +1 AA* | 210 | 269 | 272 |
| Residue(s) | A, G | N | Q, R |

*Refers to engineered meganucleases having an insertion following a position which corresponds to position 73 of I-CreI.

TABLE 49
Exemplified Residues for GCAA Center Sequence (First Subunit)

| | | | | | |
|---|---|---|---|---|---|
| I-CreI Position | 48 | 50 | 71 | 72 | 73 |
| EN Position | 48 | 50 | 71 | 72 | 73 |
| Residue(s) | K, H | R, C, K, | G, N, T, | R, P, S, N, Q, | T, V |
| | | T, L | R, S, H | G, A, M, V, T | |

TABLE 50
Additionally Exemplified Residues for
GCAA Center Sequence (First Subunit)

| | | | |
|---|---|---|---|
| I-CreI Position | 19 | 80 | 139 |
| EN Position | 19 | 80 | 139 |
| Residue(s) | A, G, S | Q, E | K, R |

TABLE 51
Exemplified Residues for GCAA Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | S, A, | R, C, | G, R, | T, G, S, | C, V, | S, A, |
| | K, T | T, K, | A, H | A, E, N, | I | T |
| | | E | | K, H, R, | | |
| | | | | C, Y | | |

TABLE 52
Additionally Exemplified Residues for
GCAA Center Sequence (Second Subunit)

| | | | | |
|---|---|---|---|---|
| I-CreI Position | 19 | 31 | 80 | 139 |
| EN Position | 210 | 222 | 271 | 330 |
| Residue(s) | G, A | Q, P | Q, E | K, R |

TABLE 53
Exemplified Residues for GCAT Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, A, | Q, V, | G, A, H, | R, T, G, | A, T, | S, |
| | H, R | R, K, | R, T, N, | S, Q, N, | V, C | A |
| | | S | S | A | | |

TABLE 54
Additionally Exemplified Residues for
GCAT Center Sequence (First Subunit)

| | | | | |
|---|---|---|---|---|
| I-CreI Position | 19 | 80 | 139 | 143 |
| EN Position | 19 | 80 | 139 | 143 |
| Residue(s) | A, G | Q, E | K, H, R | T, I |

TABLE 55
Exemplified Residues for GCAT Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 262 | 263 | 264 | 265 |
| Residue(s) | H, A, | S, R, | S, K, R, | T, A, G, | H, C, | S, C, |
| | K, T, | K, Q, | A, G, T, | N, S, R, | G, S, | A |
| | L, I | H, V | H, Y | H, Q, K | A | |

TABLE 56
Additionally Exemplified Residues for
GCAT Center Sequence (Second Subunit)

| | | | | |
|---|---|---|---|---|
| I-CreI Position | 19 | 80 | 125 | 139 |
| EN Position | 210 | 271 | 316 | 330 |
| Residue(s) | G, S, A | Q, E | V, A | K, R, H |

TABLE 57
Exemplified Residues for GCGA Center Sequence (First Subunit)

| | | | | | |
|---|---|---|---|---|---|
| I-CreI Position | 50 | 71 | 72 | 73 | 74 |
| EN Position | 50 | 71 | 72 | 73 | 74 |
| Residue(s) | K, R | G, R, S, | R, N, G, | V, T, I | S, A |
| | | A, N | A, Q | | |

TABLE 58
Additionally Exemplified Residues for
GCGA Center Sequence (First Subunit)

| | | |
|---|---|---|
| I-CreI Position | 19 | 80 |
| EN Position | 19 | 80 |
| Residue(s) | A, G, S | Q, E |

TABLE 59
Exemplified Residues for GCGA Center
Sequence (Second Subunit)

| | | | | | |
|---|---|---|---|---|---|
| I-CreI Position | 48 | 50 | 72 | 73 | 74 |
| EN Position | 239 | 241 | 263 | 264 | 265 |
| Residue(s) | K, T, S, A, Q | C, R | R | V, I | S, A |

TABLE 60
Additionally Exemplified Residues for
GCGA Center Sequence (Second Subunit)

| | | | |
|---|---|---|---|
| I-CreI Position | 19 | 80 | 139 |
| EN Position | 210 | 271 | 330 |
| Residue(s) | G, S, A | Q, E | R |

TABLE 61
Exemplified Residues for GTAA Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, S, | T, R, | G, R, S, | R, S, C, | V, C, | S, A, |
| | A, R, | A, K, | T, A, N, | N, K, A, | I, T | T |
| | N, T | C | H, K | H, G, T, | | |
| | | | | D, Y, P, | | |
| | | | | Q | | |

TABLE 62
Additionally Exemplified Residues for
GTAA Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, R |

TABLE 63
Exemplified Residues for GTAG Center Sequence (First Subunit)

| | | | | |
|---|---|---|---|---|
| I-CreI Position | 50 | 71 | 72 | 73 |
| EN Position | 50 | 71 | 72 | 73 |
| Residue(s) | R, C | D, S | G, N | R |

TABLE 64
Additionally Exemplified Residues for
GTAG Center Sequence (First Subunit)

| | | | |
|---|---|---|---|
| I-CreI Position | 19 | 80 | 139 |
| EN Position | 19 | 80 | 139 |
| Residue(s) | A, S | Q | K, R |

TABLE 65
Exemplified Residues for GTAT Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, G, | Q, V, | G, T, | R, K, | A, C, | S, A, |
| | T, A, | R, S, | A, K, | S, Y, | S, T | C |
| | M, H, | T, G, | H, R, | N, T, | | |
| | S, L, | K, C, | Y, L, | G, W, | | |
| | R | L | S, N | H, A | | |

TABLE 66
Additionally Exemplified Residues for
GTAT Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, R, T, H |

TABLE 67
Exemplified Residues for GTGA Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, A, | R, V, | G, R, | R, T, | A, V, | S, T, |
| | G, R, | C, S | V, S, | S, G, | T | A, G |
| | S, H | | A, T, | H, K, | | |
| | | | N, D, | Y | | |
| | | | H | | | |

TABLE 68
Additionally Exemplified Residues for
GTGA Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, R |

TABLE 69
Exemplified Residues for GTGC Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, L, | R, S, | G, S, N, | R, K, G, | A, V, | S, A, |
| | H, A, | V, K, | I, R, A, | H, P, S, | T, N, | T |
| | R, N, | I, G | E, Q, Y, | C, N, T, | C, L | |
| | S | | T, K, F, | A, M, D, | | |
| | | | V | Q | | |

TABLE 70
Additionally Exemplified Residues for
GTGC Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, T, S, |
| | | | R, H, V |

TABLE 11
Exemplified Residues for GTGG Center Sequence (First Subunit)

| | | | | |
|---|---|---|---|---|
| I-CreI Position | 50 | 71 | 72 | 73 |
| EN Position | 50 | 71 | 72 | 73 |
| Residue(s) | Q, R | G, S, D | G, S | R, V |

TABLE 72
Additionally Exemplified Residues for
GTGG Center Sequence (First Subunit)

| | | | |
|---|---|---|---|
| I-CreI Position | 19 | 62 | 80 |
| EN Position | 19 | 62 | 80 |
| Residue(s) | A, G, S | I, V | Q, E |

TABLE 73
Exemplified Residues for GTGT Center Sequence (First Subunit)

| | | | | | | |
|---|---|---|---|---|---|---|
| I-CreI Position | 48 | 50 | 71 | 72 | 73 | 74 |
| EN Position | 48 | 50 | 71 | 72 | 73 | 74 |
| Residue(s) | K, S, | Q, V, | G, R, | R, P, | A, S, | S, A, |
| | L, V, | R, S, | N, H, | A, Q, | C, T | T |
| | G, R, | K, A, | A, T | K, T, | | |
| | N | E, C | | G, V | | |

TABLE 74
Additionally Exemplified Residues for
GTGT Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, R |

TABLE 75
Exemplified Residues for TCAA Center Sequence (First Subunit)

| | | | | |
|---|---|---|---|---|
| I-CreI Position | 48 | 50 | 71 | 72 |
| EN Position | 48 | 50 | 71 | 72 |
| Residue(s) | K, S | R, T,C | G, R, T | R, S, P, |
| | | | | T, G |

TABLE 76
Additionally Exemplified Residues for
TCAA Center Sequence (First Subunit)

| | | | |
|---|---|---|---|
| I-CreI Position | 19 | 80 | 139 |
| EN Position | 19 | 80 | 139 |
| Residue(s) | A, S | Q, E | K, R |

TABLE 77
Exemplified Residues for TCAA Center
Sequence (Second Subunit)

| | | | | | |
|---|---|---|---|---|---|
| I-CreI Position | 48 | 50 | 72 | 73 | 74 |
| EN Position | 239 | 241 | 263 | 264 | 265 |
| Residue(s) | S, K | K, R, C, E | R, Q, N, S | I | S, A |

TABLE 78
Additionally Exemplified Residues for
TCAA Center Sequence (Second Subunit)

| | | | |
|---|---|---|---|
| I-CreI Position | 19 | 80 | 139 |
| EN Position | 210 | 271 | 330 |
| Residue(s) | G, S | Q, E | R |

TABLE 79
Exemplified Residues for TTAA Center Sequence (First Subunit)

| | | | | | |
|---|---|---|---|---|---|
| I-CreI Position | 48 | 50 | 71 | 72 | 74 |
| EN Position | 48 | 50 | 71 | 72 | 74 |
| Residue(s) | K, N, | R, V, | G, R, N, | R, T, S, N, | S, A |
| | S, R | K, S | S, A | D, Q, K, A | |

TABLE 80
Additionally Exemplified Residues for
TTAA Center Sequence (First Subunit)

| | | | |
|---|---|---|---|
| I-CreI Position | 19 | 80 | 139 |
| EN Position | 19 | 80 | 139 |
| Residue(s) | A, G, S | Q, E | K, R |

TABLE 81
Exemplified Residues for TTAA Center
Sequence (Second Subunit)

| | | | | | |
|---|---|---|---|---|---|
| I-CreI Position | 48 | 50 | 72 | 73 | 74 |
| EN Position | 239 | 241 | 257 | 263 | 264 |
| Residue(s) | K, S, A, T | C, K, R, | T, K, R, | I, V | S, A |
| | | T, E | A, S, Q | | |

TABLE 82
Additionally Exemplified Residues for
TTAA Center Sequence (Second Subunit)

| | | | | |
|---|---|---|---|---|
| I-CreI Position | 19 | 66 | 80 | 139 |
| EN Position | 210 | 257 | 271 | 330 |
| Residue(s) | G, A, S | Y, H | Q | R |

TABLE 83
Exemplified Residues for TTGG Center Sequence (First Subunit)

| | | | | |
|---|---|---|---|---|
| I-CreI Position | 50 | 71 | 72 | 73 |
| EN Position | 50 | 71 | 72 | 73 |
| Residue(s) | R | S | G | R |

TABLE 84
Additionally Exemplified Residues for
TTGG Center Sequence (First Subunit)

| | | |
|---|---|---|
| I-CreI Position | 19 | 80 |
| EN Position | 19 | 80 |
| Residue(s) | A, G | Q |

TABLE 85
Exemplified Residues for TTGG Center
Sequence (Second Subunit)

| | | | | | | |
|---|---|---|---|---|---|---|
| I-CreI Position | 48 | 50 | 71 | 72 | 73 | 74 |
| EN Position | 239 | 241 | 257 | 262 | 263 | 264 |
| Residue(s) | K, S | C, T, E, | G, K | T, Q, K, R, | I, V | S, A |
| | | K, R | | H, A, S | | |

TABLE 86
Additionally Exemplified Residues for
TTGG Center Sequence (Second Subunit)

| | | | | | |
|---|---|---|---|---|---|
| I-CreI Position | 19 | 66 | 80 | 85 | 139 |
| EN Position | 210 | 257 | 271 | 276 | 330 |
| Residue(s) | G, A | Y, H | Q | H, R | K, R |

TABLE 87
Exemplified Residues for GCAG Center Sequence (First Subunit)

| | | | | |
|---|---|---|---|---|
| I-CreI Position | 50 | 71 | 72 | 73 |
| EN Position | 50 | 71 | 72 | 73 |
| Residue(s) | R | S | G | R |

TABLE 88
Additionally Exemplified Residues for
GCAG Center Sequence (First Subunit)

| | | |
|---|---|---|
| I-CreI Position | 19 | 80 |
| EN Position | 19 | 80 |
| Residue(s) | A | Q |

TABLE 89
Exemplified Residues for GCAG Center
Sequence (Second Subunit)

| | | | | |
|---|---|---|---|---|
| I-CreI Position | 48 | 50 | 72 | 73 |
| EN Position | 239 | 241 | 262 | 263 |
| Residue(s) | K, H | Q, R | S, R | V, T |

TABLE 90
Additionally Exemplified Residues for
GCAG Center Sequence (Second Subunit)

| | |
|---|---|
| I-CreI Position | 80 |
| EN Position | 271 |
| Residue(s) | Q |

According to Tables 1-90 above there are certain common residues that may be substituted for residues 48, 50, 71, 72, 73, 73B2 and 74 corresponding to SEQ ID NO: 1 (i.e., I-CreI) to improve the cleaving of certain center sequences. The residues indicated in tables 91-110 below represent residues that may be substituted for the corresponding wild type I-CreI residues with an expectation of an improvement in cleavage activity of the indicated center sequence based on the analysis of the exemplified residues in tables 1-90 for related center sequences. In some embodiments, the engineered meganucleases described herein that cleave a center sequence selected from ACAA, ACAG, ACAT, ACGA, ACGC, ACGG, ACGT, ATAA, ATAG, ATAT, ATGA, ATGG, GCAA, GCAT, GCGA, CAG, TAA, TCAA, and TTGG comprise one or more residues in a first subunit and a second subunit at positions 48, 50, 71, 72, 73, 73B, and 74 according to table 91 and table 92 below.

TABLE 91
Common Residues for ACAA, ACAG, ACAT, ACGA, ACGC, ACGG,
ACGT, ATAA, ATAG, ATAT, ATGA, ATGG, GCAA, GCAT, GCGA,
GCAG, TTAA, TCAA, and TTGG (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | A, C, | A, C, | A, G, | A, D, | A, C, | no R | A, C, |
| | D, G, | D, E, | H, I, | G, H, | G, I, | | T, S |
| | H, I, | G, I, | K, N, | K, L, | S, T, | | |
| | K, L, | K, L, | P, R, | M, N, | V | | |
| | N, Q, | N, Q, | S, T | P, Q, | | | |
| | R, S, | R, S, | | R, S, | | | |
| | T | T, V, | | T, V | | | |
| | | W | | | | | |

TABLE 92
Common Residues for ACAA, ACAG, ACAT, ACGA, ACGC, ACGG,
ACGT, ATAA, ATAG, ATAT, ATGA, ATGG, GCAA, GCAT, GCGA,
GCAG, TTAA, TCAA, and TTGG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | A, C, | A, D, | A, C, | A, C, | R or | A, C, |
| | G, H, | E, G, | E, G, | E, G, | G, H, | no R | S, T |
| | I, K, | H, I, | H, I, | H, I, | I, R, | | |
| | L, N, | K, N, | K, N, | K, M, | S, T, | | |
| | Q, R, | P, Q, | P, Q, | N, P, | V | | |
| | S, T | R, S, | R, S, | Q, R, | | | |
| | | T, V | T, Y | S, T, | | | |
| | | | | V, Y | | | |

It was further discovered that particular identical residues in the first subunit for the same two base pairs of a center sequence of a second center sequence have similar residues that may be suitably substituted at one or more positions corresponding to positions 48, 50, 71, 72, 73, 73B, and 74 of I-Cre. For example, a first subunit for meganucleases cleaving the center sequences ACAA and ACAG having the first two base pairs AC are substituted in a more similar way. Accordingly, particular residues may be substituted for positions corresponding to positions 48, 50, 71, 72, 73, and 74 of I-CrcI to improve cleavage activity of the center sequences ACAA, ACAG, ACAT, ACGA, ACGC, ACGG, and ACGT. In some embodiments described herein are engineered meganucleases having one or more substitutions in positions corresponding to positions 48, 50, 71, 72, 73, 73B, and 74 of SEQ ID NO: 1 (i.e., I-CreI) in a first subunit and a second subunit according to table and table 94 below.

TABLE 93
Common Residues for ACAA, ACAG, ACAT, ACGA,
ACGC, ACGG, and ACGT (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | A, C, | A, C, | A, G, | H, K, | A, C, | no R | S |
| | G, H, | K, Q, | P, R | P, Q, | G, V | | |
| | I, K, | R, S, | | R, T, | | | |
| | L, N, | T, V, | | | | | |
| | Q, S | W | | | | | |

TABLE 94
Common Residues for ACAA, ACAG, ACAT, ACGA,
ACGC, ACGG, and ACGT (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | A, C, | A, D, | A, G, | A, C, | R or | A, C, |
| | G, H, | E, G, | E, G, | H, K, | G, H, | no R | S, T |
| | K, L, | I, K, | H, K, | M, N, | I, R, | | |
| | N, Q, | N, P, | N, P, | P, P, | S, T, | | |
| | R, S, | Q, R, | R, S, | Q, R, | V | | |
| | T | S, T, | T | S, T | | | |
| | | V | | | | | |

In some further embodiments, one or more residues may be substituted for positions corresponding to positions 48, 50, 71, 72, 73, 73B, and 74 of SEQ ID NO: 1 (i.e., I-Cre) to improve cleavage activity of the center sequences ATAA, ATAG, ATAT, ATGA, and ATGG as shown in table 95 and 96 below.

TABLE 95
Common Residues for ATAA, ATAG, ATAT,
ATGA, and ATGG (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | A, C, | C, D, | G, H, | A, G, | A, C, | no R | A, C, |
| | D, G, | E, G, | I, K, | H, K, | S, T | | S |
| | H, K, | I, K, | N, R, | L, N, | | | |
| | L, N, | N, R, | S | P, Q, | | | |
| | Q, S, | S, T, | | R, S, | | | |
| | T | V | | T | | | |

TABLE 96
Common Residues for ATAA, ATAG, ATAT,
ATGA, and ATGG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | A, C, | D, E, | A, G, | A, C, | R or | A, C, |
| | G, H, | E, I, | G, H, | H, K, | G, H, | no R | S, T |
| | K, N, | K, N, | I, K, | N, Q, | I, R, | | |
| | Q, R, | Q, R, | R, S, | R, S, | S, V | | |
| | S, T | S, T | T | T, V, | | | |
| | | | | Y | | | |

In some other embodiments, one or more residues may be substituted for positions corresponding to positions 48, 50, 71, 72, 73, 73B, and 74 of SEQ ID NO: 1 (i.e., I-CreI) to improve cleavage activity of the center sequences GCAA, GCAT, GCGA, and GCAG as shown in table 97 and table 98 below.

TABLE 97
Common Residues for GCAA, GCAT,
GCGA, and GCAG (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | A, H, | C, K, | A, G, | A, G, | A, C, | no R | A, S |
| | K, R | L, Q, | H, N, | H, M, | I, T, | | |
| | | R, S, | R, S, | N, P, | V | | |
| | | T, V | T | Q, R, | | | |
| | | | | S, T, | | | |
| | | | | V | | | |

TABLE 98
Common Residues for GCAA, GCAT,
GCGA, and GCAG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | C, E, | A, G, | A, C, | A, C, | no R | A, S, |
| | G, H, | H, K, | H, K, | E, G, | G, H, | | T |
| | I, K, | Q, R, | R, S, | H, K, | I, R, | | |
| | L, N, | S, T, | T, Y | N, Q, | S, V | | |
| | Q, R, | V | | R, S, | | | |
| | S, T | | | T, Y | | | |

In some particular embodiments, one or more residues may be substituted for positions corresponding to positions 48, 50, 71, 72, 73, 73B, and 74 of SEQ ID NO: 1 (i.e., I-CreI) to improve cleavage activity of the center sequences TTAA and TTGG as shown in table 99 and table 100 below.

TABLE 99
Common Residues for TTAA and TTGG (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | K, N, | C, E, | A, G, | A, D, | I, V | no R | A, S, |
| | R, S | K, R, | K, N, | H, K, | | | T |
| | | S, T, | R, S | N, Q, | | | |
| | | V | | R, S, | | | |
| | | | | T | | | |

TABLE 100
Common Residues for TTAA and TTGG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, K, | C, E, | A, D, | G, I, | I, R, | R or | A, S, |
| | S, T | K, R, | G, K, | R, S, | V | no R | T |
| | | T | Q, R, | T, V | | | |
| | | | S, T | | | | |

In some other embodiments, one or more residues may be substituted for positions corresponding to positions 48, 50, 71, 72, 73, 73B, and 74 of SEQ ID NO: 1 (i.e., I-CreI) to improve cleavage activity of the center sequence TCAA as shown in table 101 and table 102 below.

TABLE 101
Common Residues for TCAA (First Subunit)

| | | | | | | | |
|---|---|---|---|---|---|---|---|
| I-CreI Position | 48 | 50 | 71 | 72 | 73 | 73B | 74 |
| EN Position | 48 | 50 | 71 | 72 | 73 | 73B | 74 |
| Residue(s) | A, G, | C, R, | G, R, | G, H, | I, V | No R | A, S |
| | H, K, | S, T | S, T | P, R, | | | |
| | N, Q, | | | S, T | | | |
| | R, S | | | | | | |

TABLE 102
Common Residues for TCAA (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | K, S | C, K, | G, R, | G, P, | I, V | No R | A, S, |
| | | R, T | T | R, S, | | | T |
| | | | | T | | | |

It was likewise identified that particular identical residues in the second subunit for the same two base pairs of a center sequence of a second center sequence have similar residues that may be suitably substituted at positions corresponding to positions 48, 50, 71, 72, 73, 73B, and 74 of SEQ ID NO: 1 (i.e., I-CreI). For example, a second subunit for meganucleases cleaving the center sequences ACAA and ATAA both having the second two base pairs AA (reverse complement TT) are substituted in a similar way. Accordingly, in some embodiments, one or more residues may be substituted for positions corresponding to positions 48, 50, 71, 72, 73, 73B, and 74 of SEQ ID NO: 1 (i.e., I-CreI) to improve cleavage activity of the center sequences ACAA, ATAA, GCAA, TTAA, and TCAA as shown in table 103 below.

TABLE 103
Additional Common Residues for ACAA, ATAA,
GCAA, TTAA, TCAA (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, K, | A, C, | A, G, | A, C, | C, H, | — | A, S, |
| | N, S, | E, K, | H, K, | E, G, | I, V | | T |
| | T | K, R, | Q, R, | H, K, | | | |
| | | T | S, T | N, P, | | | |
| | | | | Q, R, | | | |
| | | | | S, T, | | | |
| | | | | Y | | | |

In some further embodiments, one or more residues may be substituted for positions corresponding to positions 48, 50, 71, 72, 73, 73B, and 74 of SEQ ID NO: 1 (i.e., I-CreI) to improve cleavage activity of the center sequences ACAG, ATAG, and GCAG as shown in table 104 below.

TABLE 104
Additional Common Residues for ACAG,
ATAG, and GCAG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | K | C, R | D, G, S | G, N, R, | R | — | S |
| | | | | or S | | | |

In some further embodiments, one or more residues may be substituted for positions corresponding to positions 48, 50, 71, 72, 73, 73B, and 74 of SEQ ID NO: 1 (i.e., I-CreI) to improve cleavage activity of the center sequences ACAT, ATAT, and GCAT as shown in table 105 below.

TABLE 105
Additional Common Residues for ACAT,
ATAT, and GCAT (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | C, G, | A, E, | A, G, | A, C, | — | A, C, |
| | G, H, | H, K, | G, H, | H, K, | G, H, | | S |
| | I, K, | N, Q, | I, K, | N, Q, | R, S | | |
| | L, N, | R, S, | R, S, | R, S, | | | |
| | Q, R, | V | T, Y | T | | | |
| | S, T | | | | | | |

In some alternative embodiments, one or more residues may be substituted for positions corresponding to positions 48, 50, 71, 72, 73, 73B, and 74 of SEQ ID NO: 1 (i.e., I-CreI) to improve cleavage activity of the center sequences ACGA, ATGA, and GCGA as shown in table 106 below.

TABLE 106
Additional Common Residues for ACGA,
ATGA, and GCGA (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, G, | A, C, | G, H, | H, I, | I, V | | A, S, |
| | H, K, | G, I, | R, S, | R, S, | | | T |
| | N, Q, | Q, R, | T | T, V | | | |
| | R, S, | S, V | | | | | |
| | T | | | | | | |

In some alternative embodiments, one or more residues may be substituted for positions corresponding to positions 48, 50, 71, 72, 73, 73B, and 74 of SEQ ID NO: 1 (i.e., I-CreI) to improve cleavage activity of the center sequences ACGA, ATGA, and GCGA as shown in table 107 below.

TABLE 107
Additional Common Residues for ACGC (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, H, | E, I, | A, G, | A, G, | C, H, | | S |
| | K, L, | K, | H, K, | H, M, | I, T, | | |
| | N, S | N, R, | N, R, | N, P, | V | | |
| | | S, V | S, T | Q, R, | | | |
| | | | | S, T | | | |

In some other embodiments, one or more residues may be substituted for positions corresponding to positions 48, 50, 71, 72, 73, 73B, and 74 of SEQ TD NO: 1 (i.e., I-CreI) to improve cleavage activity of the center sequences ACGA, ATGA, and GCGA as shown in table 108 below.

TABLE 108
Additional Common Residues for ACGG,
ATGG, and TTGG (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | K | P, R | G, D, | G | G, R | R or | A, S, |
| | | | S | | | no R | T |

In some embodiments, one or more residues may be substituted for positions corresponding to positions 48, 50, 71, 72, 73, 73B, and 74 of SEQ ID NO: 1 (i.e., I-CreI) to improve cleavage activity of the center sequences ACGT as shown in table 109 below.

TABLE 109
Additional Common Residues for ACGT (Second Subunit)

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | A, C, | A, G, | A, K, | A, C, | | S |
| | G, H, | E, K, | N, P, | R, T | H, S | | |
| | K, L, | Q, R, | R, S, | | | | |
| | N, Q, | S | or T | | | | |
| | S | | | | | | |

In some embodiments, the engineered meganucleases described herein that cleave a center sequence selected from GTAA, GTAG, GTAT, GTGA, GTGC, GTGG, and GTGT comprise one or more residues in a first subunit at positions 48, 50, 71, 72, 73, 73B, and 74 according to table 110 below. The GT (reverse complement AC) binding subunit for these meganucleases was not altered since the wild type SEQ ID NO: 1 (i.e., I-CreI) center sequence is GTGA.

TABLE 110
Common Residues for GTAA, GTAG,
GTAT, GTGA, GTGC, GTGG, and GTGT

| | | | | | | | |
|---|---|---|---|---|---|---|---|
| I-CreI Position | 48 | 50 | 71 | 72 | 73 | 73B | 74 |
| EN Position | 239 | 241 | 262 | 263 | 264 | 264B | 265 |
| Residue(s) | A, C, | A, C, | A, D, | A, C, | A, C, | — | A, C, |
| | G, H, | E, G, | E, F, | D, G, | I, L, | | G, S, |
| | K, L, | I, K, | G, H, | H, K, | N, R, | | T |
| | M, N, | L, Q, | I, K, | M, N, | S, T, | | |
| | Q, R, | R, S, | L, N, | P, Q, | V | | |
| | S, T, | T, V | Q, R, | R, S, | | | |
| | V | | S, T, | T, V, | | | |
| | | | V, Y | w, Y | | | |

In some embodiments described herein is an engineered meganuclease that cleaves the center sequence ATAT, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ATAT, wherein the engineered meganuclease comprises a substitution described herein in a second subunit at positions corresponding to positions 48, 50, 71, 72, 73, and 74 of SEQ ID NO: 1 (i.e., I-CreI).

In some embodiments described herein is an engineered meganuclease that cleaves the center sequence ATAA, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ATAA, wherein the engineered meganuclease comprises a substitution described herein in a second subunit at positions corresponding to positions 50 of SEQ ID NO: 1 (i.e., I-CreI).

In some embodiments described herein is an engineered meganuclease that cleaves the center sequence ATAG, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ATAG, wherein the engineered meganuclease comprises a substitution described herein in a second subunit at positions corresponding to positions 50, 72, and 73 of SEQ ID NO: 1 (i.e., I-CreI).

In some embodiments described herein is an engineered meganuclease that cleaves the center sequence ATGA, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ATGA, wherein the engineered meganuclease comprises a substitution described herein in a second subunit at positions corresponding to positions 50 and 72 of SEQ ID NO: 1 (i.e., I-CreI).

In some embodiments described herein is an engineered meganuclease that cleaves the center sequence ATGG, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ATGG, wherein the engineered meganuclease comprises a substitution described herein in a second subunit at positions corresponding to positions 50, 71, 72, 73, 73B of SEQ ID NO: 1 (i.e., I-CreI).

In some embodiments described herein is an engineered meganuclease that cleaves the center sequence ACAA, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACAA, wherein the engineered meganuclease comprises a substitution described herein in a second subunit at positions corresponding to positions 50 of SEQ ID NO: 1 (i.e., I-CreI).

In some embodiments described herein is an engineered meganuclease that cleaves the center sequence ACAG, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACAG, wherein the engineered meganuclease comprises a substitution described herein in a second subunit at positions corresponding to positions 50, 72, and 73 of SEQ ID NO: 1 (i.e., I-CreI).

In some embodiments described herein is an engineered meganuclease that cleaves the center sequence ACAT, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACAT, wherein the engineered meganuclease comprises a substitution described herein in a second subunit at positions corresponding to positions 48, 50, and 73 of SEQ ID NO: 1 (i.e., I-CreI).

In some embodiments described herein is an engineered meganuclease that cleaves the center sequence ACGA, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACGA, wherein the engineered meganuclease comprises a substitution described herein in a second subunit at positions corresponding to positions 50 and 72 of SEQ ID NO: 1 (i.e., I-CrcI).

In some embodiments described herein is an engineered meganuclease that cleaves the center sequence ACGC, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACGC, wherein the engineered meganuclease comprises a substitution described herein in a second subunit at positions corresponding to positions 50, 72, and 73 of SEQ ID NO: 1 (i.e., I-CreI).

In some embodiments described herein is an engineered meganuclease that cleaves the center sequence ACGG, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACGG, wherein the engineered meganuclease comprises a substitution described herein in a second subunit at positions corresponding to positions 50, 71, 72, 73, and 73B of SEQ ID NO: 1 (i.e., I-CreI).

In some embodiments described herein is an engineered meganuclease that cleaves the center sequence ACGT, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence ACGT, wherein the engineered meganuclease comprises a substitution described herein in a second subunit at positions corresponding to positions 50, 71, 72, and 73 of SEQ ID NO: 1 (i.e., I-CreI).

In some embodiments described herein is an engineered meganuclease that cleaves the center sequence GCAA, wherein the engineered meganuclease comprises a substitution described herein in a first subunit at positions corresponding to positions 50 of SEQ ID NO: 1 (i.e., I-CrcI). In some embodiments described herein is an engineered meganuclease that cleaves the center sequence GCAA, wherein the engineered meganuclease comprises a substitution described herein in a second subunit at positions corresponding to positions 50 of SEQ ID NO: 1 (i.e., I-CreI).

In some embodiments described herein is an engineered meganuclease that cleaves the center sequence GCAT, wherein the engineered meganuclease comprises a substitution described herein in a first subunit at positions corresponding to positions 50 of SEQ ID NO: 1 (i.e., I-CreI). In some embodiments described herein is an engineered meganuclease that cleaves the center sequence GCAT, wherein the engineered meganuclease comprises a substitution described herein in a second subunit at positions corresponding to positions 48, 50, 72, and 73 of SEQ ID NO: 1 (i.e., I-CreI).

In some embodiments described herein is an engineered meganuclease that cleaves the center sequence GCGA, wherein the engineered meganuclease comprises a substitution described herein in a first subunit at positions corresponding to positions 50 of SEQ ID NO: 1 (i.e., I-CreI). In some embodiments described herein is an engineered meganuclease that cleaves the center sequence GCGA, wherein the engineered meganuclease comprises a substitution described herein in a second subunit at positions corresponding to positions 48, 50, 72, and 73 of SEQ ID NO: 1 (i.e., I-CreI).

In some embodiments described herein is an engineered meganuclease that cleaves the center sequence GCAG, wherein the engineered meganuclease comprises a substitution described herein in a first subunit at positions corresponding to positions 50 of SEQ ID NO: 1 (i.e., I-CreI). In some embodiments described herein is an engineered meganuclease that cleaves the center sequence GCAG, wherein the engineered meganuclease comprises a substitution described herein in a second subunit at positions corresponding to positions 50, 71, 72, and 73 of SEQ ID NO: 1 (i.e., I-CreI).

In some embodiments described herein is an engineered meganuclease that cleaves the center sequence TTAA, wherein the engineered meganuclease comprises a substitution described herein in a first subunit at positions corresponding to positions 50 of SEQ ID NO: 1 (i.e., I-CreI). In some embodiments described herein is an engineered meganuclease that cleaves the center sequence TTAA, wherein the engineered meganuclease comprises a substitution described herein in a second subunit at positions corresponding to positions 50 of SEQ ID NO: 1 (i.e., I-CreI).

In some embodiments described herein is an engineered meganuclease that cleaves the center sequence TTGG, wherein the engineered meganuclease comprises a substitution described herein in a first subunit at positions corresponding to positions 50, 72, and 73 of SEQ ID NO: 1 (i.e., I-CreI). In some embodiments described herein is an engineered meganuclease that cleaves the center sequence TTGG, wherein the engineered meganuclease comprises a substitution described herein in a second subunit at positions corresponding to positions 50, 71, 72, and 73 of SEQ ID NO: 1 (i.e., I-CreI).

In some embodiments described herein is an engineered meganuclease that cleaves the center sequence TCAA, wherein the engineered meganuclease comprises a substitution described herein in a first subunit at positions corresponding to positions 50 of SEQ ID NO: 1 (i.e., I-CreI). In some embodiments described herein is an engineered meganuclease that cleaves the center sequence TCAA, wherein the engineered meganuclease comprises a substitution described herein in a second subunit at positions corresponding to positions 50, 72, 73, and 74 of SEQ ID NO: 1 (i.e., I-CreI).

In some embodiments described herein is an engineered meganuclease that cleaves the center sequence GTAT, wherein the engineered meganuclease comprises a substitution described herein in a first subunit at positions corresponding to positions 50, 72, and 73 of SEQ ID NO: 1 (i.e., I-CreI).

In some embodiments described herein is an engineered meganuclease that cleaves the center sequence GTGG, wherein the engineered meganuclease comprises a substitution described herein in a first subunit at positions corresponding to positions 50, 71, 72, and 73 of SEQ ID NO: 1 (i.e., I-CreI).

In some embodiments described herein is an engineered meganuclease that cleaves the center sequence GTGC, wherein the engineered meganuclease comprises a substitution described herein in a first subunit at positions corresponding to positions 50 of SEQ ID NO: 1 (i.e., I-CreI).

In some embodiments described herein is an engineered meganuclease that cleaves the center sequence GTAG, wherein the engineered meganuclease comprises a substitution described herein in a first subunit at positions corresponding to positions 50, 71, 72, and 73 of SEQ ID NO: 1 (i.e., I-CreI).

In some embodiments described herein is an engineered meganuclease that cleaves the center sequence GTGA, wherein the engineered meganuclease comprises a substitution described herein in a first subunit at positions corresponding to positions 48 and 50 of SEQ ID NO: 1 (i.e., I-CreI).

In some embodiments described herein is an engineered meganuclease that cleaves the center sequence GTAA, wherein the engineered meganuclease comprises a substitution described herein in a first subunit at positions corresponding to positions 50 of SEQ ID NO: 1 (i.e., I-CreI).

In some embodiments described herein is an engineered meganuclease that cleaves the center sequence GTGT, wherein the engineered meganuclease comprises a substitution described herein in a first subunit at positions corresponding to positions 50, 72, and 73 of SEQ ID NO: 1 (i.e., I-CreI).

In addition, it was discovered that certain positions corresponding to positions 48, 50, 71, 72, 73, and 74 are more commonly substituted for particular center sequences. In some embodiments described herein is an engineered meganuclease that cleaves a center sequence comprising ACAA, ACAG, ACAT, ACGA, ACGC, ACGG, ACGT, ATAA, ATAG, ATAT, ATGA, or ATGG, wherein the engineered meganuclease comprises a substitution described herein in a first subunit at positions corresponding to positions 50, 72, and 73 of SEQ ID NO: 1 (i.e., I-CreI) as described herein.

In some embodiments described herein is an engineered meganuclease that cleaves a center sequence comprising ATAA, ATAG, ATAT, ATGA, or ATGG, wherein the engineered meganuclease comprises a substitution described herein in a first subunit at positions corresponding to positions 50, 72, and 73 of SEQ ID NO: 1 (i.e., I-CreI) as described herein.

In some embodiments described herein is an engineered meganuclease that cleaves a center sequence comprising GCAA, GCAT, GCGA, or GCAG, wherein the engineered meganuclease comprises a substitution described herein in a first subunit at positions corresponding to position 50 of SEQ ID NO: 1 (i.e., I-CreI) as described herein. In some embodiments described herein is an engineered meganuclease that cleaves a center sequence comprising GCAA, GCAT, GCGA, or GCAG, wherein the engineered meganuclease comprises a substitution described herein in a first subunit at positions corresponding to positions 50, 72, and 73 of SEQ ID NO: 1 (i.e., I-CreI) as described herein.

In some embodiments described herein is an engineered meganuclease that cleaves a center sequence comprising TTAA and TTGG, wherein the engineered meganuclease comprises a substitution described herein at a position corresponding to position 50 of SEQ ID NO: 1 (i.e., I-CreI) as described herein. In some embodiments described herein is an engineered meganuclease that cleaves a center sequence comprising TCAA, wherein the engineered meganuclease comprises a substitution described herein at a position corresponding to position 50 of SEQ ID NO: 1 (i.e., I-CreI) as described herein.

In some embodiments described herein is an engineered meganuclease that cleaves a center sequence comprising ACAA, ATAA, TTAA, or TCAA, wherein the engineered meganuclease comprises a substitution described herein in a second subunit at positions corresponding to positions 50, 72, and 73 of SEQ ID NO: 1 (i.e., I-CreI) as described herein.

In some embodiments described herein is an engineered meganuclease that cleaves a center sequence comprising ACAA, ATAA, TTAA, or TCAA, wherein the engineered meganuclease comprises a substitution described herein in a second subunit at positions corresponding to positions 50 of SEQ ID NO: 1 (i.e., I-CreI) as described herein.

In some embodiments described herein is an engineered meganuclease that cleaves a center sequence comprising ACAG, ATAG, or GCAG, wherein the engineered meganuclease comprises a substitution described herein in a second subunit at positions corresponding to positions 50, 72, and 73 of SEQ ID NO: 1 (i.e., I-CreI) as described herein. In some embodiments described herein is an engineered meganuclease that cleaves a center sequence comprising ACAG, ATAG, or GCAG, wherein the engineered meganuclease comprises a substitution described herein in a second subunit at positions corresponding to positions 50, 71, 72, and 73 of SEQ ID NO: 1 (i.e., I-CreI) as described herein.

In some embodiments described herein is an engineered meganuclease that cleaves a center sequence comprising ACAT, ATAT, or GCAT, wherein the engineered meganuclease comprises a substitution described herein in a second subunit at positions corresponding to positions 50, 72, and 73 of SEQ ID NO: 1 (i.e., I-CreI) as described herein. In some embodiments described herein is an engineered meganuclease that cleaves a center sequence comprising ACAT, ATAT, or GCAT wherein the engineered meganuclease comprises a substitution described herein in a second subunit at positions corresponding to positions 48, 50, 71, 72, 73, and 74 of SEQ ID NO: 1 (i.e., I-CreI) as described herein. In some embodiments described herein is an engineered meganuclease that cleaves a center sequence comprising ACAT, ATAT, or GCAT wherein the engineered meganuclease comprises a substitution described herein in a second subunit at positions corresponding to positions 48, 50, 72, and 73, of SEQ ID NO: 1 (i.e., I-CreI) as described herein.

In some embodiments described herein is an engineered meganuclease that cleaves a center sequence comprising ACGA, ATGA, or GCGA wherein the engineered meganuclease comprises a substitution described herein in a second subunit at positions corresponding to positions 50 and 72 of SEQ ID NO: 1 (i.e., I-CreI) as described herein. In some embodiments described herein is an engineered meganuclease that cleaves a center sequence comprising ACGA, ATGA, or GCGA wherein the engineered meganuclease comprises a substitution described herein in a second subunit at positions corresponding to positions 48, 50, 72, and 73 of SEQ ID NO: 1 (i.e., I-CreI) as described herein.

In some embodiments described herein is an engineered meganuclease that cleaves a center sequence comprising ACGG, ATGG, or TTGG wherein the engineered meganuclease comprises a substitution described herein in a second subunit at positions corresponding to positions 50, 71, 72, 73, and 73B of SEQ ID NO: 1 (i.e., I-CrcI) as described herein. In some embodiments described herein is an engineered meganuclease that cleaves a center sequence comprising ACGG, ATGG, or TTGG wherein the engineered meganuclease comprises a substitution described herein in a second subunit at positions corresponding to positions 50, 71, 72, and 73 of SEQ ID NO: 1 (i.e., I-CreI) as described herein.

Although the tables above describe residues and substitutions that have been exemplified, the residues of an I-CreI-derived meganuclease can be substituted with additional amino acids to result in an increase in activity for a recognition sequence comprising a specific center sequence. In some embodiments, the modification at a given position is a conservative substitution, such as exchanging one amino acid with another having similar properties. For example, charged amino acids can be substituted with similarly charged amino acids; polar amino acids can be substituted with similarly polar amino acids; amphipathic amino acids can be substituted with similarly amphipathic amino acids; hydrophilic amino acids can be substituted with similarly hydrophilic amino acids; and hydrophobic amino acids can be substituted with similarly hydrophobic amino acids. In addition, the exemplified residues further includes amino acid analogs and non-naturally occurring amino acids, which have similar properties to the exemplified amino acids.

### 2.3 Engineered Meganuclease Variants

Embodiments of the invention encompass the engineered meganucleases described herein, and variants thereof. Further embodiments of the invention encompass isolated polynucleotides comprising a nucleic acid sequence encoding the meganucleases described herein, and variants of such polynucleotides.

Variant polypeptides encompassed by the embodiments are biologically active. That is, they continue to possess the desired biological activity of the native protein; for example, the ability to bind and cleave recognition sequences the recognition sequence, which includes the center sequences described herein, for which they were designed.

Such variants may result, for example, from human manipulation. Biologically active variants of a native polypeptide of the embodiments, or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native I-CrcI derived polypeptide, or native I-CrcI derived subunit, as determined by sequence alignment programs and parameters described elsewhere herein. In some instances, sequence identity can be determined using all positions or, alternatively, only positions other than those described herein that contribute to activity of the engineered meganuclease for a specific center sequence. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein (i.e., SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, and 504-529), such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases of the invention comprise an HVR 1 that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, or 504-529.

In certain embodiments, engineered meganucleases of the invention comprise an HVR2 that has 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 215-270 of SEQ ID NOs: 11-33, 36-43, 46-67, 70-89, 92-118, 121-135, 138-156, 159-183, 186-199, 202-219, 222-243, 246-247, 250-266, 269-291, 294-313, 316-325, 328-330, 333-340, 343-357, 360-389, 392-399, 402-433, 436-462, 465-495, 498-501, or 504-529.

A substantial number of amino acid modifications to the DNA recognition domain of the wild-type I-CreI meganuclease have previously been identified (e.g., U.S. Pat. No. 8,021,867) which, singly or in combination, result in engineered meganucleases with specificities altered at individual bases within the DNA recognition sequence half-site, such that the resulting rationally-designed meganucleases have half-site specificities different from the wild-type enzyme. Table A provides potential substitutions that can be made in an engineered meganuclease monomer or subunit to enhance specificity based on the base present at each half-site position (−1 through −9) of a recognition half-site.

TABLE A

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | 744* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein. An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CrcI (WO 2009001159), a Y, R, K, or D at a residue corresponding to position 66 of I-CreI and/or an E, Q, or K at a residue corresponding to position 80 of I-CreI (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode an engineered meganuclease, or an exogenous nucleic acid molecule, or template nucleic acid of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide its intended activity. For example, variants of an engineered meganuclease would be screened for their ability to preferentially recognize and cleave a recognition sequence comprising a certain center sequence.

### 2.4 Methods to Optimize I-CreI-Derived Meganucleases

Compositions and methods are provided herein to improve the DNA cleavage activity properties of an engineered meganuclease derived from I-CreI by modifying at least one position of an I-CreI derived meganuclease corresponding to positions 48, 50, 71, 72, 73, 73B, and 74 of I-CreI (SEQ ID NO: 1). An improvement of the DNA cleavage activity can refer to an increase of about 10%, 25%, 20%, 25%, 30%, 35%, 40%, 45%, 50%, 55%, 60%, 65%, 70%, 75%, 80%, 85%, 90%, 95%, 100% or more compared to a proper control engineered meganuclease. As used herein a control engineered meganuclease refers to an engineered meganuclease having specificity for the same recognition sequence but lacking modifications from wild type I-CreI, or modifications from an engineered I-CreI-derived meganuclease, at one or more of the positions listed herein. In specific embodiments, a control engineered meganuclease refers to an engineered I-CreI-derived meganuclease having specificity for the same recognition sequence but lacking a modification at one or more positions corresponding to positions 48, 50, 71, 72, 73, 73B, and 74 of I-CreI.

A modification of an engineered meganuclease at a given position can comprise modification of the engineered meganuclease itself, modification of the nucleic acid sequence encoding the engineered meganuclease, or synthetic production of a predetermined amino acid sequence modified from SEQ ID NO: 1 or the sequence of an I-CreI derived meganuclease. Modification of the engineered meganuclease derived from I-CreI itself can be done by any means in the art known to modify amino acid sequence in a site-specific manner.

In certain embodiments, engineered meganucleases derived from I-CreI are modified by altering, in a site-specific manner, the nucleic acid sequence encoding the I-CreI derived meganuclease. Such modifications can be performed on a nucleic acid sequence encoding the first and/or second subunit of the I-CreI derived engineered meganuclease individually. Nucleic acid sequences encoding individual modified subunits can be expressed and modified subunits subsequently assembled with a linker to produce an I-CreI derived homodimer or heterodimer engineered meganuclease. In some embodiments, the nucleic acid sequence encoding an I-CreI derived engineered meganuclease is modified, in a site-specific manner, such that expression of the modified nucleic acid sequence produces a functional modified I-CreI derived engineered meganuclease.

Site-specific modification of nucleic acid sequences can be performed by any method known in the art to produce site-specific cleavage, deletions, and/or substitutions. Methods for producing engineered I-CreI-derived nucleases modified at given sites are known in the art, and include homologous recombination, site-directed mutagenesis, and gene fusion, among others. In specific embodiments, standard techniques for gene editing can be used to engineer I-CreI-derived meganucleases at one or more positions described herein that increase the activity of an engineered meganuclease for a recognition sequence comprising a certain center sequence.

In another aspect of the invention is a method for increasing the cleavage activity of an I-CreI-derived engineered meganuclease that binds and cleaves a meganuclease recognition sequence, wherein said meganuclease recognition sequence comprises a four base pair center sequence comprising a 5′ center sequence half site and a 3′ center sequence half site, wherein the 5′ center sequence half site comprises an AC, AT, CC, CT, GC, GT, TC, or TT pair, and wherein the 3′ center sequence half site comprises an AC, AT, CC, CT, GC, GT, TC, or TT pair, wherein the engineered meganuclease comprises a first subunit and a second subunit, wherein the first subunit and the second subunit each comprise an amino acid sequence derived from SEQ ID NO: 1 (i.e., I-CreI),
wherein the method comprises modifying the first subunit to comprise one or more residues corresponding to positions 48, 50, 71, 72, 73, 73B, and 74 of SEQ ID NO: 1, wherein the modification(s) is/are based on the 5′ center half site of the center sequence, and wherein the modification(s) is/are selected from the residues provided in Table 183 for each of the 5′ center half sites,
and optionally wherein the method comprises modifying the second subunit to comprise one or more residues corresponding to positions 48, 50, 71, 72, 73, 73B, and 74 of SEQ ID NO: 1, wherein the modification(s) is/are based on the 3′ center half site of the center sequence, and wherein the modification(s) is/are selected from the residues provided in Table 183 for each of the 3′ center half sites.

In some embodiments, the 5′ center half site of the center sequence is an AC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site AC pair.

In some embodiments, the 5′ center half site of the center sequence is an AT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site AT pair.

In some embodiments, the 5′ center half site of the center sequence is an CC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site CC pair.

In some embodiments, the 5′ center half site of the center sequence is an CT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site CT pair.

In some embodiments, the 5′ center half site of the center sequence is an GC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site GC pair.

In some embodiments, the 5′ center half site of the center sequence is an GT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site GT pair.

In some embodiments, the 5′ center half site of the center sequence is an TC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site TC pair.

In some embodiments, the 5′ center half site of the center sequence is an TT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 5′ center half site TT pair.

In some embodiments, the 3′ center half site of the center sequence is an AC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site AC pair.

In some embodiments, the 3′ center half site of the center sequence is an AT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site AT pair.

In some embodiments, the 3′ center half site of the center sequence is an CC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site CC pair.

In some embodiments, the 3′ center half site of the center sequence is an CT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site CT pair.

In some embodiments, the 3′ center half site of the center sequence is an GC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site GC pair.

In some embodiments, the 3′ center half site of the center sequence is an GT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site GT pair.

In some embodiments, the 3′ center half site of the center sequence is an TC pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site TC pair.

In some embodiments, the 3′ center half site of the center sequence is an TT pair, and the first subunit is modified to comprise one or more of the residues at positions corresponding to positions 48, 50, 71, 72, 73, 73B, and 74 of SEQ ID NO: 1 (i.e., I-CreI) provided in Table 183 for a 3′ center half site TT pair.

### 2.5 Pharmaceutical Compositions

In some embodiments, the invention provides a pharmaceutical composition comprising a pharmaceutically acceptable carrier and engineered nuclease of the invention, or a pharmaceutically acceptable carrier and an isolated polynucleotide comprising a nucleic acid encoding an engineered nuclease of the invention. In particular, pharmaceutical compositions are provided that comprise a pharmaceutically acceptable carrier and a therapeutically effective amount of a nucleic acid encoding an engineered meganuclease or an engineered meganuclease peptide.

In other embodiments, the invention provides a pharmaceutical composition comprising a pharmaceutically acceptable carrier and a genetically-modified cell of the invention. The genetically modified cell can be delivered to a desired target tissue where the cell.

Pharmaceutical compositions of the invention can be useful for treating a subject having a disease in a subject in need of treatment thereof in accordance with the present invention.

Such pharmaceutical compositions can be prepared in accordance with known techniques. See. e.g., Remington, The Science And Practice of Pharmacy (21st ed., Philadelphia, Lippincott, Williams & Wilkins, 2005). In the manufacture of a pharmaceutical formulation according to the invention, nuclease polypeptides (or DNA/RNA encoding the same or cells expressing the same) are typically admixed with a pharmaceutically acceptable carrier, and the resulting composition is administered to a subject. The carrier must be acceptable in the sense of being compatible with any other ingredients in the formulation and must not be deleterious to the subject. In some embodiments, pharmaceutical compositions of the invention can further comprise one or more additional agents or biological molecules useful in the treatment of a disease in the subject. Likewise, the additional agent(s) and/or biological molecule(s) can be co-administered as a separate composition.

In particular embodiments of the invention, the pharmaceutical composition comprises viral vectors comprising a nucleic acid sequence encoding an engineered nuclease described herein. Such vectors are known in the art and include retroviral vectors, lentiviral vectors, adenoviral vectors, and adeno-associated virus (AAV) vectors (reviewed in Vannucci, et al. (2013 New Microbiol. 36:1-22). Recombinant AAV vectors useful in the invention can have any serotype that allows for transduction of the virus into a target cell type and expression of the nuclease gene by the target cell. For example, in some embodiments, recombinant AAV vectors have a serotype of AAV2, AAV6, AAV8, or AAV9. In some embodiments, the viral vectors are injected directly into target tissues. In alternative embodiments, the viral vectors are delivered systemically via the circulatory system. It is known in the art that different AAV vectors tend to localize to different tissues. In liver target tissues, effective transduction of hepatocytes has been shown, for example, with AAV scrotypes 2, 8, and 9 (Sands (2011) Methods Mol. Biol. 807:141-157). Accordingly, in some embodiments, the AAV serotype is AAV2. In alternative embodiments, the AAV serotype is AAV6. In other embodiments, the AAV serotype is AAV8. In still other embodiments, the AAV serotype is AAV9. AAV vectors can also be self-complementary such that they do not require second-strand DNA synthesis in the host cell (McCarty, et al. (2001) Gene Ther. 8:1248-54). Nucleic acids delivered by recombinant AAV vectors can include left (5′) and right (3′) inverted terminal repeats.

In particular embodiments of the invention, the pharmaceutical composition comprises one or more mRNAs described herein (e.g., mRNAs encoding engineered nucleases) formulated within lipid nanoparticles.

The selection of cationic lipids, non-cationic lipids and/or lipid conjugates which comprise the lipid nanoparticle, as well as the relative molar ratio of such lipids to each other, is based upon the characteristics of the selected lipid(s), the nature of the intended target cells, and the characteristics of the mRNA to be delivered. Additional considerations include, for example, the saturation of the alkyl chain, as well as the size, charge, pH, pKa, fusogenicity and toxicity of the selected lipid(s). Thus, the molar ratios of each individual component may be adjusted accordingly.

The lipid nanoparticles for use in the method of the invention can be prepared by various techniques which are presently known in the art. Nucleic acid-lipid particles and their method of preparation are disclosed in, for example, U.S., Patent Publication Nos. 20040142025 and 20070042031, the disclosures of which are herein incorporated by reference in their entirety for all purposes.

Selection of the appropriate size of lipid nanoparticles must take into consideration the site of the target cell and the application for which the lipid nanoparticles is being made. Generally, the lipid nanoparticles will have a size within the range of about 25 to about 500 nm. In some embodiments, the lipid nanoparticles have a size from about 50 nm to about 300 nm or from about 60 nm to about 120 nm. The size of the lipid nanoparticles may be determined by quasi-electric light scattering (QELS) as described in Bloomfield, Ann. Rev. Biophys. Bioeng., 10:421\hat{ }150 (1981), incorporated herein by reference. A variety of methods are known in the art for producing a population of lipid nanoparticles of particular size ranges, for example, sonication or homogenization. One such method is described in U.S. Pat. No. 4,737,323, incorporated herein by reference.

Some lipid nanoparticles contemplated for use in the invention comprise at least one cationic lipid, at least one non-cationic lipid, and at least one conjugated lipid. In more particular examples, lipid nanoparticles can comprise from about 50 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology. In other particular examples, lipid nanoparticles can comprise from about 40 mol % to about 85 mol % of a cationic lipid, from about 13 mol % to about 49.5 mol % of a non-cationic lipid, and from about 0.5 mol % to about 10 mol % of a lipid conjugate, and are produced in such a manner as to have a non-lamellar (i.e., non-bilayer) morphology.

Cationic lipids can include, for example, one or more of the following: palmitoyl-oleoyl-nor-arginine (PONA), MPDACA, GUADACA, ((6Z,9Z,28Z,31Z)-heptatriaconta-6,9,28,31-tetraen-19-yl 4-(dimethylamino)butanoate) (MC3), LenMC3, CP-LenMC3, γ-LenMC3, CP-γ-LenMC3, MC3MC, MC2MC, MC3 Ether, MC4 Ether, MC3 Amide, Pan-MC3, Pan-MC4 and Pan MC5, 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA; “XTC2”), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinolcoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminocthane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethy-1-(cis,cis-9′,1-2′-octadecadienoxy)propane (CpLinDMA), N,N-dimethyl-3,4-dioleyloxybenzylamine (DMOBA), 1,2-N,N′-diolcylcarbamyl-3-dimethylaminopropane (DOcarbDAP), 1,2-N,N′-dilinolcylcarbamyl-3-dimethylaminopropane (DLincarbDAP), or mixtures thereof. The cationic lipid can also be DLinDMA, DLin-K-C2-DMA (“XTC2”), MC3, LenMC3, CP-LenMC3, γ-LenMC3, CP-γ-LenMC3, MC3MC, MC2MC, MC3 Ether, MC4 Ether, MC3 Amide, Pan-MC3, Pan-MC4, Pan MC5, or mixtures thereof.

In various embodiments, the cationic lipid comprises from about 50 mol % to about 90 mol %, from about 50 mol % to about 85 mol %, from about 50 mol % to about 80 mol %, from about 50 mol % to about 75 mol %, from about 50 mol % to about 70 mol %, from about 50 mol % to about 65 mol %, or from about 50 mol % to about 60 mol % of the total lipid present in the particle.

In other embodiments, the cationic lipid comprises from about 40 mol % to about 90 mol %, from about 40 mol % to about 85 mol %, from about 40 mol % to about 80 mol %, from about 40 mol % to about 75 mol %, from about 40 mol % to about 70 mol %, from about 40 mol % to about 65 mol %, or from about 40 mol % to about 60 mol % of the total lipid present in the particle.

The non-cationic lipid may comprise, e.g., one or more anionic lipids and/or neutral lipids. In particular embodiments, the non-cationic lipid comprises one of the following neutral lipid components: (1) cholesterol or a derivative thereof; (2) a phospholipid; or (3) a mixture of a phospholipid and cholesterol or a derivative thereof. Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The phospholipid may be a neutral lipid including, but not limited to, dipalmitoylphosphatidylcholine (DPPC), distearoylphosphatidylcholine (DSPC), dioleoylphosphatidylethanolamine (DOPE), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), and mixtures thereof. In certain particular embodiments, the phospholipid is DPPC, DSPC, or mixtures thereof.

In some embodiments, the non-cationic lipid (e.g., one or more phospholipids and/or cholesterol) comprises from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle. When the non-cationic lipid is a mixture of a phospholipid and cholesterol or a cholesterol derivative, the mixture may comprise up to about 40, 50, or 60 mol % of the total lipid present in the particle.

The conjugated lipid that inhibits aggregation of particles may comprise, e.g., one or more of the following: a polyethyleneglycol (PEG)-lipid conjugate, a polyamide (ATTA)-lipid conjugate, a cationic-polymer-lipid conjugates (CPLs), or mixtures thereof. In one particular embodiment, the nucleic acid-lipid particles comprise either a PEG-lipid conjugate or an ATTA-lipid conjugate. In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEG-DAA conjugate may be PEG-dilauryloxypropyl (C12), a PEG-dimyristyloxypropyl (C14), a PEG-dipalmityloxypropyl (C16), a PEG-distearyloxypropyl (C18), or mixtures thereof.

Additional PEG-lipid conjugates suitable for use in the invention include, but are not limited to, mPEG2000-1,2-di-O-alkyl-sn3-carbomoylglyceride (PEG-C-DOMG). The synthesis of PEG-C-DOMG is described in PCT Application No. PCT/US08/88676. Yet additional PEG-lipid conjugates suitable for use in the invention include, without limitation, 1-[8′-(1,2-dimyristoyl-3-propanoxy)-carboxamido-3′,6′-dioxaoctanyl]carbamoyl-ω-methyl-poly(ethylene glycol) (2KPEG-DMG). The synthesis of 2KPEG-DMG is described in U.S. Pat. No. 7,404,969.

In some cases, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

In other embodiments, the composition comprises amphoteric liposomes, which contain at least one positive and at least one negative charge carrier, which differs from the positive one, the isoelectric point of the liposomes being between 4 and 8. This objective is accomplished owing to the fact that liposomes are prepared with a pH-dependent, changing charge.

Liposomal structures with the desired properties are formed, for example, when the amount of membrane-forming or membrane-based cationic charge carriers exceeds that of the anionic charge carriers at a low pH and the ratio is reversed at a higher pH. This is always the case when the ionizable components have a pKa value between 4 and 9. As the pH of the medium drops, all cationic charge carriers are charged more and all anionic charge carriers lose their charge.

Cationic compounds useful for amphoteric liposomes include those cationic compounds previously described herein above. Without limitation, strongly cationic compounds can include, for example: DC-Chol 3-β-[N—(N′,N′-dimethylmethane) carbamoyl]cholesterol, TC-Chol 3-β-[N—(N′,N′,N′-trimethylaminoethane) carbamoyl cholesterol. BGSC bisguanidinium-spermidine-cholesterol, BGTC bis-guadinium-tren-cholesterol, DOTAP (1,2-dioleoyloxypropyl)-N,N,N-trimethylammonium chloride, DOSPER (1,3-dioleoyloxy-2-(6-carboxy-spermyl)-propylamide, DOTMA (1,2-dioleoyloxypropyl)-N,N,N-trimethylammonium chloride) (Lipofectin®), DORIE 1,2-dioleoyloxypropyl)-3-dimethylhydroxyethylammonium bromide, DOSC (1,2-dioleoyl-3-succinyl-sn-glyceryl choline ester), DOGSDSO (1,2-dioleoyl-sn-glycero-3-succinyl-2-hydroxyethyl disulfide omithine), DDAB dimethyldioctadecylammonium bromide, DOGS ((C18)2GlySper3+) N,N-dioctadecylamido-glycol-spermin (Transfectam®) (C18)2Gly+N,N-dioctadecylamido-glycine, CTAB cctyltrimcthylarnmonium bromide, CpyC cctylpyridinium chloride. DOEPC 1,2-dioleoly-sn-glycero-3-ethylphosphocholine or other O-alkyl-phosphatidylcholine or ethanolamines, amides from lysine, arginine or ornithine and phosphatidyl ethanolamine.

Examples of weakly cationic compounds include, without limitation: His-Chol (histaminyl-cholesterol hemisuccinate), Mo-Chol (morpholine-N-ethylamino-cholesterol hemisuccinate), or histidinyl-PE.

Examples of neutral compounds include, without limitation: cholesterol, ceramides, phosphatidyl cholines, phosphatidyl ethanolamines, tetraether lipids, or diacyl glycerols.

Anionic compounds useful for amphoteric liposomes include those non-cationic compounds previously described herein. Without limitation, examples of weakly anionic compounds can include: CHEMS (cholesterol hemisuccinate), alkyl carboxylic acids with 8 to 25 carbon atoms, or diacyl glycerol hemisuccinate. Additional weakly anionic compounds can include the amides of aspartic acid, or glutamic acid and PE as well as PS and its amides with glycine, alanine, glutamine, asparagine, serine, cysteine, threonine, tyrosine, glutamic acid, aspartic acid or other amino acids or aminodicarboxylic acids. According to the same principle, the esters of hydroxycarboxylic acids or hydroxydicarboxylic acids and PS are also weakly anionic compounds.

In some embodiments, amphoteric liposomes contain a conjugated lipid, such as those described herein above. Particular examples of useful conjugated lipids include, without limitation, PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Some particular examples are PEG-modified diacylglycerols and dialkylglycerols.

In some embodiments, the neutral lipids comprise from about 10 mol % to about 60 mol %, from about 15 mol % to about 60 mol %, from about 20 mol % to about 60 mol %, from about 25 mol % to about 60 mol %, from about 30 mol % to about 60 mol %, from about 10 mol % to about 55 mol %, from about 15 mol % to about 55 mol %, from about 20 mol % to about 55 mol %, from about 25 mol % to about 55 mol %, from about 30 mol % to about 55 mol %, from about 13 mol % to about 50 mol %, from about 15 mol % to about 50 mol % or from about 20 mol % to about 50 mol % of the total lipid present in the particle.

In some cases, the conjugated lipid that inhibits aggregation of particles (e.g., PEG-lipid conjugate) comprises from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle. Typically, in such instances, the PEG moiety has an average molecular weight of about 2,000 Daltons. In other cases, the conjugated lipid that inhibits aggregation of particles (e.g., PEG-lipid conjugate) may comprise from about 5.0 mol % to about 10 mol %, from about 5 mol % to about 9 mol %, from about 5 mol % to about 8 mol %, from about 6 mol % to about 9 mol %, from about 6 mol % to about 8 mol %, or about 5 mol %, 6 mol %, 7 mol %, 8 mol %, 9 mol %, or 10 mol % (or any fraction thereof or range therein) of the total lipid present in the particle. Typically, in such instances, the PEG moiety has an average molecular weight of about 750 Daltons.

Considering the total amount of neutral and conjugated lipids, the remaining balance of the amphoteric liposome can comprise a mixture of cationic compounds and anionic compounds formulated at various ratios. The ratio of cationic to anionic lipid may selected in order to achieve the desired properties of nucleic acid encapsulation, zeta potential, pKa, or other physicochemical property that is at least in part dependent on the presence of charged lipid components.

### 2.6 Methods for Producing Recombinant Viruses

In some embodiments, the invention provides recombinant viruses (i.e., recombinant viral vectors; e.g., recombinant AAVs) for use in the methods of the invention. Recombinant AAVs are typically produced in mammalian cell lines such as HEK-293. Because the viral cap and rep genes are removed from the recombinant virus to prevent its self-replication to make room for the therapeutic gene(s) to be delivered (e.g. the nuclease gene), it is necessary to provide these in trans in the packaging cell line. In addition, it is necessary to provide the “helper” (e.g. adenoviral) components necessary to support replication (Cots et al. (2013), Curr. Gene Ther. 13(5): 370-81). Frequently, recombinant AAVs are produced using a triple-transfection in which a cell line is transfected with a first plasmid encoding the “helper” components, a second plasmid comprising the cap and rep genes, and a third plasmid comprising the viral ITRs containing the intervening DNA sequence to be packaged into the virus. Viral particles comprising a genome (ITRs and intervening gene(s) of interest) encased in a capsid are then isolated from cells by freeze-thaw cycles, sonication, detergent, or other means known in the art. Particles are then purified using cesium-chloride density gradient centrifugation or affinity chromatography and subsequently delivered to the gene(s) of interest to cells, tissues, or an organism such as a human patient.

Because recombinant AAV particles are typically produced (manufactured) in cells, precautions must be taken in practicing the current invention to ensure that the engineered nuclease is not expressed in the packaging cells. Because the viral genomes of the invention may comprise a recognition sequence for the nuclease, any nuclease expressed in the packaging cell line may be capable of cleaving the viral genome before it can be packaged into viral particles. This will result in reduced packaging efficiency and/or the packaging of fragmented genomes. Several approaches can be used to prevent nuclease expression in the packaging cells.

The nuclease can be placed under the control of a tissue-specific promoter that is not active in the packaging cells. For example, if a viral vector is developed for delivery of a nuclease gene(s) to muscle tissue, a muscle-specific promoter can be used. Examples of muscle-specific promoters include C5-12 (Liu, et al. (2004) Hum Gene Ther. 15:783-92), the muscle-specific creatine kinase (MCK) promoter (Yuasa, et al. (2002) Gene Ther. 9:1576-88), or the smooth muscle 22 (SM22) promoter (Haase, et al. (2013) BMC Biotechnol. 13:49-54). Examples of CNS (neuron)-specific promoters include the NSE, Synapsin, and MeCP2 promoters (Lentz, et al. (2012) Neurobiol Dis. 48:179-88). Examples of liver-specific promoters include, for example, albumin promoters (such as Palb), human α1-antitrypsin (such as Pa1AT), and hemopexin (such as Phpx) (Kramer et al., (2003) Mol. Therapy 7:375-85), hybrid liver-specific promoter (hepatic locus control region from ApoE gene (ApoE-HCR) and a liver-specific alpha1-antitrypsin promoter), human thyroxine binding globulin (TBG) promoter, and apolipoprotcin A-Il promoter. Examples of cyc-specific promoters include opsin, and corneal epithelium-specific K12 promoters (Martin et al. (2002) Methods (28): 267-75) (Tong et al., (2007) J Gene Med. 9:956-66). These promoters, or other tissue-specific promoters known in the art, are not highly-active in HEK-293 cells and, thus, will not be expected to yield significant levels of nuclease gene expression in packaging cells when incorporated into viral vectors of the present invention. Similarly, the recombinant viruses of the present invention contemplate the use of other cell lines with the use of incompatible tissue specific promoters (i.e., the well-known HeLa cell line (human epithelial cell) and using the liver-specific hemopexin promoter). Other examples of tissue specific promoters include: synovial sarcomas PDZD4 (cerebellum), C6 (liver), ASB5 (muscle), PPP1R12B (heart), SLC5A12 (kidney), cholesterol regulation APOM (liver), ADPRHL1 (heart), and monogenic malformation syndromes TP73L (muscle). (Jacox et al., (2010), PLoS One v.5(8):e12274).

Alternatively, the recombinant virus can be packaged in cells from a different species in which the nuclease is not likely to be expressed. For example, viral particles can be produced in microbial, insect, or plant cells using mammalian promoters, such as the well-known cytomegalovirus- or SV40 virus-early promoters, which are not active in the non-mammalian packaging cells. In a particular embodiment, viral particles are produced in insect cells using the baculovirus system as described by Gao, et al. (Gao et al. (2007). J. Biotechnol. 131(2):138-43). A nuclease under the control of a mammalian promoter is unlikely to be expressed in these cells (Airenne et al. (2013). Mol. Ther. 21(4):739-49). Moreover, insect cells utilize different mRNA splicing motifs than mammalian cells. Thus, it is possible to incorporate a mammalian intron, such as the human growth hormone (HGH) intron or the SV40 large T antigen intron, into the coding sequence of a nuclease. Because these introns are not spliced efficiently from pre-mRNA transcripts in insect cells, insect cells will not express a functional nuclease and will package the full-length genome. In contrast, mammalian cells to which the resulting recombinant AAV particles are delivered will properly splice the pre-mRNA and will express functional nuclease protein. Haifeng Chen has reported the use of the HGH and SV40 large T antigen introns to attenuate expression of the toxic proteins barnase and diphtheria toxin fragment A in insect packaging cells, enabling the production of recombinant AAV vectors carrying these toxin genes (Chen, H (2012) Mol Ther Nucleic Acids. 1(11): e57).

The nuclease gene can be operably linked to an inducible promoter such that a small-molecule inducer is required for nuclease expression. Examples of inducible promoters include the Tet-On system (Clontech; Chen et al. (2015), BMC Biotechnol. 15(1):4)) and the RheoSwitch system (Intrexon; Sowa et al. (2011), Spine, 36(10): E623-8). Both systems, as well as similar systems known in the art, rely on ligand-inducible transcription factors (variants of the Tet Repressor and Ecdysone receptor, respectively) that activate transcription in response to a small-molecule activator (Doxycycline or Ecdysone, respectively). Practicing the current invention using such ligand-inducible transcription activators includes: 1) placing the nuclease gene under the control of a promoter that responds to the corresponding transcription factor, the nuclease gene having (a) binding site(s) for the transcription factor; and 2) including the gene encoding the transcription factor in the packaged viral genome. The latter step is necessary because the nuclease will not be expressed in the target cells or tissues following recombinant AAV delivery if the transcription activator is not also provided to the same cells. The transcription activator then induces nuclease gene expression only in cells or tissues that are treated with the cognate small-molecule activator. This approach is advantageous because it enables nuclease gene expression to be regulated in a spatio-temporal manner by selecting when and to which tissues the small-molecule inducer is delivered. However, the requirement to include the inducer in the viral genome, which has significantly limited carrying capacity, creates a drawback to this approach.

In another particular embodiment, recombinant AAV particles are produced in a mammalian cell line that expresses a transcription repressor that prevents expression of the nuclease. Transcription repressors are known in the art and include the Tet-Repressor, the Lac-Repressor, the Cro repressor, and the Lambda-repressor. Many nuclear hormone receptors such as the ecdysone receptor also act as transcription repressors in the absence of their cognate hormone ligand. To practice the current invention, packaging cells are transfected/transduced with a vector encoding a transcription repressor and the nuclease gene in the viral genome (packaging vector) is operably linked to a promoter that is modified to comprise binding sites for the repressor such that the repressor silences the promoter. The gene encoding the transcription repressor can be placed in a variety of positions. It can be encoded on a separate vector; it can be incorporated into the packaging vector outside of the ITR sequences; it can be incorporated into the cap/rep vector or the adenoviral helper vector, or it can be stably integrated into the genome of the packaging cell such that it is expressed constitutively. Methods to modify common mammalian promoters to incorporate transcription repressor sites are known in the art. For example, Chang and Roninson modified the strong, constitutive CMV and RSV promoters to comprise operators for the Lac repressor and showed that gene expression from the modified promoters was greatly attenuated in cells expressing the repressor (Chang and Roninson (1996), Gene 183:137-42). The use of a non-human transcription repressor ensures that transcription of the nuclease gene will be repressed only in the packaging cells expressing the repressor and not in target cells or tissues transduced with the resulting recombinant AAV.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Characterization of Engineered Meganucleases with Specificity for Recognition Sequences Having Particular Four Base Pair Center Sequences

These studies were conducted to identify positions and residues within I-CreI-derived subunits that affect the activity of the nuclease for recognition sequences having specific four base pair center sequences. Those center sequences evaluated herein include: ACAA, ACAG, ACAT, ACGA, ACGC, ACGG, ACGT, ATAA, ATAG, ATAT, ATGA, ATGG, TTGG, GCAA, GCAT, GCGA, GCAG, TCAA, TTAA, GTAA, GTAG, GTAT, GTGA, GTGC, GTGG, and GTGT.

To perform these studies, a system was developed that utilized an I-CreI-derived meganuclease referred to as LOX 3-4x.109, the sequence of which is set forth in SEQ ID NO: 8. Previously, LOX 3-4x.109 nuclease was engineered at particular positions such that it has specificity for a recognition sequence referred to as LOX 3-4, the sequence of which is set forth in SEQ ID NO: 6. In these studies, both the LOX 3-4 recognition sequence, and the LOX 3-4x.109 meganuclease, were further modified. The LOX 3-4 recognition sequence was modified to replace its center sequence (ACAT) with one of the center sequences disclosed above. These modified LOX 3-4 recognition sequences are provided in Table 111 below.

TABLE 111
LOX 3-4 Recognition Sequence Modified
With Different Center Sequences

| Recognition Sequence | SEQ ID NO: |
|---|---|
| LOX 3-4 ACAA | 9 |
| LOX 3-4 ACAG | 34 |
| LOX 3-4 ACAT | 44 |
| LOX 3-4 ACGA | 68 |
| LOX 3-4 ACGC | 90 |
| LOX 3-4 ACGG | 119 |
| LOX 3-4 ACGT | 136 |
| LOX 3-4 ATAA | 157 |
| LOX 3-4 ATAG | 184 |
| LOX 3-4 ATAT | 200 |
| LOX 3-4 ATGA | 220 |
| LOX 3-4 ATGG | 244 |
| LOX 3-4 TTGG | 248 |
| LOX 3-4 GCAA | 267 |
| LOX 3-4 GCAT | 292 |
| LOX 3-4 GCGA | 314 |
| LOX 3-4 GCAG | 326 |
| LOX 3-4 TCAA | 331 |
| LOX 3-4 TTAA | 341 |
| LOX 3-4 GTAA | 358 |
| LOX 3-4 GTAG | 390 |
| LOX 3-4 GTAT | 400 |
| LOX 3-4 GTGA | 434 |
| LOX 3-4 GTGC | 463 |
| LOX 3-4 GTGG | 496 |
| LOX 3-4 GTGT | 504 |

The LOX 3-4x.109 meganuclease was then modified in one subunit, or in both subunits, to identify positions and residues that may affect the ability of the nuclease to recognize and cleave the modified LOX 3-4 recognition sequence. Structurally, LOX 3-4x.109 comprises an N-terminal nuclease-localization signal derived from SV40, a first I-CreI-derived subunit, a linker sequence, and a second I-CreI-derived subunit. One subunit binds to the LOX 3 recognition half-site of SEQ ID NO: 6, while the other subunit binds to the LOX4 recognition half-site of SEQ ID NO: 6. The first and second subunits of LOX 3-4x.109 each comprise a 56 base pair hypervariable region, referred to as HVR1 and HVR2, respectively. The HVR1 region in the first subunit consists of residues 24-79 of SEQ ID NO: 8, whereas the HVR2 region in the second subunit consists of residues 215-270 of SEQ ID NO: 8. In these studies, LOX 3-4x.109 was modified at positions both within the HVR regions, and outside the HVR regions, to generate novel meganucleases with altered activity, affinity, and/or specificity. Notably, the positions in the LOX 3-4x.109 meganuclease that were originally modified from wild-type I-CreI to confer specificity for each subunit for LOX 3-4 were not further modified. As such, any alterations in activity observed in these studies demonstrate are related to the center sequence.

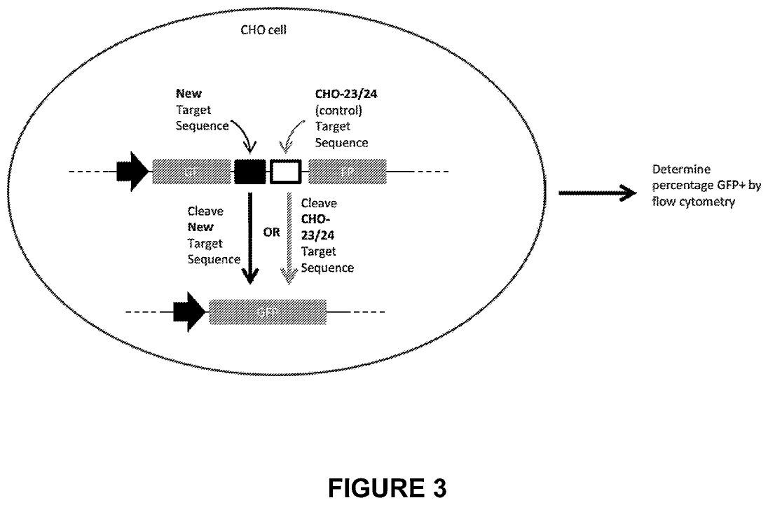

A CHO cell reporter system (see WO/2012/167192, FIG. 3) was used to determine whether the engineered meganucleases generated in these studies could recognize and cleave the modified LOX 3-4 recognition sequences in Table 87. To perform the assay, a pair of CHO cell reporter lines were produced, which carried a non-functional Green Fluorescent Protein (GFP) gene expression cassette integrated into the genome of the cell. The GFP gene in each cell line was interrupted by a pair of recognition sequences such that intracellular cleavage of either recognition sequence by a meganuclease would stimulate a homologous recombination event resulting in a functional GFP gene. In both cell lines, one of the recognition sequences was derived from the LOX 3-4 recognition sequence (i.e., those sequences disclosed in Table 87), and the second recognition sequence was specifically recognized by a control meganuclease called “CHO 23/24.” CHO reporter cells comprising a recognition sequence derived from the LOX 3-4 recognition sequence and the CHO 23/24 recognition sequence are referred to herein as “test cells.”

Test cells were transfected with plasmid DNA encoding an engineered meganuclease which had been optimized for a corresponding center sequence. For example, DNA encoding an engineered meganuclease optimized against an ATAT center sequence would be transfected into CHO cells in which the integrated LOX 3-4 recognition sequence comprises an ATAT center sequence. In some of the experiments, the LOX 3-4x.109 engineered meganuclease (SEQ ID NO: 8) was transfected as an additional control for cutting of modified LOX 3-4 recognition sequences. 4e⁵ CHO cells were transfected with 50 ng of plasmid DNA in a 96-well plate using Lipofectamine 2000 (Thermofisher) according to the manufacturer's instructions. At 48 hours post-transfection, cells were evaluated by flow cytometry to determine the percentage of GFP-positive cells compared to an untransfected negative control (LOX 3-4 bs). In some instances, substitutions of particular residues at certain positions, including one or more positions corresponding to positions 48, 50, 71, 72, 73, and 74 of I-CreI, was found to produce GFP-positive cells in cell lines comprising the modified LOX 3-4 recognition sequences provided in table 87, at frequencies significantly exceeding the negative control and comparable to or exceeding the CHO 23/24 positive control (see, Examples 2-27).

### Example 2

### Engineered Meganucleases Cleaving Recognition Sequences Containing an ACAA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ACAA center sequence (SEQ ID NO: 9) in the CHO reporter assay according to Example 1. The substitutions in each subunit are provided in Tables 112 and 113, respectively. The results of the CHO reporter assay are provided in Table 114.

Following the modifications shown below, substantial improvements in cleavage of the recognition sequence having the ACAA four base pair center sequence were observed.

TABLE 112
Meganucleases Optimized for ACAA Center
Sequence (First Subunit - Lox3)

| | | I-CreI Position | | | | | | | |
|---|---|---|---|---|---|---|---|---|---|
| | | 19 | 48 | 50 | 71 | 72 | 73 | 80 | 139 |
| Nuclease | SEQ ID | 19 | 48 | 50 | 71 | 72 | 73 | 80 | 139 |
| x.109 | 8 | A | K | Q | G | R | A | Q | K |
| m.680 | 11 | G | K | C | G | R | A | Q | K |
| m.683 | 12 | A | K | R | G | R | A | Q | K |
| m.684 | 13 | A | K | R | G | R | A | Q | K |
| m.691 | 14 | G | K | R | G | R | A | Q | K |
| m.693 | 15 | G | K | R | G | R | A | E | K |
| m.701 | 16 | G | K | R | G | R | A | Q | K |
| m.708 | 17 | G | K | C | R | Q | C | E | K |
| m.714 | 18 | A | K | R | G | R | A | Q | K |
| m.731 | 19 | A | K | R | G | R | A | Q | K |
| m.739 | 20 | G | K | R | G | R | A | Q | K |
| m.741 | 21 | G | K | T | G | R | A | Q | K |
| m.742 | 22 | G | K | R | G | R | A | Q | K |
| m.743 | 23 | G | K | T | G | R | A | Q | K |
| m.744 | 24 | G | K | R | G | R | A | Q | K |
| m.747 | 25 | G | K | K | G | R | A | E | K |
| m.750 | 26 | A | K | C | G | R | A | Q | K |
| m.756 | 27 | A | K | R | G | R | A | Q | K |
| m.757 | 28 | A | K | C | G | R | A | Q | K |
| m.759 | 29 | G | K | S | G | R | A | Q | K |
| m.762 | 30 | G | K | R | G | R | A | Q | K |
| m.765 | 31 | A | K | R | G | R | A | Q | R |
| m.770 | 32 | A | L | R | G | R | A | Q | K |
| m.771 | 33 | G | K | R | G | R | A | Q | K |

TABLE 113
Meganucleases Optimized for ACAA Center Sequence (Second Subunit - Lox4)

| | | I-CreI Position | | | | | | | | | | | |
|---|---|---|---|---|---|---|---|---|---|---|---|---|---|
| | SEQ | 19 | 48 | 50 | 66 | 71 | 72 | 73 | 74 | 80 | 92 | 117 | 139 |
| Nuclease | ID | 210 | 239 | 241 | 257 | 262 | 263 | 264 | 265 | 271 | 283 | 308 | 330 |
| x.109 | 8 | G | H | S | Y | S | T | H | S | Q | Q | E | K |
| m.680 | 11 | A | K | C | Y | G | R | V | T | E | Q | E | R |
| m.683 | 12 | G | T | R | Y | G | R | I | S | E | Q | E | K |
| m.684 | 13 | G | S | R | Y | G | T | I | T | E | Q | E | R |
| m.691 | 14 | A | S | K | Y | G | N | I | S | E | Q | E | K |
| m.693 | 15 | A | A | K | Y | G | S | V | T | Q | Q | E | R |
| m.701 | 16 | A | K | E | Y | A | G | I | S | Q | Q | E | R |
| m.708 | 17 | A | S | C | Y | G | S | V | S | Q | Q | E | R |
| m.714 | 18 | G | A | R | Y | G | R | I | S | E | Q | E | K |
| m.731 | 19 | G | S | E | Y | G | R | I | A | Q | Q | E | K |
| m.739 | 20 | A | S | T | Y | G | R | I | S | E | Q | E | R |
| m.741 | 21 | A | S | K | Y | G | R | V | A | E | R | E | R |
| m.742 | 22 | A | S | K | Y | G | S | I | S | E | Q | E | R |
| m.743 | 23 | A | A | E | Y | G | R | I | A | Q | Q | E | R |
| m.744 | 24 | S | S | K | Y | G | R | I | A | E | Q | E | R |
| m.747 | 25 | A | S | R | Y | G | A | I | S | E | Q | G | K |
| m.750 | 26 | G | A | R | Y | G | R | I | S | E | Q | E | R |
| m.756 | 27 | G | S | K | C | G | T | I | T | E | Q | E | R |
| m.757 | 28 | G | K | E | Y | G | S | V | S | E | Q | E | R |
| m.759 | 29 | A | S | K | Y | G | R | I | T | Q | Q | E | R |
| m.762 | 30 | A | A | K | Y | G | P | I | A | E | Q | E | R |
| m.765 | 31 | G | S | R | Y | G | R | I | A | E | Q | E | R |
| m.770 | 32 | G | A | K | Y | G | R | V | S | E | Q | E | K |
| m.771 | 33 | A | T | K | Y | G | P | V | A | Q | Q | E | R |

TABLE 114
CHO iGFFP Assay ATAA Center Sequence Cleavage

| | SEQ ID | |
|---|---|---|
| Meganuclease | NO: | GFP % |
| LOX 3-4bs | — | 0.25% |
| CHO 23/24 | — | 11.34% |
| x.109 | 8 | 0.68 |
| m.680 | 11 | 7.63 |
| m.683 | 12 | 7.62 |
| m.684 | 13 | 9.14 |
| m.691 | 14 | 7.10 |
| m.693 | 15 | 7.67 |
| m.701 | 16 | 8.36 |
| m.708 | 17 | 6.73 |
| m.714 | 18 | 6.62 |
| m.731 | 19 | 6.76 |
| m.739 | 20 | 6.67 |
| m.741 | 21 | 7.82 |
| m.742 | 22 | 7.79 |
| m.743 | 23 | 7.05 |
| m.744 | 24 | 6.89 |
| m.747 | 25 | 7.11 |
| m.750 | 26 | 9.32 |
| m.756 | 27 | 8.21 |
| m.757 | 28 | 9.27 |
| m.759 | 29 | 7.98 |
| m.762 | 30 | 8.87 |
| m.765 | 31 | 9.32 |
| m.770 | 32 | 7.23 |
| m.771 | 33 | 7.61 |

### Example 3

### Engineered Meganucleases Cleaving Recognition Sequences Containing an ACAG Four Base Pair Center Sequence

Novel engineered meganucleases derived from the LOX 3-4x.109 meganuclease were prepared by making amino acid substitutions at one or more positions in the first subunit and one or more positions in the second subunit. In addition, two engineered meganucleases were generated that inserted an additional R residue following position 264, which corresponds to position 73 of wild-type I-CreI. These engineered meganucleases were then evaluated for cleavage of the LOX 3-4 recognition sequence modified to have an ACAG center sequence (SEQ ID NO: 34) in the CHO reporter assay according to Example 1. The substitutions in each subunit are provided in Tables 115 and 116, respectively. The results of the CHO reporter assay are provided in Table 117.

Following the modifications shown below, substantial improvements in cleavage of the recognition sequence having the ACAG four base pair center sequence were observed.

TABLE 115
Meganucleases Optimized for ACAG Center
Sequence (First Subunit - Lox3)

| | | I-CreI Position | | | | | | |
|---|---|---|---|---|---|---|---|---|
| | SEQ | 19 | 50 | 54 | 71 | 72 | 73 | 80 |
| Nuclease | ID | 19 | 50 | 54 | 71 | 72 | 73 | 80 |
| x.109 | 8 | A | Q | F | G | R | A | Q |
| m.775 | 36 | A | R | I | G | K | A | E |
| m.776 | 37 | A | R | L | R | Q | C | E |
| m.785 | 38 | A | R | I | G | R | A | Q |
| m.788 | 39 | A | R | F | G | R | A | Q |
| m.815 | 40 | G | R | F | G | R | A | Q |
| m.831 | 41 | G | R | F | G | P | A | E |
| m.856 | 42 | A | R | F | G | T | C | Q |
| m.863 | 43 | A | R | F | G | P | A | Q |

TABLE 116
Meganucleases Optimized for ACAG Center Sequence (Second Subunit - Lox4)

| | | I-CreI Position | | | | | | | | | | |
|---|---|---|---|---|---|---|---|---|---|---|---|---|
| | | 19 | 50 | 59 | 66 | 71 | 72 | 73 | X | 80 | 81 | 139 |
| | | Nuclease | | | | | | | | | | |
| | SEQ | 210 | 241 | 250 | 257 | 262 | 263 | 264 | X | 271 | 272 | 330 |
| +1 AA* | ID | 210 | 241 | 250 | 257 | 262 | 263 | 264 | 265* | 272 | 273 | 331 |
| x.109 | 8 | G | S | V | Y | S | T | H | | Q | I | K |
| m.775 | 36 | G | C | V | Y | G | G | R | | Q | I | K |
| m.776 | 37 | G | C | V | Y | G | G | R | | Q | I | R |
| m.785 | 38 | G | C | V | Y | G | G | R | | Q | I | R |
| m.788 | 39 | G | C | V | Y | D | R | R | R | Q | I | K |
| m.815 | 40 | A | C | V | Y | S | R | R | R | Q | I | R |
| m.831 | 41 | S | C | A | H | G | G | R | | Q | I | R |
| m.856 | 42 | G | C | V | Y | G | G | R | | Q | I | R |
| m.863 | 43 | G | C | V | Y | G | G | R | | Q | T | K |

*Refers to engineered meganucleases having an insertion following a position which corresponds to position 73 of I-CreI.

TABLE 117
CHO iGFFP Assay ACAG Center Sequence Cleavage

| | SEQ ID | |
|---|---|---|
| Meganuclease | NO: | GFP % |
| LOX 3-4bs | — | 0.37 |
| CHO 23/24 | — | 11.19 |
| x.109 | 8 | 0.29 |
| m.775 | 36 | 12.15 |
| m.776 | 37 | 10.43 |
| m.785 | 38 | 10.02 |
| m.788 | 39 | 10.49 |
| m.815 | 40 | 9.94 |
| m.831 | 41 | 10.04 |
| m.856 | 42 | 10.76 |
| m.863 | 43 | 9.99 |

### Example 4

### Engineered Meganucleases Cleaving Recognition Sequences Containing an ACAT Four Base Pair Center Sequence

Novel engineered meganucleases derived from the LOX 3-4x.109 meganuclease wee prepared by making amino acid substitutions at one or more positions in the first subunit and one or more positions in the second subunit. These engineered meganucleases were then evaluated for cleavage of the LOX 3-4 recognition sequence, which normally comprises an ACAT center sequence (SEQ ID NO: 44) in the CHO reporter assay according to Example 1. The substitutions in each subunit are provided in Tables 118 and 119, respectively. The results of the CHO reporter assay are provided in Table 120.

As expected, the LOX 3-4x.109 meganuclease demonstrated activity against the ACAT center sequence normally comprised by the LOX 3-4 recognition sequence. Additionally, novel meganucleases which were modified to comprise the residues recited in the tables below continued to cleave the LOX 3-4 recognition sequence.

TABLE 118
Meganucleases Optimized for ACAT Center
Sequence (First Subunit - Lox3)

| | | I-CreI Position | | | | | | | | |
|---|---|---|---|---|---|---|---|---|---|---|
| | SEQ | 19 | 48 | 50 | 54 | 71 | 72 | 73 | 80 | 139 |
| Nuclease | ID | 19 | 48 | 50 | 54 | 71 | 72 | 73 | 80 | 139 |
| x.109 | 8 | A | K | Q | F | G | R | A | Q | K |
| M.869 | 46 | G | K | S | F | R | T | A | E | H |
| M.873 | 47 | A | S | R | F | G | R | A | Q | K |
| M.877 | 48 | A | K | S | F | G | R | A | Q | K |
| M.883 | 49 | A | K | R | F | G | R | A | Q | K |
| M.885 | 50 | G | I | R | F | G | R | A | Q | K |
| M.886 | 51 | G | K | K | F | G | R | A | Q | K |
| M.893 | 52 | A | K | R | F | G | R | A | Q | K |
| M.901 | 53 | G | K | R | F | G | R | A | Q | K |
| M.910 | 54 | A | K | R | F | G | R | A | Q | K |
| M.917 | 55 | G | K | R | F | G | R | A | Q | K |
| M.919 | 56 | A | K | Q | F | G | R | A | Q | K |
| M.922 | 57 | G | L | K | F | G | R | A | Q | K |
| M.925 | 58 | G | K | R | F | G | R | A | Q | K |
| M.929 | 59 | A | K | R | F | G | R | A | Q | K |
| M.930 | 60 | G | K | R | F | G | R | A | E | K |
| M.933 | 61 | A | L | K | F | G | R | A | Q | K |
| M.937 | 62 | A | K | R | F | G | R | A | Q | K |
| M.941 | 63 | S | K | R | F | G | R | A | Q | K |
| M.942 | 64 | A | K | R | F | G | R | A | Q | R |
| M.945 | 65 | G | K | R | F | G | R | A | Q | K |
| M.949 | 66 | A | N | R | F | G | R | A | Q | K |
| M.950 | 67 | A | K | R | I | G | R | G | Q | K |

TABLE 119
Meganucleases Optimized for ACAT Center Sequence (Second Subunit - Lox4)

| | | I-CreI Position | | | | | | | | | | | |
|---|---|---|---|---|---|---|---|---|---|---|---|---|---|
| | SEQ | 19 | 48 | 50 | 71 | 72 | 73 | 74 | 80 | 81 | 83 | 117 | 139 |
| Nuclease | ID | 210 | 239 | 241 | 262 | 263 | 264 | 265 | 271 | 272 | 274 | 308 | 330 |
| x.109 | 8 | G | H | S | S | T | H | S | Q | I | P | E | K |
| M.869 | 46 | A | H | K | G | K | A | C | Q | I | P | E | R |
| M.873 | 47 | G | H | C | G | A | A | S | Q | I | P | E | K |
| M.877 | 48 | G | H | N | R | S | C | C | Q | T | P | E | R |
| M.883 | 49 | G | H | R | T | R | C | C | E | I | P | E | R |
| M.885 | 50 | G | T | K | G | H | A | S | Q | I | P | E | K |
| M.886 | 51 | A | H | K | K | H | C | C | E | I | P | E | K |
| M.893 | 52 | G | G | K | G | K | A | C | E | I | P | E | K |
| M.901 | 53 | A | A | K | R | H | S | C | Q | I | P | E | R |
| M.910 | 54 | G | H | K | T | K | C | C | Q | I | P | E | T |
| M.917 | 55 | A | S | C | G | T | A | C | Q | I | H | G | T |
| M.919 | 56 | G | H | S | S | T | H | S | Q | I | P | E | K |
| M.922 | 57 | A | H | K | K | A | C | A | E | I | P | E | K |
| M.925 | 58 | A | H | G | G | R | G | C | Q | I | P | E | K |
| M.929 | 59 | G | S | K | R | G | G | C | Q | I | P | E | K |
| M.930 | 60 | S | A | S | G | R | A | C | Q | I | P | E | R |
| M.933 | 61 | G | A | K | R | S | C | C | Q | I | P | E | K |
| M.937 | 62 | G | A | Q | K | S | G | C | Q | I | P | E | K |
| M.941 | 63 | G | A | C | G | T | C | C | Q | I | P | E | H |
| M.942 | 64 | G | S | C | G | H | A | C | Q | I | P | E | K |
| M.945 | 65 | A | L | Q | G | N | S | C | Q | I | P | E | K |
| M.949 | 66 | G | S | K | E | R | A | C | Q | I | P | E | K |
| M.950 | 67 | G | K | C | G | G | R | S | Q | I | P | E | R |

TABLE 120
CHO iGFFP Assay ACAT Center Sequence Cleavage

| | SEQ ID | |
|---|---|---|
| Meganuclease | NO: | GFP % |
| LOX 3-4bs | — | 0.62 |
| CHO 23/24 | — | 13.07 |
| x.109 | 8 | 11.66 |
| M.869 | 46 | 11.22 |
| M.873 | 47 | 9.76 |
| M.877 | 48 | 11.18 |
| M.883 | 49 | 10.82 |
| M.885 | 50 | 11.30 |
| M.886 | 51 | 10.38 |
| M.893 | 52 | 13.13 |
| M.901 | 53 | 9.89 |
| M.910 | 54 | 9.89 |
| M.917 | 55 | 9.83 |
| M.919 | 56 | 11.46 |
| M.922 | 57 | 10.05 |
| M.925 | 58 | 12.22 |
| M.929 | 59 | 10.15 |
| M.930 | 60 | 9.74 |
| M.933 | 61 | 10.82 |
| M.937 | 62 | 12.19 |
| M.941 | 63 | 10.17 |
| M.942 | 64 | 11.76 |
| M.945 | 65 | 11.35 |
| M.949 | 66 | 12.50 |
| M.950 | 67 | 12.27 |

### Example 5

### Engineered Meganucleases Cleaving Recognition Sequences Containing an ACGA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ACGA center sequence (SEQ ID NO: 68) in the CHO reporter assay according to Example 1. The substitutions in each subunit are provided in Tables 121 and 122, respectively. The results of the CHO reporter assay are provided in Table 123.

Following the modifications shown below, substantial improvements in cleavage of the recognition sequence having the ACGA four base pair center sequence were observed.

TABLE 121
Meganucleases Optimized for ACGA Center
Sequence (First Subunit - Lox3)

| | | I-CreI Position | | | | | | | |
|---|---|---|---|---|---|---|---|---|---|
| | SEQ | 19 | 48 | 50 | 71 | 72 | 73 | 80 | 139 |
| Nuclease | ID | 19 | 48 | 50 | 71 | 72 | 73 | 80 | 139 |
| x.109 | 8 | A | K | Q | G | R | A | Q | K |
| m.956 | 70 | G | K | R | G | R | A | Q | K |
| m.961 | 71 | G | K | R | G | R | A | Q | K |
| m.962 | 72 | G | K | R | G | R | A | E | K |
| m.963 | 73 | A | K | W | P | P | A | E | K |
| m.969 | 74 | S | K | R | G | R | A | Q | K |
| m.971 | 75 | A | K | R | G | R | A | Q | K |
| m.977 | 76 | G | K | R | G | R | A | Q | K |
| m.982 | 77 | G | K | A | G | R | A | Q | K |
| m.986 | 78 | A | K | R | G | R | A | Q | K |
| m.993 | 79 | G | K | R | G | R | A | Q | K |
| m.994 | 80 | G | K | R | G | R | A | Q | K |
| m.1001 | 81 | G | K | V | G | R | A | Q | K |
| m.1013 | 82 | G | K | V | G | R | A | Q | K |
| m.1017 | 83 | G | K | R | G | R | A | Q | K |
| m.1018 | 84 | A | K | R | G | R | A | Q | K |
| m.1021 | 85 | G | K | R | G | R | A | Q | K |
| m.1029 | 86 | A | K | R | G | R | A | Q | K |
| m.1036 | 87 | A | K | R | G | R | A | Q | K |
| m.1041 | 88 | G | K | R | G | R | A | Q | K |
| m.1044 | 89 | A | K | T | G | R | A | Q | K |

TABLE 122
Meganucleases Optimized for ACGA Center Sequence (Second Subunit - Lox4)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 210 | 239 | 241 | 262 | 263 | 264 | 265 | 271 | 330 |
| x.109 | 8 | A | K | Q | G | R | A | S | Q | K |
| m.956 | 70 | A | A | R | G | R | I | S | E | K |
| m.961 | 71 | A | A | I | G | R | V | A | Q | R |
| m.962 | 72 | A | A | R | G | H | I | A | Q | K |
| m.963 | 73 | G | K | S | G | R | I | A | Q | R |
| m.969 | 74 | G | K | C | G | H | I | A | Q | K |
| m.971 | 75 | G | H | R | G | R | V | S | E | R |
| m.977 | 76 | A | K | V | G | R | I | S | E | K |
| m.982 | 77 | A | A | R | G | R | V | S | Q | R |
| m.986 | 78 | G | K | G | G | R | I | S | Q | R |
| m.993 | 79 | A | A | R | G | R | I | S | E | K |
| m.994 | 80 | A | H | C | G | R | I | S | Q | R |
| m.1001 | 81 | A | H | R | G | R | I | A | E | R |
| m.1013 | 82 | A | H | S | G | R | I | S | Q | K |
| m.1017 | 83 | A | T | R | G | R | I | A | E | R |
| m.1018 | 84 | G | A | R | G | R | I | A | E | R |
| m.1021 | 85 | A | K | C | G | R | I | S | E | K |
| m.1029 | 86 | G | G | R | G | R | I | A | Q | R |
| m.1036 | 87 | G | Q | R | G | R | I | S | Q | R |
| m.1041 | 88 | A | A | I | G | R | V | S | Q | R |
| m.1044 | 89 | G | K | S | G | R | V | S | E | R |

TABLE 123
CHO iGFFP Assay ACGA Center Sequence Cleavage

| | SEQ ID | |
|---|---|---|
| Meganuclease | NO: | GFP % |
| LOX 3-4bs | — | 0.5 |
| CHO 23/24 | — | 11.7 |
| x.109 | 8 | 0.7 |
| m.956 | 70 | 4.7 |
| m.961 | 71 | 2.9 |
| m.962 | 72 | 2.8 |
| m.963 | 73 | 3.1 |
| m.969 | 74 | 3.4 |
| m.971 | 75 | 3.9 |
| m.977 | 76 | 3.2 |
| m.982 | 77 | 2.5 |
| m.986 | 78 | 4.1 |
| m.993 | 79 | 4.1 |
| m.994 | 80 | 3.3 |
| m.1001 | 81 | 4.0 |
| m.1013 | 82 | 1.7 |
| m.1017 | 83 | 4.6 |
| m.1018 | 84 | 4.2 |
| m.1021 | 85 | 3.2 |
| m.1029 | 86 | 3.8 |
| m.1036 | 87 | 5.0 |
| m.1041 | 88 | 4.5 |
| m.1044 | 89 | 5.8 |

### Example 6

### Engineered Meganucleases Cleaving Recognition Sequences Containing an ACGC Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ACGC center sequence (SEQ ID NO: 90) in the CHO reporter assay according to Example 1. The substitutions in each subunit are provided in Tables 124 and 125, respectively. The results of the CHO reporter assay are provided in Table 126.

Following the modifications shown below, substantial improvements in cleavage of the recognition sequence having the ACGC four base pair center sequence were observed in most of the engineered nucleases, while some were comparable to LOX 3-4x.109.

TABLE 124
Meganucleases Optimized for ACGC Center
Sequence (First Subunit - Lox3)

| | | I-CreI Position | | | | | | |
|---|---|---|---|---|---|---|---|---|
| | SEQ | 19 | 48 | 50 | 71 | 72 | 73 | 80 |
| Nuclease | ID | 19 | 48 | 50 | 71 | 72 | 73 | 80 |
| x.109 | 8 | A | K | Q | G | R | A | Q |
| m.1049 | 92 | G | H | Q | G | R | A | Q |
| m.1050 | 93 | A | Q | R | G | R | A | Q |
| m.1052 | 94 | A | K | R | G | R | A | Q |
| m.1068 | 95 | G | K | R | G | R | A | Q |
| m.1069 | 96 | A | K | R | R | R | A | Q |
| m.1074 | 97 | A | L | R | G | R | A | Q |
| m.1085 | 98 | G | K | R | G | R | A | Q |
| m.1093 | 99 | A | H | K | A | P | A | Q |
| m.1095 | 100 | A | K | S | R | R | A | Q |
| m.1098 | 101 | G | L | K | G | R | A | Q |
| m.1100 | 102 | G | L | K | G | R | A | Q |
| m.1101 | 103 | A | L | R | R | H | A | Q |
| m.1107 | 104 | A | H | R | R | R | A | Q |
| m.1109 | 105 | A | K | T | G | R | A | Q |
| m.1111 | 106 | A | L | R | G | R | A | Q |
| m.1113 | 107 | G | K | R | G | R | A | Q |
| m.1116 | 108 | A | K | R | G | R | A | Q |
| m.1117 | 109 | A | K | C | G | R | A | Q |
| m.1118 | 110 | G | A | R | G | R | A | Q |
| m.1123 | 111 | A | S | R | G | R | A | E |
| m.1125 | 112 | G | K | R | G | R | A | Q |
| m.1126 | 113 | G | K | R | G | R | A | Q |
| m.1127 | 114 | G | L | R | G | R | A | Q |
| m.1129 | 115 | A | K | R | G | R | A | Q |
| m.1131 | 116 | S | K | R | G | R | A | Q |
| m.1133 | 117 | A | K | C | G | R | A | Q |
| m.1137 | 118 | G | K | R | G | R | A | Q |

TABLE 125
Meganucleases Optimized for ACGC Center Sequence (Second Subunit - Lox4)

| | | I-CreI Position | | | | | | | | | |
|---|---|---|---|---|---|---|---|---|---|---|---|
| | SEQ | 19 | 48 | 50 | 71 | 72 | 73 | 74 | 80 | 87 | 139 |
| Nuclease | ID | 210 | 239 | 241 | 262 | 263 | 264 | 265 | 271 | 278 | 330 |
| x.109 | 8 | G | H | S | S | T | H | S | Q | F | K |
| m.1049 | 92 | A | K | E | G | R | T | A | Q | F | K |
| m.1050 | 93 | G | H | K | G | R | V | A | E | F | R |
| m.1052 | 94 | G | K | S | K | R | I | A | Q | F | N |
| m.1068 | 95 | A | L | K | G | R | V | T | E | F | H |
| m.1069 | 96 | G | K | E | G | R | V | A | E | F | R |
| m.1074 | 97 | G | H | K | G | R | I | A | E | F | H |
| m.1085 | 98 | A | K | E | S | R | V | A | Q | F | R |
| m.1093 | 99 | G | H | K | G | A | I | S | E | F | H |
| m.1095 | 100 | G | K | I | G | R | I | A | Q | F | R |
| m.1098 | 101 | A | L | N | G | R | I | S | Q | F | H |
| m.1100 | 102 | A | A | K | A | R | I | S | Q | L | H |
| m.1101 | 103 | G | S | K | G | T | V | S | Q | F | R |
| m.1107 | 104 | G | A | K | G | R | T | A | E | F | K |
| m.1109 | 105 | G | K | I | G | S | V | S | E | F | R |
| m.llll | 106 | G | K | N | G | R | V | A | E | F | H |
| m.1113 | 107 | A | K | V | G | H | T | A | Q | F | H |
| m.1116 | 108 | G | H | K | R | R | V | A | E | F | R |
| m.1117 | 109 | G | L | N | G | R | V | A | Q | F | R |
| m.1118 | 110 | A | K | I | G | R | C | A | E | F | H |
| m.1123 | 111 | G | H | K | G | R | I | T | Q | F | H |
| m.1125 | 112 | A | L | E | G | R | V | A | Q | F | K |
| m.1126 | 113 | A | K | K | G | S | I | S | Q | F | A |
| m.1127 | 114 | A | L | K | G | G | I | A | Q | F | R |
| m.1129 | 115 | G | N | S | G | R | I | S | Q | F | R |
| m.1131 | 116 | G | K | I | G | R | I | A | E | F | R |
| m.1133 | 117 | G | H | R | G | R | C | A | E | F | H |
| m.1137 | 118 | A | A | K | G | W | I | A | E | F | K |

TABLE 126
CHO iGFFP Assay ACGC Center Sequence Cleavage

| | SEQ ID | |
|---|---|---|
| Meganuclease | NO: | GFP % |
| LOX 3-4bs | — | 0.27 |
| CHO 23/24 | — | 10.12 |
| x.109 | 8 | 5.14 |
| m.1049 | 92 | 7.59 |
| m.1050 | 93 | 8.90 |
| m.1052 | 94 | 9.34 |
| m.1068 | 95 | 9.00 |
| m.1069 | 96 | 10.35 |
| m.1074 | 97 | 8.56 |
| m.1085 | 98 | 7.64 |
| m.1093 | 99 | 8.32 |
| m.1095 | 100 | 5.42 |
| m.1098 | 101 | 7.42 |
| m.1100 | 102 | 9.09 |
| m.1101 | 103 | 8.44 |
| m.1107 | 104 | 8.36 |
| m.1109 | 105 | 8.62 |
| m.1111 | 106 | 8.51 |
| m.1113 | 107 | 7.68 |
| m.1116 | 108 | 8.64 |
| m.1117 | 109 | 9.13 |
| m.1118 | 110 | 7.86 |
| m.1123 | 111 | 9.56 |
| m.1125 | 112 | 10.39 |
| m.1126 | 113 | 8.46 |
| m.1127 | 114 | 8.04 |
| m.1129 | 115 | 8.70 |
| m.1131 | 116 | 7.97 |
| m.1133 | 117 | 6.94 |
| m.1137 | 118 | 7.49 |

### Example 7

### Engineered Meganucleases Cleaving Recognition Sequences Containing an ACGG Four Base Pair Center Sequence

Novel engineered meganucleases derived from the LOX 3-4x.109 meganuclease were prepared by making amino acid substitutions at one or more positions in the first subunit and one or more positions in the second subunit. In addition, an R residue was inserted following position 264, which corresponds to position 73 of wild-type I-CreI. These engineered meganucleases were then evaluated for cleavage of the LOX 3-4 recognition sequence modified to have an ACGG center sequence (SEQ ID NO: 119) in the CHO reporter assay according to Example 1. The substitutions in each subunit are provided in Tables 127 and 128, respectively. The results of the CHO reporter assay are provided in Table 129.

Following the modifications shown below, substantial improvements in cleavage of the recognition sequence having the ACGG four base pair center sequence were observed.

TABLE 127
Meganucleases Optimized for ACGG Center
Sequence (First Subunit - Lox3)

| | | I-CreI Position | | | | |
|---|---|---|---|---|---|---|
| | SEQ | 50 | 54 | 72 | 73 | 80 |
| Nuclease | ID | 50 | 54 | 72 | 73 | 80 |
| x.109 | 8 | Q | F | R | A | Q |
| m.1876 | 121 | R | F | R | A | Q |
| m.1894 | 122 | K | L | R | A | Q |
| m.1898 | 123 | R | F | R | A | Q |
| m.1904 | 124 | R | F | R | A | Q |
| m.1910 | 125 | R | F | R | A | Q |
| m.1914 | 126 | R | F | R | A | Q |
| m.1930 | 127 | R | F | R | A | Q |
| m.1938 | 128 | R | F | R | A | Q |
| m.1941 | 129 | R | F | R | A | Q |
| m.1944 | 130 | R | F | R | A | Q |
| m.1946 | 131 | R | F | R | A | Q |
| m.1947 | 132 | R | F | R | A | Q |
| m.1950 | 133 | R | F | R | A | Q |
| m.1952 | 134 | R | F | R | A | Q |
| m.1960 | 135 | R | F | R | A | Q |

TABLE 128
Meganucleases Optimized for ACGG Center
Sequence (Second Subunit - Lox4)

| | | I-CreI Position | | | | | | | |
|---|---|---|---|---|---|---|---|---|---|
| | | 19 | 48 | 50 | 71 | 72 | 73 | X | 19 |
| | | Nuclease | | | | | | | |
| | SEQ | 210 | 239 | 241 | 262 | 263 | 264 | X | 271 |
| +1 AA* | ID | 210 | 239 | 241 | 262 | 263 | 264 | 265* | 272 |
| x.109 | 8 | G | H | S | S | T | H | | Q |
| m.1876 | 121 | A | K | R | D | G | R | R | Q |
| m.1894 | 122 | A | K | R | D | G | R | R | Q |
| m.1898 | 123 | A | K | R | D | G | R | R | Q |
| m.1904 | 124 | A | K | R | D | G | R | R | Q |
| m.1910 | 125 | A | K | R | D | G | R | R | Q |
| m.1914 | 126 | A | K | R | D | G | R | R | Q |
| m.1930 | 127 | A | K | R | D | G | R | R | Q |
| m.1938 | 128 | A | K | P | D | G | R | R | Q |
| m.1941 | 129 | A | K | R | D | G | R | R | Q |
| m.1944 | 130 | A | K | R | D | G | R | R | Q |
| m.1946 | 131 | A | K | R | D | G | R | R | Q |
| m.1947 | 132 | A | K | R | D | G | R | R | Q |
| m.1950 | 133 | A | K | R | D | G | R | R | Q |
| m.1952 | 134 | A | K | P | D | G | G | R | Q |
| m.1960 | 135 | A | K | R | D | G | R | R | Q |

*Refers to engineered meganucleases having an insertion following a position which corresponds to position 73 of I-CreI.

TABLE 129
CHO iGFFP Assay ACGG Center Sequence Cleavage

| | SEQ ID | |
|---|---|---|
| Meganuclease | NO: | GFP % |
| LOX 3-4bs | — | 0.7 |
| CHO 23/24 | — | 13.6 |
| x.109 | 8 | 0.5 |
| m.1876 | 121 | 9.6 |
| m.1894 | 122 | 6.0 |
| m.1898 | 123 | 9.2 |
| m.1904 | 124 | 6.1 |
| m.1910 | 125 | 9.3 |
| m.1914 | 126 | 9.5 |
| m.1930 | 127 | 9.1 |
| m.1938 | 128 | 9.4 |
| m.1941 | 129 | 10.1 |
| m.1944 | 130 | 8.3 |
| m.1946 | 131 | 10.8 |
| m.1947 | 132 | 10.5 |
| m.1950 | 133 | 9.3 |
| m.1952 | 134 | 6.8 |
| m.1960 | 135 | 8.5 |

### Example 8

### Engineered Meganucleases Cleaving Recognition Sequences Containing an ACGT Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ACGT center sequence (SEQ ID NO: 136) in the CHO reporter assay according to Example 1. The substitutions in each subunit are provided in Tables 130 and 131, respectively. The results of the CHO reporter assay are provided in Table 132. Novel meganucleases which were modified to comprise the residues recited in the tables below continued to cleave the LOX 3-4 recognition sequence having an ACGT four base pair center sequence or were more active than the LOX 3-4x.109 meganuclease.

TABLE 130
Meganucleases Optimized for ACGT Center
Sequence (First Subunit - Lox3)

| | | I-CreI Position | | | | | | | |
|---|---|---|---|---|---|---|---|---|---|
| | SEQ | 19 | 48 | 50 | 71 | 72 | 73 | 80 | 139 |
| Nuclease | ID | 19 | 48 | 50 | 71 | 72 | 73 | 80 | 139 |
| x.109 | 8 | A | K | Q | G | R | A | Q | K |
| m.1145 | 138 | G | L | R | G | R | A | Q | K |
| m.1149 | 139 | A | K | R | G | R | A | Q | K |
| m.1152 | 140 | A | K | R | G | R | A | Q | K |
| m.1153 | 141 | A | K | C | G | R | A | Q | K |
| m.1157 | 142 | G | K | R | G | R | A | E | K |
| m.1158 | 143 | A | K | R | G | R | A | E | K |
| m.1176 | 144 | G | K | S | G | R | A | Q | K |
| m.1191 | 145 | G | K | S | G | R | A | Q | K |
| m.1198 | 146 | A | K | R | G | R | A | Q | K |
| m.1201 | 147 | G | L | R | G | R | A | Q | R |
| m.1205 | 148 | A | K | R | G | R | A | Q | K |
| m.1206 | 149 | G | K | C | G | R | A | Q | K |
| m.1208 | 150 | G | K | R | G | R | A | Q | K |
| m.1212 | 151 | A | S | R | G | R | A | Q | K |
| m.1218 | 152 | A | H | R | G | R | A | Q | K |
| m.1224 | 153 | G | K | V | G | R | A | Q | K |
| m.1225 | 154 | A | K | C | G | R | A | Q | K |
| m.1226 | 155 | A | K | R | G | R | A | Q | K |
| m.1227 | 156 | A | K | R | G | R | A | Q | K |

TABLE 131
Meganucleases Optimized for ACGT Center Sequence (Second Subunit - Lox4)

| | | I-CreI Position | | | | | | | | | |
|---|---|---|---|---|---|---|---|---|---|---|---|
| | SEQ | 19 | 48 | 50 | 71 | 72 | 73 | 74 | 80 | 85 | 139 |
| Nuclease | ID | 210 | 239 | 241 | 262 | 263 | 264 | 265 | 271 | 276 | 330 |
| x.109 | 8 | G | H | S | S | T | H | S | Q | H | K |
| m.1145 | 138 | A | K | C | P | R | C | S | Q | H | K |
| m.1149 | 139 | G | L | C | G | K | A | S | Q | H | K |
| m.1152 | 140 | G | S | Q | G | A | A | S | Q | H | K |
| m.1153 | 141 | G | S | E | G | K | A | A | Q | H | K |
| m.1157 | 142 | A | K | C | P | K | C | A | Q | H | K |
| m.1158 | 143 | G | S | C | G | K | A | S | Q | H | K |
| m.1176 | 144 | A | K | E | T | R | C | A | Q | H | K |
| m.1191 | 145 | A | K | C | A | K | C | A | Q | H | K |
| m.1198 | 146 | G | S | E | G | R | A | A | Q | H | K |
| m.1201 | 147 | A | K | E | G | R | A | A | Q | H | K |
| m.1205 | 148 | G | K | E | R | R | A | A | Q | H | R |
| m.1206 | 149 | A | S | E | G | K | C | T | Q | H | K |
| m.1208 | 150 | A | S | E | G | K | C | T | Q | H | K |
| m.1212 | 151 | G | L | E | G | R | C | A | Q | H | K |
| m.1218 | 152 | G | K | E | G | R | A | S | E | H | K |
| m.1224 | 153 | A | S | A | G | R | A | A | Q | Y | K |
| m.1225 | 154 | G | S | A | G | R | A | A | Q | H | K |
| m.1226 | 155 | G | K | E | N | R | C | S | Q | H | K |
| m.1227 | 156 | G | K | C | G | K | S | S | Q | H | K |

TABLE 132
CHO iGFFP Assay ACGT Center Sequence Cleavage

| | SEQ ID | |
|---|---|---|
| Meganuclease | NO: | GFP % |
| LOX 3-4bs | — | 0.35 |
| CHO 23/24 | — | 10.54 |
| x.109 | 8 | 8.34 |
| m.1145 | 138 | 8.70 |
| m.1149 | 139 | 8.91 |
| m.1152 | 140 | 7.91 |
| m.1153 | 141 | 10.28 |
| m.1157 | 142 | 7.22 |
| m.1158 | 143 | 9.19 |
| m.1176 | 144 | 7.70 |
| m.1191 | 145 | 8.00 |
| m.1198 | 146 | 8.30 |
| m.1201 | 147 | 7.98 |
| m.1205 | 148 | 7.68 |
| m.1206 | 149 | 9.84 |
| m.1208 | 150 | 7.57 |
| m.1212 | 151 | 7.31 |
| m.1218 | 152 | 8.48 |
| m.1224 | 153 | 7.84 |
| m.1225 | 154 | 10.64 |
| m.1226 | 155 | 8.02 |
| m.1227 | 156 | 8.44 |

### Engineered Meganucleases Cleaving Recognition Sequences Containing an ATAA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ATAA center sequence (SEQ ID NO: 157) in the CHO reporter assay according to Example 1. The substitutions in each subunit are provided in Tables 133 and 134, respectively. The results of the CHO reporter assay are provided in Table 135.

Following the modifications shown below, substantial improvements in cleavage of the recognition sequence having the ATAA four base pair center sequence were observed.

TABLE 133
Meganucleases Optimized for ATAA Center
Sequence (First Subunit - Lox3)

| | | I-CreI Position | | | | | | | | | |
|---|---|---|---|---|---|---|---|---|---|---|---|
| | SEQ | 19 | 48 | 50 | 71 | 72 | 73 | 74 | 80 | 100 | 139 |
| Nuclease | ID | 19 | 48 | 50 | 71 | 72 | 73 | 74 | 80 | 100 | 139 |
| x.109 | 8 | A | K | Q | G | R | A | S | Q | K | K |
| m.1232 | 159 | G | K | T | G | R | A | S | E | K | R |
| m.1235 | 160 | S | A | R | G | R | A | S | Q | K | R |
| m.1236 | 161 | A | H | T | G | R | A | S | Q | K | K |
| m.1237 | 162 | A | A | I | G | R | A | S | Q | K | K |
| m.1240 | 163 | A | S | R | G | R | A | S | E | K | R |
| m.1250 | 164 | A | H | R | G | A | A | S | Q | E | K |
| m.1253 | 165 | A | S | R | K | G | T | A | E | K | R |
| m.1255 | 166 | A | K | G | G | R | A | S | Q | K | K |
| m.1256 | 167 | G | K | K | S | G | A | S | Q | K | R |
| m.1260 | 168 | G | K | R | G | R | C | S | E | K | K |
| m.1261 | 169 | A | S | R | H | A | A | S | Q | K | R |
| m.1262 | 170 | A | K | D | G | R | A | S | Q | K | R |
| m.1268 | 171 | A | K | T | N | Q | A | A | Q | K | R |
| m.1269 | 172 | A | H | R | G | H | C | S | Q | K | R |
| m.1278 | 173 | A | K | C | G | L | C | S | E | K | K |
| m.1284 | 174 | G | A | C | G | R | A | S | E | K | K |
| m.1293 | 175 | G | K | V | G | A | C | S | E | K | R |
| m.1301 | 176 | A | K | C | G | S | A | S | E | K | R |
| m.1308 | 177 | A | S | R | H | R | C | S | E | K | R |
| m.1309 | 178 | S | K | K | G | R | A | S | Q | K | R |
| m.1311 | 179 | A | K | R | G | R | A | S | Q | K | K |
| m.1317 | 180 | A | L | R | G | R | A | S | Q | K | K |
| m.1319 | 181 | A | S | Q | G | R | A | S | Q | K | K |
| m.1322 | 182 | G | Q | R | G | R | A | S | Q | K | R |
| m.1300 | 183 | A | A | R | G | R | A | S | Q | K | R |

TABLE 134
Meganucleases Optimized for ATAA Center Sequence (Second Subunit - Lox4)

| | | I-CreI Position | | | | | | | | | | | | |
|---|---|---|---|---|---|---|---|---|---|---|---|---|---|---|
| | SEQ | 19 | 48 | 50 | 59 | 71 | 72 | 73 | 74 | 78 | 79 | 80 | 118 | 139 |
| Nuclease | ID | 210 | 239 | 241 | 250 | 262 | 263 | 264 | 265 | 269 | 270 | 271 | 309 | 330 |
| x.109 | 8 | G | H | S | V | S | T | H | S | L | S | Q | S | K |
| m.1232 | 159 | S | S | R | V | G | T | I | A | L | S | E | S | K |
| m.1235 | 160 | G | T | R | V | G | R | C | A | L | S | E | S | R |
| m.1236 | 161 | G | A | K | V | G | Q | V | A | L | S | Q | S | R |
| m.1237 | 162 | G | K | E | V | G | G | V | A | L | S | Q | S | K |
| m.1240 | 163 | G | K | E | V | K | A | I | A | L | S | Q | S | R |
| m.1250 | 164 | G | A | A | V | G | G | V | A | L | S | Q | S | R |
| m.1253 | 165 | G | S | K | V | G | R | I | A | L | S | Q | S | R |
| m.1255 | 166 | G | A | C | A | G | R | V | S | L | S | Q | S | R |
| m.1256 | 167 | A | N | K | V | G | R | I | A | L | S | E | S | R |
| m.1260 | 168 | A | S | T | V | G | Y | V | A | L | S | Q | S | R |
| m.1261 | 169 | G | S | R | V | G | R | I | A | L | S | E | S | R |
| m.1262 | 170 | G | K | T | V | S | G | V | S | L | S | Q | S | R |
| m.1268 | 171 | G | S | T | V | G | R | I | A | L | S | Q | S | R |
| m.1269 | 172 | G | T | R | V | G | G | I | S | L | S | E | S | R |
| m.1278 | 173 | G | S | K | V | G | R | V | T | L | S | Q | S | R |
| m.1284 | 174 | A | K | K | V | G | S | V | S | L | S | Q | S | R |
| m.1293 | 175 | S | S | K | V | G | N | V | S | L | S | Q | S | R |
| m.1301 | 176 | G | S | R | V | G | S | V | S | L | S | E | S | K |
| m.1308 | 177 | G | T | R | V | G | R | C | A | L | S | E | S | R |
| m.1309 | 178 | G | S | K | V | G | K | V | S | L | S | E | S | K |
| m.1311 | 179 | G | S | R | V | G | Q | V | S | L | S | Q | S | R |
| m.1317 | 180 | G | S | K | A | R | S | I | A | L | S | E | S | R |
| m.1319 | 181 | G | K | T | V | G | S | V | S | L | S | E | S | R |
| m.1322 | 182 | G | S | R | V | G | R | V | S | L | S | Q | S | K |
| m.1300 | 183 | A | S | K | V | G | Y | V | A | L | S | Q | F | R |

TABLE 135
CHO iGFFP Assay ATAA Center Sequence Cleavage

| | SEQ ID | |
|---|---|---|
| Meganuclease | NO: | GFP % |
| LOX 3-4bs | — | 0.59 |
| CHO 23/24 | — | 10.40 |
| x.109 | 8 | 1.11 |
| m.1232 | 159 | 6.82 |
| m.1235 | 160 | 6.54 |
| m.1236 | 161 | 9.28 |
| m.1237 | 162 | 8.13 |
| m.1240 | 163 | 7.02 |
| m.1250 | 164 | 6.44 |
| m.1253 | 165 | 6.87 |
| m.1255 | 166 | 7.23 |
| m.1256 | 167 | 7.22 |
| m.1260 | 168 | 6.96 |
| m.1261 | 169 | 7.84 |
| m.1262 | 170 | 6.45 |
| m.1268 | 171 | 7.44 |
| m.1269 | 172 | 8.64 |
| m.1278 | 173 | 6.55 |
| m.1284 | 174 | 6.81 |
| m.1293 | 175 | 8.00 |
| m.1301 | 176 | 7.37 |
| m.1308 | 177 | 6.76 |
| m.1309 | 178 | 6.34 |
| m.1311 | 179 | 6.36 |
| m.1317 | 180 | 8.30 |
| m.1319 | 181 | 6.30 |
| m.1322 | 182 | 6.92 |
| m.1300 | 183 | 7.57 |

### Example 10

### Engineered Meganucleases Cleaving Recognition Sequences Containing an ATAG Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ATAG center sequence (SEQ ID NO: 184) in the CHO reporter assay according to Example 1. The substitutions in each subunit are provided in Tables 136 and 137, respectively. The results of the CHO reporter assay are provided in Table 138.

Following the modifications shown below, substantial improvements in cleavage of the recognition sequence having the ATAG four base pair center sequence were observed.

TABLE 136
Meganucleases Optimized for ATAG Center
Sequence (First Subunit - Lox3)

| | | I-CreI Position | | | | | | | |
|---|---|---|---|---|---|---|---|---|---|
| | | 19 | 48 | 50 | 71 | 72 | 73 | 80 | 139 |
| Nuclease | SEQ ID | 19 | 48 | 50 | 71 | 72 | 73 | 80 | 139 |
| x.109 | 8 | A | K | Q | G | R | A | Q | K |
| m.1329 | 186 | A | H | R | G | R | A | Q | K |
| m.1338 | 187 | G | K | R | G | R | A | Q | R |
| m.1343 | 188 | A | K | R | G | R | A | Q | K |
| m.1345 | 189 | A | K | R | G | G | A | Q | K |
| m.1347 | 190 | A | K | R | G | S | A | Q | K |
| m.1353 | 191 | A | K | R | G | R | A | Q | K |
| m.1361 | 192 | A | K | R | G | G | C | Q | R |
| m.1369 | 193 | A | K | R | R | A | C | Q | K |
| m.1391 | 194 | A | K | R | G | R | A | Q | K |
| m.1392 | 195 | A | H | R | G | R | A | Q | K |
| m.1394 | 196 | A | H | R | G | R | A | Q | K |
| m.1396 | 197 | A | K | R | G | P | A | Q | K |
| m.1405 | 198 | A | H | R | H | Q | A | Q | K |
| m.1415 | 199 | A | K | R | G | R | A | E | K |

TABLE 137
Meganucleases Optimized for ATAG Center Sequence (Second Subunit - Lox4)

| | | I-CreI Position | | | | | | | |
|---|---|---|---|---|---|---|---|---|---|
| | SEQ | 19 | 36 | 50 | 59 | 72 | 73 | 80 | 139 |
| Nuclease | ID | 210 | 227 | 241 | 250 | 263 | 264 | 271 | 330 |
| x.109 | 8 | G | K | S | V | T | H | Q | K |
| m.1329 | 186 | G | K | C | V | G | R | Q | K |
| m.1338 | 187 | A | K | C | V | G | R | Q | K |
| m.1343 | 188 | G | K | C | A | G | R | Q | R |
| m.1345 | 189 | G | K | C | V | G | R | Q | K |
| m.1347 | 190 | G | K | R | A | G | R | Q | R |
| m.1353 | 191 | G | R | C | A | S | R | Q | R |
| m.1361 | 192 | G | K | C | V | G | R | Q | R |
| m.1369 | 193 | G | K | C | V | G | R | Q | R |
| m.1391 | 194 | G | K | C | V | G | R | Q | R |
| m.1392 | 195 | G | K | C | A | G | R | Q | K |
| m.1394 | 196 | G | K | — | A | G | R | Q | K |
| m.1396 | 197 | G | K | C | V | G | R | Q | R |
| m.1405 | 198 | G | K | C | A | G | R | Q | R |
| m.1415 | 199 | G | K | C | A | G | R | Q | R |

TABLE 138
CHO iGFFP Assay ATAG Center Sequence Cleavage

| | SEQ ID | |
|---|---|---|
| Meganuclease | NO: | GFP % |
| LOX 3-4bs | — | 0.67 |
| CHO 23/24 | — | 10.22 |
| x.109 | 8 | 0.45 |
| m.1329 | 186 | 15.54 |
| m.1338 | 187 | 12.01 |
| m.1343 | 188 | 11.40 |
| m.1345 | 189 | 13.99 |
| m.1347 | 190 | 12.28 |
| m.1353 | 191 | 15.61 |
| m.1361 | 192 | 12.85 |
| m.1369 | 193 | 13.03 |
| m.1391 | 194 | 11.45 |
| m.1392 | 195 | 11.38 |
| m.1394 | 196 | 11.49 |
| m.1396 | 197 | 11.35 |
| m.1405 | 198 | 12.51 |
| m.1415 | 199 | 13.13 |

### Example 11

### Engineered Meganucleases Cleaving Recognition Sequences Containing an ATAT Four Bas Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ATAT center sequence (SEQ ID NO: 200) in the CHO reporter assay according to Example 1. The substitutions in each subunit are provided in Tables 139 and 140, respectively. The results of the CHO reporter assay are provided in Table 141.

Following the modifications shown below, substantial improvements in cleavage of the recognition sequence having the ATAT four base pair center sequence were observed.

TABLE 139
Meganucleases Optimized for ATAT Center
Sequence (First Subunit - Lox3)

| | | I-CreI Position | | | | | | | |
|---|---|---|---|---|---|---|---|---|---|
| | SEQ | 19 | 48 | 50 | 71 | 72 | 73 | 80 | 139 |
| Nuclease | ID | 19 | 48 | 50 | 71 | 72 | 73 | 80 | 139 |
| x.109 | 8 | A | K | Q | G | R | A | Q | K |
| m.2244 | 202 | A | H | N | G | R | A | Q | R |
| m.2248 | 203 | G | H | C | G | R | A | Q | R |
| m.2254 | 204 | A | C | R | G | R | A | Q | R |
| m.2263 | 205 | A | A | R | G | R | A | Q | R |
| m.2273 | 206 | A | K | R | G | R | A | Q | R |
| m.2274 | 207 | A | A | K | G | R | A | Q | R |
| m.2313 | 208 | A | A | K | G | R | A | Q | R |
| m.2316 | 209 | A | K | C | G | R | A | Q | K |
| m.2327 | 210 | A | K | C | G | R | A | Q | R |
| m.2318 | 211 | A | K | S | G | R | A | E | R |
| m.2319 | 212 | A | S | R | H | A | A | Q | R |
| m.2320 | 213 | A | K | T | I | N | C | Q | R |
| m.2322 | 214 | — | D | K | G | R | A | Q | R |
| m.2324 | 215 | A | K | R | G | Q | S | Q | K |
| m.2326 | 216 | A | K | V | G | R | A | Q | S |
| m.2329 | 217 | A | T | R | G | R | A | Q | R |
| m.2330 | 218 | A | K | K | G | R | A | E | R |
| m.2258 | 219 | A | K | V | G | A | A | Q | R |

TABLE 140
Meganucleases Optimized for ATAT Center Sequence (Second Subunit - Lox4)

| | | I-CreI Position | | | | | | | | | |
|---|---|---|---|---|---|---|---|---|---|---|---|
| | Seq | 19 | 48 | 50 | 59 | 71 | 72 | 73 | 74 | 80 | 139 |
| Nuclease | ID | 210 | 239 | 241 | 250 | 262 | 263 | 264 | 265 | 271 | 330 |
| x.109 | 8 | G | H | S | V | S | T | H | S | Q | K |
| m.2244 | 202 | G | K | C | V | K | T | C | C | Q | K |
| m.2248 | 203 | A | H | K | V | K | A | C | C | E | R |
| m.2254 | 204 | G | A | C | V | K | R | C | A | Q | R |
| m.2263 | 205 | G | H | S | V | K | S | A | S | Q | R |
| m.2273 | 206 | G | H | R | V | E | R | C | C | Q | R |
| m.2274 | 207 | G | H | K | V | I | K | C | C | Q | R |
| m.2313 | 208 | G | A | K | V | G | K | C | A | Q | P |
| m.2316 | 209 | G | K | C | V | K | A | C | A | E | R |
| m.2327 | 210 | G | H | Q | V | R | K | C | C | Q | N |
| m.2318 | 211 | G | H | K | V | E | G | C | C | Q | R |
| m.2319 | 212 | G | A | R | V | R | R | C | C | E | R |
| m.2320 | 213 | G | S | N | V | R | G | S | C | Q | K |
| m.2322 | 214 | G | S | K | V | R | G | G | S | Q | K |
| m.2324 | 215 | G | S | S | V | G | R | C | C | Q | K |
| m.2326 | 216 | G | R | C | A | K | T | C | C | K | N |
| m.2329 | 217 | G | T | N | V | G | G | C | C | Q | R |
| m.2330 | 218 | G | A | R | V | R | N | C | C | Q | R |
| m.2258* | 219 | — | — | — | — | — | — | — | — | — | — |

*Sequencing of the second subunit was incomplete for the m.2258 meganuclease.

TABLE 141
CHO iGFFP Assay ATAT Center Sequence Cleavage

| | SEQ ID | |
|---|---|---|
| Meganuclease | NO: | GFP % |
| LOX 3-4 bs | | 0.50 |
| CHO 23-24 | | 11.80 |
| x.109 | 8 | 12.2 |
| m.2244 | 202 | 12.9 |
| m.2248 | 203 | 13.8 |
| m.2254 | 204 | 10.1 |
| m.2263 | 205 | 10.1 |
| m.2273 | 206 | 8.4 |
| m.2274 | 207 | 10.4 |
| m.2313 | 208 | 13.1 |
| m.2316 | 209 | 12.1 |
| m.2327 | 210 | 11.8 |
| m.2318 | 211 | 9.5 |
| m.2319 | 212 | 9.3 |
| m.2320 | 213 | 12.1 |
| m.2322 | 214 | 11.4 |
| m.2324 | 215 | 11.8 |
| m.2326 | 216 | 11.5 |
| m.2329 | 217 | 14.9 |
| m.2330 | 218 | 13.9 |
| m.2258 | 219 | 11.0 |

### Example 12

### Engineered Meganucleases Cleaving Recognition Sequences Containing an ATGA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have an ATGA center sequence (SEQ ID NO: 220) in the CHO reporter assay according to Example 1. The substitutions in each subunit are provided in Tables 142 and 143, respectively. The results of the CHO reporter assay are provided in Table 144.

Following the modifications shown below, substantial improvements in cleavage of the recognition sequence having the ATGA four base pair center sequence were observed.

TABLE 142
Meganucleases Optimized for ATGA Center
Sequence (First Subunit - Lox3)

| | | I-CreI Position | | | | | | | | |
|---|---|---|---|---|---|---|---|---|---|---|
| | SEQ | 19 | 48 | 50 | 72 | 73 | 80 | 87 | 92 | 139 |
| Nuclease | ID | 19 | 48 | 50 | 72 | 73 | 80 | 87 | 92 | 139 |
| x.109 | 8 | A | K | Q | R | A | Q | F | Q | K |
| m.1417 | 222 | A | A | R | T | A | Q | F | R | K |
| m.1421 | 223 | A | K | T | R | A | Q | F | Q | K |
| m.1432 | 224 | G | K | R | S | A | E | F | Q | K |
| m.1436 | 225 | S | K | E | R | A | Q | F | Q | K |
| m.1437 | 226 | G | K | S | R | A | E | F | Q | K |
| m.1441 | 227 | A | K | C | R | A | Q | F | Q | K |
| m.1450 | 228 | A | K | T | R | A | E | F | Q | K |
| m.1451 | 229 | A | H | R | A | A | E | F | Q | R |
| m.1453 | 230 | A | K | T | T | A | E | F | Q | R |
| m.1468 | 231 | G | L | R | R | A | E | F | Q | K |
| m.1469 | 232 | G | K | R | R | A | Q | F | Q | K |
| m.1477 | 233 | A | L | R | R | A | Q | F | Q | K |
| m.1478 | 234 | A | K | R | R | A | Q | F | Q | K |
| m.1485 | 235 | G | A | R | R | A | E | F | Q | R |
| m.1486 | 236 | A | K | T | T | A | E | F | Q | R |
| m.1488 | 237 | A | K | V | R | A | E | F | Q | K |
| m.1491 | 238 | A | K | R | R | A | Q | F | Q | K |
| m.1500 | 239 | A | K | R | S | S | Q | F | Q | R |
| m.1501 | 240 | A | K | T | S | A | E | F | Q | K |
| m.1502 | 241 | A | L | R | R | A | Q | E | Q | K |
| m.1505 | 242 | A | K | S | R | A | Q | F | Q | K |
| m.1506 | 243 | A | K | T | K | A | E | F | Q | R |

TABLE 143
Meganucleases Optimized for ATGA Center Sequence (Second Subunit - Lox4)

| | | I-CreI Position | | | | | | | | |
|---|---|---|---|---|---|---|---|---|---|---|
| | SEQ | 19 | 48 | 50 | 59 | 72 | 73 | 74 | 80 | 139 |
| Nuclease | ID | 210 | 239 | 241 | 250 | 263 | 264 | 265 | 271 | 330 |
| x.109 | 8 | G | H | S | V | T | H | S | Q | K |
| m.1417 | 222 | G | K | S | V | R | I | S | E | K |
| m.1421 | 223 | G | R | I | A | H | I | A | Q | K |
| m.1432 | 224 | A | K | S | V | R | I | S | E | K |
| m.1436 | 225 | G | A | R | V | R | I | S | Q | R |
| m.1437 | 226 | S | K | R | V | R | I | A | Q | K |
| m.1441 | 227 | G | K | I | V | R | I | S | E | R |
| m.1450 | 228 | G | K | C | V | R | V | A | E | R |
| m.1451 | 229 | G | K | S | A | H | I | S | E | K |
| m.1453 | 230 | G | K | I | V | R | I | A | Q | R |
| m.1468 | 231 | A | K | A | V | R | V | S | Q | R |
| m.1469 | 232 | A | K | S | V | H | V | S | E | R |
| m.1477 | 233 | G | A | R | V | R | V | A | E | K |
| m.1478 | 234 | G | K | C | V | H | I | S | E | K |
| m.1485 | 235 | A | K | C | V | R | V | S | Q | K |
| m.1486 | 236 | G | K | S | V | R | I | S | E | R |
| m.1488 | 237 | G | K | Q | V | H | I | A | Q | R |
| m.1491 | 238 | G | K | C | V | R | I | T | Q | R |
| m.1500 | 239 | G | S | R | V | H | V | S | Q | K |
| m.1501 | 240 | G | K | S | V | R | V | A | E | K |
| m.1502 | 241 | G | K | S | V | R | V | A | E | K |
| m.1505 | 242 | G | H | C | V | R | V | S | Q | R |
| m.1506 | 243 | G | K | I | A | R | I | S | E | R |

TABLE 144
CHO iGFFP Assay ATGA Center Sequence Cleavage

| | SEQ ID | |
|---|---|---|
| Meganuclease | NO: | GFP % |
| LOX 3-4bs | — | 0.30 |
| CHO 23/24 | — | 10.15 |
| x.109 | 8 | 0.32 |
| m.1417 | 222 | 5.89 |
| m.1421 | 223 | 8.37 |
| m.1432 | 224 | 5.97 |
| m.1436 | 225 | 6.18 |
| m.1437 | 226 | 6.33 |
| m.1441 | 227 | 6.83 |
| m.1450 | 228 | 6.59 |
| m.1451 | 229 | 6.13 |
| m.1453 | 230 | 6.96 |
| m.1468 | 231 | 6.40 |
| m.1469 | 232 | 6.60 |
| m.1477 | 233 | 6.45 |
| m.1478 | 234 | 6.46 |
| m.1485 | 235 | 6.03 |
| m.1486 | 236 | 9.23 |
| m.1488 | 237 | 7.40 |
| m.1491 | 238 | 6.43 |
| m.1500 | 239 | 3.60 |
| m.1501 | 240 | 7.35 |
| m.1502 | 241 | 6.26 |
| m.1505 | 242 | 6.24 |
| m.1506 | 243 | 8.20 |

### Example 13

### Engineered Meganucleases Cleaving Recognition Sequences Containing an ATGG Four Base Pair Center Sequence

Novel engineered meganucleases derived from the LOX 3-4x.109 meganuclease were prepared by making amino acid substitutions at one or more positions in the rust subunit and one or more positions in the second subunit. In addition, an engineered meganuclease was generated that inserted an additional R residue following position 264, which corresponds to position 73 of wild-type I-CreI. These engineered meganucleases were then evaluated for cleavage of the LOX 3-4 recognition sequence modified to have an ATGG center sequence (SEQ ID NO: 244) in the CHO reporter assay according to Example 1. The substitutions in each subunit are provided in Tables 145 and 146, respectively. The results of the CHO reporter assay are provided in Table 147.

Following the modifications shown below, substantial improvements in cleavage of the recognition sequence having the ATGG four base pair center sequence were observed.

TABLE 145
Meganucleases Optimized for ATGG Center
Sequence (First Subunit - Lox3)

| | | I-CreI Position | | | | | | | | |
|---|---|---|---|---|---|---|---|---|---|---|
| | SEQ | 19 | 50 | 71 | 72 | 74 | 74 | 80 | 82 | 139 |
| Nuclease | ID | 19 | 50 | 71 | 72 | 73 | 74 | 80 | 82 | 139 |
| x.109 | 8 | A | Q | G | R | A | S | Q | K | K |
| m.1508 | 246 | G | R | G | P | A | S | E | E | R |
| m.1515 | 247 | A | R | S | G | C | C | Q | K | K |

TABLE 146
Meganucleases Optimized for ATGG Center Sequence (Second Subunit - Lox4)

| | | I-CreI Position | | | | | | | | |
|---|---|---|---|---|---|---|---|---|---|---|
| | | 19 | 48 | 50 | 71 | 72 | 73 | X | 77 | 80 |
| | | Nuclease | | | | | | | | |
| | SEQ | 210 | 239 | 241 | 262 | 263 | 264 | X | 268 | 271 |
| +1 AA* | ID | 210 | 239 | 241 | 262 | 263 | 264 | 265* | 269 | 272 |
| x.109 | 8 | G | H | S | S | T | H | | T | Q |
| m.1508 | 246 | A | K | R | D | G | R | R | N | Q |
| m.1515 | 247 | G | K | R | G | G | R | | N | R |

*Refers to engineered meganucleases having an insertion following a position which corresponds to position 73 of I-CreI.

TABLE 147
CHO iGFFP Assay ACAG Center Sequence Cleavage

| | SEQ ID | |
|---|---|---|
| Meganuclease | NO: | GFP % |
| LOX 3-4bs | — | 0.18 |
| CHO 23/24 | — | 12.43 |
| x.109 | 8 | 0.35 |
| m.1508 | 246 | 11.77 |
| m.1515 | 247 | 8.37 |

### Example 14

### Engineered Meganucleases Cleaving Recognition Sequences Containing an GCAA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have a GCAA center sequence (SEQ ID NO: 267) in the CHO reporter assay according to Example 1. The substitutions in each subunit are provided in Tables 148 and 149, respectively. The results of the CHO reporter assay are provided in Table 150.

Following the modifications shown below, substantial improvements in cleavage of the recognition sequence having the GCAA four base pair center sequence were observed.

TABLE 148
Meganucleases Optimized for GCAA Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| x.109 | 8 | A | K | Q | G | R | A | S | Q | K |
| m.1784 | 269 | A | K | R | G | P | T | S | E | K |
| m.1785 | 270 | A | K | R | N | S | T | S | Q | K |
| m.1787 | 271 | A | K | R | T | N | T | S | E | K |
| m.1789 | 272 | A | K | C | R | Q | V | S | E | K |
| m.1798 | 273 | G | K | R | S | G | V | C | Q | K |
| m.1805 | 274 | G | K | K | R | G | V | S | Q | K |
| m.1809 | 275 | A | K | R | G | A | T | S | E | K |
| m.1812 | 276 | S | K | R | R | N | V | S | E | K |
| m.1814 | 277 | A | K | T | R | G | V | A | Q | K |
| m.1820 | 278 | A | K | R | G | A | T | S | E | K |
| m.1827 | 279 | — | K | R | S | T | V | S | E | K |
| m.1836 | 280 | A | K | R | S | G | T | S | Q | R |
| m.1837 | 281 | A | K | R | S | G | T | S | Q | K |
| m.1838 | 282 | G | K | C | R | M | V | S | Q | K |
| m.1846 | 283 | G | K | L | R | M | V | S | Q | K |
| m.1853 | 284 | G | K | R | H | A | T | S | Q | K |
| m.1854 | 285 | A | H | R | T | R | V | S | E | K |
| m.1858 | 286 | G | K | R | R | V | T | S | E | K |
| m.1862 | 287 | A | K | R | S | S | T | S | E | K |
| m.1868 | 288 | A | K | R | G | P | T | S | Q | K |
| m.1870 | 289 | G | K | R | T | N | V | S | E | K |
| m.1873 | 290 | S | K | R | R | N | T | S | Q | K |
| m.1875 | 291 | G | K | R | R | G | V | S | Q | K |

TABLE 149
Meganucleases Optimized for GCAA Center Sequence (Second Subunit - Lox4)

| | | I-CreI Position | | | | | | | | | |
|---|---|---|---|---|---|---|---|---|---|---|---|
| | SEQ | 19 | 31 | 48 | 50 | 71 | 72 | 73 | 74 | 80 | 139 |
| Nuclease | ID | 210 | 222 | 239 | 241 | 262 | 263 | 264 | 265 | 271 | 330 |
| x.109 | 8 | G | Q | H | S | S | T | H | S | Q | K |
| m.1784 | 269 | G | Q | S | R | G | G | C | A | Q | R |
| m.1785 | 270 | G | Q | S | C | G | S | V | S | Q | R |
| m.1787 | 271 | G | P | A | R | G | A | C | A | E | K |
| m.1789 | 272 | G | Q | K | C | G | T | C | S | E | R |
| m.1798 | 273 | A | Q | S | R | G | E | V | S | E | R |
| m.1805 | 274 | A | Q | A | R | R | N | I | A | Q | R |
| m.1809 | 275 | G | Q | S | T | G | S | V | S | Q | R |
| m.1812 | 276 | G | Q | A | R | G | G | V | A | Q | R |
| m.1814 | 277 | G | Q | K | K | G | G | I | S | E | R |
| m.1820 | 278 | G | Q | S | R | G | G | V | A | Q | R |
| m.1827 | 279 | G | Q | K | E | G | K | I | T | Q | R |
| m.1836 | 280 | G | Q | S | R | G | H | I | S | E | K |
| m.1837 | 281 | G | Q | K | C | A | H | V | S | Q | R |
| m.1838 | 282 | A | Q | T | K | G | R | V | S | Q | K |
| m.1846 | 283 | A | Q | S | R | G | R | I | S | E | K |
| m.1853 | 284 | A | Q | S | R | G | C | C | S | E | K |
| m.1854 | 285 | G | Q | T | K | G | K | V | A | Q | K |
| m.1858 | 286 | A | Q | S | K | G | S | I | S | Q | K |
| m.1862 | 287 | G | Q | S | R | G | Y | V | A | Q | K |
| m.1868 | 288 | G | Q | K | C | G | R | I | A | E | R |
| m.1870 | 289 | A | Q | S | R | G | R | I | A | E | R |
| m.1873 | 290 | G | Q | T | R | G | K | I | A | E | K |
| m.1875 | 291 | A | Q | K | C | H | K | I | A | E | R |

TABLE 150
CHO iGFFP Assay GCAA Center Sequence Cleavage

| | SEQ ID | |
|---|---|---|
| Meganuclease | NO: | GFP % |
| LOX 3-4bs | — | 0.72 |
| CHO 23/24 | — | 13.78 |
| x.109 | 8 | 0.52 |
| m.1784 | 269 | 6.19 |
| m.1785 | 270 | 5.02 |
| m.1787 | 271 | 5.33 |
| m.1789 | 272 | 8.90 |
| m.1798 | 273 | 5.25 |
| m.1805 | 274 | 6.65 |
| m.1809 | 275 | 4.83 |
| m.1812 | 276 | 5.13 |
| m.1814 | 277 | 7.38 |
| m.1820 | 278 | 5.88 |
| m.1827 | 279 | 5.10 |
| m.1836 | 280 | 6.29 |
| m.1837 | 281 | 6.07 |
| m.1838 | 282 | 4.75 |
| m.1846 | 283 | 5.44 |
| m.1853 | 284 | 4.75 |
| m.1854 | 285 | 7.67 |
| m.1858 | 286 | 4.75 |
| m.1862 | 287 | 5.44 |
| m.1868 | 288 | 7.56 |
| m.1870 | 289 | 6.67 |
| m.1873 | 290 | 4.77 |
| m.1875 | 291 | 8.14 |

### Example 15

### Engineered Meganucleases Cleaving Recognition Sequences Containing an GCAT Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have a GCAT center sequence (SEQ ID NO: 292) in the CHO reporter assay according to Example 1. The substitutions in each subunit are provided in Tables 151 and 152, respectively. The results of the CHO reporter assay are provided in Table 153. Novel meganucleases which were modified to comprise the residues recited in the tables below continued to cleave the LOX 3-4 recognition sequence having a GCAT four base pair center sequence.

TABLE 151
Meganucleases Optimized for GCAT Center
Sequence (First Subunit - Lox3)

| | | I-CreI Position | | | | | | | | | |
|---|---|---|---|---|---|---|---|---|---|---|---|
| | SEQ | 19 | 48 | 50 | 71 | 72 | 73 | 74 | 80 | 139 | 143 |
| Nuclease | ID | 19 | 48 | 50 | 71 | 72 | 73 | 74 | 80 | 139 | 143 |
| x.109 | 8 | A | K | Q | G | R | A | S | Q | K | T |
| m.1600 | 294 | A | K | V | A | T | T | S | E | K | T |
| m.1601 | 295 | A | K | V | H | G | V | S | Q | K | T |
| m.1605 | 296 | A | K | R | H | S | T | S | Q | H | T |
| m.1606 | 297 | G | K | R | R | Q | T | S | Q | K | T |
| m.1623 | 298 | A | A | R | T | R | T | S | E | K | T |
| m.1660 | 299 | A | K | R | H | Q | T | S | Q | K | I |
| m.1661 | 300 | A | K | R | R | T | T | S | Q | K | T |
| m.1665 | 301 | A | H | R | R | R | V | S | Q | K | T |
| m.1667 | 302 | A | K | R | G | T | V | S | E | K | T |
| m.1669 | 303 | A | K | K | N | G | T | A | Q | K | T |
| m.1672 | 304 | A | H | R | S | R | T | S | Q | K | T |
| m.1674 | 305 | A | K | R | G | R | T | S | Q | K | T |
| m.1676 | 306 | A | K | S | R | N | C | S | Q | R | T |
| m.1677 | 307 | G | H | R | N | G | T | S | E | K | T |
| m.1679 | 308 | A | K | R | R | N | T | S | Q | K | T |
| m.1684 | 309 | G | R | R | T | G | V | S | Q | K | T |
| m.1685 | 310 | A | K | R | H | A | T | S | Q | K | T |
| m.1687 | 311 | G | K | R | R | G | V | S | E | K | T |
| m.1689 | 312 | A | K | R | A | A | T | S | Q | K | T |
| m.1691 | 313 | G | K | R | A | S | T | S | E | K | T |

TABLE 152
Meganucleases Optimized for GCAT Center Sequence (Second Subunit - Lox4)

| | | I-CreI Position | | | | | | | | | |
|---|---|---|---|---|---|---|---|---|---|---|---|
| | SEQ | 19 | 48 | 50 | 71 | 72 | 73 | 74 | 80 | 125 | 139 |
| Nuclease | ID | 210 | 239 | 241 | 262 | 263 | 264 | 265 | 271 | 316 | 330 |
| x.109 | 8 | G | H | S | S | T | H | S | Q | V | K |
| m.1600 | 294 | G | H | R | K | A | C | S | Q | V | R |
| m.1601 | 295 | G | A | R | R | G | G | C | E | V | K |
| m.1605 | 296 | G | K | S | A | N | C | C | Q | V | R |
| m.1606 | 297 | S | A | R | K | T | G | C | E | V | K |
| m.1623 | 298 | G | H | K | K | G | S | S | Q | V | K |
| m.1660 | 299 | G | A | R | K | T | C | A | E | A | K |
| m.1661 | 300 | G | A | R | G | S | G | S | E | V | R |
| m.1665 | 301 | G | T | R | K | G | A | S | E | V | K |
| m.1667 | 302 | G | T | Q | G | R | S | C | E | V | K |
| m.1669 | 303 | G | A | H | R | R | C | A | E | V | K |
| m.1672 | 304 | G | L | R | G | H | S | C | E | V | K |
| m.1674 | 305 | G | K | K | T | Q | C | C | E | V | K |
| m.1676 | 306 | G | H | K | R | R | S | C | E | V | K |
| m.1677 | 307 | A | L | K | R | A | C | C | E | V | K |
| m.1679 | 308 | G | I | K | G | N | S | C | E | V | K |
| m.1684 | 309 | S | A | K | K | A | C | C | E | V | K |
| m.1685 | 310 | G | K | V | R | K | C | S | Q | V | K |
| m.1687 | 311 | A | A | S | H | G | A | C | Q | V | H |
| m.1689 | 312 | G | A | H | G | T | S | A | Q | V | K |
| m.1691 | 313 | A | H | K | Y | R | C | C | E | V | K |

TABLE 153
CHO iGFFP Assay GCAT Center Sequence Cleavage

| | SEQ ID | |
|---|---|---|
| Meganuclease | NO: | GFP % |
| LOX 3-4bs | — | 0.26 |
| CHO 23/24 | — | 14.21 |
| x.109 | 8 | 9.25 |
| m.1600 | 294 | 9.68 |
| m.1601 | 295 | 10.90 |
| m.1605 | 296 | 8.70 |
| m.1606 | 297 | 8.33 |
| m.1623 | 298 | 8.18 |
| m.1660 | 299 | 8.70 |
| m.1661 | 300 | 9.13 |
| m.1665 | 301 | 9.08 |
| m.1667 | 302 | 8.16 |
| m.1669 | 303 | 9.90 |
| m.1672 | 304 | 8.32 |
| m.1674 | 305 | 8.32 |
| m.1676 | 306 | 8.29 |
| m.1677 | 307 | 8.80 |
| m.1679 | 308 | 10.78 |
| m.1684 | 309 | 8.01 |
| m.1685 | 310 | 10.10 |
| m.1687 | 311 | 10.88 |
| m.1689 | 312 | 11.18 |
| m.1691 | 313 | 9.24 |

### Example 16

### Engineered Meganucleases Cleaving Recognition Sequences Containing an GCGA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have a GCGA center sequence (SEQ ID NO: 314) in the CHO reporter assay according to Example 1. The substitutions in each subunit are provided in Tables 154 and 155, respectively. The results of the CHO reporter assay are provided in Table 156.

Following the modifications shown below, substantial improvements in cleavage of the recognition sequence having the GCGA four base pair center sequence were observed.

TABLE 154
Meganucleases Optimized for GCGA Center
Sequence (First Subunit - Lox3)

| | | I-CreI Position | | | | | | |
|---|---|---|---|---|---|---|---|---|
| | SEQ | 19 | 50 | 71 | 72 | 73 | 74 | 80 |
| Nuclease | ID | 19 | 50 | 71 | 72 | 73 | 74 | 80 |
| x.109 | 8 | A | Q | G | R | A | S | Q |
| m.1694 | 316 | G | K | R | N | V | S | E |
| m.1745 | 317 | A | R | S | G | T | S | Q |
| m.1752 | 318 | S | R | S | G | T | S | Q |
| m.1753 | 319 | G | R | R | A | V | S | E |
| m.1765 | 320 | S | K | A | G | I | A | E |
| m.1770 | 321 | G | R | R | G | V | S | E |
| m.1774 | 322 | G | R | G | G | V | S | Q |
| m.1780 | 323 | A | R | S | G | V | S | E |
| m.1781 | 324 | A | R | N | R | V | S | Q |
| m.1782 | 325 | G | R | R | Q | V | S | E |

TABLE 155
Meganucleases Optimized for GCGA Center Sequence (Second Subunit - Lox4)

| | | I-CreI Position | | | | | | | |
|---|---|---|---|---|---|---|---|---|---|
| | SEQ | 19 | 48 | 50 | 72 | 73 | 74 | 80 | 139 |
| Nuclease | ID | 210 | 239 | 241 | 263 | 264 | 265 | 271 | 330 |
| x.109 | 8 | G | H | S | T | H | S | Q | K |
| m.1694 | 316 | S | K | C | R | V | S | Q | R |
| m.1745 | 317 | G | T | R | R | I | S | Q | R |
| m.1752 | 318 | G | S | R | R | I | S | E | R |
| m.1753 | 319 | A | A | R | R | I | S | Q | R |
| m.1765 | 320 | G | S | R | R | I | S | Q | R |
| m.1770 | 321 | G | Q | R | R | I | S | Q | R |
| m.1774 | 322 | G | A | R | R | I | A | Q | R |
| m.1780 | 323 | G | Q | R | R | I | S | Q | R |
| m.1781 | 324 | G | S | R | R | I | S | E | R |
| m.1782 | 325 | A | A | R | R | I | S | Q | R |

TABLE 156
CHO iGFFP Assay GCGA Center Sequence Cleavage

| | SEQ ID | |
|---|---|---|
| Meganuclease | NO: | GFP % |
| LOX 3-4bs | — | 0.73 |
| CHO 23/24 | — | 14.45 |
| x.109 | 8 | 0.48 |
| m.1694 | 316 | 4.93 |
| m.1745 | 317 | 5.13 |
| m.1752 | 318 | 4.66 |
| m.1753 | 319 | 4.75 |
| m.1765 | 320 | 4.64 |
| m.1770 | 321 | 4.69 |
| m.1774 | 322 | 4.58 |
| m.1780 | 323 | 6.53 |
| m.1781 | 324 | 6.58 |
| m.1782 | 325 | 5.45 |

### Example 17

### Engineered Meganucleases Cleaving Recognition Sequences Containing an GTAA Four Base Pair Center Sequence

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTAA center sequence (SEQ ID NO: 358) in the CHO reporter assay according to Example 1. The substitutions in the first subunit are provided in Table 157. The results of the CHO reporter assay are provided in Table 158. Novel meganucleases which were modified to comprise the residues recited in the tables below were capable of cleaving the LOX 3-4 recognition sequence having a GTAA four base pair center sequence.

TABLE 157
Meganucleases Optimized for GTAA Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| M.1 | 360 | A | K | R | S | Q | V | T | Q | R |
| M.2 | 361 | A | A | R | T | N | V | S | Q | R |
| M.3 | 362 | S | K | A | A | S | V | S | Q | R |
| M.4 | 363 | S | R | R | R | S | V | S | E | R |
| M.5 | 364 | S | A | R | A | K | V | S | Q | R |
| M.6 | 365 | A | R | R | N | A | V | S | Q | R |
| M.7 | 366 | S | S | R | S | H | C | S | Q | R |
| M.8 | 367 | S | S | R | A | H | V | S | Q | R |
| M.9 | 368 | S | R | K | T | G | V | A | Q | R |
| M.10 | 369 | S | A | R | H | S | V | S | E | R |
| M.11 | 370 | S | N | R | K | R | V | S | Q | R |
| M.12 | 371 | S | R | R | R | A | V | S | E | K |
| M.13 | 372 | S | K | A | N | R | V | S | Q | R |
| M.14 | 373 | A | S | K | H | S | V | S | Q | R |
| M.15 | 374 | A | S | A | G | T | V | S | Q | K |
| M.16 | 375 | S | S | R | A | H | V | S | Q | R |
| M.17 | 376 | S | S | R | S | T | V | S | E | R |
| M.18 | 377 | S | S | R | S | D | V | S | Q | R |
| M.19 | 378 | S | A | R | S | G | V | A | Q | R |
| M.20 | 379 | S | A | R | S | S | V | S | Q | R |
| M.21 | 380 | A | K | C | R | T | V | A | Q | R |
| M.22 | 381 | S | N | R | G | R | V | S | Q | R |
| M.23 | 382 | S | A | R | R | N | V | A | Q | R |
| M.24 | 383 | S | T | R | G | T | V | S | Q | R |
| M.25 | 384 | A | S | R | R | S | V | A | E | R |
| M.26 | 385 | S | A | R | R | H | V | S | Q | R |
| M.27 | 386 | S | S | R | N | Y | I | S | Q | R |
| M.28 | 387 | A | R | R | A | T | C | S | E | K |
| M.29 | 388 | S | R | C | S | N | V | S | Q | R |
| M.30 | 389 | A | K | R | H | P | T | S | Q | R |

TABLE 158
CHO iGFFP Assay GTAA Center Sequence Cleavage

| | SEQ ID | |
|---|---|---|
| Meganuclease | NO: | GFP % |
| LOX 3-4bs | — | 0.47 |
| CHO 23/24 | — | 13.64 |
| M.1 | 360 | 5.94 |
| M.2 | 361 | 8.28 |
| M.3 | 362 | 9.40 |
| M.4 | 363 | 9.32 |
| M.5 | 364 | 9.37 |
| M.6 | 365 | 11.44 |
| M.7 | 366 | 9.99 |
| M.8 | 367 | 8.07 |
| M.9 | 368 | 6.66 |
| M.10 | 369 | 9.02 |
| M.11 | 370 | 7.34 |
| M.12 | 371 | 9.80 |
| M.13 | 372 | 7.81 |
| M.14 | 373 | 8.29 |
| M.15 | 374 | 11.52 |
| M.16 | 375 | 9.88 |
| M.17 | 376 | 8.22 |
| M.18 | 377 | 8.46 |
| M.19 | 378 | 7.59 |
| M.20 | 379 | 7.35 |
| M.21 | 380 | 7.28 |
| M.22 | 381 | 7.45 |
| M.23 | 382 | 13.19 |
| M.24 | 383 | 8.03 |
| M.25 | 384 | 8.02 |
| M.26 | 385 | 8.06 |
| M.27 | 386 | 6.42 |
| M.28 | 387 | 6.67 |
| M.29 | 388 | 7.89 |
| M.30 | 389 | 7.51 |

### Example 18

### Engineered Meganucleases Cleaving Recognition Sequences Containing an GTAG Four Base Pair Center Sequence

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTAG center sequence (SEQ ID NO: 390) in the CHO reporter assay according to Example 1. The substitutions in the first subunit are provided in Table 159. The results of the CHO reporter assay are provided in Table 160. Novel meganucleases which were modified to comprise the residues recited in the tables below were capable of cleaving the LOX 3-4 recognition sequence having a GTAG four base pair center sequence.

TABLE 159
Meganucleases Optimized for GTAG Center
Sequence (First Subunit - Lox3)

| | | I-CreI Position | | | | | | |
|---|---|---|---|---|---|---|---|---|
| | SEQ | 19 | 50 | 71 | 72 | 73 | 80 | 139 |
| Nuclease | ID | 19 | 50 | 71 | 72 | 73 | 80 | 139 |
| m.95 | 392 | A | R | S | N | R | Q | K |
| m.96 | 393 | S | R | D | G | R | Q | K |
| m.97 | 394 | A | R | S | N | R | Q | K |
| m.102 | 395 | A | R | S | G | R | Q | K |
| m.108 | 396 | A | R | S | G | R | Q | K |
| m.111 | 397 | A | R | S | N | R | Q | K |
| m.114 | 398 | S | C | S | N | R | Q | R |
| m.123 | 399 | S | R | D | G | R | Q | K |

TABLE 160
CHO iGFFP Assay GTAG Center Sequence Cleavage

| | SEQ ID | |
|---|---|---|
| Meganuclease | NO: | GFP % |
| LOX 3-4bs | — | 0.73 |
| CHO 23/24 | — | 15.76 |
| m.95 | 392 | 18.56 |
| m.96 | 393 | 15.46 |
| m.97 | 394 | 17.25 |
| m.102 | 395 | 13.85 |
| m.108 | 396 | 19.70 |
| m.111 | 397 | 16.98 |
| m.114 | 398 | 14.18 |
| m.123 | 399 | 13.82 |

### Example 19

### Engineered Meganucleases Cleaving Recognition Sequences Containing an GTAT Four Bas Pair Center Sequence

Novel engineered meganucleases derived from the LOX 3-4x.109 meganuclease were prepared by making amino acid substitutions at one or more positions in the rust subunit. These engineered meganucleases were then evaluated for cleavage of the LOX 3-4 recognition sequence modified to have a GTAT center sequence (SEQ ID NO: 400) in the CHO reporter assay according to Example 1. The substitutions in the first subunit are provided in Table 161. The results of the CHO reporter assay are provided in Table 162. Novel meganucleases which were modified to comprise the residues recited in the tables below were capable of cleaving the LOX 3-4 recognition sequence having a GTAT four base pair center sequence.

TABLE 161
Meganucleases Optimized for GTAT Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| m.124 | 402 | S | K | V | T | K | C | S | Q | R |
| m.125 | 403 | S | H | S | G | Y | C | S | E | R |
| m.126 | 404 | S | K | C | G | W | C | S | Q | R |
| m.127 | 405 | S | H | S | G | R | A | S | Q | R |
| m.128 | 406 | S | G | R | G | R | A | S | Q | K |
| m.129 | 407 | S | A | R | K | N | C | C | Q | R |
| m.130 | 408 | S | A | Q | G | R | A | S | Q | R |
| m.131 | 409 | A | T | R | G | R | A | S | E | K |
| m.132 | 410 | S | A | R | A | S | C | A | Q | K |
| m.133 | 411 | S | K | T | H | T | A | S | Q | R |
| m.134 | 412 | S | S | R | Y | G | S | S | Q | R |
| m.135 | 413 | S | M | R | G | R | A | S | Q | R |
| m.136 | 414 | A | S | G | G | R | A | S | Q | K |
| m.137 | 415 | S | A | K | R | S | A | S | E | K |
| m.138 | 416 | S | A | R | H | T | A | S | Q | K |
| m.139 | 417 | A | L | K | R | K | A | S | E | R |
| m.140 | 418 | S | A | R | G | R | A | S | Q | R |
| m.141 | 419 | S | K | R | T | H | C | S | Q | R |
| m.142 | 420 | A | H | R | G | R | A | S | Q | T |
| m.143 | 421 | S | K | K | R | A | A | S | Q | H |
| m.144 | 422 | S | A | R | L | G | C | A | Q | R |
| m.145 | 423 | S | K | K | S | G | S | S | E | R |
| m.146 | 424 | S | K | R | T | H | C | S | Q | R |
| m.147 | 425 | S | R | R | G | R | A | S | Q | K |
| m.148 | 426 | A | L | R | R | R | A | S | Q | R |
| m.149 | 427 | S | A | K | G | R | A | S | Q | R |
| m.150 | 428 | S | K | R | G | N | A | S | E | R |
| m.151 | 429 | S | T | R | G | R | A | S | Q | K |
| m.152 | 430 | S | A | R | N | R | A | A | Q | K |
| m.153 | 431 | S | L | S | K | R | C | S | Q | K |
| m.154 | 432 | S | L | R | N | R | T | S | Q | R |
| m.155 | 433 | S | K | L | G | R | A | S | Q | R |

TABLE 162
CHO iGFFP Assay GTAT Center Sequence Cleavage

| | SEQ ID | |
|---|---|---|
| Meganuclease | NO: | GFP % |
| LOX 3-4bs | — | 0.55 |
| CHO 23/24 | — | 16.59 |
| m.124 | 402 | 10.13 |
| m.125 | 403 | 12.37 |
| m.126 | 404 | 8.27 |
| m.127 | 405 | 11.88 |
| m.128 | 406 | 6.59 |
| m.129 | 407 | 9.93 |
| m.130 | 408 | 14.03 |
| m.131 | 409 | 10.81 |
| m.132 | 410 | 8.81 |
| m.133 | 411 | 10.15 |
| m.134 | 412 | 8.65 |
| m.135 | 413 | 4.58 |
| m.136 | 414 | 9.67 |
| m.137 | 415 | 10.69 |
| m.138 | 416 | 6.70 |
| m.139 | 417 | 11.52 |
| m.140 | 418 | 13.07 |
| m.141 | 419 | 8.70 |
| m.142 | 420 | 9.32 |
| m.143 | 421 | 6.73 |
| m.144 | 422 | 4.43 |
| m.145 | 423 | 11.43 |
| m.146 | 424 | 9.14 |
| m.147 | 425 | 9.21 |
| m.148 | 426 | 12.40 |
| m.149 | 427 | 12.94 |
| m.150 | 428 | 9.61 |
| m.151 | 429 | 9.43 |
| m.152 | 430 | 4.43 |
| m.153 | 431 | 9.14 |
| m.154 | 432 | 9.28 |
| m.155 | 433 | 8.29 |

### Example 20

### Engineered Meganucleases Cleaving Recognition Sequences Containing an GTGA Four Base Pair Center Sequence

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTGA center sequence (SEQ ID NO: 434) in the CHO reporter assay according to Example 1. The substitutions in the first subunit are provided in Table 163. The results of the CHO reporter assay are provided in Table 164. Novel meganucleases which were modified to comprise the residues recited in the tables below were capable of cleaving the LOX 3-4 recognition sequence having a GTGA four base pair center sequence.

TABLE 163
Meganucleases Optimized for GTGA Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| M.31 | 436 | S | A | R | R | T | V | S | E | K |
| M.32 | 437 | A | S | R | R | T | V | A | E | R |
| M.33 | 438 | A | G | R | G | S | V | S | Q | R |
| M.35 | 439 | A | R | R | R | G | V | S | Q | R |
| M.36 | 440 | A | K | R | R | H | V | T | Q | R |
| M.37 | 441 | S | S | R | G | T | V | S | E | R |
| M.38 | 442 | S | S | R | V | S | V | S | E | K |
| M.39 | 443 | S | A | R | S | K | V | S | Q | R |
| M.40 | 444 | S | G | R | S | G | V | S | Q | K |
| M.41 | 445 | S | S | R | A | K | V | A | Q | K |
| M.42 | 446 | S | S | R | R | S | V | A | Q | R |
| M.43 | 447 | A | S | R | T | R | V | S | E | K |
| M.44 | 448 | S | A | R | R | R | V | A | Q | K |
| M.46 | 449 | S | R | V | N | G | V | S | Q | R |
| M.47 | 450 | A | R | R | G | S | V | S | E | K |
| M.48 | 451 | S | S | R | R | R | V | S | Q | R |
| M.49 | 452 | S | S | R | R | H | V | S | Q | R |
| M.50 | 453 | A | S | R | R | K | V | G | E | R |
| M.51 | 454 | A | G | R | S | R | V | S | Q | K |
| M.52 | 455 | S | K | C | T | G | V | S | Q | R |
| M.53 | 456 | S | S | R | D | T | V | S | Q | R |
| M.54 | 457 | S | S | R | H | R | V | A | Q | K |
| M.56 | 458 | S | S | R | H | T | V | S | Q | R |
| M.57 | 459 | A | S | R | R | Y | V | S | Q | R |
| M.58 | 460 | S | H | R | R | R | V | A | E | R |
| M.59 | 461 | S | S | R | S | R | T | S | Q | K |
| M.61 | 462 | A | K | S | A | T | A | S | Q | R |

TABLE 164
CHO iGFFP Assay GTGA Center Sequence Cleavage

| | SEQ ID | |
|---|---|---|
| Meganuclease | NO: | GFP % |
| LOX 3-4bs | — | 0.88 |
| CHO 23/24 | — | 14.31 |
| M.31 | 436 | 8.62 |
| M.32 | 437 | 5.92 |
| M.33 | 438 | 5.17 |
| M.35 | 439 | 5.68 |
| M.36 | 440 | 2.75 |
| M.37 | 441 | 6.33 |
| M.38 | 442 | 6.03 |
| M.39 | 443 | 8.66 |
| M.40 | 444 | 4.47 |
| M.41 | 445 | 3.08 |
| M.42 | 446 | 4.21 |
| M.43 | 447 | 4.61 |
| M.44 | 448 | 3.56 |
| M.46 | 449 | 7.53 |
| M.47 | 450 | 10.09 |
| M.48 | 451 | 8.48 |
| M.49 | 452 | 6.21 |
| M.50 | 453 | 2.94 |
| M.51 | 454 | 4.59 |
| M.52 | 455 | 4.42 |
| M.53 | 456 | 5.55 |
| M.54 | 457 | 6.22 |
| M.56 | 458 | 6.66 |
| M.57 | 459 | 7.12 |
| M.58 | 460 | 4.04 |
| M.59 | 461 | 4.93 |
| M.61 | 462 | 7.06 |

### Example 21

### Engineered Meganucleases Cleaving Recognition Sequences Containing an GTGC Four Base Pair Center Sequenced

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTGC center sequence (SEQ ID NO: 463) in the CHO reporter assay according to Example 1. The substitutions in the first subunit are provided in Table 165. The results of the CHO reporter assay are provided in Table 166. Novel meganucleases which were modified to comprise the residues recited in the tables below were capable of cleaving the LOX 3-4 recognition sequence having a GTGC four base pair center sequence.

TABLE 165
Meganucleases Optimized for GTGC Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| m.156 | 465 | S | L | R | S | K | V | S | Q | K |
| m.157 | 466 | S | K | R | S | S | T | A | Q | K |
| m.158 | 467 | S | R | R | K | M | C | S | E | K |
| m.159 | 468 | S | L | R | G | P | C | S | E | K |
| m.160 | 469 | S | H | R | N | G | T | S | Q | T |
| m.161 | 470 | S | A | R | A | C | T | S | Q | K |
| m.162 | 471 | A | K | K | T | T | T | A | Q | K |
| m.163 | 472 | S | A | R | S | H | T | S | Q | K |
| m.164 | 473 | A | K | R | I | P | T | S | Q | K |
| m.165 | 474 | A | K | S | E | P | T | S | Q | K |
| m.166 | 475 | S | R | R | Y | T | T | S | Q | K |
| m.167 | 476 | S | H | R | R | S | A | S | E | K |
| m.168 | 477 | S | K | R | Q | S | N | S | Q | K |
| m.169 | 478 | S | K | R | Y | N | N | S | Q | K |
| m.170 | 479 | A | K | V | R | A | T | T | E | K |
| m.171 | 480 | S | R | K | S | S | T | S | Q | K |
| m.172 | 481 | S | K | R | S | M | V | S | Q | R |
| m.173 | 482 | A | L | K | T | A | T | S | Q | K |
| m.174 | 483 | S | N | R | R | S | T | A | Q | K |
| m.175 | 484 | A | K | R | Y | R | T | S | Q | H |
| m.176 | 485 | S | K | R | G | P | T | S | Q | V |
| m.177 | 486 | S | R | V | N | S | T | S | E | K |
| m.178 | 487 | A | L | R | S | Q | T | S | E | K |
| m.179 | 488 | A | K | R | F | R | T | S | E | K |
| m.180 | 489 | A | R | I | G | A | V | S | Q | K |
| m.181 | 490 | S | S | R | R | N | C | S | Q | T |
| m.182 | 491 | S | N | G | V | G | V | T | Q | H |
| m.183 | 492 | A | S | R | R | M | L | S | E | K |
| m.184 | 493 | S | H | R | S | R | T | S | Q | S |
| m.185 | 494 | S | K | K | T | D | T | A | Q | K |
| m.186 | 495 | S | K | S | S | K | T | S | Q | K |

TABLE 166
CHO iGFFP Assay GTGC Center Sequence Cleavage

| | SEQ ID | |
|---|---|---|
| Meganuclease | NO: | GFP % |
| LOX 3-4bs | — | 0.98 |
| CHO 23/24 | — | 18.47 |
| m.156 | 465 | 8.44 |
| m.157 | 466 | 7.01 |
| m.158 | 467 | 10.07 |
| m.159 | 468 | 8.26 |
| m.160 | 469 | 11.82 |
| m.161 | 470 | 10.49 |
| m.162 | 471 | 10.57 |
| m.163 | 472 | 10.92 |
| m.164 | 473 | 9.52 |
| m.165 | 474 | 11.73 |
| m.166 | 475 | 8.58 |
| m.167 | 476 | 8.49 |
| m.168 | 477 | 10.87 |
| m.169 | 478 | 11.88 |
| m.170 | 479 | 10.93 |
| m.171 | 480 | 10.69 |
| m.172 | 481 | 9.02 |
| m.173 | 482 | 9.31 |
| m.174 | 483 | 9.77 |
| m.175 | 484 | 9.74 |
| m.176 | 485 | 12.33 |
| m.177 | 486 | 11.16 |
| m.178 | 487 | 10.64 |
| m.179 | 488 | 10.71 |
| m.180 | 489 | 13.69 |
| m.181 | 490 | 13.83 |
| m.182 | 491 | 12.24 |
| m.183 | 492 | 12.32 |
| m.184 | 493 | 10.48 |
| m.185 | 494 | 9.23 |
| m.186 | 495 | 13.44 |

### Example 22

### Engineered Meganucleases Cleaving Recognition Sequences Containing an GTGG Four Base Pair Center Sequence

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTGG center sequence (SEQ ID NO: 496) in the CHO reporter assay according to Example 1. The substitutions in the first subunit are provided in Table 167. The results of the CHO reporter assay are provided in Table 168. Novel meganucleases which were modified to comprise the residues recited in the tables below were capable of cleaving the LOX 3-4 recognition sequence having a GTGG four base pair center sequence.

TABLE 167
Meganucleases Optimized for GTGG Center
Sequence (First Subunit - Lox3)

| | | I-CreI Position | | | | | | |
|---|---|---|---|---|---|---|---|---|
| | SEQ | 19 | 50 | 62 | 71 | 72 | 73 | 80 |
| Nuclease | ID | 19 | 50 | 62 | 71 | 72 | 73 | 80 |
| m.187 | 498 | A | R | I | S | G | R | Q |
| m.192 | 499 | G | Q | V | G | S | V | Q |
| m.201 | 500 | G | Q | I | G | S | V | E |
| m.203 | 501 | S | R | I | D | G | R | Q |

TABLE 168
CHO iGFFP Assay GTGG Center Sequence Cleavage

| | SEQ ID | |
|---|---|---|
| Meganuclease | NO: | GFP % |
| Lox 3-4 bs | — | 0.71 |
| CHO 23/24 | — | 9.33 |
| m.187 | 498 | 17.61 |
| m.192 | 499 | 11.88 |
| m.201 | 500 | 10.96 |
| m.203 | 501 | 12.14 |

### Example 23

### Engineered Meganucleases Cleaving Recognition Sequences Containing an GTGT Four Base Pair Center Sequence

Novel engineered meganucleases derived from the LOX 3-4x.109 meganuclease were prepared by making amino acid substitutions at one or more positions in the first subunit. These engineered meganucleases were then evaluated for cleavage of the LOX 3-4 recognition sequence modified to have a GTGT center sequence (SEQ ID NO: 502) in the CHO reporter assay according to Example 1. The substitutions in the first subunit are provided in Table 169. The results of the CHO reporter assay are provided in Table 170. Novel meganucleases which were modified to comprise the residues recited in the tables below were capable of cleaving the LOX 3-4 recognition sequence having a GTGT four base pair center sequence.

TABLE 169
Meganucleases Optimized for GTGT Center
Sequence (First Subunit - Lox3)

| | | I-CreI Position | | | | | | | | |
|---|---|---|---|---|---|---|---|---|---|---|
| | SEQ | 19 | 48 | 50 | 71 | 72 | 73 | 74 | 80 | 139 |
| Nuclease | ID | 19 | 48 | 50 | 71 | 72 | 73 | 74 | 80 | 139 |
| M.63 | 504 | S | K | V | G | R | A | S | Q | R |
| M.64 | 505 | S | K | R | G | P | S | S | Q | K |
| M.65 | 506 | S | S | R | G | R | A | S | Q | K |
| M.66 | 507 | S | L | R | G | R | A | S | Q | R |
| M.67 | 508 | S | L | R | R | A | C | S | Q | R |
| M.68 | 509 | S | K | R | R | Q | A | S | E | K |
| M.69 | 510 | S | K | Q | G | K | A | A | Q | K |
| M.70 | 511 | S | V | R | G | R | A | S | Q | K |
| M.71 | 512 | S | K | S | G | R | A | S | Q | R |
| M.73 | 513 | S | L | K | N | R | A | S | Q | R |
| M.74 | 514 | S | K | A | G | R | A | S | Q | K |
| M.75 | 515 | S | L | R | G | R | A | S | Q | K |
| M.77 | 516 | S | K | E | G | R | A | S | E | K |
| M.78 | 517 | S | K | K | H | R | A | S | Q | R |
| M.80 | 518 | S | K | R | A | T | A | S | Q | R |
| M.83 | 519 | S | K | R | T | G | T | S | Q | R |
| M.84 | 520 | A | L | R | N | K | A | S | E | K |
| M.85 | 521 | S | K | R | R | Q | A | S | E | K |
| M.86 | 522 | A | G | R | G | R | A | S | Q | K |
| M.87 | 523 | S | R | R | G | R | A | S | Q | K |
| M.88 | 524 | S | K | C | A | R | A | S | Q | R |
| M.89 | 525 | S | S | V | G | R | A | S | Q | K |
| M.90 | 526 | A | S | R | G | V | A | T | E | K |
| M.91 | 527 | A | K | Q | G | R | A | S | E | K |
| M.92 | 528 | S | N | R | G | R | A | S | Q | R |
| M.93 | 529 | S | G | R | G | R | A | S | Q | K |

TABLE 170
CHO iGFFP Assay GTGT Center Sequence Cleavage

| | SEQ ID | |
|---|---|---|
| Meganuclease | NO: | GFP % |
| LOX 3-4bs | — | 0.53 |
| CHO 23/24 | — | 16.40 |
| M.63 | 504 | 11.21 |
| M.64 | 505 | 10.02 |
| M.65 | 506 | 7.79 |
| M.66 | 507 | 6.08 |
| M.67 | 508 | 4.78 |
| M.68 | 509 | 10.46 |
| M.69 | 510 | 11.86 |
| M.70 | 511 | 7.47 |
| M.71 | 512 | 12.25 |
| M.73 | 513 | 6.62 |
| M.74 | 514 | 11.48 |
| M.75 | 515 | 7.17 |
| M.77 | 516 | 12.41 |
| M.78 | 517 | 7.75 |
| M.80 | 518 | 7.95 |
| M.83 | 519 | 8.17 |
| M.84 | 520 | 10.16 |
| M.85 | 521 | 11.61 |
| M.86 | 522 | 9.81 |
| M.87 | 523 | 14.04 |
| M.88 | 524 | 9.32 |
| M.89 | 525 | 9.51 |
| M.90 | 526 | 7.53 |
| M.91 | 527 | 10.94 |
| M.92 | 528 | 11.53 |
| M.93 | 529 | 10.69 |

### Example 24

### Engineered Meganucleases Cleaving Recognition Sequences Containing an TCAA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have a TCAA center sequence (SEQ ID NO: 331) in the CHO reporter assay according to Example 1. The substitutions in each subunit are provided in Tables 171 and 172, respectively. The results of the CHO reporter assay are provided in Table 173.

Following the modifications shown below, substantial improvements in cleavage of the recognition sequence having the TCAA four base pair center sequence were observed.

TABLE 171
Meganucleases Optimized for TCAA Center
Sequence (First Subunit - Lox3)

| | | I-CreI Position | | | | | | |
|---|---|---|---|---|---|---|---|---|
| | SEQ | 19 | 48 | 50 | 71 | 72 | 80 | 139 |
| Nuclease | ID | 19 | 48 | 50 | 71 | 72 | 80 | 139 |
| x.109 | 8 | A | K | Q | G | R | Q | K |
| m.2157 | 333 | S | S | R | G | S | E | K |
| m.2165 | 334 | S | S | R | R | S | E | K |
| m.2189 | 335 | A | K | R | G | S | E | K |
| m.2207 | 336 | A | K | T | G | P | Q | K |
| m.2225 | 337 | S | S | R | G | T | E | K |
| m.2229 | 338 | A | K | T | G | R | Q | R |
| m.2235 | 339 | A | K | C | T | G | Q | R |
| m.2238 | 340 | S | S | R | G | S | E | K |

TABLE 172
Meganucleases Optimized for TCAA Center Sequence (Second Subunit - Lox4)

| | | I-CreI Position | | | | | | | |
|---|---|---|---|---|---|---|---|---|---|
| | SEQ | 19 | 48 | 50 | 72 | 73 | 74 | 80 | 139 |
| Nuclease | ID | 210 | 239 | 241 | 263 | 264 | 265 | 271 | 330 |
| x.109 | 8 | G | H | S | T | H | S | Q | K |
| m.2157 | 333 | G | S | K | R | I | A | Q | R |
| m.2165 | 334 | G | S | R | Q | I | A | Q | R |
| m.2189 | 335 | G | K | C | R | I | A | E | R |
| m.2207 | 336 | G | S | K | R | I | A | E | R |
| m.2225 | 337 | G | K | E | R | I | A | Q | R |
| m.2229 | 338 | G | K | C | N | I | A | Q | R |
| m.2235 | 339 | G | S | K | Q | I | S | Q | R |
| m.2238 | 340 | S | S | K | S | I | A | Q | R |

TABLE 173
CHO iGFFP Assay TCAA Center Sequence Cleavage

| | SEQ ID | |
|---|---|---|
| Meganuclease | NO: | GFP % |
| LOX 3-4bs | — | 0.91 |
| CHO 23/24 | — | 27.71 |
| x.109 | 8 | 0.31 |
| m.2157 | 333 | 9.79 |
| m.2165 | 334 | 10.32 |
| m.2189 | 335 | 11.69 |
| m.2207 | 336 | 8.52 |
| m.2225 | 337 | 8.77 |
| m.2229 | 338 | 11.81 |
| m.2235 | 339 | 8.99 |
| m.2238 | 340 | 9.39 |

### Example 25

### Engineered Meganucleases Cleaving Recognition Sequences Containing an TTAA Four Base Pair Center Sequence

Novel engineered meganucleases derived from the LOX 3-4x.109 meganuclease were prepared by making amino acid substitutions at one or more positions in the first subunit and one or more positions in the second subunit. These engineered meganucleases were then evaluated for cleavage of the LOX 3-4 recognition sequence modified to have a TTAA center sequence (SEQ ID NO: 341) in the CHO reporter assay according to Example 1. The substitutions in each subunit are provided in Tables 174 and 175, respectively. The results of the CHO reporter assay are provided in Table 176.

Following the modifications shown below, substantial improvements in cleavage of the recognition sequence having the TTAA four base pair center sequence were observed.

TABLE 174
Meganucleases Optimized for TTAA Center
Sequence (First Subunit - Lox3)

| | | I-CreI Position | | | | | | | |
|---|---|---|---|---|---|---|---|---|---|
| | SEQ | 19 | 48 | 50 | 71 | 72 | 74 | 80 | 139 |
| Nuclease | ID | 19 | 48 | 50 | 71 | 72 | 74 | 80 | 139 |
| x.109 | 8 | A | K | Q | G | R | S | Q | K |
| m.2071 | 343 | G | N | R | G | T | S | Q | R |
| m.2077 | 344 | S | K | V | R | S | S | Q | R |
| m.2082 | 345 | S | S | R | N | N | A | E | R |
| m.2086 | 346 | G | K | K | G | S | S | E | R |
| m.2087 | 347 | A | K | K | N | R | A | Q | R |
| m.2102 | 348 | S | R | R | S | D | S | Q | R |
| m.2111 | 349 | G | K | R | G | T | S | Q | R |
| m.2116 | 350 | A | K | V | R | R | S | Q | R |
| m.2125 | 351 | G | K | R | A | Q | A | E | R |
| m.2132 | 352 | A | K | K | G | T | S | E | R |
| m.2138 | 353 | G | K | R | N | K | S | E | R |
| m.2141 | 354 | S | S | R | G | A | S | Q | K |
| m.2142 | 355 | S | S | R | N | N | A | E | R |
| m.2145 | 356 | G | N | R | G | T | S | Q | R |
| m.2151 | 357 | G | K | S | G | S | S | Q | R |

TABLE 175
Meganucleases Optimized for TTAA Center Sequence (Second Subunit - Lox4)

| | | I-CreI Position | | | | | | | | |
|---|---|---|---|---|---|---|---|---|---|---|
| | SEQ | 19 | 48 | 50 | 66 | 72 | 73 | 74 | 80 | 139 |
| Nuclease | ID | 210 | 239 | 241 | 257 | 263 | 264 | 265 | 271 | 330 |
| x.109 | 8 | G | H | S | Y | T | H | S | Q | K |
| m.2071 | 343 | A | K | C | Y | K | I | S | Q | R |
| m.2077 | 344 | G | S | K | Y | R | I | A | Q | R |
| m.2082 | 345 | G | S | R | Y | K | I | S | Q | R |
| m.2086 | 346 | A | S | K | Y | A | I | S | Q | R |
| m.2087 | 347 | G | A | K | Y | K | I | A | Q | R |
| m.2102 | 348 | G | S | R | H | S | I | A | Q | R |
| m.2111 | 349 | A | S | T | Y | R | V | A | Q | R |
| m.2116 | 350 | G | A | K | Y | R | I | S | Q | R |
| m.2125 | 351 | A | S | K | Y | R | I | S | Q | R |
| m.2132 | 352 | G | K | E | Y | Q | I | A | Q | R |
| m.2138 | 353 | S | S | K | Y | S | I | A | Q | R |
| m.2141 | 354 | G | A | R | Y | T | I | A | Q | R |
| m.2142 | 355 | G | S | R | Y | K | I | S | Q | R |
| m.2145 | 356 | A | K | C | Y | K | I | S | Q | R |
| m.2151 | 357 | A | T | K | Y | R | V | A | Q | R |

TABLE 176
CHO iGFFP Assay TTAA Center Sequence Cleavage

| | SEQ ID | |
|---|---|---|
| Meganuclease | NO: | GFP % |
| LOX 3-4bs | — | 3.03 |
| CHO 23/24 | — | 16.58 |
| x.109 | 8 | 4.60 |
| m.2071 | 343 | 11.22 |
| m.2077 | 344 | 11.75 |
| m.2082 | 345 | 11.62 |
| m.2086 | 346 | 11.95 |
| m.2087 | 347 | 11.65 |
| m.2102 | 348 | 12.42 |
| m.2111 | 349 | 12.01 |
| m.2116 | 350 | 12.36 |
| m.2125 | 351 | 12.70 |
| m.2132 | 352 | 11.81 |
| m.2138 | 353 | 12.60 |
| m.2141 | 354 | 11.71 |
| m.2142 | 355 | 13.94 |
| m.2145 | 356 | 12.89 |
| m.2151 | 357 | 13.04 |

### Example 26

### Engineered Meganucleases Cleaving Recognition Sequences Containing an TTGG Four Base Pair Center Sequence

Novel engineered meganucleases derived from the LOX 3-4x.109 meganuclease were prepared by making amino acid substitutions at one or more positions in the first subunit and one or more positions in the second subunit. The N-terminal subunit recognizes the reverse complement of the AG portion of the four base pair center sequence, which is CT, and the C-terminal subunit recognizes the GC portion of the two base pair center sequence. These engineered meganucleases were then evaluated for cleavage of the LOX 3-4 recognition sequence modified to have a TTGG center sequence (SEQ ID NO: 248) in the CHO reporter assay according to Example 1. The substitutions in each subunit are provided in Tables 177 and 178, respectively. The results of the CHO reporter assay are provided in Table 179.

Following the modifications shown below, substantial improvements in cleavage of the recognition sequence having the TTGG four base pair center sequence were observed.

TABLE 177
Meganucleases Optimized for TTGG Center
Sequence (First Subunit - Lox3)

| | | I-CreI Position | | | | | | | |
|---|---|---|---|---|---|---|---|---|---|
| | SEQ | 19 | 50 | 71 | 72 | 73 | 80 | 160 | 171 |
| Nuclease | ID | 19 | 50 | 71 | 72 | 73 | 80 | 160 | 171 |
| x.109 | 8 | A | Q | G | R | A | Q | G | A |
| m.1970 | 250 | G | R | S | G | R | Q | G | A |
| m.1973 | 251 | G | R | S | G | R | Q | G | A |
| m.1974 | 252 | G | R | S | G | R | Q | G | A |
| m.1975 | 253 | G | R | S | G | R | Q | G | A |
| m.1979 | 254 | G | R | S | G | R | Q | G | A |
| m.1980 | 255 | G | R | S | G | R | Q | G | A |
| m.1981 | 256 | G | R | S | G | R | Q | G | A |
| m.1982 | 257 | G | R | S | G | R | Q | G | A |
| m.1986 | 258 | G | R | S | G | R | Q | G | A |
| m.1997 | 259 | G | R | S | G | R | Q | E | A |
| m.2051 | 260 | G | R | S | G | R | Q | G | A |
| m.2052 | 261 | G | R | S | G | R | Q | G | A |
| m.2059 | 262 | G | R | S | G | R | Q | G | T |
| m.1995 | 263 | A | R | S | G | R | Q | G | A |
| m.2045 | 264 | A | R | S | G | R | Q | G | A |
| m.2050 | 265 | A | R | S | G | R | Q | G | A |
| m.2053 | 266 | A | R | S | G | R | Q | G | A |

TABLE 178
Meganucleases Optimized for TTGG Center Sequence (Second Subunit - Lox4)

| | | I-CreI Position | | | | | | | | | | |
|---|---|---|---|---|---|---|---|---|---|---|---|---|
| | SEQ | 19 | 48 | 50 | 66 | 71 | 72 | 73 | 74 | 80 | 85 | 139 |
| Nuclease | ID | 210 | 239 | 241 | 257 | 262 | 263 | 264 | 265 | 271 | 276 | 330 |
| x.109 | 8 | G | H | S | Y | S | T | H | S | Q | H | K |
| m.1970 | 250 | A | K | C | Y | G | Q | I | S | Q | H | R |
| m.1973 | 251 | A | K | C | H | K | K | I | S | Q | H | K |
| m.1974 | 252 | A | K | T | Y | G | T | I | A | Q | H | R |
| m.1975 | 253 | A | K | E | Y | G | R | I | S | Q | H | R |
| m.1979 | 254 | A | K | C | Y | G | H | I | A | Q | H | R |
| m.1980 | 255 | A | S | K | Y | G | A | I | A | Q | H | R |
| m.1981 | 256 | A | S | R | Y | G | S | V | S | Q | H | R |
| m.1982 | 257 | A | S | K | Y | G | T | I | S | Q | H | R |
| m.1986 | 258 | A | K | C | Y | G | R | I | S | Q | H | R |
| m.1997 | 259 | A | S | K | Y | G | K | I | S | Q | H | R |
| m.2051 | 260 | A | K | T | Y | G | A | I | S | Q | H | R |
| m.2052 | 261 | A | S | R | Y | G | R | I | S | Q | R | R |
| m.2059 | 262 | A | S | R | Y | G | A | V | A | Q | H | R |
| m.1995 | 263 | G | K | E | Y | G | K | I | A | Q | H | R |
| m.2045 | 264 | G | S | K | Y | G | K | I | S | Q | H | R |
| m.2050 | 265 | G | S | K | Y | G | S | V | S | Q | H | R |
| m.2053 | 266 | G | S | K | Y | G | T | I | S | Q | H | R |

TABLE 179
CHO iGFFP Assay TTGG Center Sequence Cutting

| | SEQ ID | |
|---|---|---|
| Meganuclease | NO: | GFP % |
| LOX 3-4bs | — | 0.4 |
| CHO 23/24 | — | 16.0 |
| x.109 | 8 | 0.4 |
| m.1970 | 250 | 13.5 |
| m.1973 | 251 | 14.3 |
| m.1974 | 252 | 11.6 |
| m.1975 | 253 | 11.8 |
| m.1979 | 254 | 12.4 |
| m.1980 | 255 | 12.5 |
| m.1981 | 256 | 13.5 |
| m.1982 | 257 | 11.0 |
| m.1986 | 258 | 11.5 |
| m.1997 | 259 | 12.3 |
| m.2051 | 260 | 13.5 |
| m.2052 | 261 | 13.1 |
| m.2059 | 262 | 12.2 |
| m.1995 | 263 | 11.2 |
| m.2045 | 264 | 14.2 |
| m.2050 | 265 | 12.2 |
| m.2053 | 266 | 12.3 |

### Example 27

### Engineered Meganucleases Cleaving Recognition Sequences Containing an GCAG Four Base Pair Center Sequence

Novel engineered meganucleases derived from the LOX 3-4x.109 meganuclease were prepared by making amino acid substitutions at one or more positions in the first subunit and one or more positions in the second subunit. The N-terminal subunit recognizes the reverse complement of the AG portion of the four base pair center sequence, which is CT, and the C-terminal subunit recognizes the GC portion of the three base pair center sequence. These engineered meganucleases were then evaluated for cleavage of the LOX 3-4 recognition sequence modified to have a GCAG center sequence (SEQ ID NO: 326) in the CHO reporter assay according to Example 1. The substitutions in each subunit are provided in Tables 180 and 181, respectively. The results of the CHO reporter assay are provided in Table 182.

Following the modifications shown below, substantial improvements in cleavage of the recognition sequence having the GCAG four base pair center sequence were observed.

TABLE 180
Meganucleases Optimized for GCAG Center Sequence
(CT recognizing first Subunit - Lox4)

| | | I-CreI Position | | | | | |
|---|---|---|---|---|---|---|---|
| | SEQ | 19 | 50 | 71 | 72 | 73 | 80 |
| Nuclease | ID | 19 | 50 | 71 | 72 | 73 | 80 |
| x.109 | 8 | A | Q | G | R | A | Q |
| m.494 | 328 | A | R | S | G | R | Q |
| m.509 | 329 | A | R | S | G | R | Q |
| m.524 | 330 | A | R | S | G | R | Q |

TABLE 181
Meganucleases Optimized for GCAG Center Sequence
(GC recognizing second subunit - Lox3)

| | | I-CreI Position | | | | | |
|---|---|---|---|---|---|---|---|
| | SEQ | 48 | 50 | 71 | 72 | 73 | 80 |
| Nuclease | ID | 239 | 241 | 262 | 263 | 264 | 271 |
| x.109 | 8 | H | Q | S | T | H | Q |
| m.494 | 328 | K | Q | G | S | V | Q |
| m.509 | 329 | H | R | G | S | V | Q |
| m.524 | 330 | H | R | G | R | T | Q |

TABLE 182
CHO iGFFP Assay GCAG Center Sequence Cutting

| | SEQ ID | |
|---|---|---|
| Meganuclease | NO: | GFP % |
| LOX 3-4bs | — | 0.35 |
| x.109 | 8 | 0.66 |
| CHO 23-24 | — | 15.17 |
| m.494 | 328 | 13.8 |
| m.509 | 329 | 13.4 |
| m.524 | 330 | 16.6 |

### Example 28

### Substitutions for the N-Terminal and C-Terminal Recognizing Portions of an I-CreI Derived Meganuclease

The substitution patterns observed in Examples 1-27 were compiled to determine a subset of amino acid substitutions that can be made to improve cutting of a four base pair center sequence by I-CreI derived meganucleases. Because each subunit of the meganuclease recognizes two of the four bases present in the center sequence, it was discovered that the substitutions made for a first subunit may be paired with the substitutions made in a second subunit. Amino acid residues, which may be substituted for the WT I-CreI residue at the corresponding positions of 48, 50, 71, 72, 73, 73B, and 74 are provided in Table 183 below.

Using this methodology, it is possible to derive amino acid residues, which enhance the cutting of a given center sequence, for each subunit of an I-CreI meganuclease. Preparing an I-CreI meganuclease having the indicated amino acids at the corresponding position will be expected to cut a given center sequence. For example, a meganuclease, which cleaves the center sequence ATAG, the residues corresponding to positions 48, 50, 71, 72, 73, 73B, and 74 of I-CreI provided in Table 183 for AT for the first subunit may be combined with residues corresponding to positions 48, 50, 71, 72, 73, 73B, and 74 of I-CreI provided in Table 183 for CT (the reverse complement of AG) for the second subunit. The exemplary predicted substitution of one or more residues in a first subunit and/or in a second subunit corresponding to positions 48, 50, 71, 72, 73, 73B, and 74 of I-CreI for the four base pair centers ATAG, ATAA, ATGA, ATGG, ACAA, ACAG, ACGA, ACGC, ACGG, TTGG, TCAA, GCAA, GCAT, GCGA, GCAG, GTAA, GTGA, GTGG, GTAG, GTAT, and GTGC that were all experimentally tested are provided in Tables 184-205 below. These simplified predicted positions correspond with the positions that were experimentally tested described herein. The exemplary predicted substitution of one or more residues in a first subunit and/or in a second subunit at positions corresponding to positions 48, 50, 71, 72, 73, 73B, and 74 of I-CreI for the four base pair centers CCAG, CCGA, CCGC, CTAA, CTGA are provided in Tables 206-210 below. These centers were not experimentally tested but would be expected to be cleaved by an engineered meganuclease described herein with the modifications shown in Tables 206-210.

TABLE 183
Pairwise Center Sequence Half-Site Amino Acid Residues
I-CreI Position

| Center Half | 48 | 50 | 71 | 72 | 73 | 73B | 74 |
|---|---|---|---|---|---|---|---|
| AC | K, N, Q, H, | K, R, C | G | R | A | — | S |
| | S, C, A, G | | | | | | |
| AT | K, N, Q, H, | K, R, C | G | R | A | — | S |
| | S, C, A, G | | | | | | |
| CC | K | R | D, S, G | G | R | R or no R | S |
| CT | K | C, R | S, G | N, G | R | N/A | S |
| GC | K | K, R | G, R, S, | G, N, S, | T, V | — | S |
| | | | T, N, H | A, R, T, | | | |
| | | | | Q, M, P, | | | |
| | | | | H | | | |
| GT | K | Q | G | S | V | — | S |
| TC | K, N, R, S, | S, R | R, G, S, | T, S, R, | I, V | — | A, S |
| | Q, H, G, A | | T | H | | | |
| TT | K | K, R | G | R, S, T | I, V | — | A, S, T |

TABLE 184
Center Sequence Half-Site Amino Acid Residues for ATAG
I-CreI Position

| Center Half | 48 | 50 | 71 | 72 | 73 | 73B | 74 |
|---|---|---|---|---|---|---|---|
| AT first | K, N, Q, H, | K, R, C | G | R | A | — | S |
| subunit | S, C, A, G | | | | | | |
| AG(CT) | K | C, R | S, G | N, G | R | — | S |
| second | | | | | | | |
| subunit | | | | | | | |

TABLE 185
Center Sequence Half-Site Amino Acid Residues for ATAA
I-CreI Position

| Center Half | 48 | 50 | 71 | 72 | 73 | 73B | 74 |
|---|---|---|---|---|---|---|---|
| AT first | K, N, Q, H, | K, R, | G | R | A | — | S |
| subunit | S, C, A, G | C | | | | | |
| AA(TT) | K | K, R | G | R, S, | I, V | — | A, S, |
| second | | | | T | | | T |
| subunit | | | | | | | |

TABLE 186
Center Sequence Half-Site Amino Acid Residues for ATGA
I-CreI Position

| Center Half | 48 | 50 | 71 | 72 | 73 | 73B | 74 |
|---|---|---|---|---|---|---|---|
| AT first | K, N, Q, H, | K, R, | G | R | A | — | S |
| subunit | S, C, A, G | C | | | | | |
| GA(TC) | K, N, R, S, | S, R | R, G, | T, S, | I, V | — | A, S |
| second | Q, H, G, A | | S, T | R, H | | | |
| subunit | | | | | | | |

TABLE 187
Center Sequence Half-Site Amino Acid Residues for ATGG
I-CreI Position

| Center Half | 48 | 50 | 71 | 72 | 73 | 73B | 74 |
|---|---|---|---|---|---|---|---|
| AT first | K, N, Q, H, | K, R, | G | R | A | — | S |
| subunit | S, C, A, G | C | | | | | |
| GG(CC) | K | R | D, S, | G | R | R or | S |
| second | | | G | | | no R | |
| subunit | | | | | | | |

TABLE 188
Center Sequence Half-Site Amino Acid Residues for ACAA
I-CreI Position

| Center Half | 48 | 50 | 71 | 72 | 73 | 73B | 74 |
|---|---|---|---|---|---|---|---|
| AC first | K, N, Q, H, | K, R, | G | R | A | — | S |
| subunit | S, C, A, G | C | | | | | |
| AA(TT) | K | K, R | G | R, S, | I, V | — | A, S, |
| second | | | | T | | | T |
| subunit | | | | | | | |

TABLE 189
Center Sequence Half-Site Amino Acid Residues for ACAG
I-CreI Position

| Center Half | 48 | 50 | 71 | 72 | 73 | 73B | 74 |
|---|---|---|---|---|---|---|---|
| AC first | K, N, Q, H, | K, R, | G | R | A | — | S |
| subunit | S, C, A, G | C | | | | | |
| AG(CT) | K | C | G >> S | G > R | R | — | S |
| second | | | | | | | |
| subunit | | | | | | | |

TABLE 190
Center Sequence Half-Site Amino Acid
Residues for ACGA
I-CreI Position

| Center | | | | | | | |
|---|---|---|---|---|---|---|---|
| Half | 48 | 50 | 71 | 72 | 73 | 73B | 74 |
| AC first | K, N, Q, | K, R, C | G | R | A | — | S |
| subunit | H, S, C, | | | | | | |
| | A, G | | | | | | |
| GA(TC) | K, N, R, | S, R | R, G, S, | T, S, R, | I, V | — | A, S |
| second | S, Q, H, | | T | H | | | |
| subunit | G, A | | | | | | |

TABLE 191
Center Sequence Half-Site Amino Acid Residues for ACGC
I-CreI Position

| Center Half | 48 | 50 | 71 | 72 | 73 | 73B | 74 |
|---|---|---|---|---|---|---|---|
| AC first | K, N, Q, H, | K, R, | G | R | A | — | S |
| subunit | S, C, A, G | C | | | | | |
| AG(CT) | K | K, R | G, R, | G, N, S, | T, V | — | S |
| second | | | S, T, | A, R, T, | | | |
| subunit | | | N, H | Q, M, P, | | | |
| | | | | H | | | |

TABLE 192
Center Sequence Half-Site Amino Acid Residues for ACGG
I-CreI Position

| Center Half | 48 | 50 | 71 | 72 | 73 | 73B | 74 |
|---|---|---|---|---|---|---|---|
| AC first | K, N, Q, H, | K, R, | G | R | A | — | S |
| subunit | S, C, A, G | C | | | | | |
| AG(CT) | K | R | D, S, | G | R | R or | S |
| second | | | G | | | no R | |
| subunit | | | | | | | |

TABLE 193
Center Sequence Half-Site Amino Acid Residues for TTAA
I-CreI Position

| Center Half | 48 | 50 | 71 | 72 | 73 | 73B | 74 |
|---|---|---|---|---|---|---|---|
| AC first | R, K, | K, R, | S, N, | S, N, | V | — | A, S |
| subunit | S, N | I, E, | G, D, | R, H | | | |
| | | V, G | R, H | | | | |
| AG(CT) | S, K, | K, R | G | R, S, | I, V | — | A, S, |
| second | A | | | T | | | T |
| subunit | | | | | | | |

TABLE 194
Center Sequence Half-Site Amino Acid Residues for TTGG
I-CreI Position

| Center Half | 48 | 50 | 71 | 72 | 73 | 73B | 74 |
|---|---|---|---|---|---|---|---|
| AC first | K | K, R | G | R, S, | I, V | — | A, S, |
| subunit | | | | T | | | T |
| AG(CT) | K | R | S, G | G | R | R or | S |
| second | | | | | | no R | |
| subunit | | | | | | | |

TABLE 195
Center Sequence Half-Site Amino Acid Residues for TCAA
I-CreI Position

| Center Half | 48 | 50 | 71 | 72 | 73 | 73B | 74 |
|---|---|---|---|---|---|---|---|
| TC first | K, N, R, | S, R | R, G, | T, S, | I, V | — | A, S |
| subunit | S, Q, H, | | S, T | R, H | | | |
| | G, A | | | | | | |
| TT(AA) | K | K, R | G | R, S, | I, V | — | A, S, |
| second | | | | T | | | T |
| subunit | | | | | | | |

TABLE 196
Center Sequence Half-Site Amino Acid Residues for GCAA
I-CreI Position

| Center Half | 48 | 50 | 71 | 72 | 73 | 73B | 74 |
|---|---|---|---|---|---|---|---|
| GC first | K | K, R | G, R, S, T, | G, N, S, | T, V | — | S |
| subunit | | | N, H | A, R, T, | | | |
| | | | | Q, M, | | | |
| | | | | P, H | | | |
| TT(AA) | K | K, R | G | R, S, T | I, V | — | A, S, T |
| second | | | | | | | |
| subunit | | | | | | | |

TABLE 197
Center Sequence Half-Site Amino Acid
Residues for GCAT I-CreI Position

| Center | | | | | | | |
|---|---|---|---|---|---|---|---|
| Half | 48 | 50 | 71 | 72 | 73 | 73B | 74 |
| GC first | K | K, R | G, R, S, | G, N, S, | T, V | — | S |
| subunit | | | T, N, H | A, R, T, | | | |
| | | | | Q, M, | | | |
| | | | | P, H | | | |
| AT(AT) | K, N, Q, | K, R | G | R | A | — | S |
| second | H, S, C, | | | | | | |
| subunit | A, G | | | | | | |

TABLE 198
Center Sequence Half-Site Amino Acid
Residues for GCGA I-CreI Position

| Center | | | | | | | |
|---|---|---|---|---|---|---|---|
| Half | 48 | 50 | 71 | 72 | 73 | 73B | 74 |
| GC first | K | K, R | G, R, S, | G, N, S, | T, V | — | S |
| subunit | | | T, N, H | A, R, T, | | | |
| | | | | Q, M, | | | |
| | | | | P, H | | | |
| GA(TC) | K, N, R, | S, R | R, G, S, T | T, S, R, | I, V | — | A, S |
| second | S, Q, H, | | | H | | | |
| subunit | G, A | | | | | | |

TABLE 199
Center Sequence Half-Site Amino Acid Residues for GCAG
I-CreI Position

| Center Half | 48 | 50 | 71 | 72 | 73 | 73B | 74 |
|---|---|---|---|---|---|---|---|
| GC | K | K, R | G, R, S, T, | G, N, S, | T, V | — | S |
| subunit | | | N, H | A, R, T, | | | |
| | | | | Q, M, | | | |
| | | | | P, H | | | |
| AG(CT) | K | C, R | S, G | N, G | R | — | S |
| second | | | | | | | |
| subunit | | | | | | | |

TABLE 200
Predicted Center Sequence Half-Site Amino Acid Residues for GTAA
I-CreI Position

| Center Half | 48 | 50 | 71 | 72 | 73 | 73B | 74 |
|---|---|---|---|---|---|---|---|
| AA (TT) | K | K, R | G | R, S, T | I, V | — | A, S, T |
| subunit | | | | | | | |

TABLE 201
Center Sequence Half-Site Amino Acid Residues for GTGA
I-CreI Position

| Center Half | 48 | 50 | 71 | 72 | 73 | 73B | 74 |
|---|---|---|---|---|---|---|---|
| TC subunit | K, N, R, | S, R | R, G, S, | T, S, R, | I, V | — | A, S |
| | S, Q, H, | | T | H | | | |
| | G, A | | | | | | |

TABLE 202
Center Sequence Half-Site Amino Acid Residues for GTGG
I-CreI Position

| Center Half | 48 | 50 | 71 | 72 | 73 | 73B | 74 |
|---|---|---|---|---|---|---|---|
| GG (CC) | K | R | D, S, G | G | R | R or | S |
| subunit | | | | | | no R | |

TABLE 203
Center Sequence Half-Site Amino Acid Residues for GTAG
I-CreI Position

| Center Half | 48 | 50 | 71 | 72 | 73 | 73B | 74 |
|---|---|---|---|---|---|---|---|
| AG (CT) | K | C, R | S, G | N, G | R | — | S |
| subunit | | | | | | | |

TABLE 204
Center Sequence Half-Site Amino Acid
Residues for GTAT I-CreI Position

| Center | | | | | | | |
|---|---|---|---|---|---|---|---|
| Half | 48 | 50 | 71 | 72 | 73 | 73B | 74 |
| AT (AT) | K, N, Q, | K, R | G | R | A | — | S |
| subunit | H, S, C, | | | | | | |
| | A, G | | | | | | |

TABLE 205
Center Sequence Half-Site Amino Acid Residues for GTGC
I-CreI Position

| Center Half | 48 | 50 | 71 | 72 | 73 | 73B | 74 |
|---|---|---|---|---|---|---|---|
| GC (GC) | K | K, R | G, R, S, | G, N, S, | T, V | — | S |
| subunit | | | T, N, H | A, R, T, | | | |
| | | | | Q, M, | | | |
| | | | | P, H | | | |

TABLE 206
Center Sequence Half-Site Amino Acid Substitutions for CCAG
I-CreI Position

| Center Half | 48 | 50 | 71 | 72 | 73 | 73B | 74 |
|---|---|---|---|---|---|---|---|
| CC | K | R | D, S, G | G | R | R or | S |
| | | | | | | no R | |
| AG(CT) | K | C, R | S, G | N, G | R | N/A | S |

TABLE 207
Center Sequence Half-Site Amino Acid Substitutions for CCGA
I-CreI Position

| Center Half | 48 | 50 | 71 | 72 | 73 | 73B | 74 |
|---|---|---|---|---|---|---|---|
| CC | K | R | D, S, G | G | R | R or | S |
| | | | | | | no R | |
| GA(TC) | K, N, R, | S, R | R, G, S, | T, S, R, | I, V | — | A, S |
| | S, Q, H, | | T | H | | | |
| | G, A | | | | | | |

TABLE 208
Center Sequence Half-Site Amino Acid Substitutions for CCGC
I-CreI Position

| Center Half | 48 | 50 | 71 | 72 | 73 | 73B | 74 |
|---|---|---|---|---|---|---|---|
| CC | K | R | D, S, G | G | R | R or | S |
| | | | | | | no R | |
| GC(GC) | K | K, R | G, R, S, | G, N, S, | T, V | — | S |
| | | | T, N, H | A, R, T, | | | |
| | | | | Q, M, | | | |
| | | | | P, H | | | |

TABLE 209
Center Sequence Half-Site Amino Acid Substitutions
for CTAA I-CreI Position

| Center | | | | | | | |
|---|---|---|---|---|---|---|---|
| Half | 48 | 50 | 71 | 72 | 73 | 73B | 74 |
| CT | K | C, R | S, G | N, G | R | — | S |
| AA(TT) | K | K, R | G | R, S, T | I, V | — | A, S, T |

TABLE 210
Center Sequence Half-Site Amino Acid Substitutions for CTGA
I-CreI Position

| Center Half | 48 | 50 | 71 | 72 | 73 | 73B | 74 |
|---|---|---|---|---|---|---|---|
| CT | K | C, R | S, G | N, G | R | — | S |
| GA(TC) | K, N, R, | S, R | R, G, S, | T, S, R, | I, V | — | A, S |
| | S, Q, H, | | T | H | | | |
| | G, A | | | | | | |

1-78. (canceled)

79. An engineered I-CreI derived meganuclease that binds and cleaves a 22 base pair recognition sequence comprising a center sequence selected from the group consisting of ACAA, ACAG, ACAT, ACGA, ACGC, ACGG, ACGT, ATAA, ATAG, ATAT, ATGA, ATGG, TTGG, GCAT, GCGA, or TCAA, wherein said engineered meganuclease comprises a first subunit and a second subunit, wherein said first subunit and said second subunit each comprise an amino acid sequence that comprises at least an 85% sequence identity to SEQ ID NO: 1, with the exception of an amino acid substitution at one or more positions corresponding to positions 48, 50, 71, 72, 73, and 74 of SEQ ID NO: 1;
(A) wherein, when said center sequence consists of ACAA, ACAG, ACAT, ACGC, ACGG, or ACGT, said first subunit comprises the following residues:
(a) an A, C, G, H, I, K, L, N, Q, or S residue at a position corresponding to position 48 of SEQ ID NO: 1;
(b) an A, C, K, R, S, T, V, or W residue at a position corresponding to position 50 of SEQ ID NO: 1;
(c) an A, G, P, or R residue at a position corresponding to position 71 of SEQ ID NO: 1;
(d) an H, K, P, Q, R, or T residue at a position corresponding to position 72 of SEQ ID NO: 1;
(e) an A, C, or G residue at a position corresponding to position 73 of SEQ ID NO: 1; and
(f) a S residue at a position corresponding to position 74 of SEQ ID NO: 1 and
said second subunit comprises the following residues:
(a) an A, C, G, H, K, L, N, Q, R, S, or T residue at a position corresponding to position 48 of SEQ ID NO: 1;
(b) an A, C, G, H, K, L, N, Q, R, S, or T residue at a position corresponding to position 50 of SEQ ID NO: 1;
(c) an A, D, E, G, H, K, N, P, R, S, or T residue at a position corresponding to position 71 of SEQ ID NO: 1;
(d) an A, G, H, K, M, N, P, P, Q, R, S, or T residue at a position corresponding to position 72 of SEQ ID NO: 1;
(e) an A, C, G, H, I, R, S, T, or V residue at a position corresponding to position 73 of SEQ ID NO: 1;
(f) optionally an R residue at a position directly following position corresponding to position 73 of SEQ ID NO: 1 (designated as position 73B); and
(g) an A, C, S, or T residue at a position corresponding to position 74 of SEQ ID NO: 1
(B) wherein, when said center sequence consists of ATAA, ATAG, ATAT, ATGA, ATGG, said first subunit comprises the following residues:
(a) an A, C, D, G, H, K, L, N, Q, S, or T residue at a position corresponding to position 48 of SEQ ID NO: 1;
(b) a C, D, E, G, I, K, N, R, S, T, or V residue at a position corresponding to position 50 of SEQ ID NO: 1;
(c) a G, H, I, K, N, R, or S residue at a position corresponding to position 71 of SEQ ID NO: 1;
(d) an A, G, H, K, L, N, P, Q, R, or T residue at a position corresponding to position 72 of SEQ ID NO: 1;
(e) an A, C, S, or T residue at a position corresponding to position 73 of SEQ ID NO: 1; and
(f) an A, C, or S residue at a position corresponding to position 74 of SEQ ID NO: 1; and
said second subunit comprises the following residues:
(a) an A, C, G, H, K, N, Q, R, S, or T residue at a position corresponding to position 48 of SEQ ID NO: 1;
(b) an A, C, E, I, K, N, Q, R, S, or T residue at a position corresponding to position 50 of SEQ ID NO: 1;
(c) an A, C, E, I, K, N, Q, R, S, or T residue at a position corresponding to position 71 of SEQ ID NO: 1;
(d) an A, G, H, K, N, Q, R, S, T, V, or Y residue at a position corresponding to position 72 of SEQ ID NO: 1;
(e) an A, C, G, H, I, R, S, or V residue at a position corresponding to position 73 of SEQ ID NO: 1;
(f) optionally an R residue at a position directly following position corresponding to position 73 of SEQ ID NO: 1 (designated as position 73B); and
(g) an A, C, S, or T residue at a position corresponding to position 74 of SEQ ID NO: 1;
(C) wherein, when said center sequence consists of GCAT or GCGA said first subunit comprises the following residues:
(a) an A, H, K, or R residue at a position corresponding to position 48 of SEQ ID NO: 1;
(b) a C, K, L, Q, R, S, T, or V residue at a position corresponding to position 50 of SEQ ID NO: 1;
(c) an A, G, H, N, R, S, or T residue at a position corresponding to position 71 of SEQ ID NO: 1;
(d) an A, G, H, M, N, P, Q, R, S, T, or V residue at a position corresponding to position 72 of SEQ ID NO: 1;
(e) an A, C, I, T, or V residue at a position corresponding to position 73 of SEQ ID NO: 1; and
(f) an A or S residue at a position corresponding to position 74 of SEQ ID NO: 1; and
said second subunit comprises the following residues:
(a) an A, C, G, H, I, L, N, Q, R, S, or T residue at a position corresponding to position 48 of SEQ ID NO: 1;
(b) a C, E, H, K, R, S, T, or V residue at a position corresponding to position 50 of SEQ ID NO: 1;
(c) an A, G, H, K, R, S, T, or Y residue at a position corresponding to position 71 of SEQ ID NO: 1;
(d) an A, C, E, G, H, K, N, Q, R, T, or Y residue at a position corresponding to position 72 of SEQ ID NO: 1;
(e) an A, C, G, H, I, R, or S, residue at a position corresponding to position 73 of SEQ ID NO: 1; and
(f) an A, S, or T residue at a position corresponding to position 74 of SEQ ID NO: 1;
(D) wherein, when said center sequence consists of TTGG said first subunit comprises the following residues:
(a) a K, N, R, or S residue at a position corresponding to position 48 of SEQ ID NO: 1;
(b) a C, E, K, R, S, T, or V residue at a position corresponding to position 50 of SEQ ID NO: 1;
(c) an A, G, K, N, R, or S residue at a position corresponding to position 71 of SEQ ID NO: 1;
(d) an A, D, H, K, N, Q, R, or T residue at a position corresponding to position 72 of SEQ ID NO: 1;
(e) an I residue at a position corresponding to position 73 of SEQ ID NO: 1; and
(f) an A, S or T residue at a position corresponding to position 74 of SEQ ID NO: 1; and
said second subunit comprises the following residues:
(a) an A, K, S, or T residue at a position corresponding to position 48 of SEQ ID NO: 1;
(b) a C, E, K, R, or T residue at a position corresponding to position 50 of SEQ ID NO: 1;
(c) an A, D, G, K, Q, R, S, or T residue at a position corresponding to position 71 of SEQ ID NO: 1;
(d) a G, I, R, S, T, or V residue at a position corresponding to position 72 of SEQ ID NO: 1;
(e) an I, R, or V residue at a position corresponding to position 73 of SEQ ID NO: 1; and
(f) an A, S, or T residue at a position corresponding to position 74 of SEQ ID NO: 1;
(E) wherein, when said center sequence consists of TCAA, said first subunit comprises the following residues:
(a) an A, G, H, K, N, Q, R, or S residue at a position corresponding to position 48 of SEQ ID NO: 1;
(b) a C, R, S, or T residue at a position corresponding to position 50 of SEQ ID NO: 1;
(c) a G, R, S, or T residue at a position corresponding to position 71 of SEQ ID NO: 1;
(d) a G, H, P, R, S, or T residue at a position corresponding to position 72 of SEQ ID NO: 1;
(e) an I or V residue at a position corresponding to position 73 of SEQ ID NO: 1; and
(f) an A or S residue at a position corresponding to position 74 of SEQ ID NO: 1; and
said second subunit comprises the following residues:
(a) a K or S residue at a position corresponding to position 48 of SEQ ID NO: 1;
(b) a C, K, R, or T residue at a position corresponding to position 50 of SEQ ID NO: 1;
(c) a G, R, or T residue at a position corresponding to position 71 of SEQ ID NO: 1;
(d) a G, P, R, or T residue at a position corresponding to position 72 of SEQ ID NO: 1;
(e) an I residue at a position corresponding to position 73 of SEQ ID NO: 1; and
(f) an A or T residue at a position corresponding to position 74 of SEQ ID NO: 1;
(F) wherein, when said center sequence consists of ACGA, said first subunit comprises the following residues:
(a) a K residue at a position corresponding to position 48 of SEQ ID NO: 1;
(b) a V, R, T, W, or A residue at a position corresponding to position 50 of SEQ ID NO: 1;
(c) a G or P residue at a position corresponding to position 71 of SEQ ID NO: 1;
(d) an R or P residue at a position corresponding to position 72 of SEQ ID NO: 1; and
(e) an A residue at a position corresponding to position 73 of SEQ ID NO: 1; and
said second subunit comprises the following residues:
(a) a K, H, T, A, G, or Q residue at a position corresponding to position 48 of SEQ ID NO: 1;
(b) an R, S, C, I, V, or G residue at a position corresponding to position 50 of SEQ ID NO: 1;
(c) a G residue at a position corresponding to position 71 of SEQ ID NO: 1;
(d) an R or H residue at a position corresponding to position 72 of SEQ ID NO: 1;
(e) an I or V residue at a position corresponding to position 73 of SEQ ID NO: 1; and
(f) an S or A residue at a position corresponding to position 74 of SEQ ID NO: 1

80. The engineered meganuclease of claim 79, wherein:
(a) said center sequence is ACAA and said first subunit comprises residues corresponding to residues 48, 50, 71, 72, and 73 of any one of SEQ ID NOs: 11-33,
(b) said center sequence is ACAG and said first subunit comprises residues corresponding to residues 50, 71, 72, and 73 of any one of SEQ ID NOs: 36-43,
(c) said center sequence is ACAT and said first subunit comprises residues corresponding to residues 48, 50, 71, 72, and 73 of any one of SEQ ID NOs: 46-55 and 57-67,
(d) said center sequence is ACGA and said first subunit comprises residues corresponding to residues 48, 50, 71, 72, and 73 of any one of SEQ ID NOs: 70-89,
(e) said center sequence is ACGC and said first subunit comprises residues corresponding to residues 48, 50, 71, 72, and 73 of any one of SEQ ID NOs: 93-118,
(f) said center sequence is ACGG and said first subunit comprises residues corresponding to residues 50, 72, and 73 of any one of SEQ ID NOs: 121-135,
(g) said center sequence is ACGT and said first subunit comprises residues corresponding to residues 48, 50, 71, 72, and 73 of any one of SEQ ID NOs: 138-156,
(h) said center sequence is ATAA and said first subunit comprises residues corresponding to residues 48, 50, 71, 72, 73, and 74 of any one of SEQ ID NOs: 159-175, 177-180 and 182-183,
(i) said center sequence is ATAG and said first subunit comprises residues corresponding to residues 48, 50, 71, 72, and 73 of any one of SEQ ID Nos: 186-199,
(j) said center sequence is ATAT and said first subunit comprises residues corresponding to residues 48, 50, 71, 72, and 73 of any one of SEQ ID Nos: 202-219,
(k) said center sequence is ATGA and said first subunit comprises residues corresponding to residues 48, 50, 72, and 734 of any one of SEQ ID Nos: 222-223, 225-238, and 241-243,
(l) said center sequence is ATGG and said first subunit comprises residues corresponding to residues 50, 71, 72, 73, and 74 of any one of SEQ ID NOs: 246-247,
(m) said center sequence is TTGG and said first subunit comprises residues corresponding to residues 50, 71, 72, and 73, of any one of SEQ ID NOs: 250-266,
(n) said center sequence is GCAT and said first subunit comprises residues corresponding to residues 48, 50, 71, 72, 73, and 74 of any one of SEQ ID NOs: 294-313,
(o) said center sequence is GCGA and said first subunit comprises residues corresponding to residues 50, 71, 72, 73, and 74 of any one of SEQ ID NOs: 316-325,
(p) said center sequence is TCAA and said first subunit comprises residues corresponding to residues 48, 50, 71, and 72 of any one of SEQ ID NOs: 333-340, or
wherein:
(a) said center sequence is ACAA and said second subunit comprises residues corresponding to residues 239, 241, 262, 263, 264, and 265 of any one of SEQ ID NOs: 11-33,
(b) said center sequence is ACAG and said second subunit comprises residues corresponding to residues 241, 262, 263, and 264 of any one of SEQ ID NOs: 36-43,
(c) said center sequence is ACAT and said second subunit comprises residues corresponding to residues 239, 241, 262, 263, 264, and 265 of any one of SEQ ID NOs: 46-67,
(d) said center sequence is ACGA and said second subunit comprises residues corresponding to residues 239, 241, 262, 263, 264, and 265 of any one of SEQ ID NOs: 70-89,
(e) said center sequence is ACGC and said second subunit comprises residues corresponding to residues 239, 241, 262, 263, 264, and 265 of any one of SEQ ID NOs: 92-118,
(f) said center sequence is ACGG and said second subunit comprises residues corresponding to residues 239, 241, 262, 263, and 264 of any one of SEQ ID NOs: 121-135,
(g) said center sequence is ACGT and said second subunit comprises residues corresponding to residues 239, 241, 262, 263, 264, and 265 of any one of SEQ ID NOs: 138-156,
(h) said center sequence is ATAA and said second subunit comprises residues corresponding to residues 239, 241, 262, 263, 264, and 265 of any one of SEQ ID NOs: 159-183,
(i) said center sequence is ATAG and said second subunit comprises residues corresponding to residues 241, 263, and 264 of any one of SEQ ID NOs: 186-199,
(j) said center sequence is ATAT and said second subunit comprises residues corresponding to residues 239, 241, 262, 263, 264, and 265 of any one of SEQ ID NOs: 202-219,
(k) said center sequence is ATGA and said second subunit comprises residues corresponding to residues 239, 241, 263, 264, and 265 of any one of SEQ ID NOs: 222-243,
(l) said center sequence is ATGG and said second subunit comprises residues corresponding to residues 239, 241, 262, 263, and 264 of any one of SEQ ID NOs: 246-247,
(m) said center sequence is TTGG and said second subunit comprises residues corresponding to residues 239, 241, 262, 263, 264, and 265 of any one of SEQ ID NOs: 250-266,
(n) said center sequence is GCAT and said second subunit comprises residues corresponding to residues 239, 241, 262, 263, 264, and 265 of any one of SEQ ID NOs: 294, 295, 297-299, 301, 303-304, and 306-309, and 311-313,
(o) said center sequence is GCGA and said second subunit comprises residues corresponding to residues 239, 241, 263, 264, and 265 of any one of SEQ ID NOs: 376-325,
(p) said center sequence is TCAA and said second subunit comprises residues corresponding to residues 239, 241, 263, 264, and 265 of any one of SEQ ID NOs: 333-339.

81. A polynucleotide comprising a nucleic acid sequence encoding said engineered meganuclease of claim 79.

82. A recombinant DNA construct comprising a polynucleotide comprising a nucleic acid sequence encoding said engineered meganuclease of claim 79.

83. A recombinant virus comprising a polynucleotide comprising a nucleic acid sequence encoding said engineered meganuclease of claim 79, wherein said wherein said recombinant virus is a recombinant adenovirus, a recombinant lentivirus, a recombinant retrovirus, or a recombinant AAV.

84. A method for producing a genetically-modified eukaryotic cell having a disrupted target sequence in a chromosome of said genetically-modified eukaryotic cell, said method comprising:
introducing into a eukaryotic cell said engineered meganuclease of claim 79; or a polynucleotide comprising a nucleic acid sequence encoding said engineered meganuclease of claim 79, wherein said engineered meganuclease is expressed in said eukaryotic cell;
wherein said engineered meganuclease produces a cleavage site in said chromosome at a recognition sequence, and wherein said target sequence is disrupted by non-homologous end-joining at said cleavage site.

85. A method for producing a genetically-modified eukaryotic cell comprising an exogenous sequence of interest inserted into a chromosome of said genetically-modified eukaryotic cell, said method comprising
(a) introducing into a eukaryotic cell one or more polynucleotides comprising a first nucleic acid sequence encoding said engineered meganuclease of claim 79, wherein said engineered meganuclease is expressed in said eukaryotic cell, and a second nucleic acid sequence comprising said sequence of interest; or
(b) introducing said engineered meganuclease of claim 79 into a eukaryotic cell and introducing a polynucleotide comprising a nucleic acid sequence comprising said sequence of interest into said eukaryotic cell;
wherein said engineered meganuclease produces a cleavage site in said chromosome at a recognition sequence;
and wherein said sequence of interest is inserted into said chromosome at said cleavage site.

86. An engineered meganuclease that binds and cleaves a 22 base pair recognition sequence comprising a center sequence consisting of GTAG, GTAT, GTGA, GTGC, GTGG, or GTGT, wherein said engineered meganuclease comprises a first subunit and a second subunit, wherein said first subunit comprises an amino acid sequence that comprises at least an 85% sequence identity to SEQ ID NO: 1, with the exception of an amino acid substitution at one or more positions corresponding to positions 48, 50, 71, 72, 73, and 74 of SEQ ID NO: 1,
(A) wherein, when said center sequence is GTAT or GTGT, said first subunit comprises the following residues:
(a) an A, C, G, H, K, L, M, N, Q, R, S, T, or V residue at a position corresponding to position 48 of SEQ ID NO: 1;
(b) an A, C, E, G, I, K, L, R, S, T, or V residue at a position corresponding to position 50 of SEQ ID NO: 1;
(c) an A, D, E, F, G, H, I, K, L, N, Q, R, S, T, V, or Y residue at a position corresponding to position 71 of SEQ ID NO: 1;
(d) an A, C, D, G, H, K, M, N, P, Q, R, T, V, W, or Y residue at a position corresponding to position 72 of SEQ ID NO: 1;
(e) an A, C, I, L, N, R, S, or T residue at a position corresponding to position 73 of SEQ ID NO: 1; and
(f) an A, C, G, S, or T residue at a position corresponding to position 74 of SEQ ID NO: 1;
(B) wherein, when said center sequence is GTGG or GTAG, said first subunit comprises the following residues:
(a) an A, C, G, H, K, L, M, N, Q, R, S, T, or V residue at a position corresponding to position 48 of SEQ ID NO: 1;
(b) an A, C, E, G, I, K, L, R, S, T, or V residue at a position corresponding to position 50 of SEQ ID NO: 1;
(c) an A, D, E, F, H, I, K, L, N, Q, R, S, T, V, or Y residue at a position corresponding to position 71 of SEQ ID NO: 1;
(d) an A, C, D, G, H, K, M, N, P, Q, R, T, V, W, or Y residue at a position corresponding to position 72 of SEQ ID NO: 1;
(e) an A, C, I, L, N, R, S, or T residue at a position corresponding to position 73 of SEQ ID NO: 1; and
(f) an A, C, G, S, or T residue at a position corresponding to position 74 of SEQ ID NO: 1;
(C) wherein, when said center sequence is GTGA, said first subunit comprises the following residues:
(a) an A, C, G, H, L, M, N, Q, R, S, T, or V residue at a position corresponding to position 48 of SEQ ID NO: 1;
(b) an A, C, E, G, I, K, L, R, S, T, or V residue at a position corresponding to position 50 of SEQ ID NO: 1;
(c) an A, D, E, F, G, H, I, K, L, N, Q, R, S, T, V, or Y residue at a position corresponding to position 71 of SEQ ID NO: 1;
(d) an A, C, D, G, H, K, M, N, P, Q, R, S, T, V, W, or Y residue at a position corresponding to position 72 of SEQ ID NO: 1;
(e) an A, C, I, L, N, R, S, T, or V residue at a position corresponding to position 73 of SEQ ID NO: 1.

87. The engineered meganuclease of claim 86, wherein:
(a) said center sequence is GTAG and said first subunit comprises residues corresponding to residues 50, 71, 72, and 73 of any one of SEQ ID NOs: 392-399,
(b) said center sequence is GTAT and said first subunit comprises residues corresponding to residues 48, 50, 71, 72, 73, and 74 of any one of SEQ ID NOs: 402-407, 409, 411-414, and 416-433,
(c) said center sequence is GTGA and said first subunit comprises residues corresponding to residues 48, 50, 71, 72, 73, and 74 of any one of SEQ ID NOs: 436-439, 441-454, 456-461,
(d) said center sequence is GTGG and said first subunit comprises residues corresponding to residues 50, 71, 72, and 73 of any one of SEQ ID NOs: 498 and 501, or
(e) said center sequence is GTGT and said first subunit comprises residues corresponding to residues 48, 50, 71, 72, 73, and 74 of any one of SEQ ID NOs: 504-509, 511-526, and 528-529.

88. A polynucleotide comprising a nucleic acid sequence encoding said engineered meganuclease of claim 86.

89. A recombinant DNA construct comprising a polynucleotide comprising a nucleic acid sequence encoding said engineered meganuclease of claim 86.

90. A recombinant virus comprising a polynucleotide comprising a nucleic acid sequence encoding said engineered meganuclease of claim 86, wherein said wherein said recombinant virus is a recombinant adenovirus, a recombinant lentivirus, a recombinant retrovirus, or a recombinant AAV.

91. A method for producing a genetically-modified eukaryotic cell having a disrupted target sequence in a chromosome of said genetically-modified eukaryotic cell, said method comprising:
introducing into a eukaryotic cell said engineered meganuclease of claim 86; or a polynucleotide comprising a nucleic acid sequence encoding said engineered meganuclease of claim 86, wherein said engineered meganuclease is expressed in said eukaryotic cell;
wherein said engineered meganuclease produces a cleavage site in said chromosome at a recognition sequence, and wherein said target sequence is disrupted by non-homologous end-joining at said cleavage site.

92. A method for producing a genetically-modified eukaryotic cell comprising an exogenous sequence of interest inserted into a chromosome of said genetically-modified eukaryotic cell, said method comprising
(a) introducing into a eukaryotic cell one or more polynucleotides comprising a first nucleic acid sequence encoding said engineered meganuclease of claim 86, wherein said engineered meganuclease is expressed in said eukaryotic cell, and a second nucleic acid sequence comprising said sequence of interest; or
(b) introducing said engineered meganuclease of claim 86 into a eukaryotic cell and introducing a polynucleotide comprising a nucleic acid sequence comprising said sequence of interest into said eukaryotic cell;
wherein said engineered meganuclease produces a cleavage site in said chromosome at a recognition sequence;
and wherein said sequence of interest is inserted into said chromosome at said cleavage site.
